(19) United States
(12) Patent Application Publication
(10) Pub. No.: US 2024/0182549 A1
(43) Pub. Date: Jun. 6, 2024

(54) PEPTIDES AND ANTIBODY FUSIONS THAT BIND TO SARS-COV-2 AND METHODS OF USING SAME

(71) Applicants: Sachdev SIDHU, Toronto (CA); Shane MIERSCH, Toronto (CA); Gang CHEN, Vaughan (CA); Jonathan LABRIOLA, Whitby (CA)

(72) Inventors: Sachdev SIDHU, Toronto (CA); Shane MIERSCH, Toronto (CA); Gang CHEN, Vaughan (CA); Jonathan LABRIOLA, Whitby (CA)

(21) Appl. No.: 18/284,756

(22) PCT Filed: Mar. 31, 2022
(86) PCT No.: PCT/IB2022/053050

§ 371 Date: Sep. 28, 2023

Related U.S. Application Data
(60) Provisional application No. 63/264,598, filed on Nov. 26, 2021.
(60) Provisional application No. 63/168,916, filed on Mar. 31, 2021.

(51) Int. Cl. C07K 16/10; C07K 14/47

(52) U.S. Cl. CPC C07K 16/1003; C07K 14/4703; C07K 2317/35; C07K 2317/55; C07K 2319/30

(57) ABSTRACT

Described herein are peptides and peptide-antibody fusions that bind to the SARS-CoV-2 S protein and the receptor binding domain (RED), and methods for using the compositions containing these antibodies in the treatment of COVID-19, Specifically, we developed novel CoV-2 S protein binding peptides making use of a naïve phage-displayed 16mer peptide library—wherein the CoV-2 S protein binding peptides bind epitopes in both the RBD and S protein outside of the RBD. In addition, we show that fusion of these peptides to the N-terminus of nAbs improves their apparent affinity, potency and breadth of neutralization, without compromising their developability profile, Thus, these peptides represent a novel class of SARS-CoV-2 S protein binders, which can be formatted in various ways to improve functional properties of nAbs to be used in the treatment of many different diseases.

### CROSS-REFERENCE TO RELATED APPLICATIONS

This application is a U.S. national phase entry of PCT International Application Serial No. PCT/IB2022/053050, filed on Mar. 31, 2022, which claims priority to U.S. Provisional Application Ser. No. 63/168,916, filed on Mar. 31, 2021, and U.S. Provisional Application Ser. No. 63/264,598, filed on Nov. 26, 2021, all of which are incorporated herein by reference in their entirety.

### TECHNICAL FIELD

The present invention relates to peptides and peptide-antibody fusions that bind to the SARS-CoV-2 spike (S protein) and the receptor binding domain (RBD), and methods for using the compositions containing these antibodies in the treatment of COVID-19.

### I. BACKGROUND

COVID-19, the disease caused by the SARS-CoV-2 beta-coronavirus has caused a global pandemic, infecting more than 487 million people worldwide and causing the deaths of over 6 million people and the hospitalization of scores more as of the time of this application. SARS-CoV-2 is also mutating, creating new variants of the original virus that are resistant against treatments and vaccines. Despite the granting of Emergency Use Authorization by the U.S. Food and Drug Administration to three antibody treatments (casirivimab/imdevimab, bamlanivimab/etesevimab, sotrovimab) in addition to convalescent plasma therapy, as well as the emergence of a few more promising antivirals (molnupiravir and paxlovid) than the approved remdesivir there is still no pharmaceutical standard of care treatment for the infection creating a massive unmet medical need for safe, specific and effective anti-viral therapeutics against the original virus and variants of the virus.

SARS-CoV-2 emerged in the city of Wuhan, Hubei province of China sometime in late 2019 and rapidly spread throughout the world, becoming pandemic in March of 2020. Sequence analysis revealed it is most closely related to two coronaviruses from bat (bat-SL-CoVZC45 and bat-SL-CoVZXC21) as opposed to the recent human coronaviruses SARS-CoV and MERS-CoV (Lu, 2020, PMID: 32007145), suggesting it is a zoonotic coronavirus strain.

As with other coronaviruses, SARS-CoV-2 uses its surface spike glycoprotein (S protein) to interact with and enter host cells. The SARS-CoV-2 S protein gene has the lowest sequence identity compared to related coronaviruses. However, like other SARS-CoV strains, it makes use of angiotensin converting enzyme 2 (ACE2) found on the surface of alveolar, endothelial, and smooth muscle cells in the lower respiratory tract to enter the host cell. (Hoffman, Cell, 2020, PMID: 32142651). The S protein is a hetero-trimeric class I viral fusion glycoprotein (Bosch, 2003, PMID: 12885899), which contains a large ectodomain, a single-pass transmembrane segment, and a small intracapsid tail. The ectodomain is subdivided into N-terminal (S1) and C-terminal (S2) regions. In the absence of host cell target, the S protein exists in a metastable prefusion conformation, where it adopts a stubby, clover-like structure. The S1 domain is responsible for ACE2 recognition through its “receptor binding domain” (RBD). Binding of the RBD to ACE2 induces proteolytic cleavage at the S1/S2 interface. The S2 domain then undergoes large conformational changes, forming an extended “postfusion” structure, which is believe to promote fusion of the viral capsid to the host cell membrane (Walls, 2017, PMID: 32155444), allowing the virus access to the intracellular space. One significant change in the SARS-CoV-2 S protein compared to SARS-CoV is an insertion of a furin cleavage site at the S1/S2 interface, which allows for more efficient proteolytic processing facilitating more efficient infection of the host cell, (Coutard, 2020, PMID:32057769). Along with the RBD, S1 also contains an N-terminal domain (NTD).

Since the zoonotic spillover of the original Wuhan strain of SARS-CoV-2, a variant of the virus (D614G) has become globally dominant (Korber, Cell, 2020, PMID: 32697968). Additional variants have emerged with mutations in the S protein in various regions around the world including a B.1.1.7 variant from UK (https://virological.org/t/preliminary-genomic-characterisation-of-an-emergent-sars-cov-2-lineage-in-the-uk-defined-by-a-novel-set-of-spike-mutations/563), the B.1.351 variant from South Africa (Tegally, Nat Med, 2021, PMID: 33531709) the P.1 variant from Manaus, Brazil (https://virological.org/t/genomic-characterisation-of-an-emergent-sars-cov-2-lineage-in-manaus-preliminary-findings/586), a MB61 variant from Italy (Fiorentini, Lancet, Inf Dis, 2020) and others (Arora:2021jy). These variants have been noted to confer resistance to neutralization by therapeutic antibodies, convalescent plasma and vaccine sera (Wang, Nature, 2021, PMID: 33684923). Resistance can arise from mutations in the S protein and RBD that impair binding of neutralizing antibodies and strategies for overcoming resistance are required. One strategy that has been used to overcome mutational resistance in the context of other viruses is the development of multi-specific, broadly neutralizing antibodies (Khan, J Virol, 2018, PMID: 29976677, Steinhardt, Nat Comm, 2018, PMID: 29491415).

Development of therapeutics and vaccines to treat COVID-19 is essential toward restoring normal economic and social function. Indeed, several therapies have already been developed in many jurisdictions around the world, including recent approval of novel mRNA vaccines, (Jackson, 2020, PMID: 32663912) monoclonal antibody (mAb) therapeutics, and small molecular antivirals (remdesivir, molnupiravir, and paxlovid). While these developments have curbed the pandemic significantly, the recurring nature of coronaviruses necessitates interventions applicable to novel coronavirus variants and strains, which have already emerged and spread.

### II. BRIEF DESCRIPTION OF THE FIGURES

FIG. 1—Evaluation of the structural constraints for devising N-terminal peptide fusions to SARS-CoV-2 S protein binding antibodies.

FIG. 2—Peptides isolated from selections against SARS-CoV-2 S protein and receptor binding domain. Clones obtained from biopanning a 16mer phage-displayed peptide library against SARS-CoV-2 S protein ectodomain and RBD as indicated above the column headings. Column headings for each subgroup indicates the antigen against which selection was run, type of library from which the peptides originated as well as the sequence motif identified following alignment. Each peptide was assigned a unique identifying number (far left in each column) to be used throughout the application. Residues that constitute identified motifs in aligned sequences are highlighted in black and shades of grey. Peptides that were chosen for fusion and additional analysis are indicated by an asterisk (*).

FIG. 3—Peptide phage binning against structurally characterized SARS-CoV-2-S protein-binding IgGs. Peptides p16, p62 and p96 bind the NTD while peptides p102 and p103 bind the RBD. (A) Phage-displayed SARS-CoV-2 S protein-binding peptides were titrated and incubated in RBD-Fc (top) or S protein ectodomain (bottom)-coated wells of a 384-well ELISA plate. Bound phage was visualized by incubation with HRP-conjugated anti-M13 antibody followed by colorimetric development and absorbance measurements at 450 nm. (B) Structure of SARS-CoV-2 S protein indicating the epitopes of Abs used (C). (C) Epitope grouping—S protein-coated wells of a 384-well ELISA plate were pre-incubated with SARS-CoV-2 S protein-binding antibodies indicated on the left axis. Phage displayed S protein-binding peptide indicated on top axis was subsequently added. Bound phage was detected as in (A), and normalized against signal from a well incubated with non-binding IgG isotype (4275).

FIG. 4—Characterization of epitopes for phage-displayed S protein-binding peptides.—(A) Phage ELISAs for peptide-phage binding to immobilized S protein ectodomain (black bars) or RBD (white bars). Binding was quantified by optical density at 450 nm (y-axis) for 0.5 nM phage displaying the indicated peptide (x-axis). (B) Phage ELISAs for peptide-phage (x-axis) binding to immobilized S protein ECD in the presence of saturating concentrations of antibodies that bind to the NTD (5-24, black; 4-8, grey) or RBD (15033-7, light grey; RGN10933, white). The binding signal (y-axis) was normalized to binding in the absence of antibody.

FIG. 5—Characterization of parent IgG and peptide-IgG fusions. (A) Illustrations depicting the architecture of CoV-2 binding peptide-fused IgG's. (B) SDS-PAGE of purified parental and peptide-fused IgG's. (C) Size exclusion chromatography profiles for S protein-binding IgG and peptide-IgG fusions. (D) ELISA measuring non-specific interaction of S protein-binding parental IgG and peptide-IgG fusions to common biomolecules, typically an indicator of developability for Ab therapeutic leads. (E) The binding of parental IgG and peptide-IgG fusions to SARS-CoV-2 RBD, S protein ectodomain and S1 domain antigens as measured by ELISA.

FIG. 6—Expression yields, binding kinetics, and affinities of peptide-IgG fusions to SARS-CoV-2 S protein. Peptide-IgG fusion IDs are provided indicating fusion to either the light (L) or heavy (H) chains with superscripts denoting the peptide (pX) where the number corresponds to sequences in FIG. 2 and shown in column 3. For each, the antigen indicates the protein against which they were selected. Yields were determined by extrapolating the yield obtained from a 40 mL culture after Protein A purification. Binding kinetics were obtained by biolayer interferometry of serially diluted parental IgG or peptide-IgG fusion to immobilized S protein and globally fit with a 1:1 binding model. Note,^{a} ‘L’ and ‘H’ indicates fusion to light and heavy chain of IgGs, respectively. Superscript indicates peptide number indicated in FIG. 2; GS=(G4S)4 linker alone.^{b} All peptides were fused to light or heavy chain of antibody 15033 (TRAC ID) through a (G4S)4 linker.

FIG. 7—Size exclusion chromatography (SEC) parameters of peptide-IgGs. Protein samples (50 μg) were injected onto a TSKgel BioAssist G3SWxl column (Tosoh) preequilibrated in a PBS mobile phase and protein retention was monitored by absorbance at 215 nm during a 1.5 CV isocratic elution in PBS. Key parameters of the major peak from the resultant SEC profile for heavy (H) or light (L) chain fusions of the peptide indicated in superscript to IgG 15033 are reported relative to a Herceptin control or to IgG 15033 with (G4S4)₄ linker alone.

FIG. 8—S protein-binding peptide fusion enhances IgG potency in SARS-CoV-2 pseudovirus assay. Serial dilutions of IgG with S protein-binding peptides (indicated in superscript) fused to either the light (L) or heavy (H) chain of anti-SARS-CoV-2 IgG 15033 were evaluated in a pseudoviral infection assay for comparison to parental IgG 15033. Neutralization profiles were generated by plotting the antibody concentrations versus the luminescent signal obtained after exposure to S protein-pseudotyped virus-like particles pre-incubated with IgG or peptide-IgG fusion. Peptide IDs are as per FIG. 2 and IC₅₀ values were extracted from fitting the neutralization curves by non-linear regression.

FIG. 9—Functional characterization of peptide-IgG fusions compared to parental antibody. Serial dilutions of IgG with S protein-binding peptides fused to either the light (dashed lines) or heavy chain (solid lines) of IgG 15033 were evaluated in a focus reduction neutralization test with authentic SARS-CoV-2 virus (Washington strain (2019 n-CoV/USA_WA1/2020)) for comparison to parental IgG. Foci were measured on an ImmunoSpot analyzer 24 hrs following exposure of ACE2-expressing VeroE6 cells to antibody-virus mixtures and counts normalized to an untreated control. Solid grey curve and solid black curve show neutralization curves for 15033 IgG and tetravalent Fab-IgG, respectively.

FIG. 10—Characteristics of peptide-fused tetravalent diabody formats. (A) N-terminal peptide fusions of various tetravalent 15033-7 diabody-Fc-Fab and Fab-diabody-Fc molecules electrophoretically separated by SDS-PAGE then visualized by staining with Coomassie blue. Under non-reducing conditions, a band with the expected intact molecular weight of ˜210 kDa was observable. Under reducing conditions both the ˜80 kDa heavy chain (˜85 kDa for peptide fused heavy chain) and the ˜25 kDa light chain (˜30 kDa for peptide fused light chain) bands were observable. (B) An ELISA conducted against a panel of non-specific biomolecules to which binding is known to be predictive of poor PK was conducted using 100 nM of the indicated peptide-fused, diabody-based tetravalent antibody and revealed a general absence of binding, similar to the clinical benchmark—herceptin. Binding signals from an immobilized anti-human Fc antibody confirms positive binding indicative of an intact Fc and a negative control antibody (6066) known to be ‘sticky’ was included for reference. (C) Analytical size exclusion chromatograms of a panel of N-terminal peptide fusions of diabody-Fc-Fab and Fab-diabody-Fc antibodies previously isolated by 2-step purification on a Protein A sepharose then a Superdex 200 gel filtration column.

FIG. 11—Schematic of peptide-IgG fusions. Peptides selected from peptide-phage libraries are fused to the N-terminus of the heavy (H) and/or light (L) chain of human IgG via molecular cloning of genes encoding the peptides in to constructs for expression of the antibody. Antibody CDRs encode specificity for the SARS-CoV-2 S protein. The parent IgG is shown in (A). Fusion of an unconstrained peptide (x) is shown (B), whereas a disulphide constrained peptide fusion (y) is shown in (C). Section (D) illustrates fusion of both constrained and unconstrained peptides to an IgG.

FIG. 12—Peptide fusion enhances IgG potency and resistance to mutational escape in an authentic SARS-CoV-2 virus infection assay. S protein-binding peptides (NTD-binding p16 or RBD-binding p102) were fused to the N-terminus of (A) IgG 15033-7 or (B) an anti-SARS-CoV-2 S protein-binding antibody (REGN 10933) made by Regeneron to a similar epitope. Antibody-mediated neutralization in a focus reduction neutralization test against either D614G (upper panels) or B.1.351 (lower panels) authentic SARS-CoV-2 confirmed that both enhanced potency and increased resistance to mutational escape versus parent IgG. Peptide IDs are as per FIG. 2 and notation indicates fusion with either IgG (H) or light (L) chain or both (HL).

FIG. 13—Peptides fused to tetravalent diabody-based formats enhances breadth of neutralization against SARS-CoV-2 escape variants. S protein-binding peptides (NTD-binding p16 or RBD-binding p102) were fused to the N-terminus of either the heavy (H) or light (L) chains of a tetravalent diabody-Fc-Fab (db-Fc-Fab) analog of antibody 15033-7 (see FIG. 16). Antibody-mediated neutralization in a focus reduction neutralization test against the D614G (upper panels) or B.1.351 (lower panels) authentic SARS-CoV-2 variants revealed that a second epitope conferred by addition of the peptides effectively overcame resistance arising from mutations in the B.1.351 variant and enhanced potency versus parent db-Fc-Fab 15033-7.

FIG. 14—Peptide fusions of tetravalent IgG-Fab antibodies. Schematics illustrate possible architectures of peptide-IgG-Fab fusions. Peptides selected from peptide-phage libraries can be fused to either the N-terminus of the heavy (H) and/or light (L) chain of IgG-Fab via molecular cloning of genes encoding the peptides in to constructs for expression of the antibody. The parent IgG-Fab is shown in (A). Fusion of an unconstrained peptide (x) is shown (B), whereas a disulphide constrained peptide fusion (y) is shown in (C). Section (D) illustrates possible fusions of both constrained and unconstrained peptides to an IgG-Fab.

FIG. 15—Peptide fusions of tetravalent Fab-IgG antibodies. Schematics illustrate possible architectures of peptide-Fab-IgG fusions. Peptides selected from peptide-phage libraries can be fused to either the N-terminus of the heavy (H) and/or light (L) chain of Fab-IgG via molecular cloning of genes encoding the peptides in to constructs for expression of the antibody. The parent Fab-IgG is shown in (A). Fusion of an unconstrained peptide (x) is shown (B), whereas a disulphide constrained peptide fusion (y) is shown in (C). Section (D) illustrates possible fusions of both constrained and unconstrained peptides to a Fab-IgG.

FIG. 16—Peptide fusions of tetravalent diabody-Fc-Fab antibodies. Schematics illustrate possible architectures of peptide-diabody-Fc-Fab fusions Peptides selected from peptide-phage libraries are fused to the N-terminus of the heavy (H) and/or light (L) chain of diabody-Fc-Fab via molecular cloning of genes encoding the peptides in to constructs for expression of the antibody. The parent diabody-Fc-Fab is shown in (A). Fusion of an unconstrained peptide (x) is shown (B), whereas a disulphide constrained peptide fusion (y) is shown in (C). Section (D) illustrates fusion of both constrained and unconstrained peptides to a diabody-Fc-Fab.

FIG. 17—Peptide fusions of tetravalent Fab-diabody-Fc antibodies. Schematics illustrate possible architectures of peptide-Fab-diabody-Fc fusions. Peptides selected from peptide-phage libraries are fused to the N-terminus of the heavy (H) and/or light (L) chain of Fab-diabody-Fc via molecular cloning of genes encoding the peptides in to constructs for expression of the antibody. The parent Fab-diabody-Fc is shown in (A). Fusion of an unconstrained peptide (x) is shown (B), whereas a disulphide constrained peptide fusion (y) is shown in (C). Section (D) illustrates fusion of both constrained and unconstrained peptides to a Fab-diabody-Fc.

FIG. 18—Binding kinetics for IgGs to SARS-CoV-2 S protein._BLI was used to evaluate antibody binding kinetics to immobilized S protein over a range of IgG concentrations (67-0.8 nM). Evolved sensor signals (dark grey) were fit (light grey) and binding kinetic constants were extracted, with values shown in the table at the bottom, along with EC₅₀ values derived from ELISA curves in FIG. 21C. As shown, the antibodies herein have subnanomolar dissociation constants (KD) for the SARS-CoV-2 S protein.

FIG. 19—Neutralization of authentic virus by IgGs. SARS-CoV-2 (Washington strain (2019 n-CoV/USA_WA1/2020) was assessed by a focus reduction neutralization test, (A) performed in St Louis (Miersch, S., et al., 2021, Tetravalent SARS-CoV-2 Neutralizing Antibodies Show Enhanced Potency and Resistance to Escape Mutations. Journal of Molecular Biology 433, 167177) or (B) an authentic SARS-CoV-2 B.1 clinical isolate by microneutralization assay (Manenti, A., et al., 2020, Evaluation of SARS-CoV-2 neutralizing antibodies using a CPE-based colorimetric live virus micro-neutralization assay in human serum samples. J Med Virol 92, 2096-2104.) in Rome. In both cases, samples to evaluate IgG-mediated neutralization were run in duplicate and expressed as % infection relative to an isotype or no antibody control. Neutralization curves were fitted by non-linear regression to extract IC₅₀ values, which are shown in parentheses (in ng/mL and nM).

FIG. 20—Antibody formats and binding fragments. Various possible formats of the antibody, or combinations thereof and binding fragments of this invention. Black lines indicate linkers. Light grey dots are the cysteines of an intermolecular disulphide bond.

FIG. 21—Characterization of RBD-binding antibody clones by ELISA. (A) Binding of unique Fab-phage clones (x-axis) to immobilized RBD blocked by solution-phase ACE2 (y-axis). Signal was normalized to the signal in the absence of ACE2. (B) CDR sequences of the SARS-CoV2-binding clones isolated from naive phage-displayed antibody library that target the ACE2 epitope (C) Serial dilutions of IgGs (x-axis) binding to immobilized SARS-CoV-2 S protein (y-axis) by ELISA. (D) Binding of biotinylated ACE2 (y-axis) to immobilized S protein in the presence of 100 nM IgG (x-axis). Signal was normalized to the signal in the presence of control IgG. (E) Assessment of non-specific binding of IgGs to immobilized antigens or a goat anti-human Fc Ab (positive control).

FIG. 22—Structural analysis of the 15033-7 antibody-RBD complex. (A) The complex of 15033-7 Fab with the CoV2 RBD (dark grey) is shown (B) Rotating the structure in (A) and making the Fab invisible, the epitope in the complex highlights the overlapping region of the Fab and ACE2 epitopes (dark grey) and the exclusively ACE2 (light grey) and Fab epitopes. The paratope on the Fab in the complex is shown to highlight the RBD-buried area on the VL (dark grey) and VH (light grey) with the residues optimized in 15033-7 shown as spheres. (C) Fab 15033-7 superimposed upon RBDs of SARS-CoV-2 S protein (PDB: 6X2B) in the ‘up’ position are shown to visualize simultaneous binding of two Fab arms of an IgG. (D) The position of RBD residues that interact with CDRL3 residues mutated in the affinity matured 15033-7 clone are shown (dark grey) relative to the same residues in the 15033 complex (light grey).

FIG. 23—Tetravalent antibody design and physicochemical characterization. (A) Schematic of the IgG, and tetravalent Fab-IgG and IgG-Fab antibodies generated with linkers shown in black and the intermolecular disulfide shown as light grey spheres. Atomic dimensions are shown for reference. (B) BLI sensor traces for parent and matured IgG and tetravalent antibodies binding to SARS-CoV2 S protein. (C) SEC traces for parent and affinity matured IgG and tetravalent antibodies with trastuzumab as control. (D) SDS-PAGE gels of parent 15033 and affinity matured 15033-7 IgG and tetravalent antibodies under non-reducing (top panel) and reducing (bottom panel) conditions.

FIG. 24—Neutralization of SARS-CoV-2 virus and a panel of Ala-scanning RBD variants. (A) Neutralization of clinically isolated, authentic SARS-CoV-2 virus (Washington strain (2019 n-CoV/USA_WA1/2020)) by bivalent and tetravalent formats of clones 15033 and 15033-7 in a focal reduction neutralization test. (B) Neutralization of a panel of alanine scanning mutants of SARS-CoV-2 S protein pseudotyped virus-like particles in an uptake assay with 50 nM of the indicated antibody. Signals were normalized to signals in the absence of antibody such that a signal of 1 indicates complete infection and a signal of 0 indicates no infection.

FIG. 25—Depiction of the architecture of various antibody fragments and multivalent binding molecules comprising combinations of Fabs, scFvs, diabodies and VHHs. Light chain and heavy variable domains are designated respectively as VL and VH. Light chain constant domain is designated CL. Heavy chain constant domains 1, 2 and 3 are designated respectively as CH1, CH2, and CH3. Black lines=linkers. Light grey dots are the cysteines of an intermolecular disulphide bond. Although the orientation of the scFv from its amino to carboxy terminus is depicted as VL-linker-VH, the scFv may be attached to the C-terminus or N-terminus another antibody fragment by either its VL or VH domain. As such the scFv orientation in the molecules from its amino to carboxy terminus may be either VL-linker-VH or VH-linker-VL.

FIG. 26—Free and occluded epitopes of Fab 15033-7 and ACE2 in closed S protein. The RBD surface area buried by binding of Fab 15033 is shown as a highlighted surface in (A) where the RBD surface area buried by binding of ACE2 is shown as a highlighted surface in (B) Fab 15033-7 superimposed on 1 RBD of the SARS-CoV-2 S protein in the ‘down’ position (PDB: 6ZP0) (C) illustrates the extent of steric clash with adjacent S1 domain when in the closed confirmation.

FIG. 27—Binding kinetics and CDRL3 sequences of affinity matured 15033 IgG clones. Apparent affinity and binding kinetics obtained by BLI against immobilized SARS-CoV-2 S protein are shown for 17 antibodies obtained from affinity optimization selections conducted using sub-libraries constructed by diversification of the CDR-L3 of clone 15033. CDR-L3 sequence of the 17 antibodies are shown to correlate the sequences to affinity improvements.

FIG. 28—Binding kinetics of lead anti-SARS-CoV2 S protein-binding IgGs and their tetravalent Fab-IgG and IgG-Fab analogs. Sensor traces for the association and dissociation of 5 nM IgG or tetravalent antibody (shown right of traces), obtained by BLI upon exposure to immobilized trimeric S protein or buffer respectively, are shown in dark grey with fits in light grey.

FIG. 29—Thermostability of IgG 15033, and tetravalent analogs and affinity matured variants of IgG 15033. (A) Thermal melt curves were obtained by monitoring the fluorescence of Sypro orange in the presence of 1 μM antibody in duplicate from 44-100° C. and the relative fluorescence intensity baseline shifted to zero. (B) The second inflection in point of each curve shown in (A) corresponding to CH3/Fab melting temperature (T_{M}2) was extracted and plotted as a bar graph to compare antibody formats.

FIG. 30—Validation of SARS-CoV-2 S protein pseudovirus infection assay. (A) Cellular uptake of virus-like particles pseudo-typed with SARS-COV-2 S protein in the presence or absence of a range of [Fc-tagged SARS-CoV-2 RBD] was measured via internalized luciferase activity in ACE2-expressing HEK293T cells. (B) Similarly, inhibition of uptake was observed using 15033 IgG over a range of [antibody], thus establishing both the RBD-dependence and susceptibility of uptake to antibody-mediated neutralization. Arbitrary luminescence signals were normalized to signals obtained in the absence of antibody. Samples were in triplicate with error bars representing the standard deviation of the average normalized signal.

FIG. 31—Illustration of 15033-7 contacts with RBD Phe486. Antibody residues within 4 Å of RBD residue Phe486 (identified as a mutational vulnerability in FIG. 24) are shown from the solved complex of 15033-7 Fab with SARS-CoV2 RBD. Inspection of the complex revealed antibody residues from CDRs L3, H2 and H3 in close proximity such that Phe486 inserts in to a hydrophobic pocket formed between the heavy and light chain of antibody 15033.

FIG. 32—Sequence alignments of phage-derived S protein-binding peptides. (A) Peptides selected for binding to the S protein ectodomain originated from either the X16 library (left) or the C-X4-C library (right). (B) Peptides selected for binding to the S protein RBD originated from either the C-X10-C library (left) or the CX12-C library (right). The positions are numbered at the top, followed by the consensus sequence for positions that exhibited high sequence conservation (>25% for X16, >50% for others), and the unique selected sequences are aligned below the numbering and consensus. Sequences that match the consensus are shaded grey and fixed cysteines are shaded black. Peptides that were characterized in detailed (p16, p62, p102, p103) are labeled and shown at the top each alignment.

FIG. 33—Optimization of S protein-binding peptides. Biased peptide-phage libraries were designed to optimize (A) peptide p16 or (B) peptide p102. For each peptide, the sequence logo at the top was derived from the family of peptides selected from naïve peptide-phage libraries (FIG. 2). Below the logo, the parent peptide sequence is shown with positions that were fixed or diversified in the biased library shaded black or grey, respectively. For each diversified position, the amino acids incorporated in to the displayed peptide biased library are shown are shown below the parent sequence. The sequence logo at the bottom was derived from output peptides from the biased library selection against the SARS-CoV-2 S protein ectodomain (FIG. 34).

FIG. 34—Sequence alignments of S protein-binding peptides derived from biased phage-displayed libraries. Peptides selected for binding to the S protein ectodomain originated from the biased library based on the sequence of (A) peptide p16 or (B) peptide p102 (see FIG. 2). The positions are numbered at the top, followed by the sequence of the parent peptide. Sequences that match the parental sequence at each position are shaded black. Sequences that do not match the parental sequence but are most prevalent at each position are shaded grey. Asterisks indicate positions at which sequences diverged from the parental sequence, suggesting that sequence differences here may enhance affinity of the variants relative to the parent. The sequences of peptide variants that were chosen for chemical synthesis and further characterization are labeled and shown at the top of each alignment.

FIG. 35—BLI sensorgrams of SARS-CoV-2 S protein binding to synthetic peptide. Peptides p16 and p102 and selected daughters (FIG. 34) were synthesized with a GKGK linker followed by biotin on its C-terminal end. Peptide was captured on a streptavidin coated sensor, indicated at the top of each graph, followed by blocking with free biotin. Peptide-coated sensors were dipped into concentrations of SARS-CoV-2 S protein indicated to the right of each trace. Response at the sensor was recorded for 600s followed by transfer into buffer lacking S protein and recording for another 600s. Traces were fit globally to a kinetic binding model, shown as solid line, from which kₒₙ, k_{off} and K_{D} values were derived (Table 6).

FIG. 36—Binding of p102 peptide to B.1 (Wuhan) and B.1.1.529 (Omicron) S protein RBD measured by ELISA. His tagged (A) B.1 or (B) B.1.1.529 RBD was coated onto the wells of an ELISA plate. Varying concentrations of biotinylated peptides were added to wells in triplicate. After washing, bound peptide was measured by addition of HRP-tagged neutravidin and quantification of the colour evolved from TMB reagent as the OD at 450 nm. Error bars shown represent the standard deviation of the average binding signals.

FIG. 37—Characterization of peptide-IgG fusion proteins. Peptides were fused to the N-terminus of the heavy (H) or light (L) chain of IgG 15033, and the resulting peptide-IgG fusions were named in the superscript according to the location of the fusion (L or H) and using the peptide IDs found in FIG. 2. (A) Peptide-IgG fusion proteins were electrophoretically separated on a 4-20% Tris glycine gel under non-reducing (top) or reducing conditions (bottom) and then visualized with Coomassie stain. (B) Assessment of non-specific binding of peptide-IgG fusion proteins to immobilized antigens or a goat anti-human Fc Ab (positive control). (C) BLI sensor traces for 20 nM IgG 15033 or peptide-IgG fusions binding to immobilized S protein ECD. Association and dissociation kinetics were monitored for 600 seconds each. The derived binding constants are shown in Table 7.

FIG. 38—Raw BLI traces of p16, p62, p102, or p103 peptides fused to either the heavy (H) or light (L) chain of IgG 15033 binding to SARS-CoV-2 S protein. Biotinylated S protein was loaded onto a streptavidin coated sensor, and subsequently dipped into a solution of either 4 nM or 20 nM peptide-fused IgG, as indicated. Response at the sensor was recorded for 600s followed by transfer into buffer lacking S protein and recorded for another 600s. Traces were fit globally to a kinetic binding model, shown as solid line, from which kₒₙ, k_{off}, and K_{D} values were derived (Table 7).

FIG. 39—Neutralization of authentic SARS-CoV-2 variants. (A) Microneutralization assays were conducted to evaluate the potency of IgG 15033-7 with p102 fused to the heavy chain N-terminus (33-7^{Hp102}) relative to the parental IgG 15033-7 against isolated a panel of SARS-CoV-2 variants. Colorimetric signals were read in duplicate at 590 nm and normalized relative to untreated control. Neutralization curves for the seven SARS-CoV-2 variants treated with IgG 15033-7 (black symbols, dashed lines) or peptide-IgG 33-7^{Hp102} (grey symbols, solid lines) are shown (top panel). The symbols for each curve, the country of origin for each virus variant and IC₅₀ values calculated from fit curves are shown in table format (bottom panel). (B) A focus reduction neutralization test conducted to evaluate IgG REGN10933 and peptide-IgG REGN10933^{Hp102} (REGN10933 with peptide p102 fused to the N-terminus of its HC) against isogenic SARS-CoV-2 variants D641G and B.1.351. The number of foci were counted and normalized relative to untreated virus. Each value represents the mean of duplicate measurements with error bars shown as the standard deviation of the mean.

FIG. 40—Neutralization of SARS-CoV-2 B.1.1.529 (omicron) pseudovirus by parental IgG 15033-7. A pseudovirus neutralization assay was conducted to test the relative efficacy of IgG 15033-7 against pseudotyped SARS-CoV-2 B.1 (black open circle) and B.1.1.529 (grey open circle) variants. Cellular luminescence activity generated 72 hrs following infection with antibody-treated pseudovirus was measured in triplicate and normalized relative to untreated pseudovirus. Error bars represent the standard deviation of triplicate readings.

FIG. 41—Evaluation of free peptide-mediated neutralization in the pseudovirus infection assay. To evaluate whether peptides alone neutralized pseudovirus, increasing concentrations of the indicated chemically synthesized, free peptide (up to 10 μM) was pre-incubated with VLPs pseudotyped with the SARS-CoV-2 B.1 S protein, then used to infect ACE2-expressing HEK293 cells. Serial five-fold dilutions of IgG 15033A2 from 250 nM (open inverted triangles) was evaluated in parallel as a positive control. Cellular luminescence activity generated 72 hrs following infection with antibody-treated pseudovirus was measured in triplicate and normalized relative to untreated pseudovirus. Error bars represent the standard deviation of triplicate readings.

FIG. 42—Structural models of multivalent ligands binding to the S protein trimer. Structural models are shown for (A) two 15033-7 Fabs or (B) two RGN10933 Fabs bound to the S1 subunit of the S protein of SARS-CoV-2. The S1 subunit protein is shown as a surface colored in light grey, except for the following regions. The bound RBDs in the “up” position are colored dark grey. Fabs are shown as ribbons. The C-termini and N-termini of the Fab HCs are shown as spheres, respectively. Distances are shown as dashed black lines and are labeled accordingly.

### III. DETAILED DESCRIPTION OF THE INVENTION

### A. Definitions

The term “amino acid” includes all of the naturally occurring amino acids as well as modified amino acids.

An “affinity matured” antibody or “maturation of an antibody” refers to an antibody with one or more alterations in one or more hypervariable regions (HVRs), compared to a parent or source antibody which does not possess such alterations, such alterations resulting in an improvement in the affinity of the antibody for antigen or to other desired properties of the molecule.

The term “antibody” as used herein is intended to include human antibodies, humanized antibodies, monoclonal antibodies, polyclonal antibodies, antibody binding fragments, single chain and other chimeric antibodies. The antibody may be from recombinant sources and/or produced in transgenic animals. The basic antibody structural unit is known to comprise a tetramer. Each tetramer is composed of two pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. A “whole antibody” refers to a glycoprotein comprising at least two heavy (H) chains and two light (L) chains inter-connected by disulfide bonds, or an antigen binding portion thereof. Each heavy chain is comprised of a heavy chain variable region (VH) and a heavy chain constant region. The heavy chain constant region is comprised of three domains, CH1, CH2 and CH3. Each light chain is comprised of a light chain variable region (VL) and a light chain constant region. The light chain constant region is comprised of one domain, CL or CL1. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. Herein, the assignment of amino acids to each domain is as defined in accordance with INTERNATIONAL IMMUNOGENETICS INFORMATION SYSTEM (IMGT) numbering (Lefranc et al., IMGT unique numbering for immunoglobulin and T cell receptor variable domains and Ig superfamily V-like domains, Development and Comparative Immunology. 2003; 27:55-77). In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. The exemplary antibody clones described herein are generally referred to with a number starting with 150 followed by a two-digit number (e.g., 15033), and sometimes may have a number following a dash (e.g., 15033-7). The nomenclature used herein could also be shortened to just the two-digit number, with the number following the dash, if applicable (e.g., 33 or 33-7, respectively).

The term “antibody binding fragment”, “binding fragment” or “antigen binding fragment” as used herein is intended to include without limitations Fab, Fab′, F(ab′)2, scFv, scFab, dsFv, ds-scFv, dimers, minibodies, diabodies, and multimers thereof, multispecific antibody fragments and single-domain antibodies, e.g., VHH. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, scFab, VHH, dsFv, ds-scFv, dimers, minibodies, diabodies, bispecific antibody fragments and other fragments can also be synthesized by recombinant techniques, see e.g., Bird et al. (1988) Science 242:423-426; and Huston et al. (1988) Proc. Natl. Acad. Sci. USA 85:5879-5883 and Holliger et al. (1993) PNAS. USA 90:6444-6448, and “Single domain antibodies,” Methods in Molecular Biology, Eds. Saerens and Muyldermans, 2012, Vol 911.

An antigen binding site or “binding site” is formed by amino acid residues of the N-terminal variable regions of the heavy chain (VH) and N-terminal variable regions of the light chain (VL). The three CDRs of a light chain and the CDRs of a heavy chain are disposed relative to each other in three-dimensional space to form an antigen-binding surface. The antigen-binding surface is complementary to the three-dimensional surface of a bound antigen. In single domain antibodies, also known as nanobodies, the binding site is formed by only one heavy variable domain (VH). “VHH” as used herein refers to a human VH that has been engineered to be independent of the light chain (Nilvebrant et al. Curr Pharm Des. (2016) 22(43):6527-6537; Barthelemy et al. Journal of Biological Chemistry (2007) 283:3639-3654).

The term “capture antibody” as used herein refers to an antibody or binding fragment thereof bound to a solid support and used to capture the target antigen in a sample, for example a COVID-19 polypeptide, e.g., an S polypeptide, and its S1 and S2 subunits, by forming a complex with the target antigen.

By “consisting essentially of”, it is meant a limitation of the scope of composition or method described to the specified materials or steps that do not materially affect the basic and novel characteristic(s) of the subject invention. For example, in some cases antibodies or binding fragments or multivalent binding molecules “consisting essentially of” a disclosed sequence has the amino acid sequence of the disclosed sequence plus or minus about 5 amino acid residues at the boundaries of the sequence based upon the sequence from which it was derived, e.g. about 5 residues, 4 residues, 3 residues, 2 residues or about 1 residue less than the recited bounding amino acid residue, or about 1 residue, 2 residues, 3 residues, 4 residues, or 5 residues more than the recited bounding amino acid residue.

By “consisting of”, it is meant the exclusion from the composition, method, or kit of any element, step, or ingredient not specified in the claim. For example, antibodies or binding fragments thereof or multivalent binding molecules “consisting of” a disclosed sequence consists only of the disclosed amino acid sequence.

A “conservative amino acid substitution” as used herein, is one in which one amino acid residue is replaced with another amino acid residue without abolishing the protein's desired properties. Suitable conservative amino acid substitutions can be made by substituting amino acids with similar hydrophobicity, polarity, and R-chain length for one another. Acidic amino acids include aspartate, glutamate; basic amino acids include histidine, lysine, arginine; aliphatic amino acids include isoleucine, leucine and valine; aromatic amino acids include phenylalanine, tyrosine and tryptophan; polar amino acids include aspartate, glutamate, histidine, lysine, asparagine, glutamine, arginine, serine, threonine and tyrosine; and hydrophobic amino acids include alanine, cysteine, phenylalanine, glycine, isoleucine, leucine, methionine, proline, valine and tryptophan; and conservative substitutions include substitution among amino acids within each group. Amino acids may also be described in terms of relative size; alanine, cysteine, aspartate, glycine, asparagine, proline, threonine, serine, and valine are all typically considered to be small.

The “COVID-19 S polypeptide” as used herein is an extensively glycosylated S protein of SARS-CoV-2 that protrudes from the viral membrane, to mediate host-cell entry. The S polypeptide contains 1,282 amino acids organized into 2 subunits (S1 and S2) and the receptor binding domain (RBD), is located in the S1 subunit.

The term “COVID variant” or “variants of SARS-CoV-2” as used herein refer to variants of the original Wuhan strain of SARS-CoV-2. For example, variants include coronaviruses with mutations in the S protein and/or RBD that may impair binding of neutralizing antibodies. These variants include the ones already identified, such as B.1, D614G (Plante, J. A., et al., 2021, Spike mutation D614G alters SARS-CoV-2 fitness. Nature, 592(7852), 116-121.), B.1.1.7 (Davies, N. G., et al., 2021, Estimated transmissibility and impact of SARS-CoV-2 lineage B.1.1.7 in England. Science (New York, N.Y.), 372(6538), eabg3055.), B.1.351 (Yadav, P. D., et al., 2021, Imported SARS-CoV-2 V501Y.V2 variant (B.1.351) detected in travelers from South Africa and Tanzania to India. Travel medicine and infectious disease, 41, 102023.), B.1.128 (P.1) (Faria, N. R., et al., 2021, Genomics and epidemiology of the P.1 SARS-CoV-2 lineage in Manaus, Brazil. Science (New York, N.Y.), 372(6544), 815-821.), MB61 (Fiorentini, S., et al., 2021, First detection of SARS-CoV-2 spike protein N501 mutation in Italy in August, 2020. The Lancet. Infectious diseases, 21(6), e147.), B.1.617.1 (Melloul, M., et al., 2021, Genome Sequences of the Delta Variant (B.1.617.2) and the Kappa Variant (B.1.617.1) Detected in Morocco. Microbiology resource announcements, 10(39), e0072721,), B.1.617.2 (Melloul, M., et al., 2021, Genome Sequences of the Delta Variant (B.1.617.2) and the Kappa Variant (B.1.617.1) Detected in Morocco. Microbiology resource announcements, 10(39), e0072721.), B.1.617.2+(Kannan, S. R., et al., 2021, Evolutionary analysis of the Delta and Delta Plus variants of the SARS-CoV-2 viruses. Journal of autoimmunity, 124, 102715.), B.1.427/429 (Carroll, T., et al., 2022, The B.1.427/1.429 (epsilon) SARS-CoV-2 variants are more virulent than ancestral B.1 (614G) in Syrian hamsters. PLoS pathogens, 18(2), el 009914.), and B.1.1.529 (Rahmani, S., & Rezaei, N. (2022). Omicron (B.1.1.529) variant: Development, dissemination, and dominance. Journal of medical virology, 94(5), 1787-1788.), and any yet to be identified variants with mutations to the S protein and/or RBD. The term “denatured” as used herein refers to a polypeptide that has lost tertiary and/or secondary structure (e.g. fully unfolded protein), for example when exposed to denaturing conditions in SDS sample loading buffer.

The term “detectable tag” or “detectable label” as used herein refers to moieties such as peptide sequences, a radio-opaque or a radioisotope label, such as ³H, ¹⁴C; ³²P; ³⁵S; ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase (HRP); an imaging agent; or a metal ion that can be appended or introduced into recombinant protein.

The term “detection antibody” as used herein refers to an antibody or binding fragment thereof that binds a capture antibody or a target antigen, or optionally binds a target antigen already in a complex with a capture antibody. For example, the detection antibody binds the capture antibody or a coronavirus polypeptide, or binds a coronavirus:capture antibody complex at an epitope on the coronavirus that is different than the one recognized by the capture antibody. The coronavirus may be, e.g. SARS-CoV-2 and the coronavirus polypeptide may be, e.g., a SARS-CoV-2 S protein

“Diabody” or “Diabodies” as used herein are dimeric antibody fragments. In each polypeptide of the diabody, a heavy-chain variable domain (VH) is linked to a light-chain variable domain (VL) but unlike single-chain Fv fragments, the linker between the VL and VH is too short for intramolecular pairing and as such each antigen-binding site is formed by pairing of the VH and VL of one polypeptide with the VH and VL of the other polypeptide. Diabodies thus have two antigen-binding sites, and can be monospecific or bispecific. (see e.g., Holliger, P., et al. (1993) Proc. Natl. Acad. Sci. USA 90:6444-6448; Poljak, R. J., et al. (1994) Structure 2:1121-1123; Kontermann and Dubel eds., Antibody Engineering (2001) Springer-Verlag. New York. 790 pp. (ISBN 3-540-41354-5) incorporated herein by reference.

As used herein an “effective amount” of an agent, e.g., the antibodies, binding fragments thereof, and multivalent binding molecules described herein or a pharmaceutical composition comprising the antibodies or molecules, refers to an amount effective, at dosages and for periods of time necessary, to achieve the desired result e.g., to ameliorate at least one physical adverse effect of COVID-19 infection or proliferation. This might be an amount sufficient to interfere with the binding of COVID-19 or a COVID-19 protein to its target, and/or delay onset of, one or more symptoms of the disease. The amount required to be administered will depend on the binding affinity of the antibody or multivalent binding molecule for its specific antigen, and will also depend on the rate at which an administered antibody or molecule is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight and from about 2 mg/kg to about 30 mg/kg. Common dosing frequencies may range, for example, from twice daily to once a week.

The term “epitope” as used herein refers to the site on the antigen that is recognized by the antibodies, antibody binding fragments, or multivalent binding molecule disclosed herein. Epitopic determinants usually consist of chemically active surface groupings of molecules such as amino acids or sugar side chains and usually have specific three-dimensional structural characteristics, as well as specific charge characteristics. An antibody is said to specifically bind an antigen when the dissociation constant is ≤10 μM; e.g., ≤100 nM, preferably ≤10 nM and more preferably ≤1 nM.

The term the “Fe region” or “Fe domain” refers to the constant region of immunoglobulin molecules is also called the fragment crystallizable region. The Fc domain is composed of two identical protein fragments, derived from the second and third constant domains of the antibody's two heavy chains and the Fc domains of IgGs bear a highly conserved N-glycosylation site. Glycosylation of the Fc fragment is essential for Fc receptor-mediated activity.

The term “heavy chain complementarity determining region” as used herein refers to regions of hypervariability within the heavy chain variable region of an antibody molecule. The heavy chain variable region has three complementarity determining regions termed heavy chain complementarity determining region 1 (CDR-H1), heavy chain complementarity determining region 2 (CDR-H2) and heavy chain complementarity determining region 3 (CDR-H3) from the amino terminus to carboxy terminus. All CDRs and framework regions (FRs) disclosed herein, amino acid sequences of CDRs and FRs disclosed herein, and CDR-encoding or FR-encoding nucleic acid sequences disclosed herein, are defined herein in accordance with IMGT numbering (Lefranc et al. IMGT unique numbering for immunoglobulin and T cell receptor variable domains and Ig superfamily V-like domains. Development and Comparative Immunology. 2003; 27:55-77).

The term “heavy chain variable region” or “heavy chain variable domain” as used herein refers to the variable domain of the heavy chain comprising the heavy chain complementarity determining region 1 (CDR1), heavy chain complementarity determining region 2 (CDR2) and heavy chain complementarity determining region 3 (CDR3).

The term “host cell” refers to a cell into which a recombinant DNA expression cassette or vector can be introduced to produce a recombinant cell. Host cell includes but is not limited to, prokaryotic cells such as Escherichia coli and Bacillus subtilis, fungal cells such as yeast and Aspergillus, insect cells such as S2 drosophila cells and Sf9, or animal cells, including human cells, e.g., fibroblast cells, CHO cells, COS cells, NSO cells, HeLa cells, BHK cells, or HEK293 cells.

The term “isolated antibody” or “isolated binding fragment thereof” or “isolated and purified antibody” or “isolated and purified binding fragment thereof” refers to an antibody or binding fragment thereof that is substantially free of cellular material or culture medium when produced by recombinant DNA techniques, or chemical precursors, or other chemicals when chemically synthesized, and/or other antibodies, for example antibodies directed to a different epitope.

The term “K_{D}” refers to the dissociation constant of a complex for example of a particular antibody-antigen interaction.

The term “light chain complementarity determining region” as used herein refers to regions of hypervariability within the light chain variable region of an antibody molecule. Light chain variable regions have three complementarity determining regions termed light chain complementarity determining region 1 (CDR-L1), light chain complementarity determining region 2 (CDR2) and light chain complementarity determining region 3 (CDR-L3) from the amino terminus to the carboxy terminus.

The term “light chain variable region” or “light chain variable domain” as used herein refers to the variable domain of the light chain comprising the light chain CDR1, light chain CDR2 and light chain CDR3.

The term “monoclonal antibody” (MAb), as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

The term “native” or “natively folded” as used herein refers to a protein in its native conformation (e.g. 3D conformation) or in a conformation sufficient to confer functionality, including for example partially unfolded protein capable of binding a receptor or ligand.

The term “nucleic acid sequence” as used herein refers to a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid can be either double stranded or single stranded, and represents the sense or antisense strand. Further, the term “nucleic acid” includes the complementary nucleic acid sequences as well as codon optimized or synonymous codon equivalents. The term “isolated nucleic acid sequences” as used herein refers to a nucleic acid substantially free of cellular material or culture medium when produced by recombinant DNA techniques, or chemical precursors, or other chemicals when chemically synthesized. An isolated nucleic acid is also substantially free of sequences which naturally flank the nucleic acid (i.e. sequences located at the 5′ and 3′ ends of the nucleic acid) from which the nucleic acid is derived.

“Operatively linked” is intended to mean that the nucleic acid is linked to regulatory sequences in a manner which allows expression of the nucleic acid. Suitable regulatory sequences may be derived from a variety of sources, including bacterial, fungal, viral, mammalian, or insect genes. Selection of appropriate regulatory sequences is dependent on the host cell chosen and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

As used herein the term “paratope” includes the antigen binding site in the variable region of an antibody or a multivalent binding molecule of this invention that binds to an epitope.

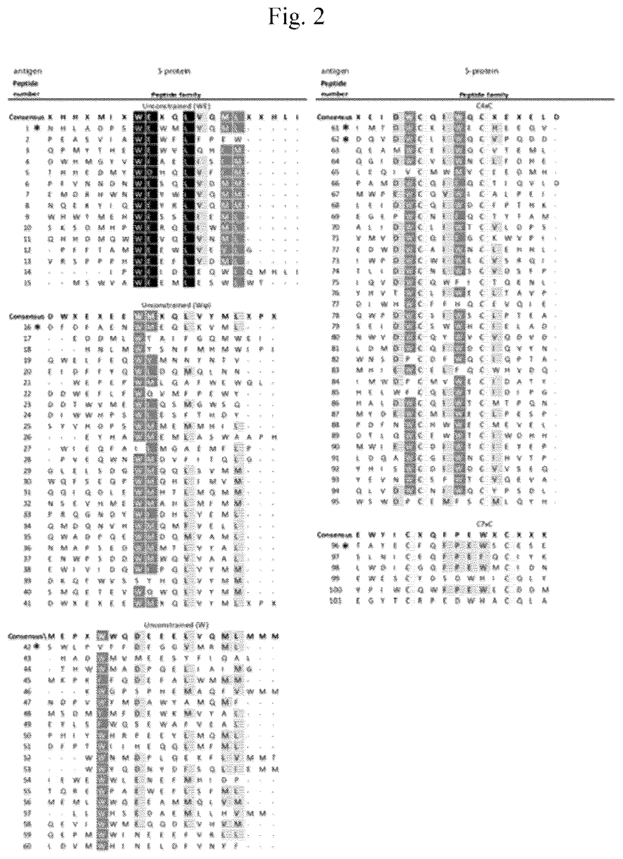

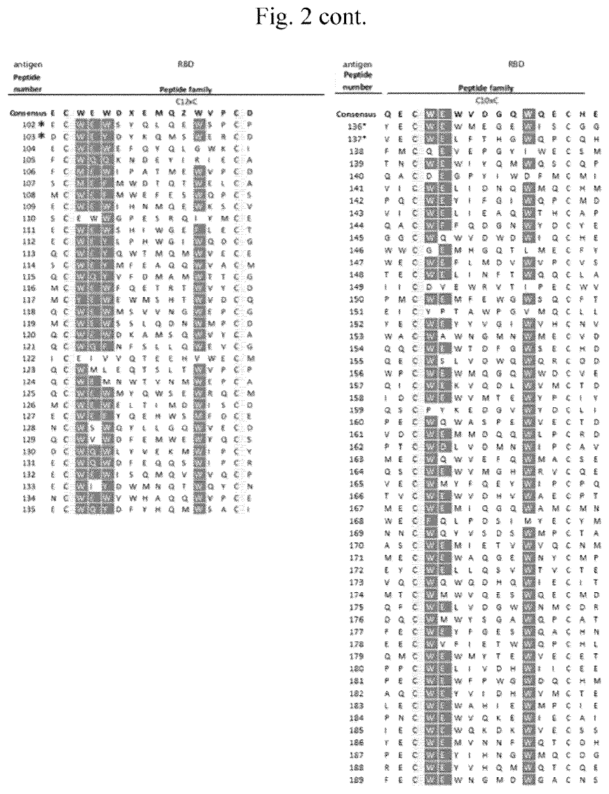

The term “peptide” as used herein refers to a chain of amino acid residues linked by peptide bonds. The peptides can be as short as 8 amino acids in length, and as long as 24 amino acids. The peptides described herein can be identified by a lowercase “p” followed by the number identified in FIG. 2, Table 8, or Table 9 (for example, p1, p2, p3, etc.). Alternatively, peptide p16 can be identified herein as N1, peptide p62 can be identified herein as N2, peptide p102 can be identified herein as R1, and peptide p103 can be identified herein as R2.

The term “polypeptide” as used herein refers to a polymer consisting of a large number of amino acid residues bonded together in a chain. The polypeptide can form a part or the whole of a protein. The polypeptide may be arranged in a long, continuous and unbranched peptide chain. The polypeptide may also be arranged in a biologically functional way. The polypeptide may be folded into a specific three-dimensional structure that confers it a defined activity. The term “polypeptide” as used herein is used interchangeably with the term “protein”.

The term “polypeptide fragment” as used herein refers to a polypeptide that has an amino terminal and/or carboxy-terminal deletion, but where the remaining amino acid sequence is identical to the corresponding positions in the naturally-occurring sequence deduced, for example, from a full-length cDNA sequence.

The term “isolated polypeptide” as used herein refers to substantially free of cellular material or culture medium when produced by recombinant DNA techniques, or chemical precursors, or other chemicals when chemically synthesized.

The term “reference agent” as used herein refers to an agent that can be used in an assay and that can be, e.g., a standard amount of COVID-19 or a COVID-19 polypeptide, e.g., a S polypeptide, used as a reference, e.g., for detecting COVID-19 or a COVID 19 polypeptide in a sample or for screening or for diagnosing a subject having or suspected of having a COVID-19 infection.

The term “sample from a subject” or “biological sample” as used herein refers to any biological fluid, cell or tissue sample from a subject, which can be assayed for a COVID-19 polypeptide. For example, the sample can comprise lung or nasal secretions, lung lavage, urine, serum, plasma or cerebrospinal fluid. The sample can for example be a “post-treatment” sample wherein the sample is obtained after one or more treatments, or a “base-line sample” which is for example used as a base line for assessing disease progression.

The term “sandwich ELISA” as used herein refers to an ELISA comprising a solid support and a capture antibody or binding fragment thereof, or a multivalent binding molecule described herein, immobilized onto the solid support. In such an ELISA an amount of target antigen (e.g. a COVID-19 S polypeptide or fragment thereof) in a sample is bound by the capture antibody. The bound antigen is detected by a second antibody or binding fragment thereof or a multivalent binding molecule described herein, which recognizes an epitope that is different from the one recognized by the capture antibody. The capture antibody:target complex (e.g., multivalent binding molecule:COVID-19 polypeptide complex) is detected by the detection antibody which can be covalently linked to an enzyme or can itself be detected by addition of a secondary antibody which is linked to an enzyme. For example, the capture antibody and/or the detection antibody can comprise CDR regions disclosed herein.

“SARS-CoV-2” or “COVID-19” as used herein is the novel coronavirus belonging to Coronaviridae family, beta-coronavirus genus and Sarbecovirus subgenus and the cause of the ongoing pandemic of Coronavirus Disease 2019 (COVID-19).

“Single-chain Fv” or “scFv” antibody fragments comprise the VH and VL domains of an antibody, wherein these domains are present in a single polypeptide chain. Generally, the Fv polypeptide (Fv fragment has the antigen-binding site made of the VH and VL regions, but they lack the constant regions of Fab (CH1 and CL) regions) further comprises a polypeptide linker between the VH and VL domains which enables the scFv to form the desired structure for antigen binding. For a review of scFv and other antibody fragments, see James D. Marks, Antibody Engineering, Chapter 2, Oxford University Press (1995) (Carl K. Borrebaeck, Ed.).

The “COVID-19 S polypeptide” as used herein is an extensively glycosylated S protein of SARS-CoV-2 that protrudes from the viral membrane, to mediate host-cell entry. The S polypeptide contains 1,282 amino acids organized into 2 subunits (S1 and S2) and the receptor binding domain (RBD), is located in the S1 subunit.

“Single-domain antibody” (sdAb), or “nanobody”, is an antibody fragment consisting of a single monomeric variable antibody domain. “VHH” or “VHH fragment” as used herein refers to a human VH that has been engineered to be independent of the light chain (Nilvebrant et al. Curr Pharm Des. (2016) 22(43):6527-6537; Barthelemy et al., Journal of Biological Chemistry (2007) 283:3639-3654).

The terms “subject,” “individual,” “host,” and “patient,” are used interchangeably herein and refer to any mammalian subject, e.g., a human being, a monkey, an ape, or a dog or cat, for whom diagnosis, treatment, or therapy is desired.

The terms “treatment”, “treating” and the like are used herein to generally means obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., slowing or arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered before the symptomatic stage of the disease, during the symptomatic stage of the disease, and in some cases after the symptomatic stage of the disease.

The term “variant” as used herein includes one or more amino acid and/or nucleotide modifications in a sequence (polypeptide or nucleic acid respectively) for example, one or more modifications of a light chain or a heavy chain complementarity determining region (CDR) disclosed herein that perform substantially the same function as the light chain and heavy chain CDRs disclosed herein in substantially the same way. For instance, variants of the CDRs disclosed herein have the same function of being able to specifically bind to an epitope on a coronavirus, e.g., a COVID-19 polypeptide, e.g., an epitope on the RBD of the S protein, or in the case of nucleotide modifications, encode CDRs that have same function of being able to specifically bind to an epitope on the coronavirus polypeptide. For example, codon optimized and degenerate sequences are included. Variants of CDRs disclosed herein include, without limitation, amino acid substitutions, including conservative amino acid substitutions, as well as additions and deletions to the CDR sequences disclosed herein. For example, the substitution, addition or deletion can be of 1, 2, 3, 4, 5, 6, 7, or 8 amino acids and/or the corresponding number of nucleotides. The substitutions may be conservative amino acid substitutions.

As used in this invention, the term “vector” refers to a nucleic acid delivery vehicle or plasmid that can be engineered to contain a nucleic acid molecule, e.g., a nucleic acid sequence encoding the antibody binding molecules or the multivalent binding molecules described herein. The vector that can express protein when inserted with a polynucleotide is called an expression vector. Vectors can be inserted into the host cell by transformation, transduction, or transfection, so that the carried genetic substances can be expressed in the host cell. Vectors are well known to the technical personnel in the field, including but not limited to: plasmid; phagemid; cosmid; artificial chromosome such as yeast artificial chromosome (YAC), bacterial artificial chromosome (BAC), or P1 derived artificial chromosome (PAC); phage such as λphage or M13 phage and animal viruses etc. Animal viruses may include but not limited to, reverse transcriptase virus (including lentivirus), adenovirus, adeno-associated virus, herpes virus (e. g. herpes simplex virus), chicken pox virus, baculovirus, papilloma virus, and papova virus (such as SV40). A vector can contain multiple components that control expression of the antibody or multivalent binding molecules described herein, including but not limited to, promoters, e.g., viral or eukaryotic promoters, e.g., a CMV promoter, signal peptides, e.g., TRYP2 signal peptide, transcription initiation factor, enhancer, selection element, and reporter gene. In addition, the vector may also contain replication initiation site(s).

The term “level” as used herein refers to an amount (e.g. relative amount or concentration) of COVID-19 or a COVID-19 S polypeptide that is detectable or measurable in a sample. For example, the COVID-19 or a COVID-19 S polypeptide level can be a concentration such as pM or a relative amount such as 1.2, 1.3, 1.4, 1.5, 1.6, 1.7, 1.8, 1.9, 2.0, 2.2, 2.4, 2.6, 2.8, 3.0, 3.2, 3.4, 3.6, 3.8, 4.0, 4.2, 4.4, 4.6, 4.8, 5.0 and/or 10 times a control level, where for example, the control level is the level of COVID-19 or a COVID-19 S polypeptide in a healthy or asymptomatic subject.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. For example, a composition comprising antibodies or binding fragments is a composition that may comprise other elements in addition to the antibodies or binding fragments, e.g. functional moieties such as polypeptides, small molecules, or nucleic acids bound, e.g. covalently bound, to antibodies or binding fragments; agents that promote the stability of the antibodies or binding fragments composition, agents that promote the solubility of the antibodies or binding fragments composition, adjuvants, etc. as will be readily understood in the art, with the exception of elements that are encompassed by any negative provisos. The foregoing also applies to words having similar meanings such as the terms, “including”, “having” and their derivatives.

In understanding the scope of the present disclosure, the term “consisting” and its derivatives, as used herein, are intended to be close ended terms that specify the presence of stated features, elements, components, groups, integers, and/or steps, and also exclude the presence of other unstated features, elements, components, groups, integers and/or steps.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention. It is also to be understood that all numbers and fractions thereof are presumed to be modified by the term “about.” Further, it is to be understood that “a,” “an,” and “the” include plural referents unless the content clearly dictates otherwise. The term “about” means plus or minus 0.1 to 10%, 1-10%, or preferably 1-5%, of the number to which reference is being made.

Further, the definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### B. Peptides

Considerable effort has gone towards isolation and characterization of neutralizing mAbs (nAbs) (Hansen, 2020, PMID: 32540901) (Liu, 2020, PMID: 32698192) (Kreye, 2020, PMID: 33058755) (Zhou, 2020, PMID: 33657424) (Du, 2020, PMID: 32243608) (Ju, 2020) (Wu, 2020, PMID: 33275640) (Hurlburt, 2020, PMID: 33110068). nAbs as therapeutics are an intriguing strategy, because they can be rapidly isolated and characterized from the peripheral blood of previously infected humans. Moreover, since these mAbs are produced by human immune systems and their nature is such that they are highly specific for their target, chances of side effects due to broad targeting and patient immune response are limited. This significantly reduces time from lead identification to clinical approval. Within months of the emergence of COVID-19, several nAbs leads were isolated and characterized, and three have recently received approval for clinical use. These nAbs represent the first non-repurposed therapeutics approved for treatment of COVID-19, demonstrating the reduced timeline from lead development to approval compared to other drug classes such as small molecules. Isolating nAbs from human subjects, however, necessitates infection, and is thus inherently limited in its developmental pathway.

One way around the issue of requiring human infection is through the use of recombinant phage-displayed mAb libraries. All that is necessary for this process is the genetic sequence of the viral antigen to be targeted. This allows for the production of critical antigens through recombinant protein production technology, against which the phage-displayed libraries are bio-panned to isolate specific high-affinity nAbs. Our lab was able to implement this technique to isolate nAbs of comparable affinity and quality to those isolated from peripheral blood of COVID-19 infection patients over the same timeline.

Intriguingly, despite divergence in methodology, nearly all of the nAbs against CoV-2 bind highly overlapping epitopes found on the S protein RBD. The mode of action of these nAbs is through steric blocking of the ACE2-RBD interaction to prevent infection. One exception are a set of nAbs that bind S protein in the NTD, and likely neutralize the virus through allosteric effects which stabilize CoV-2 S protein confirmations unable to bind ACE2, or unable to be cleaved and transition to the post-fusion state (Yuan, 2020, PMID: 32661058). Binding of nAbs to such a limited set of epitopes raises of the possibility of mutations which allow the virus to escape blocking by a large set of nAbs through a reduction or elimination of binding to these Abs. Already, a covid variant of CoV-2 which is more transmissible has emerged where in a residue in the RBD, N501, is mutated to a tyrosine (https://cmmid.github.io/topics/covid19/reports/uk-novel-variant/2020_12_23_Transmissibility_and_severity_of_VOC_202012_01_in_England.pd f). This position is significant for ACE2 binding (Starr, 2020, PMID: 32841599), and thus one explanation for increased transmissibility is that the N501Y mutation increases affinity for ACE2. It is unclear how N501Y will affect neutralization efficiency of nAbs, however location of the mutation and mechanism of the vast majority of nAb action suggests it will hinder neutralization efficiency of at least of subset of nAbs, and that other RBD mutations that impact nAb binding to SARS-CoV-2 S protein are sure to emerge in the future.

One strategy for overcoming resistance conferred by escape mutations is to devise recombinant nAbs into formats that increase avidity, potency and breadth of neutralization against viruses, such as variants of SARS-CoV-2. Our lab and others have shown this strategy to have promise (Miersch et al, https://doi.org/10.1101/2020.10.15.341636.). Taking it a step further, we can expand the number of epitopes of high valency formats via the fusion of peptides to neutralizing antibodies. Recent work has shown that peptide fragments are able to bind both RBD and non-RBD epitopes on the S protein (Cao, 2020, PMID: 32425270) also (Pomplun ACS, 2021, PMID: 33527085).

Previous studies have fused a single inhibitory peptide (HR2P) derived from the heptad repeat 2 (HR2) domain of MERS-CoV to the C-terminus of recombinant antibody fragments that targeted the receptor-binding site (Wang, 2019, PMID: 31690009 and 30621343). The peptide was identified by a structure-based approach for its ability to form a stable six-helical bundle by binding the MERS-CoV S protein independently to block viral fusion and replication (Lu, Nat Commun, 2014). Authors concluded from their study that bispecific inhibitors, formed by fusion of this peptide to the C-terminus of a recombinant antibody targeting the receptor-binding site increased the efficacy against MERS-CoV.

We developed novel CoV-2 S protein binding peptides making use of a naïve phage-displayed 16mer peptide library. We show that these peptides bind epitopes in both the RBD and S protein outside of the RBD. Fusion of these peptides to the N-terminus of nAbs improves their apparent affinity, potency and breadth of neutralization, without compromising their developability profile. Thus, these peptides represent a novel class of SARS-CoV-2 S protein binders, which can be formatted in various ways to improve functional properties of nAbs.

In a first embodiment of the invention, at least one peptide that binds the CoV-2-S protein is fused to an anti-CoV-2 antibody. In another embodiment, the fusion includes at least one peptide fused to the N-terminus of at least one of the light chains of the anti-CoV-2 antibody. In another embodiment, the fusion includes at least one peptide fused to the N-terminus of at least one of the heavy chains of the anti-CoV-2 antibody. In another embodiment, the fusion includes at least one peptide fused to the N-terminus of each of the heavy chains of the anti-CoV-2 antibody. In another embodiment, the fusion includes at least one peptide fused to the N-terminus of each of the light chains of the anti-CoV-2 antibody. In another embodiment, the fusion molecule comprises an anti-CoV-2 antibody and two different peptides. In another embodiment, the fusion molecule comprises an anti-CoV-2 antibody and at least one peptide fused on each of the heavy chain and the light chains, where the peptide fused to the heavy chain can be the same or different than the peptide fused to the light chain.

In another embodiment of the invention, at least one peptide that binds to the protein of a virus of interest is fused to an antibody that binds to the same virus of interest. In another embodiment of the invention, the antibody is a neutralizing antibody. An embodiment of this invention is that the epitope to which the antibody binds is within proximity to the binding site of the peptide so as to enable avidity effects by tethering the peptide to the antibody N-terminus with a linker peptide of reasonable length. Suitable linkers are described herein. Identification of peptides that enhance antibody avidity for the viral target can be screened by any method capable of resolving functional differences, for example, by fusing the peptide to the N-terminus of the antibody light or heavy chain and screening their binding kinetics to reveal enhancements relative to the parental antibody without the linker and with the linker alone, or by neutralization assays of authentic or pseudovirus, or the methods described herein for CoV-2.

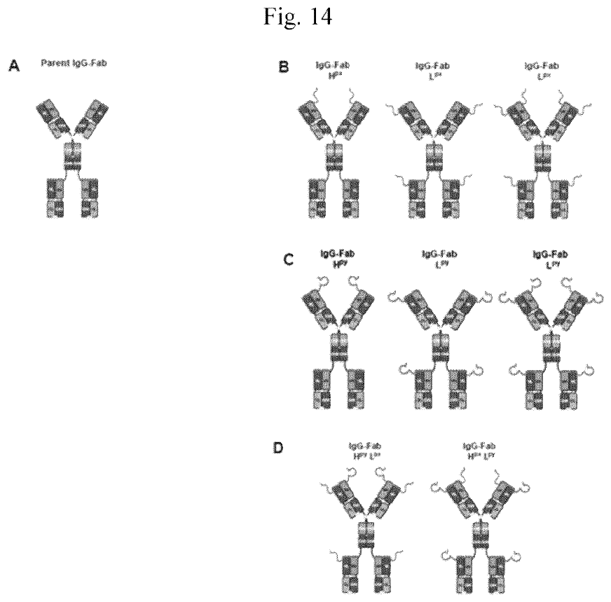

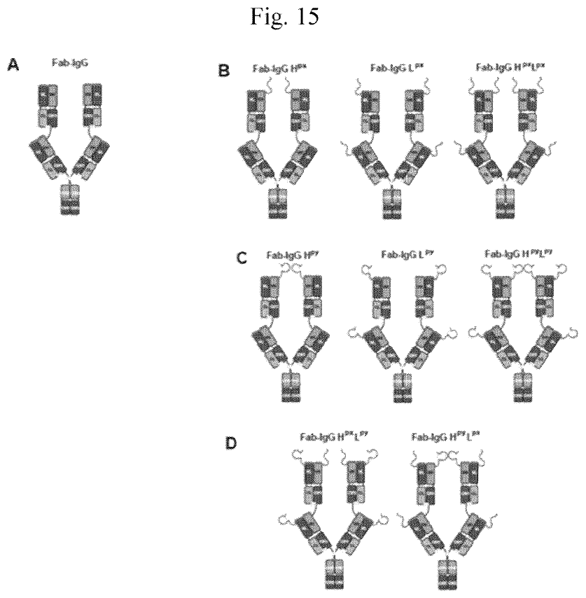

In another embodiment of the invention, at least one peptide that binds the CoV-2-S protein is fused to an anti-viral synthetic antibody. In another embodiment, the fusion comprises at least one peptide fused to a multivalent binding molecule comprising two or more antibody binding fragments that bind a coronavirus, wherein the binding fragments of the multivalent binding molecule are joined by a peptide linker. In an embodiment at least one peptide described herein is fused to the multivalent binding molecule, where the multivalent binding molecule is at least bivalent or at least trivalent. In an embodiment of the invention, the peptide can be fused to any free N-terminus. The peptides fused to each N-terminus can be the same or different. The peptides can be fused to all of the free N-terminus locations, or to any combination of the free N-terminus locations. In one embodiment, there is a peptide attached to each free N-terminus of the multivalent binding molecule. Exemplary embodiments using anti-SARS-CoV-2 S protein IgG-Fab multivalent molecules are shown in FIGS. 14, 15, and 16 and 17.

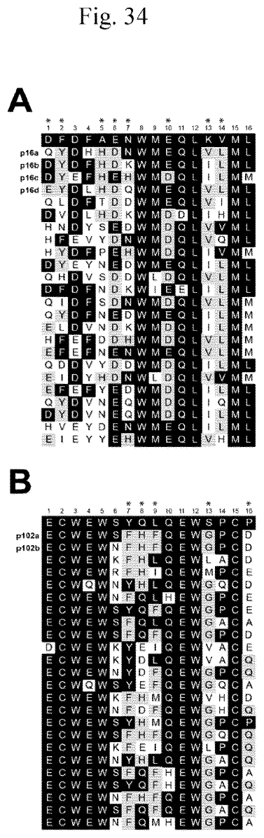

The peptide can comprise any peptide that is found to bind to the CoV-2 S protein in either the RBD or non-RBD regions of the S protein. The peptide can be fused to the N-terminus of the anti-CoV-2 antibody. The peptide embodied by this invention can range in size from 8 to 24 amino acids. The peptide is a suitable size to allow for binding to the S protein. For example, the peptide can be 16 amino acids, like those described in FIG. 2, Table 8. In another embodiment of the invention, the peptide is selected from the peptides included in FIG. 34, or Table 9.

In another embodiment of the invention, the peptide comprises a sequence that is at least 50%, 60%, 70%, 75%, 80%, 85%, 90%, 91%, 92%, 93%, 94%, 95%, 95%, 97%, 98%, 99%, or 100% identical to the peptides identified in FIG. 2, Table 8, or Table 9.

The peptide and the antibody can be joined via a linker (i.e., a flexible molecular connection, such as a flexible polypeptide chain). The linker can be any suitable linker, such that the fusion protein can bind to the RBD or non-RBD regions of the S protein. The linker can be any suitable length, but is preferably at least about 10 (e.g., at least about 15, at least about 20, at least about 25, at least about 30, at least about 35, at least about 40, at least about 45, at least about 50, or ranges thereof) amino acids in length. In another embodiment, the linker is in excess of 70 Å in order for a peptide tethered to the N-terminus of either the heavy or the light chain to reach adjacent S protein epitopes. In one embodiment, the linker is a flexible non-cleavable linker. An example of a suitable linker includes, but is not limited to, (G₄S)₄. The average extended length of a non-structural peptide is ˜3.5 Å per residue. Given this (G4S)4 linker is 20 residues in length and that is glycine serine rich content would render it unstructured, it should have a length of approximately 75 Å, making it a suitable linker to tether a peptide and antibody, such as Antibody 15033 described herein.

The anti-CoV-2 antibody can be any antibody with anti-CoV-2 properties, for example, the antibodies described herein. In one embodiment, the anti-CoV-2 antibody is one that binds the RBD. In one embodiment, the anti-CoV-2 antibody is an antibody or antibody binding fragment that binds SARs-CoV-2 and/or SARs-CoV-2 S polypeptide, e.g., S protein subunits S1 or S2. In another embodiment, the anti-CoV-2 antibody is a synthetic multivalent antibody that binds SARS-CoV-2.

In another embodiment of the invention, the fusion molecule comprises at least one peptide described in FIG. 2, Table 8, or Table 9 and an anti-CoV-2 antibody described herein. In another embodiment, the peptide is at least one described in FIG. 2, Table 8, or Table 9, and Antibody 15033, described herein. In another embodiment, the peptide is at least one described in FIG. 2, Table 8, or Table 9, and Antibody 15033-7, described herein. In another embodiment, the peptide is at least one described in FIG. 2, Table 8, or Table 9, and Antibody 15033A2, described herein. In another embodiment, the peptide is at least one described in FIG. 2, Table 8, or Table 9, and any of the antibodies in Tables 2 and 3, described herein.

### EXAMPLES

Structural analysis of 15033 Fabs bound to CoV-2 S protein reveals a strategy for neutralization efficiency improvement through peptide tethering.

We developed a panel of nAbs against CoV-2 through binding of various epitopes on the S protein (Miersch, S., et al., 2021, Tetravalent SARS-CoV-2 Neutralizing Antibodies Show Enhanced Potency and Resistance to Escape Mutations. Journal of molecular biology, 433(19), 167177.). The most efficient neutralizer with ideal drug-like properties was Ab 15033, which was subsequently improved to yield 15033-7. The structure of 15033-7 Fabs bound to CoV-2 S protein ectodomain was solved by cryo-EM. These structures revealed that 15033 Fabs are able to bind S protein in a variety of ways. Structures with 2 or 3 Fabs bound were observed, and with 3 Fabs bound, it was observed that on RBD could adopt the ‘up’ or ‘down’ conformation. Negative stain EM revealed that a 15033 IgG could bind one S protein, and the orientation of the class average of this structure suggested that each Fab of the IgG bound to an ‘up’ RBD. Thus, we selected the two Fab bound to two ‘up’ cryo EM structure as a model for IgG binding to the S protein for analysis.

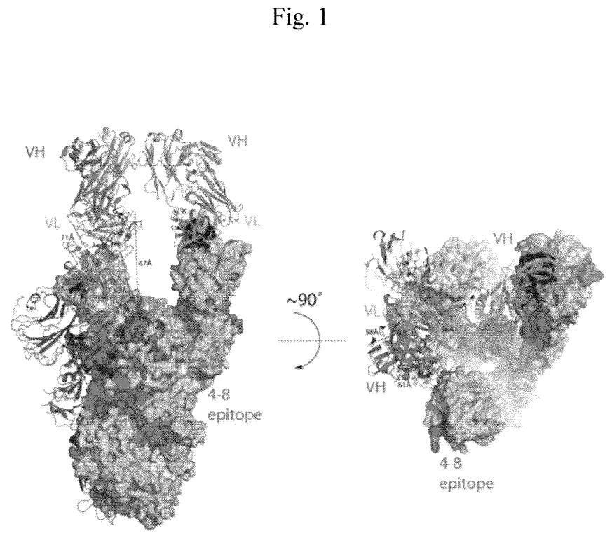

The N-terminus of the Fabs fall more closely to the NTD region of the S protein monomer adjacent to the one to which they are bound. The N-terminus of the heavy chain is slightly closer to the adjacent NTD than the light chain (67 Å versus 71 Å, respectively) (FIG. 1, side view). One the other hand, the N-terminus of the light and heavy chains are approximately the same distance to the RBD of the adjacent monomer (FIG. 1, top view).

Based on this we determine that we would need a linker in excess of 70 Å in order for a peptide tethered to the N-terminus of either the heavy or the light chain to reach adjacent RBD or non-RBD epitopes. The average extended length of a non-structured peptide is ˜3.5 Å per residue. In previous work we have made use of a four repeat, four glycine plus one serine linker (G₄S)₄. Given this linker is 20 residues in length and that is glycine serine rich content would render it unstructured, it should have a length of approximately 75 Å, making it suitable to tether a peptide to 15033 such that both the 15033 paratope and the peptide epitopes were within the range of distances conferred by the linker;

Isolation of CoV-2 S protein-binding peptides from a phage displayed peptide library.

In order to isolate novel SARS-CoV-2 S protein binding peptides, a randomized 16mer phage-displayed library was panned against purified CoV-2 S protein RBD or CoV-2-S protein-ECD antigens. The library was an equal mixture of 16mers fully randomized at each position, and libraries where two positions in the 16mer were fixed as cysteines, with the remaining position were fully randomized, resulting in a closed loop (constrained) peptide through intramolecular disulphide bridge formation. The distance between cysteines ranged from 4 to 12, thus the library was a mixture of 10 libraries total, in equal amounts. After four rounds of selection, 160 unique clones against CoV-2 S protein ECD and 128 unique clones against CoV-2 S protein RBD were sequenced and characterized. Table 1 shows the number of unique clones identified for SARS-CoV-2-S protein ectodomain and SARS-CoV-2 RBD and the particular families in which they fall.

TABLE 1

| | number of unique clones | | | | | | |
|---|---|---|---|---|---|---|---|
| Antigen | Total | 16X | X₅—C—X₄—C—X₅ | X₄—C—X₅—C—X₅ | X₄—C—X₆—C—X₄ | X₃—C—X₇—C—X₄ | X₃—C—X₈—C—X₃ |
| SARS-COV-2 | 160 | 101 | 35 | 9 | 3 | 6 | 1 |
| S protein - | | | | | | | |
| ectodomain | | | | | | | |
| SARS-COV-2 | 128 | 1 | 5 | 5 | 1 | 1 | 11 |
| RBD | | | | | | | |

| | number of unique clones | | | |
|---|---|---|---|---|
| Antigen | X₂—C—X₉—C—X₃ | X₂—C—X₁₀—C—X₂ | X—C—X₁₁—C—X₂ | X—C—X₁₂—C—X |
| SARS-CoV-2 | 0 | 5 | 0 | 0 |
| S protein - | | | | |
| ectodomain | | | | |
| SARS-CoV-2 | 5 | 54 | 11 | 34 |
| RBD | | | | |

Each unique SARS-CoV-2 S protein antigen favored binding from specific library families. Table 1 shows that of the 160 unique clones from SARS-CoV-2 S protein ectodomain, about 63% belonged to the unconstrained family (U) and about 22% belonged to the X₅-C-X₄-C-X₅ family (C4). The term “unconstrained” means that the peptide does not contain cysteine residues and is therefore not constrained by a disulfide bond. With respect to SARS-CoV-2 RBD selections about 27% belonged to the X—C—X₁₂-C-X family (C12) and about 42% belonged to the X₂-C—X₁₀-C—X₂ family (C10).

The unconstrained family of peptides can be subdivided into 3 distinct subfamilies. The first, herein referred to as the WΦ family, is characterized by a WΦxxLxxMM motif. The second, herein referred to as the WE family, contained a WExxLVxML motif. And the final, herein referred to as the W family, contains a WxxDxxxLxxML motif. In terms of cysteine constrained peptides there were 4 families. The S-C4 family contains a DWCXΦWXC consensus from position 4, the S-C7 family contained predominantly one peptide with a FPEW motif in between the flanking cysteines, S-C10 contains a CWExxxxxWxC motif, and finally the S-C12 family contains a CWEWxxxxxxWxxC motif from position 2. As can be seen in FIG. 2, the peptides described fall into these categories. Both the unconstrained and constrained peptides can be placed on either the light chain or the heavy chain.

### Characterization of CoV-2 Binding Peptides

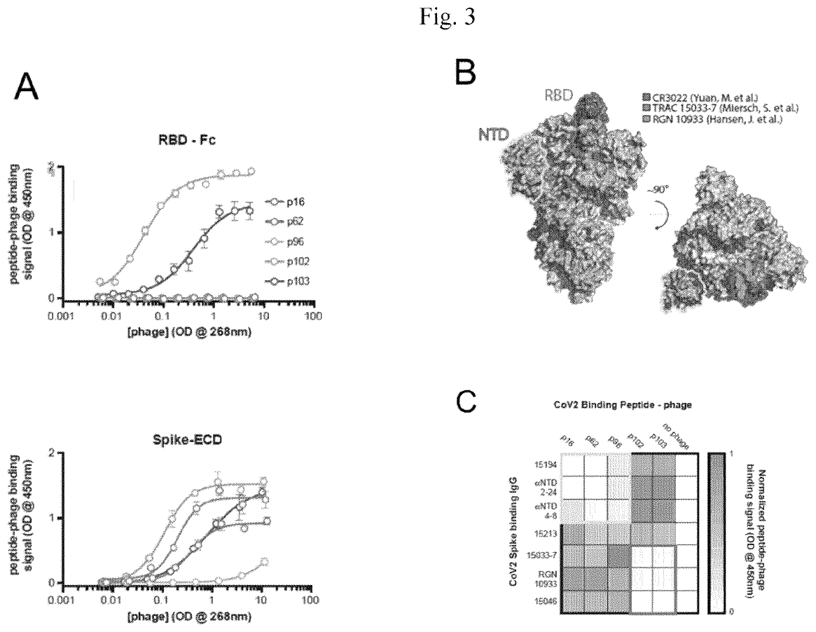

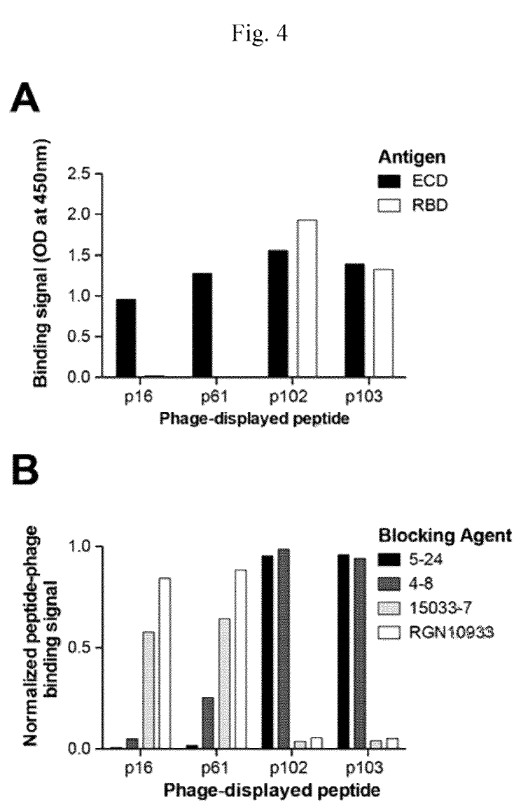

We sought to characterize the epitopes targeted by these peptides. The RBD fused to an Fe tag (RBD-Fc) as well as the full-length S protein ectodomain fused to a His tag (S protein ectodomain) were cloned and purified from Expi293 cells, and the EC₅₀ of phage-displayed peptides to these antigens was determined through ELISA (FIG. 3). As expected, C-12 family members (e.g. p102, p103 etc), which were isolated from biopanning against the RBD, bound only to the RBD with EC50's of 0.04 and 0.4 A_{268 nm} absorbance units (FIG. 3A—upper), respectively, while no measurable interaction was observed between RBD-Fc and p16, p62, and p96 peptides (FIG. 3A—upper). On the other hand, all peptides bound S protein ectodomain with varying efficiency, suggesting the latter group of peptides bind the S protein outside the RBD (FIG. 3A—lower). Next, we made use of a panel of anti-SARS-CoV-2 S protein-binding antibodies, developed by our lab and others, to map the epitopes for these SARS-CoV-2 S protein-binding peptides. Our antibody 15033-7 is well-characterized SARS-CoV-2 neutralizer, which binds to the RBD, making significant contacts with F486 (FIG. 3B). We selected other antibodies from the literature (CR3022 and RGN 10933), which bind different epitopes on the RBD (FIG. 3B). We also made use of well characterized nAbs which bind to the NTD region of the S protein (FIG. 3B) (Liu, Nature, 2020). We performed a competition ELISA to determine which of these antibodies interfered with phage-displayed peptide binding from representatives of each family. We found two main epitopes bins (FIG. 3C). The C-12 epitope (e.g. p102, p103) overlapped with the 15033-7 epitope on the RBD, while the U(WΦ) (e.g. p16), C4 (e.g. p62), C7 epitope(s) (e.g. p96) overlapped with the NTD binders.

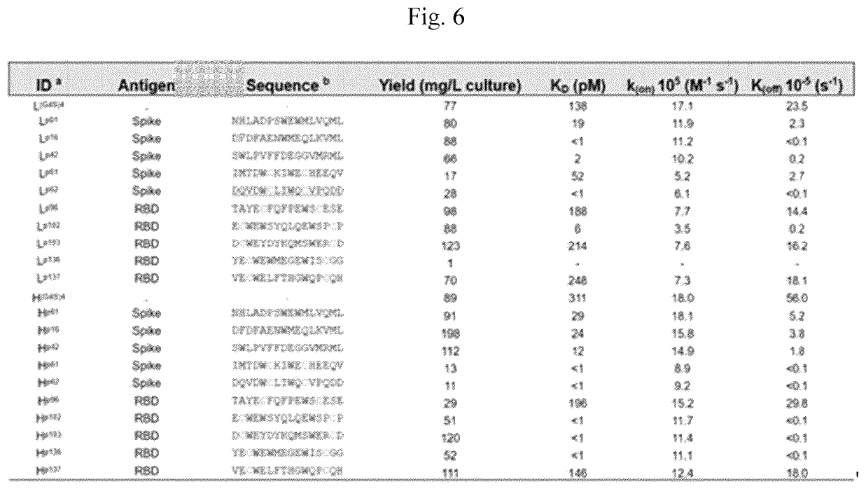

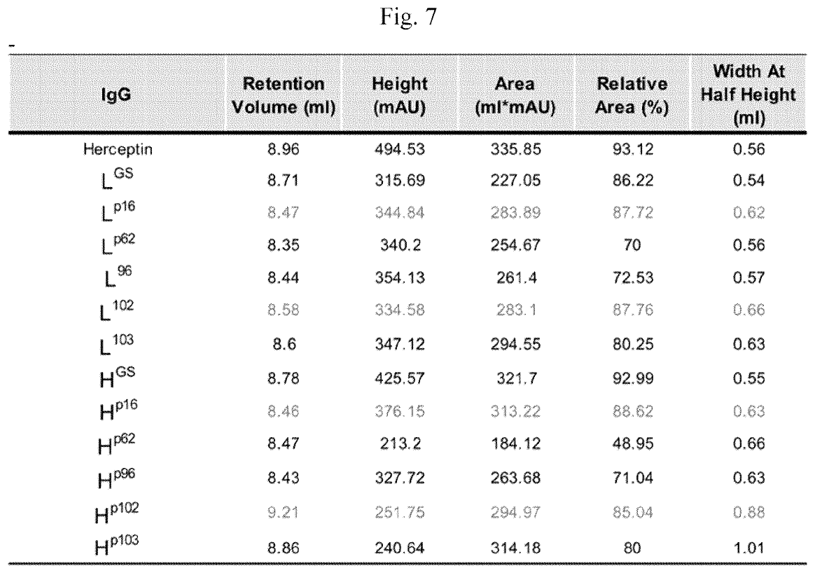

Given these results, we selected a panel of peptides from each family, 10 peptides in total, to build peptide antibody fusions to either the heavy or light chain. Peptides sequences are as per FIG. 2 (asterisks). Peptides were fused to the N-terminus of either the heavy chain or the light chain of the 15033 IgG, which we recently developed as nAb that targets an epitope on the SARS-CoV-2 RBD, through a (Gly₄Ser)₄ linker. These constructs were cloned, overexpressed in Expi293 cells, and purified using protein A resin. Comparison of the yields obtained from each purification shows overall little change compared to the parental 15033 IgG yield, save for some exceptions which either significantly reduced or abolished expression, or improved expression about two-fold (FIG. 6).

We next determined whether these peptides improved affinity of 15033 for purified recombinant SARS-CoV-2 S protein. Results from FIG. 6 show that fusion of some peptides to 15033 improved affinity for recombinant SARS-CoV-2 S protein ectodomain by as much as >150 fold (FIG. 6) as measured by biolayer interferometry (BLI), while some showed no improvement, or even impaired binding. Improvements in affinity invariably came as a result of improvements in off rates by as much as 1000-fold (FIG. 6). The addition of a peptide would impose entropic penalties in binding to the S protein, while at the same time increasing the overall enthalpic energy of binding. Thus, these results are expected, as the entropic penalty would reduce or not affect the on rate of binding, and the increased enthalpy would reduce the off rate of binding. Comparison of the same peptide fused to either the light or heavy chain reveals that heavy chain fusions tend to perform better than light chain fusions, although not in all contexts. For example, p61 and p103 showed a 50- and ˜200-fold improvement in affinity, respectively, when fused to the heavy chain compared to the light chain. On the other hand, p16 and p42 showed significant affinity improvement when fused to either light or heavy chain, but light chain fusions were slightly better. Inspection of the 15033-IgG/CoV-2-RBD crystal structure reveals that the light chain CDRs provide a majority of the contacts. Thus, higher performance of heavy chain fusions could be due to the increased availability of the peptide when fused to the heavy chain, interference of light chain binding upon fusion of the peptide, or a combination of both.

### Biophysical Characterization of CoV-2-Binding Peptides to Antibody 15033

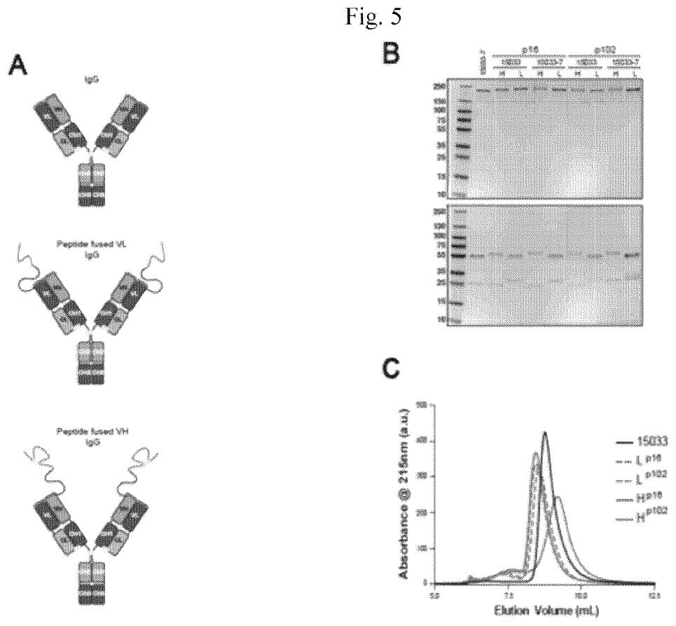

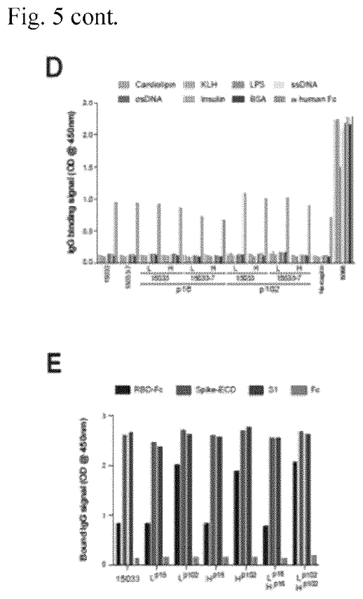

From this panel of antibodies we selected those that improved affinity over 100-fold when fused to either the light or heavy chain, or both, for further characterization. Since the end goal of these molecules is to serve as lead therapeutics for treatment of COVID infection, we performed a series of experiments to determine their drug-like properties. SDS-PAGE analysis revealed that the IgG's were purified to homogeneity and that proper disulphide bridge formation within the IgG's molecules had occurred (FIG. 5B). We implemented Size-exclusion chromatography (SEC) to determine whether these molecules were mono-disperse and stable in solution. As shown in FIG. 5C, attachment of CoV-2-binding peptides had varying degrees of biophysical effects on 15033. The most typical effect was for these peptides to induce aggregation upon fusion to 15033 compared to the native. This effect is contingent on several variables. The first is the presence of cysteines, likely due to intermolecular disulphide bridge formation. Interestingly, it appears as though a larger distance between flanking cysteines reduced the extent of aggregation, perhaps due putative a favoring of intra-versus intermolecular disulphide bridge formation.

Another variable is the IgG chain to which the peptide was fused. Peptide fusion to the light chain appears to have a less disruptive effect on IgG structure compared to fusion to the heavy chain (FIG. 6). This is readily observed comparing the SEC profile of p62 to either the heavy or the light chain of 15033 (FIG. 5C) where heavy chain fusion significantly increased aggregation. Fusion of C-12 peptides to the heavy chain resulted in peak broadening and slight retention on the SEC matrix compared to the 15033 parent or either of these peptides fused to the light chain. Next, we sought to determine whether addition of peptides to the N-terminus of IgG's resulted in an increase of non-specific binding to a set of ubiquitous biomolecules through an ELISA. Binding to these molecules is a flag another marker for poor developability (Jain, 2017, PMID: 28096333). We did not observe binding to any of these common biomolecules above the parental 15033 in either light or heavy chain context (FIG. 5D). Thus, overall, fusion of CoV-2 peptides to the N-terminus had negligible to moderate effect on developability of the 15033 IgG, depending on the identity of the peptide. Effects tended to be structural disruption resulting in aggregation or SEC peak broadening, while nonspecific binding did not appear to be affected at all. Specificity for SARS-CoV-2 was also maintained upon fusion of either single or dual peptides to the IgG (FIG. 5E).

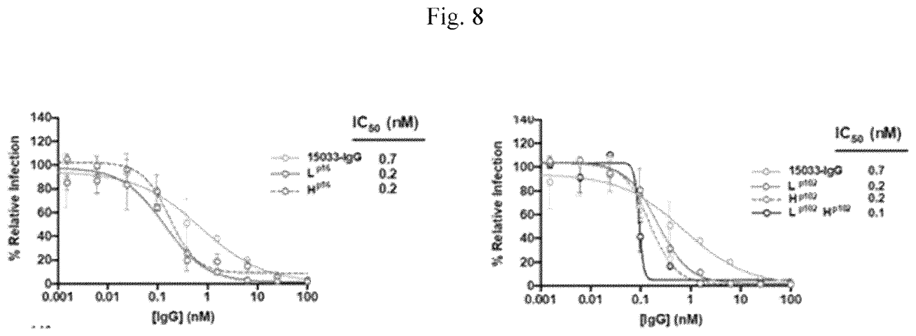

Improvement in CoV-2 neutralization efficiency through fusion of S protein binding peptides to nAbs. Fusion of S protein-binding peptides enhanced IgG potency in a SARS-CoV-2 pseudoviral infection assay. Following confirmation of enhanced avidity, which is known to often correlate with enhanced anti-viral potency, the functional activity was assessed in a pseudovirus infection assay that uses an HIV-gag virus-like particle pseudotyped with SARS-CoV-2 S protein that also encodes a luciferase gene (Miersch BioRxiv, 2020). Uptake of the particle results in the translation of an active luciferase enzyme that can be detected upon cell lysis in the presence substrate and whose activity is proportional to the amount of internalized VLP. Pre-incubation of VLP's with either p16 or p102 peptide-fused IgG 15033 on either the heavy or light chain revealed a marked improvement in neutralization potency in comparison to parent IgG without peptide (FIG. 8). Importantly, dual labelling of IgG 15033 with p102 on both the heavy and light chains, improved potency over the singly-labelled neutralizing antibody suggested that the effects of peptide fusion on avidity and potency are additive (FIG. 8—right panel).

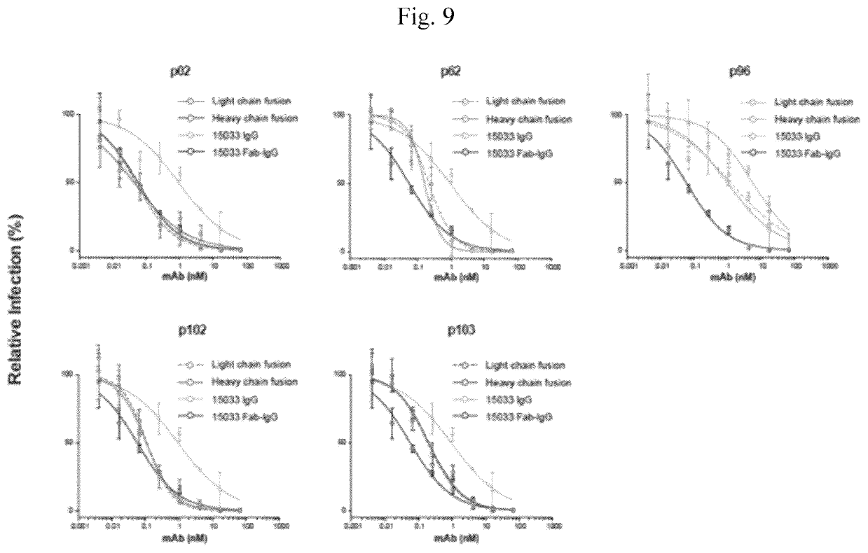

We next sought to determine whether fusion of these peptides could improve SARS-CoV-2 potency against authentic virus (Washington strain). Authentic SARS-CoV-2 virus was incubated with increasing concentrations of our peptide-fused anti-CoV-2 anti-bodies and then added to ACE2 expressing Vero E6 cell cultures. Vero E6 infection was measured and plotted as a function of incubating peptide-Ab concentration. These plots were used to determine IC₅₀ values for comparison of neutralization efficiency. We used 15033 IgG with no peptide fusion as a baseline comparison, and for upper bound comparison, we included 15033 in a Fab-IgG tetravalent format, previously shown to dramatically improve neutralization efficiency. We found significant improvements in the IC₅₀ values of all our peptide fusions compared to the 15033 parental alone, except for p96, which showed no apparent improvement in potency when fused to the light chain, and decreased potency when fused to the heavy chain (FIG. 9). Fusion of p16 to either the light or heavy chain showed the most marked improvement in 15033 neutralization efficiency. Peptide p102 performed second best in terms of improvement in neutralization efficiency of 15033, again upon fusion to either the light or the heavy chain. These results mirror the BLI data, showing that it is a reliable proxy to determine viral binding.

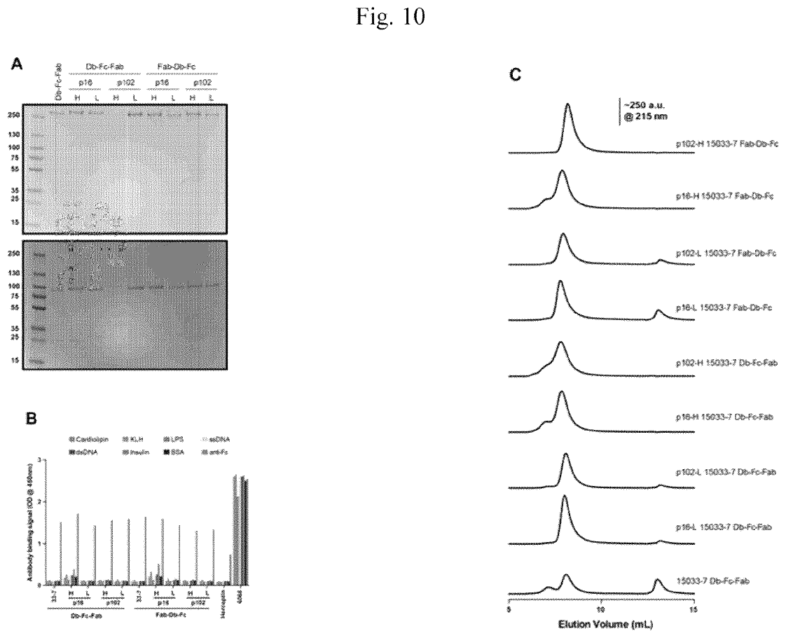

Encouraged by these results we opted to fuse p16 and p102 to the 15033-7 tetravalent formats and express the fusions with our diabody-Fc-Fab (db-Fc-Fab), and Fab-diabody-Fc (Fab-db-Fc) modalities to determine if it would improve neutralization performance of either of these molecules. SEC and non-specificity profiles revealed that fusion of p16 or p102 to either 15033-7 light or heavy chain, or 15033 Fab-IgG light chain did not disrupt these molecules structure, stability, or non-specific reactivity profile. (FIG. 10).

### Additional Figures:

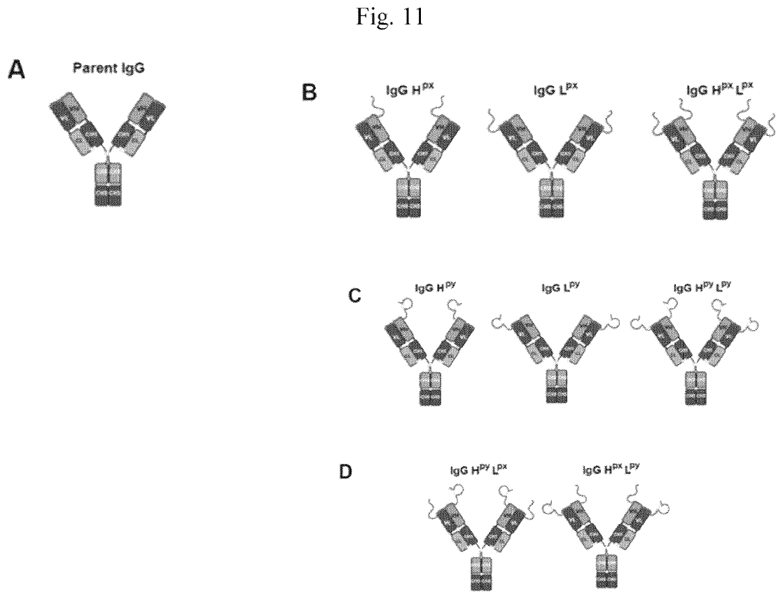

Conceived of as a tool to rapidly enhance the affinity of virtually any anti-S protein antibody, we identified a small subset of 10 candidates (indicated by an asterisk* in FIG. 2) based on apparent favourable physicochemical properties and consensus sequences, and fused them (with a 20 (G₄S)₄ amino acid linker) to the N-terminus of the light and heavy chains of a highly potent CoV-2 neutralizing antibody (IgG 15033) (FIG. 11).

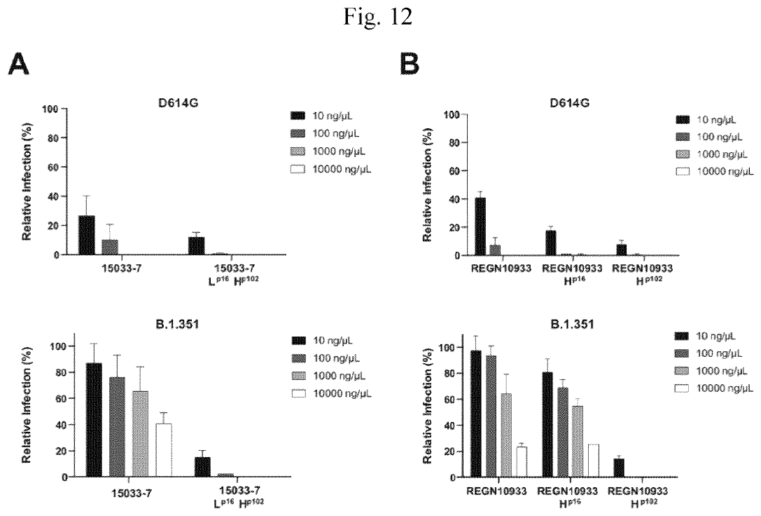

Virtually identical results were obtained and comparison to a previously assayed tetravalent antibody known to possess higher potency demonstrated that even a small peptide can enhance avidity and anti-viral potency similar to larger modular formats (FIG. 12). This data was confirmed in a focus reduction neutralization assay using both the now globally dominant D614G SARS-CoV-2 variant (FIG. 12A—upper panel) as well as the isogenic B.1.351 strain that contains 3 mutations in the RBD (K417N/E484K/N501Y) (FIG. 12A—lower panel). By comparing the neutralization of each variant at 4 concentrations of either parent IgG 15033-7 or a modified version of 15033-7 IgG with N-terminal peptide fusions on both the heavy and light chains revealed that the reduced effectiveness of the parent IgG against the B.1.351 variant could be overcome such that both variants were equally neutralized by the peptide-fused IgG. To demonstrate that this is a general strategy for enhancing potency and overcoming escape mutations that can be used for antibody that targets a similar epitope, we fused the same peptides to the heavy chain of an antibody isolated from B cells of SARS-CoV-2-infect patients that targets a similar epitope on the SARS-CoV-2 RBD (REGN 10933 IgG) (Hansen, PMID: 32540901) and fused either the p16 or p102 peptide to its heavy chain. Peptide fusions of REGN 10933 were compared to the parent REGN 10993 IgG in authentic virus neutralization assays against both the D614G (FIG. 12B—upper panel) and B.1.351 (FIG. 12B—lower panel) variants as above and were observed to enhance both potency and resistance to mutational escape.

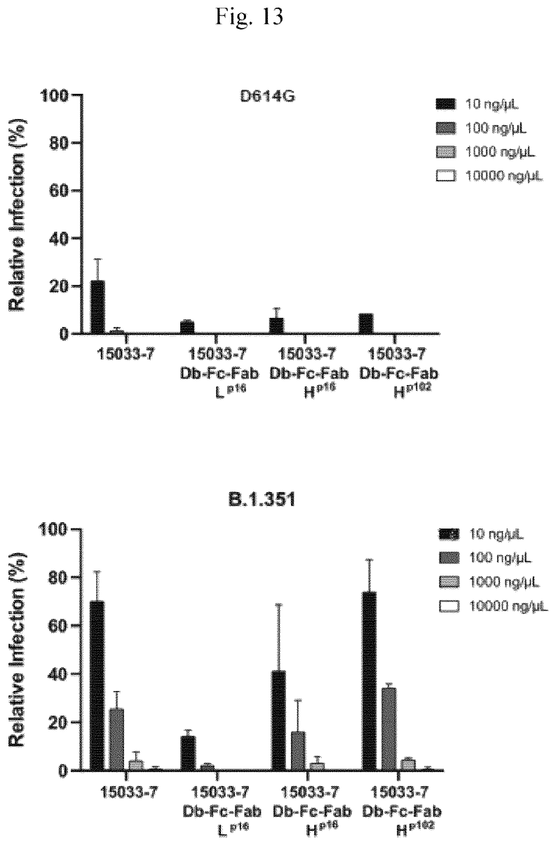

Results demonstrated that even a single peptide fused to the N-terminus of the antibody was capable of enhancing both potency and dramatically overcoming resistance to antibody-mediated neutralization conferred by mutation in the B.1.351 variant (FIG. 13). This strategy could also be extended to tetravalent formats, and peptides fused to the N-terminus of either the tetravalent 15033-7 db-Fc-Fab or Fab-db-Fc again dramatically enhanced resistance to mutational escape observed in the parent db-Fc-Fab 15033-7.

Peptides selected from peptide-phage libraries are fused to the N-terminus of the heavy (H) and/or light (L) chain of IgG-Fab via molecular cloning of genes encoding the peptides in to constructs for expression of the antibody (FIG. 14). Antibody CDRs encode specificity for the SARS-CoV-2 S protein. The parent IgG-Fab is shown in (A). Fusion of an unconstrained peptide (x) is shown (B), whereas a disulphide constrained peptide fusion (y) is shown in (C). Section (D) illustrates fusion of both constrained and unconstrained peptides to an IgG-Fab.

Peptides selected from peptide-phage libraries are fused to the N-terminus of the heavy (H) and/or light (L) chain of Fab-IgG via molecular cloning of genes encoding the peptides in to constructs for expression of the antibody (FIG. 15). Antibody CDRs encode specificity for the SARS-CoV-2 S protein. The parent Fab-IgG is shown in (A). Fusion of an unconstrained peptide (x) is shown (B), whereas a disulphide constrained peptide fusion (y) is shown in (C). Section (D) illustrates fusion of both constrained and unconstrained peptides to a Fab-IgG.

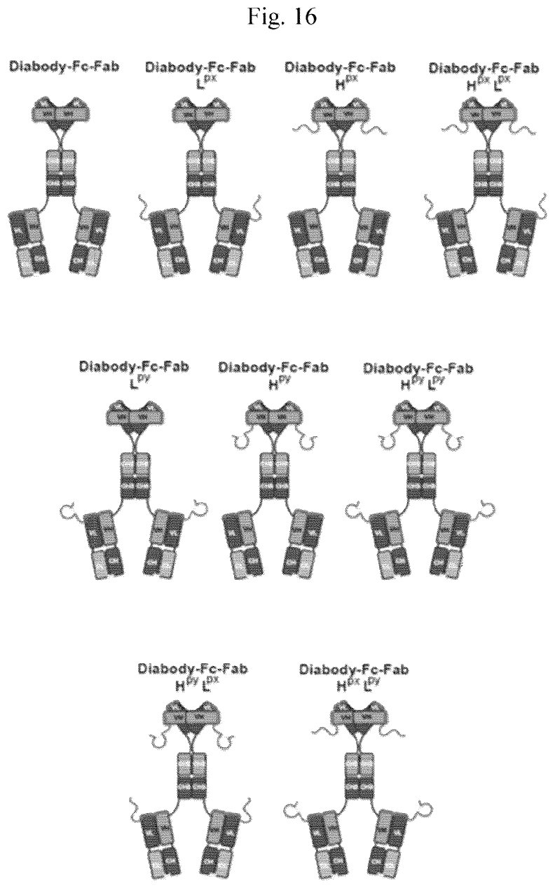

Peptides selected from peptide-phage libraries are fused to the N-terminus of the heavy (H) and/or light (L) chain of diabody-Fc-Fab via molecular cloning of genes encoding the peptides in to constructs for expression of the antibody (FIG. 16). Antibody CDRs (in red) encode specificity for the SARS-CoV-2 S protein. The parent diabody-Fc-Fab is shown in (A). Fusion of an unconstrained peptide (x) is shown (B), whereas a disulphide constrained peptide fusion (y) is shown in (C). Section (D) illustrates fusion of both constrained and unconstrained peptides to a diabody-Fc-Fab.

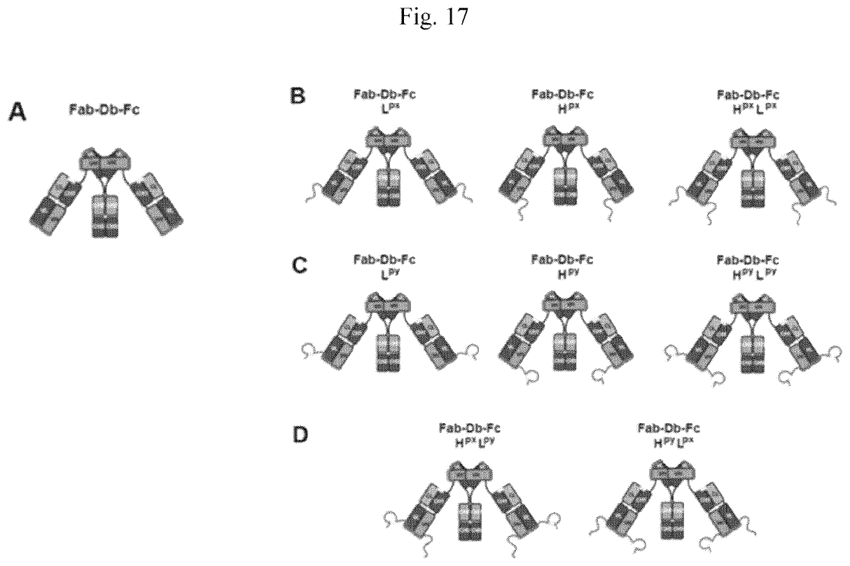

Peptides selected from peptide-phage libraries are fused to the N-terminus of the heavy (H) and/or light (L) chain of Fab-diabody-Fc via molecular cloning of genes encoding the peptides in to constructs for expression of the antibody (FIG. 17). Antibody CDRs (in red) encode specificity for the SARS-CoV-2 S protein. The parent Fab-diabody-Fc is shown in (A). Fusion of an unconstrained peptide (x) is shown (B), whereas a disulphide constrained peptide fusion (y) is shown in (C). Section (D) illustrates fusion of both constrained and unconstrained peptides to a Fab-diabody-Fc.

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

### C. Antibodies and/or Binding Fragments Thereof

The present disclosure relates to an antibody and/or binding fragment thereof as well as methods of making and use for example for diagnosing and/or prognosing COVID-19 infection and/or proliferation.

Described herein are antibodies and antibody binding fragments that specifically bind COVID-19 or a COVID-19 S polypeptide, e.g., S protein subunits S1 and S2. The antibodies described herein exhibited neutralization potencies at sub-nanomolar concentrations against SARS-CoV-2/USA/WA1 in Vero E6 cells, and also bound to the receptor binding domain (RBD).

Accordingly, an embodiment of this invention is an antibody and/or binding fragment thereof that specifically binds COVID-19 or a COVID-19 S polypeptide, e.g., S protein subunits S1 or S2.

In some embodiments, the heavy chain CDRs of the antibodies and antigen-binding fragments of this invention may be at least 50%, at least 55%, at least 60%, at least 75%, at least 80%, at least 85%, at least 90%, at least 95%, at least 96%, at least 97%, at least 98% or at least 99% identical to the amino acid sequence of the heavy chain (hIg1) CDRs of an antibody of Table 2. In some embodiments, the antibodies and antigen-binding fragments thereof contain a heavy chain variable region having a CDR amino acid sequence at least 90%, at least 91%, at least 92%, at least 93%, at least 94%, at least 95%, at least 96%, at least 97% 98%, or at least 99% identical to the amino acid sequence of the heavy chain (hIg1) CDRs of an antibody of Table 2. In some embodiments, the amino acid sequence of the heavy chain CDRs of the antibodies and antigen-binding fragments of this invention consist essentially of the amino acid sequence of the heavy chain (hIg1) CDRs of an antibody of Table 2.

In some embodiments, the light chain CDRs of the antibodies and antigen-binding fragments of this invention may be at least 50%, at least 55%, at least 60%, at least 75%, at least 80%, at least 85%, at least 90%, at least 95%, at least 96%, at least 97%, at least 98% or at least 99% identical to the amino acid sequence of the light chain (hk) CDRs of an antibody of Table 2. In some embodiments, the antibodies and antigen-binding fragments thereof contain a light chain variable region having an amino acid sequence at least 90%, at least 91%, at least 92%, at least 93%, at least 94%, at least 95%, at least 96%, at least 97%, at least 98%, at least 99% identical to the amino acid sequence of the light chain (hk) CDRs of an antibody in Table 2. In some embodiments, the amino acid sequence of the heavy chain CDRs of the antibodies and antigen-binding fragments of this invention consist essentially of the amino acid sequence of light chain (hk) CDRs of an antibody of Table 2.

In some embodiments, the antibodies and antigen-binding fragments thereof comprise (a) a VH domain comprising three heavy chain variable region CDRs, the CDRs having an amino acid sequence at least 90%/o, at least 91%, at least 92%, at least 93%, at least 94%, 95%, at least 96%, at least 97%, at least 98%, at least 99% identical to the amino acid sequence of three heavy chain (hIg1) CDRs of an antibody of Table 2, and (b) a VL domain comprising three light chain CDRs having an amino acid sequence at least 90%, at least 91%, at least 92%, at least 93%, at least 94%, at least 95%, at least 96%, at least 97%, at least 98%, at least 99% or more identical to the amino acid sequence of the light chain (hk) CDRs of an antibody in Table 2.

In some embodiments, the antibodies and antigen-binding fragments thereof comprise (a) a VH domain comprising three heavy chain variable region CDRs, the CDRs having an amino acid sequence of three heavy chain (hIg1) CDRs of an antibody of Table 2, and/or (b) a VL domain comprising three light chain CDRs having an amino acid sequence of the light chain (hk) CDRs of an antibody in Table 2.

In an embodiment of the invention the antibody comprises an amino acid sequence at least 90%/o, at least 91%, at least 92%, at least 93%, at least 94%, at least 95%, at least 96%, at least 97%, at least 98%, at least 99% identical to the amino acid sequence of a molecule of Table 4.

In an embodiment of the invention the antibody comprises an amino acid sequence of the molecules of Table 4. In an embodiment of the invention the antibody consists essentially of an amino acid sequence of the molecules of Table 4. Although the amino acid and nucleotide sequences of the molecules in Table 4 comprise the heavy chain and light chain CDRs of antibody 15031, embodiments of this invention include the amino acid and nucleotide of the molecules in Table 4 wherein the CDRs of 15031 are replaced with the CDRs of the antibodies set forth in Tables 2 and 3, for example, 15032, 15033, 15033-7, 15034, 15046, and 15049.

A further aspect is an antibody and/or binding fragment of this invention, wherein the antibody and/or binding fragment thereof specifically binds to COVID-19 or a COVID-19 S polypeptide in an unfixed or mildly fixed sample.

In an embodiment, the antibody and/or binding fragment thereof comprises a VL domain comprising a CDR3 and a VH domain comprising a CDR1, a CDR2 and a CDR3, wherein the amino acid sequences of the CDRs comprise one or more of the CDR sequences set forth in Table 2.

In an embodiment, the antibody and/or binding fragment thereof comprises a light chain variable region comprising an hk CDR3 of Table 2 and the heavy chain variable region comprises a hIg1 CDR1, a CDR2 and a CDR3 of an antibody set forth in Table 2.

In an embodiment, the antibody, binding fragment thereof, or CDRs disclosed herein may have one or more amino acid substitutions, including but not limited to conservative amino acid substitutions.

The antibody comprising the light chain and heavy chain CDRs set forth in Table 2 can be any class of immunoglobulins including: IgM, IgG, IgD, IgA or IgE; and any isotype, including: IgG1, IgG₂, IgG₃ and IgG₄.

Chimeric antibodies can be prepared using routine recombinant techniques. For example, a Fab of an IgG of Table 4 may be reformatted into another full length IgG by subcloning the variable domains of the immunoglobulin's light and heavy chains into mammalian expression vectors and producing the IgG protein using human embryonic kidney cells (HEK293T). As described elsewhere any cell type suitable for expressing an antibody can be used.

In one embodiment, the antibody may be a full-length immunoglobulin molecule, e.g., a chimeric antibody, a human antibody, a humanized antibody, a polyclonal antibody, or a minibody, and the antibody binding fragment may be a Fab, a Fab′, a F(ab)₂, a F(ab′)₂, a Fv, a disulfide linked Fv, a scFv, a disulfide linked scFv, a single chain domain antibody scFab or VHH, or a diabody, that does not comprise an Fc domain or fragment thereof.

The antigen binding fragments, e.g., Fab, Fab′ and F(ab′)₂, scFv, scFab, dsFv, ds-scFv, VHH, or diabodies, and other fragments can be synthesized or expressed by recombinant techniques. Antibodies can also be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments.

In an embodiment, the antibody is a synthetic monoclonal antibody or a chimeric antibody comprising one or more CDRs selected from the CDRs of Table 2. In an embodiment, the antibody is a chimeric antibody comprising a VL domain comprising the hk CDR1, CDR2 and CDR3 from an antibody of Table 2 and/or a VH domain comprising the hIg1 CDR1, CDR2 and CDR3 from an antibody of Table 2.

In an embodiment, the COVID-19 binding molecule is a VHH fragment, a scFv, an Fab or a diabody that comprises the hIg1 CDR1, CDR2 and CDR3 and/or the hk CDR1, CDR2 or CDR3 from an antibody of Table 2.

The antibodies and antibody fragments described herein may be isolated antibodies and antibody fragments.

In an embodiment of the full-length antibodies of this invention the Fc domain of the antibody is engineered such that it does not target the cell that binds the antibody for ADCC or CDC-dependent death. In an embodiment of the invention the Fc domain is dimerized via a knob-in-hole configuration. Methods are well known in the art for mitigating antibody effector function, including for example amino acid substitutions in the Fc regions, e.g., the N297G (NG) and D265A, N297G (DANG) variants or L234A, L235A, P329G (LALA-PG) substitutions, see e.g., Lo et al. “Effector Attenuating Substitutions that Maintain Antibody Stability and Reduce Toxicity in Mice. The Journal of Biological Chemistry Vol. 292, No. 9, pp. 3900-3908, Mar. 3, 2017, incorporated herein by reference.

In an embodiment of this invention the antibody and/or binding fragment thereof is linked, e.g., conjugated or recombinantly fused, to a therapeutic moiety, e.g., a cytotoxin, a chemotherapeutic drug, an immunosuppressant or a radioactive metal ion.

In a further embodiment, the antibody and/or binding fragment is labelled and/or conjugated or fused to a tag, for example to produce a detection tool or a diagnostic agent. For example, the detectable tag can be a purification tag such as a His-tag, a HA-tag, a GST-tag, biotin or a FLAG-tag.

The label is preferably capable of producing, either directly or indirectly, a detectable signal. For example, the label may be radio-opaque or a radioisotope, such as ³H, ¹⁴C; ³²P; ³⁵S; ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase (HRP); an imaging agent; or a metal ion.

Another aspect of the disclosure relates to an antibody complex comprising the antibody and/or binding fragment described herein and COVID-19 or a COVID-19 S polypeptide.

Yet another aspect of this invention is a nucleic acid encoding an antibody and/or binding fragment described herein. In an embodiment, the nucleic acid encodes an antibody and/or binding fragment comprising a light chain variable region comprising a CDR1, CDR2 and CDR3 and a heavy chain variable region comprising a CDR1 CDR2 and CDR3, wherein one or more of the CDRs are the light chain and heavy chain CDRs set forth in Table 2.

The degeneracy of the genetic code allows for different nucleic acids to encode the same amino acid sequence. Accordingly, also included are nucleotide sequences that encode an antibody disclosed herein that specifically binds COVID-19 or a COVID-19 S polypeptide.

Also included in another embodiment are codon degenerate or optimized sequences. In another embodiment, the nucleic acid sequences are at least 70%, at least 75%, at least 80%, at least 90% and at least 95% identical to nucleic acid sequences of Table 3 or Table 4 encoding the molecules set forth in Table 2 or Table 4.

In an embodiment of this invention, the nucleic acid is an isolated nucleic acid.

Another aspect of this invention is a vector comprising the nucleic acid herein disclosed. In an embodiment, the vector is an isolated vector.

The vector can be any vector suitable for producing an antibody and/or binding fragment thereof, including for example vectors described herein. Possible expression vectors include but are not limited to cosmids, plasmids, or modified viruses (e.g. replication defective retroviruses, adenoviruses and adeno-associated viruses).

A further aspect of this invention is a recombinant cell producing the antibody and/or binding fragment thereof herein disclosed or the vector herein disclosed.

The recombinant cell can be generated using any cell suitable for producing a polypeptide, for example suitable for producing an antibody and/or binding fragment thereof.

Suitable host cells include a wide variety of prokaryotic and eukaryotic host cells. For example, the proteins of the invention may be expressed in bacterial cells such as E. coli, insect cells (using baculovirus), yeast cells or mammalian cells.

More particularly, bacterial host cells suitable for producing recombinant antibody producing cells include E. coli, B. subtilis, Salmonella typhimurium, and various species within the genus Pseudomonas, Streptomyces, and Staphylococcus, as well as many other bacterial species well known to one of ordinary skill in the art. Suitable bacterial expression vectors preferably comprise a promoter which functions in the host cell, one or more selectable phenotypic markers, and a bacterial origin of replication. Representative promoters include the R-lactamase (penicillinase) and lactose promoter system, the trp promoter and the tac promoter. Representative selectable markers include various antibiotic resistance markers such as the kanamycin or ampicillin resistance genes. Suitable expression vectors include but are not limited to bacteriophages such as lambda derivatives or plasmids such as pBR322, the pUC plasmids pUC18, pUC19, pUC118, pUC119, and pNH8A, pNH16a, pNH18a, and Bluescript M13 (Stratagene, La Jolla, Calif.).

Suitable yeast and fungi host cells include, but are not limited to Saccharomyces cerevisiae, Schizosaccharomyces pombe, the genera Pichia or Kluyveromyces and various species of the genus Aspergillus. Examples of vectors for expression in yeast S. cerevisiae include pYepSecl, pMFa, pJRY88, and pYES2 (Invitrogen Corporation, San Diego, Calif.). Protocols for the transformation of yeast and fungi are well known to those of ordinary skill in the art.

Suitable mammalian cells include, among others: COS (e.g., ATCC No. CRL 1650 or 1651), BHK (e.g. ATCC No. CRL 6281), CHO (ATCC No. CCL 61), HeLa (e.g., ATCC No. CCL 2), 293 (ATCC No. 1573), NS-1 cells and any derivatives of these lines.

In an embodiment, the mammalian cells used to produce a recombinant antibody are selected from CHO, HEK293 cells or FREESTYLE™ 293-F cells (Life technologies). FREESTYLE 293-F cell line is derived from the 293 cell line and can be used with the FREESTYLE™ MAX 293 Expression System, FREESTYLE™ 293 Expression System or other expression systems.

Suitable expression vectors for directing expression in mammalian cells generally include a promoter (e.g., derived from viral material such as polyoma, Adenovirus 2, cytomegalovirus and Simian Virus 40), as well as other transcriptional and translational control sequences.

In an embodiment, the vector is designed for production of a light chain or a heavy chain, e.g., an IgG1 heavy chain.

Suitable insect cells include cells and cell lines from Bombyx or Spodotera species. Baculovirus vectors available for expression of proteins in cultured insect cells (SF 9 cells) include the pAc series and the pVL series.

The recombinant expression vectors may also contain genes which encode a fusion moiety (i.e. a “fusion protein”) which provides increased expression or stability of the recombinant peptide; increased solubility of the recombinant peptide; and aid in the purification of the target recombinant peptide by acting as a ligand in affinity purification, including for example tags and labels described herein. Further, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

“Operatively linked” is intended to mean that the nucleic acid is linked to regulatory sequences in a manner which allows expression of the nucleic acid. Suitable regulatory sequences may be derived from a variety of sources, including bacterial, fungal, viral, mammalian, or insect genes. Selection of appropriate regulatory sequences is dependent on the host cell chosen and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

In an embodiment, expression of the antibody or binding fragment thereof is under the control of an inducible promoter. Examples of inducible non-fusion expression vectors include pTrc (28) and pET 11d.

The recombinant expression vectors may also contain a marker gene which facilitates the selection of host cells transformed or transfected with a recombinant molecule of the invention. Examples of selectable marker genes are genes encoding a protein such as G418 and hygromycin which confer resistance to certain drugs, β-galactosidase, chloramphenicol acetyltransferase, or firefly luciferase. Transcription of the selectable marker gene is monitored by changes in the concentration of the selectable marker protein suchas—galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of recombinant expression vectors of the invention and in particular to determine the effect of a mutation on expression and phenotype. It will be appreciated that selectable markers can be introduced on a separate vector from the nucleic acid of interest. Other selectable markers include fluorescent proteins such as GFP which may be cotransduced with the nucleic acid of interest.

Yet another aspect is a composition, e.g., a pharmaceutical comprising the antibody and/or binding fragment thereof, the nucleic acid herein disclosed or the recombinant cell herein disclosed, optionally in combination with a suitable diluent or carrier or excipient. The pharmaceutical composition may consist or consist essentially of the antibodies or binding fragment or the nucleic acid molecules expression cassettes and vectors encoding the antibodies or binding fragment described herein, and a pharmaceutically acceptable diluent, carrier or excipient. Suitable carriers, diluents and excipients, and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the agonist, which matrices are in the form of shaped articles, e.g., films, liposomes or microparticles. Delivery systems for administering therapeutic agents, e.g., antibodies and fragments thereof, are known in the art, see e.g., Wu and Wu, J. Biol. Chem. 262:4429-4432 (1987)), Langer, 1990, Science 249:1527-1533; Sefton, 1987, CRC Crit. Ref. Biomed. Eng. 14:20; Buchwald et al., 1980, Surgery 88:507; Saudek et al., 1989, N. Engl. J. Med. 321:574; Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; PCT Publication No. WO 99/15154; and PCT Publication No. WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides, poly(lactide-co-glycolides) (PLGA), and polyorthoesters. In one embodiment, the polymer used in a sustained release formulation is inert, free of leachable impurities, stable on storage, sterile, and biodegradable. It will be apparent to those persons skilled in the art that certain carriers may be more preferable depending upon, for instance, the route of administration and concentration of the antibodies or binding fragment being administered.

The composition can be a lyophilized powder or aqueous or non-aqueous solution or suspensions, which may further contain antioxidants, buffers, bacteriostats and solutes. Other components that may be present in such compositions include, e.g., water, surfactants (such as TWEEN™), alcohols, polyols, glycerin and vegetable oils.

Suitable diluents for nucleic acids include but are not limited to water, saline solutions and ethanol. Suitable diluents for polypeptides, including antibodies or fragments thereof and/or cells include but are not limited to saline solutions, pH buffered solutions and glycerol solutions or other solutions suitable for freezing polypeptides and/or cells.

The composition can further comprise stabilizing agents, for example reducing agents, hydrophobic additives, and protease inhibitors which are added to physiological buffers.

### Methods

Additional aspects of the invention are methods for using, producing or isolating an antibody and/or binding fragment thereof described herein with specific binding affinity to COVID-19 or a COVID-19 S polypeptide.

It has been reported the symptoms of COVID-19 infections include, e.g., fever, cough, shortness of breath, chills, repeated shaking with chills, muscle pain, headache, sore throat, loss of taste or smell, and can lead to physical complications, e.g., pneumonia and trouble breathing, organ failure in several organs, heart problems, severe lung condition, e.g., acute respiratory distress syndrome, blood clots, acute kidney injury and additional viral and bacterial infections. An embodiment of the invention relates to a method for treating, preventing, mitigating, or delaying the progression of a COVID-19 infection, or ameliorating a symptom or complication of a COVID-19 infection, comprising administering an effective amount of an antibody or binding fragment of this invention to a subject having or suspected of having a COVID-19 infection.

A method for treating, preventing, mitigating, or delaying the progression of a COVID-19 infection includes reducing viral load in a subject in need thereof. The antibody or binding fragment of the present invention may be administered systemically or locally, e.g., by injection (e.g. subcutaneous, intravenous, intraperitoneal, intrathecal, intraocular, etc.), inhalation, implantation, topically, or orally. Depending on the route of administration, the antibody or binding fragment may be coated in a material to protect the molecules from the action of acids and other natural conditions which may inactivate the molecules. The antibody or binding fragment described herein may be dissolved or suspended in a pharmaceutically acceptable, preferably aqueous carrier. In addition, the composition comprising the binding molecule can contain excipients, such as buffers, binding agents, blasting agents, diluents, flavors, lubricants, etc. An extensive listing of excipients that can be used in such a composition, can be, for example, taken from A. Kibbe, Handbook of Pharmaceutical Excipients (Kibbe, 2000). The antibody or binding fragment of this invention can also be administered in conjunction with other therapeutic regimens or agents for treatment of COVID-19 infections and the associated symptoms or complications, e.g., convalescent serum therapy, small molecules, oleandrin, neutralizing monoclonal antibodies, recombinant antibodies, cytokines, or antivirals, e.g., LY-CoV555 (Eli Lilly), e.g., recombinant anti-CD47/PD-L1 bispecific antibody (IBI322)(Innovent Biologics, Inc.), remdesivir, Actemra, Oluminant (baricitinib), merimepodib, dexamethaone (glucocorticoid), Aviptadil (RLF-100), hydroxycloropuine, Avigan (favipiravir, Avifavir), Bucillamine, niclosamide, aviptadil, PlKfyve kinase inhibitor, tyrosine kinase inhibitor efineptakin alfa, interferon beta, or SLV213 (Selva Therapeutics).

Effective dosages and schedules for administering the antibodies and fragments thereof of this invention and nucleic acids that encode them described herein may be determined empirically, and making such determinations is within the skill in the art. Those skilled in the art will understand that the dosage of such antibodies and fragments thereof that must be administered will vary depending on, for example, the subject that will receive the binding molecule, the route of administration, the particular type of antibody or fragment thereof used and other drugs being administered. Guidance in selecting appropriate doses for antibodies and fragments thereof of this invention is found in the literature on therapeutic uses of antibodies, e.g., Handbook of Monoclonal Antibodies, Ferrone, eds., Noges Publications, Park Ridge, N.J., (1985) ch. 22 and pp. 303-357; Smith, Antibodies in Human Diagnosis and Therapy, Haber, eds., Raven Press, New York (1977) pp. 365-389. The dosage ranges for the administration of the compositions are those large enough to produce the desired effect. The dosage should not be so large as to cause adverse side effects, such as unwanted cross-reactions, anaphylactic reactions, and the like. Generally, the dosage will vary with the age, condition, sex and extent of the inflammation in the patient and can be determined by one of skill in the art. The dosage can be adjusted by the individual physician in the event of any contraindications. Dosage can vary, and can be administered in one or more dose administrations daily, for one or several days. While individual needs vary, determination of optimal ranges of effective amounts of the antibody, fragments thereof, nucleic acid and vector is within the skill of the art.

An embodiment of the invention relates to a method for preventing or delaying the cleavage of the S2 subunit, or the fusion of COVID-19 with the cell and internalization of COVID-19 by a cell, e.g., a cell having an ACE2 receptor, by contacting the cell with an effective amount of an antibody or antibody binding fragment of this invention.

Yet another aspect relates to a method for screening or for diagnosing a subject having or suspected of having a COVID-19 infection, the method comprising measuring the level of COVID-19 or a COVID-19 S polypeptide in a sample from a subject by contacting the sample with an antibody or binding fragment of this invention, wherein detecting a level of COVID-19 or a COVID-19 S polypeptide bound by the antibody or binding fragment is indicative that the subject is infected with COVID-19. The method may further comprise the step of comparing the level of COVID-19 or a COVID-19 S polypeptide in the sample bound by the antibody or binding fragment with a negative or positive control, wherein an increased level of COVID-19 or a COVID-19 S polypeptide in the sample bound by the antibody or binding fragment as compared to a negative control, e.g., a value derived from a group of subjects without COVID-19 infection, or convalescing from a COVID-19 infection, is indicative that the subject is infected with COVID-19 or wherein a level of COVID-19 similar to or exceeding the level of COVID-19 of the positive control, e.g., a value derived from a group of subjects known to have a COVID-19 infection, is indicative that the subject is infected with COVID-19.

An additional aspect of the invention is a method for prognosing COVID-19 progression or lack thereof (e.g. recovery or worsening of disease). Accordingly an aspect is a method of prognosing a likelihood of recovery after COVID-19 infection, the method comprising: a) measuring a level of COVID-19 or a COVID-19 S polypeptide in a sample from a subject by contacting the sample with the antibodies or antibody fragments described herein; and b) comparing the level of COVID-19 or a COVID-19 S polypeptide bound by the antibodies or fragments thereof in the sample with a control, for example a control value derived from a group of subjects who recovered from or in whom the COVID-19 disease symptoms regressed, wherein a decreased level of COVID-19 in the sample bound by the antibodies or binding fragments compared to the control is indicative that the subject has an increased likelihood of recovery after COVID-19 infection. The level of COVID 19 may be measured by, e.g., measuring the amount of COVID-19 or a COVID-19 S polypeptide bound to the antibodies or antibody fragments of this invention by e.g., immunoprecipitation, an immunoassay, e.g. ELISA, immunoblot detection. The antibody-based detection can also be combined with a mass spectrophometric assay, for example as in the case of a particle-based flow cytometric assay.

In an embodiment, the control is a control value derived from a group of subjects recovered from COVID-19 infection and an increased level of COVID-19 or a COVID-19 S polypeptide in the sample compared to the control is indicative that the subject has a decreased likelihood of recovery after COVID-19 infection and/or an increased likelihood of COVID-19 infection and associated adverse effects progressing.

A further embodiment of the invention is a method for prognosing the likelihood of progression of a COVID-19 infection and associated adverse effects, the method comprising: a) measuring a level of COVID-19 or a COVID-19 S polypeptide in a sample from a subject by contacting the sample with the antibodies or antibody fragments described herein; and b) comparing the level of COVID-19 or a COVID-19 S polypeptide in the sample bound to the antibodies or antibody fragments described herein with a control, wherein an increased level of COVID-19 or a COVID-19 S polypeptide in the sample compared to the control, for example wherein the control is a control value derived from a group of subjects who did not recover or progressed is indicative that the subject has an decreased likelihood of recovering from a COVID-19 infection.

In an embodiment, the control is a control value derived from a group of subjects who recovered and an increased level of COVID-19 or a COVID-19 S polypeptide in the sample compared to the control is indicative that the subject has a decreased likelihood of recovery after COVID-19 and/or an increased likelihood of COVID-19 infection and associated adverse effects progressing.

The sample can for example be taken after the subject has received a treatment and compared for example to a pre-treatment sample. Alternatively, the patient can be monitored after a repeating interval to assess for example if treatment or other intervention is necessary. In an embodiment, the test is repeated and plotted to assess the subject's progression.

The sample may be a tissue sample, e.g. lung tissue, or a biological fluid such as blood, or a fraction thereof such as plasma or serum, or a lung secretion or a pulmonary lavage. In an embodiment the biological fluid is saliva or a nasal secretion.

In another embodiment, the sample is selected from a fresh sample such as a fresh (e.g. not frozen) biological fluid sample or tissue sample, a onetime frozen biological fluid sample or tissue sample (e.g. frozen a single time at the time of obtaining the sample)) or a repeat frozen sample (e.g. frozen and thawed and frozen biological fluid sample or repeat frozen tissue sample). In an embodiment, the sample comprises native or natively folded COVID-19 polypeptide. In an embodiment, the sample is a fixed sample such as a mildly fixed sample wherein the fixation induces limited denaturation and/or unfolding.

In another embodiment, the isolated and purified antibody and/or binding fragment thereof is affinity matured. Affinity maturation can be performed as described for the initial selection, with antigen adsorbed to plastic plates, using a for example a phage library comprising variants of the CDR sequences.

A person skilled in the art will appreciate that several methods can be used to isolate and produce antibodies and/or binding fragments thereof with specific binding affinity to COVID-19 or a COVID-19 polypeptide, e.g. COVID-19 S polypeptide. A method that can be used is a phage display method. For example, COVID-19 or a COVID-19 S polypeptide is produced in order to isolate and characterize the antibody and/or binding fragment thereof. Phage from a human Fab phage-displayed library are selected following several rounds of panning. Phage with specific binding affinity to the COVID-19 or a COVID-19 S polypeptide, as determined by ELISA, are sequenced and cloned into vectors designed for production of light chain or heavy chain. The heavy chain can be for example an IgG, or an IgG isotype such as an IgG1 or an IgG4. Antigen binding fragments and IgG polypeptides are then affinity purified by using, for example, Protein A affinity columns.

In another embodiment, a nucleic acid encoding an antibody or antibody binding fragment described herein is expressed in a host cell to make the antibody and/or binding fragment. In an embodiment, the method comprises: a) expressing in a host cell a nucleic acid encoding an antibody and/or binding fragment; b) culturing the host cell to produce the antibody and/or binding fragment; and c) isolating and/or purifying the antibody and/or binding fragment from the host cell.

In some embodiments, a nucleic acid encoding a single chain antibody is expressed. In other embodiments, multiple nucleic acids are expressed, for example a nucleic acid encoding an antibody light chain and a nucleic acid encoding an antibody heavy chain.

Suitable host cells and vectors are described above. Vectors and nucleic acids encoding an antibody or antibody biding fragment described herein may be introduced into mammalian cells via conventional techniques such as calcium phosphate or calcium chloride co-precipitation, DEAE-dextran mediated transfection, lipofection and other liposome-based transfection agents, electroporation or microinjection.

Nucleic acid encoding an antibody or antibody biding fragment described herein may be directly introduced into mammalian cells using delivery vehicles such as retroviral vectors, adenoviral vectors and DNA virus vectors.

### Assays

The COVID-19 or COVID-19 S polypeptide specific antibodies disclosed herein can be used in a variety of assays for binding, detecting and measuring COVID-19 or a COVID-19 S polypeptide in a sample. For example, the antibodies can be used in an ELISA, as well as immunoprecipitation, immunoblot, immunohistochemistry or immunocytochemistry, proximity ligation assay (PLA) (Muhammad S. Alam, November 2018) Current Protocols in Immunology Vol 123, Issue 1., mass spectroscopy-based techniques, particle-based flow cytometric assay, fluorescence-activated cell sorting (FACS) and ELISA.

Immunodetection methods as described herein generally involve the detection or measuring of antibody:COVID-19 or a COVID-19 S polypeptide, e.g. an S1 of S2 subunit, complexes using antibodies and/or binding fragments thereof disclosed herein. The detection of such complexes is well known in the art and may be achieved through different methods, for example by using a detectable label or marker, such as a radioactive, fluorescent or enzymatic tag. Detection of these complexes may also involve the use of a ligand such as a secondary antibody and/or binding fragment thereof specific for COVID-19 or a COVID-19 S polypeptide or for the antibody:COVID-19 or a COVID-19 S polypeptide complex.

They can also be used to make detection assays, for example a sandwich ELISA, as one antibody can be used as a capture reagent to isolate the COVID-19 or a COVID-19 S polypeptide while another antibody binding a distinct epitope can be used as a detection reagent.

Accordingly, another aspect is an immunoassay comprising one or more antibodies and/or binding fragments thereof described herein.

In an embodiment, the immunoassay is an enzyme linked immunosorbent assay (ELISA). Antibodies and/or binding fragments thereof may be used in the context of detection assays such as ELISAs, for example sandwich ELISAs. The antibodies and binding fragments of this invention may be used as capture and detection antibodies for the detection of COVID-19 or a COVID-19 S polypeptide in solution. For example, the full-length antibodies or binding fragments of this invention that recognize the S protein, e.g., the S1 subunit, are immobilized and incubated with COVID-19 or a COVID-19 S polypeptide or subunit. The samples are then incubated with biotinylated IgGs recognizing epitopes on the virus, polypeptide, or subunit.

In an embodiment, the ELISA is a sandwich ELISA comprising a capture antibody and a detection antibody that bind to different epitopes. For example, the capture antibody is an antibody or binding fragment which specifically binds a first epitope of COVID-19 or a COVID-19 S polypeptide, e.g., a S1 subunit or the RBD, and/or the detection antibody is an antibody or binding fragment which specifically binds a second epitope of the COVID-19 or a COVID-19 S polypeptide, e.g., the S1 subunit or the RBD, wherein the first and second epitopes are different. In an embodiment, one or both of the capture or detection antibodies is an antibody and/or binding fragment thereof herein.

In one embodiment, the immunoassay is for the detection and/or measuring of COVID-19 or a COVID-19 S polypeptide, e.g., the S1 or S2 subunit, in a sample, wherein the method of making the immunoassay comprises: a) coating a solid support with the capture antibody; b) contacting the capture antibody with the sample under conditions to form a capture antibody:COVID-19 or a COVID-19 S polypeptide complex; c) removing unbound sample; d) contacting the capture antibody:COVID-19 or a COVID-19 S polypeptide complex with the detection antibody; e) removing unbound detection antibody; and f) detecting and/or measuring the capture antibody:COVID-19 or a COVID-19 S polypeptide complex, wherein one or both of the capture or detection antibodies is an antibody and/or binding fragment thereof herein.

In an embodiment, the ELISA is a competitive ELISA. In an embodiment, the ELISA is a direct ELISA. In an embodiment, the ELISA is an indirect ELISA.

As used herein, “solid supports” include any material to which COVID-19 or a COVID-19 S polypeptide and antibodies and/or binding fragments thereof herein disclosed are capable of binding to. For example, the solid support can include plastic, glass, polystyrene, nylon, polypropylene, nylon, polyethylene, dextran, amylases, natural and modified celluloses and polyacrylamides. For example, the solid support is a microtiter plate, magnetic beads, latex beads or array surfaces.

In embodiments of the immunoassays described herein, the sample may be contacted with an antibody and/or binding fragment thereof under appropriate conditions, for example, at a given temperature and for a sufficient period of time, to allow effective binding of COVID-19 or a COVID-19 S polypeptide to the antibody or binding fragment, thus forming an antibody:COVID-19 or a COVID-19 S polypeptide complex, such as a capture antibody:COVID-19 or a COVID-19 S polypeptide complex. For example, the contacting step is carried out at room temperature for about 30 minutes, about 60 minutes, about 2 hours or about 4 hours. For example, the contacting step is carried out at about 4° C. overnight.

The antibody and/or binding fragment as described herein may be, e.g., complexed with COVID-19 or a COVID-19 S polypeptide in a suitable buffer. For example, the buffer has a pH of about 5.0 to about 10.0. For example, the buffer has a pH of 4.5, 6.5 or 7.4. For example, the buffer is a HBS-EP buffer, a KRH buffer or Tris-buffered saline. For example, the buffer comprises BSA and/or TWEEN™ 20.

Any unbound material in a sample may be removed by washing so that only the formed antibody:COVID-19 or a COVID-19 S polypeptide complex remains on the solid support. For example, the unbound sample is washed with phosphate-buffered saline, optionally comprising bovine serum albumin (BSA).

In an embodiment, the detection antibody is labelled and/or conjugated to a tag.

For example, the detection antibody directly labelled and/or conjugated. For example, the detection antibody is indirectly labelled and/or conjugated. Indirect labels include for example fluorescent or chemiluminescent tags, metals, dyes or radionuclides attached to the antibody. Indirect labels include for example horseradish peroxidase, alkaline phosphatase (AP), beta-galactosidase and urease. For example, HRP can be used with a chromogenic substrate, for example tetramethybenzidine, which produces a soluble product in the presence of hydrogen peroxide that is detectable at 450 nm.

An embodiment of the invention is an assay for detecting and/or measuring level of COVID-19 or a COVID-19 S polypeptide in a sample, the assay comprising: a) contacting a sample with the antibody and/or binding fragment described herein under conditions to form an antibody:COVID-19 or a COVID-19 S polypeptide complex; and b) detecting and/or measuring the antibody:COVID-19 or a COVID-19 S polypeptide complex. A further embodiment is an assay for detecting and/or measuring COVID-19 or a COVID-19 S polypeptide the method comprising: a) contacting a sample, the sample being a biological fluid, with the antibody and/or binding fragment of the antibody as described herein under conditions to form an antibody:COVID-19 or a COVID-19 S polypeptide complex; and b) detecting and/or measuring the antibody:COVID-19 or a COVID-19 S polypeptide complex. The assay for detecting and/or measuring the level of COVID-19 or a COVID-19 S polypeptide is performed under non-denaturing or mildly denaturing conditions.

In an embodiment, the antibody:COVID-19 or a COVID-19 S polypeptide complex is detected directly for example by using an antibody labeled with a detectable tag or fusion moiety. In an embodiment, the complex is detected indirectly using a secondary antibody specific for the antibody:COVID-19 or a COVID-19 S polypeptide complex.

In an embodiment, the assay for detecting or measuring the level of COVID-19 or a COVID-19 S polypeptide is an immunoprecipitation, immunoblot, immunohistochemistry or immunocytochemistry, proximity ligation assay (PLA), mass spectroscopy-based techniques and fluorescence-activated cell sorting (FACS), and ELISA.

Detecting can be performed using methods that are qualitative or measured using quantitative methods, for example by comparing to a standard or standard curve.

The sample used in the methods described herein may be a biological sample from a subject, e.g., a human or other primate, or the sample may be from another source. The biological sample from a subject may be a tissue or a fluid, e.g., blood and its component parts, serum or plasma, saliva, a lung secretion or a lung lavage, or urine etc. Other samples may be, e.g., wastewater or a body of water where wastewater drains, e.g., a river, canal, pond, bay etc.

### Kits

A further aspect of this invention relates to a kit comprising i) an antibody and/or binding fragment described herein, ii) a nucleic acid encoding the an antibody and/or binding fragment described herein, iii) a composition comprising the an antibody and/or binding fragment described herein or the nucleic acid encoding the antibody or fragment, or iv) a recombinant cell described herein, comprised in a vial such as a sterile vial or other housing and optionally a reference agent, and/or instructions for use thereof.

In an embodiment, the kit comprises components and/or is for use in performing an assay described herein.

For example, the kit is an ELISA kit and can comprise a first antibody, e.g. a capture antibody, for example attached to a solid support, and a second antibody, e.g. a detection antibody, that binds to COVID-19 or a COVID-19 S polypeptide and/or binds the capture antibody: COVID-19 or a COVID-19 S polypeptide complex, and that is conjugated to a detectable label. The first and/or second antibody may be an antibody or antibody fragment of this invention. Any combination of antibodies or antibody fragments described herein can be used.

In an embodiment, the kit is a diagnostic kit and the instructions are directed to a method described herein.

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The following non-limiting examples are illustrative of the present disclosure:

### EXAMPLES

To identify antibodies that neutralize SARS-CoV-2 as a basis for therapeutic development, we conducted phage-display selections against the RBD of the S protein, isolated numerous clones that bound to the SARS-CoV-2 RBD or the full-length S protein ectodomain, and screened to identify those that blocked ACE2 binding. In these studies, a library containing 10¹⁰ Fab-phage clones was used for selections (Persson et al. (2013) CDR-H3 diversity is not required for antigen recognition by synthetic antibodies. J. Mol. Biol. 425, 803-811.). Among the high affinity Fab-phage clones, those that blocked ACE2 were selected for further characterization and expressed as full-length human IgG proteins with a framework engineered to possess high thermostability and low immunogenicity for therapeutic applications (Persson et al. (2013)). Several IgGs were found to exhibit sub-nanomolar affinities for the SARS-CoV-2 S protein and from this group antibody number 15033 emerged as a best neutralizer with an ability to neutralize SARS-CoV-2 with high potency.

### Expression and Purification of Antigens

The previously reported piggyBac transposase-based expression plasmid PB-T-PAF (Li et al., (2013) Simple piggyBac transposon-based mammalian cell expression system for inducible protein production. Proc. Natl. Acad. Sci. U.S.A. 110, 5004-5009) was modified to generate two new vectors, one containing a CMV promotor (PB-CMV) and the other a TRE promotor (PB-TRE). To facilitate nuclear export of mRNA, a woodchuck hepatitis virus posttranscriptional regulatory element (WPRE) was also added to each of these plasmids. Each of the protein ORFs was cloned into both expression vectors. The PB-CMV version was used for large-scale transient expression. The PB-TRE version was used for establishing inducible stable cell lines using the piggyBac transposase-based method (Li, Z., et al. (2013) Simple piggyBac transposon-based mammalian cell expression system for inducible protein production. Proc. Natl. Acad. Sci. U.S. A. 110, 5004-5009.).

cDNAs encoding the SARS-CoV and SARS-CoV-2 S protein proteins were human codon optimized and synthesized by Genscript. cDNA encoding human ACE2 was obtained from MGC clone 47598. The soluble SARS-CoV-2 S protein ectodomain trimer included residues 1-1211, followed by a foldon trimerization motif (Tao et al. (1997) Structure of bacteriophage T4 fibritin: A segmented coiled coil and the role of the C-terminal domain. Structure 5, 789-798), a 6×His tag and an AviTag biotinylation motif (Fairhead, M., and Howarth, M. (2015) Site-specific biotinylation of purified proteins using BirA. Methods Mol. Biol. 1266, 171-184.). Residues 682-685 (RRAR) were mutated to SSAS to remove the furin cleavage site on the SARS-CoV-2 S protein protein. Residues 986-987 (KV) were mutated to two proline residues to stabilize the pre-fusion form of the S protein (Pallesen et al. (2017) Immunogenicity and structures of a rationally designed prefusion MERS-CoV S protein antigen. Proc. Natl. Acad. Sci. U. S.A. 114, E7348-E7357.). The SARS-CoV-2 receptor binding domain (RBD, residues 328-528), the soluble human ACE2 construct (residues 19-615), and the SARS-CoV RBD (residues 315-514) were each followed by a 6×His tag and an AviTag.

Freestyle 293-F cells were grown in Freestyle 293 expression medium (Thermo Fisher) in suspension culture. Upon transfection, 300 mL cells were seeded to 1-L shaker flasks at a cell density of 10⁶ cells/mL. 15 mL Opti-MEM medium (Thermo Fisher) containing 300 μg of PB-CMV plasmid DNA was mixed with 15 mL Opti-MEM medium containing 400 μL 293fectin reagent (Thermo Fisher). The mixture was incubated for 5 minutes before being added to the shaker flask. Two days post transfection, each 300 mL culture was expanded to 3 shaker flasks containing a total of 900 mL medium, and the expression was continued for another 4 days.

Freestyle 293-F cells or a GnT1-knockout Freestyle 293-F cell line was used for generating the stable cell lines. The cells were transfected on 6-well plates. Each well contained 10⁶ cells growing in 2 mL Freestyle 293 expression medium. For producing doxycycline-inducible cell lines, 2 μg of the PB-TRE expression plasmid, 0.5 μg of the PB-rtTA-neomycin helper plasmid (Li et al. (2013)) and 0.5 μg of the piggyBac transposase plasmid pCyL43 (Wang et al. (2008) Chromosomal transposition of PiggyBac in mouse embryonic stem cells. Proc. Natl. Acad. Sci. U.S.A 105, 9290-9295.) were co-transfected to each well using the Lipofectamine 2000 reagent (Thermo Fisher). These cells were then selected using 2 μg/mL puromycin and 200 μg/mL G418 for two weeks. The ACE2-expressing cell line was constructed by co-transfecting the Freestyle 293-F cells with the PB-CMV plasmid encoding the full length human ACE2 and the pCyL43 plasmid. This cell line was selected using 2 μg/mL puromycin.

For stable expression, the cell lines were grown as suspension cultures in 1-L shaker flasks. Each flask contained 300 mL Freestyle 293 expression medium supplemented with 1 μg/mL doxycycline and 1 μg/mL aprotinin. Half of the culture was harvested and replaced by fresh medium every other day.

The proteins were purified from the harvested expression medium using Ni-NTA affinity chromatography. The proteins were eluted with phosphate buffered saline containing 300 mM imidazole and 0.1% (v/v) protease inhibitor cocktail (Sigma, P-8849). The proteins were further purified using size-exclusion chromatography. For the RBDs and ACE2, a Superdex 200 Increase (GE healthcare) column was used. For the S protein ectodomain, a Superose 6 Increase (GE healthcare) column was used.

Each biotinylation reaction contained 200 μM biotin, 500 μM ATP, 500 μM MgCl2, 30 μg/mL BirA, 0.1% (v/v) protease inhibitor cocktail and not more than 100 μM of the protein-AviTag substrate. The reactions were incubated at 30° C. for 2 hours. The biotinylated proteins were then purified by size-exclusion chromatography.

### Fab-Phage Selections

Fab-phage clones specific for the SARS-CoV-2 S protein were isolated from phage-displayed antibody libraries (Persson et al. (2013) J Mol Biol 22; 425(4):803-11) by multiple rounds of binding selections with the RBD immobilized in wells of neutravidin-coated microwell plates, as described (Persson et al. (2013)). Individual colonies were isolated from Escherichia coli infected with phage outputs from rounds 3 and 4, and individual Fab-phage clones were amplified. Antibody variable domains were sequenced by PCR amplification from phage supernatants for extraction of DNA sequences encoding antibody complementarity determining regions.

### Expression and Purification of IgG Proteins

Phage clone variable domain DNA was amplified by PCR and subcloned into pSCSTa-hIg1 and pSCST1-hk vectors. Vectors for the heavy and light chains were transfected into HEK293F cells (Invitrogen, Grand Island, NY) using FectoPro according to the manufacturer's instructions (Polyplus Transfection, NY). Cell cultures were incubated at 37° C. for 4-5 days post-transfection. The cell cultures were centrifuged, and the supernatants were applied to a protein-A affinity column (˜2 mL packed beads per 600 mL culture) (Pierce, ThermoScientific, Rockford, IL). IgG proteins were eluted with 100 mM glycine, pH 2.0 and neutralized with 2 M Tris, pH 7.5. The eluent underwent buffer exchange and concentration in to PBS, pH 7.4 by centrifugation in a 50 kDa centrifugal concentrator.

### Binding and Competition ELISAs

For capture of biotinylated antigens, 384-well microplate wells were coated overnight at 4° C. with 2 μg/mL neutravidin in PBS pH 7.4. After coating, wells were blocked with 0.2% BSA in phosphate buffered saline (PBS) for one hour and washed 4 times with 0.05% TWEEN™ in PBS (PT buffer). Solutions of S protein (10 nM) or RBD (50 nM) or biotinylated control (50 nM) in PBS were used to immobilize target by incubating for 15 min at room temperature and washing with PT buffer. IgGs were diluted into PBT, applied to the wells, and incubated at room temperature for 30 min. The plates were washed with PBST (3.2 mM Na₂HPO₄, 0.5 mM KH₂PO₄, 1.3 mM KCl, 135 mM NaCl, 0.05% TWEEN™ 20, pH 7.4.) and incubated for 30 min with anti-k-HRP (horse radish peroxidase) antibody conjugate (1:7500 dilution in PBT). The wells were washed 4-6 times with PBST and developed as described above. The absorbance at 450 nm was determined. To estimate EC₅₀ values, data were fit to standard four-parameter logistic equations using Graphpad Prism (GraphPad Software, La Jolla, CA).

For competition ELISAs, S protein was immobilized in 384-well plates as above but at a concentration of 5 μg/well, then wells were quenched with 100 μg/mL biotin. Immobilized S protein was blocked with 100 nM IgG for 30 min and 100 nM biotinylated ACE2 was added to both IgG-blocked and non-blocked wells in parallel. Following a 30 min incubation, the plates were washed with PBST, and HRP/streptavidin conjugate (1:10000 dilution in PBST) was added and incubated for 30 min. The plates were washed with PBST, developed with TMB substrate, and quenched with 0.5 M H₂SO₄ before measuring absorbance at 450 nm.

The results of these ELISAs are presented in FIG. 17. FIG. 17A depicts binding of unique Fab-phage clones to immobilized RBD blocked by solution-phase ACE2. The signal was normalized to the signal in the absence of ACE2 (control). FIG. 17B depicts serial dilutions of IgGs binding to immobilized SARS-CoV-2 S protein. The EC₅₀ values for each curve are shown in parentheses (in ηg/mL and ηM). FIG. 17C depicts binding of Fab-phage to immobilized RBD or negative controls, as indicated. FIG. 17D depicts binding of ACE2 to immobilized S protein the presence (white bars) or absence (black bars) of 100 ηM IgG.

### Binding Kinetics

To determine the affinity and binding kinetics of IgGs for the S protein, BLI experiments were performed on an Octet HTX instrument (ForteBio) at 1000 rpm and 25° C. Biotinylated S protein was first captured on SA biosensors (ForteBio) from a 2 μg/mL solution in PBT, in parallel with an identical concentration of an unrelated biotinylated control protein, followed by a 180 s quench step with 100 μg/mL biotin. After equilibrating with PBT, loaded biosensors were dipped for 600 s into wells containing serial 3-fold dilutions of IgG and subsequently were transferred back into assay buffer for 600 s dissociation. Binding response data were reference subtracted and were fitted with 1:1 binding model using ForteBio's Data Analysis software 9.0.

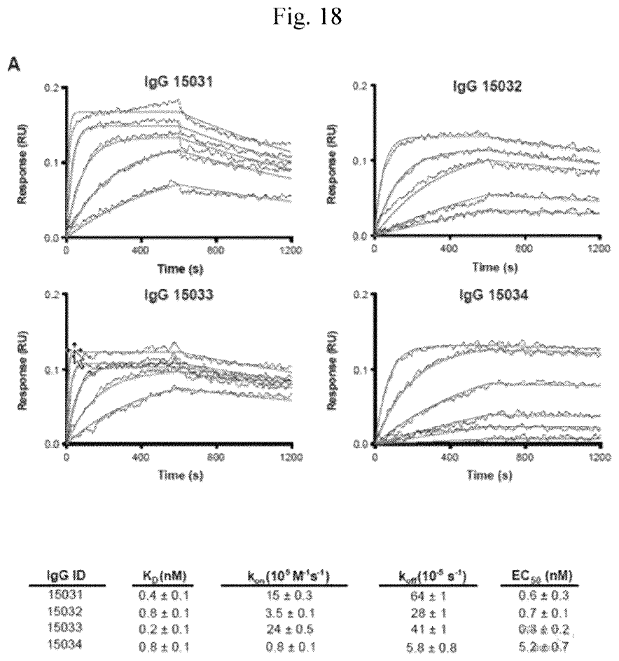

The binding kinetics for IgGs and SARS-CoV-2 S protein are presented in FIG. 18.

### Virus Neutralization Assays

For assays conducted in St Louis, SARS-CoV-2 strain 2019 n-CoV/USA_WA1/2020 was obtained from the Centers for Disease Control and Prevention (gift of Natalie Thornburg). Virus stocks were produced in Vero CCL81 cells (ATCC) and titrated by focus-forming assay on Vero E6 cells. Serial dilutions of mAbs were incubated with 102 focus-forming units (FFU) of SARS-CoV-2 for 1 h at 37° C. MAb-virus complexes were added to Vero E6 cell monolayers in 96-well plates and incubated at 37° C. for 1 h. Subsequently, cells were overlaid with 1% (w/v) methylcellulose in MEM supplemented with 2% FBS. Plates were harvested 30 h later by removing overlays and fixed with 4% PFA in PBS for 20 min at room temperature. Plates were washed and sequentially incubated with 1 μg/mL anti-S antibody CR3022 (Yan et al. (2020) A highly conserved cryptic epitope in the receptor binding domains of SARS-CoV-2 and SARS-CoV. Science 368, 630-633) and HRP-conjugated goat anti-human IgG in PBS supplemented with 0.1% saponin and 0.1% BSA. SARS-CoV-2-infected cell foci were visualized using TrueBlue peroxidase substrate (KPL) and quantitated on an ImmunoSpot microanalyzer (Cellular Technologies). Data were processed using Prism software (GraphPad Prism 8.0).

For assays conducted in Rome, SARS-CoV-2 strain 2019-nCoV/Italy-INMI was used. Antibodies were diluted to a concentration of 10 μg/ml in serum-free medium, and titrated in duplicate in two-fold serial dilutions. Equal volumes (50 p1) of approximately 100 TCID50/well virus and antibody dilutions, were mixed and incubated at 37° C. for 30 min. Subsequently, 96-well tissue culture plates with sub-confluent Vero E6 cell monolayers were infected with 100 μl/well of virus-serum mixtures in duplicate and incubated at 37° C. and 5% CO₂ for two days. Then, the supernatant of each plate was carefully discarded and 120 p1 of a Crystal Violet solution containing 2% Formaldehyde was added to each well. After 30 min fixation, the fixing solution was removed and cell viability was measured by photometer at 595 nm (Synergy HTX Biotek).

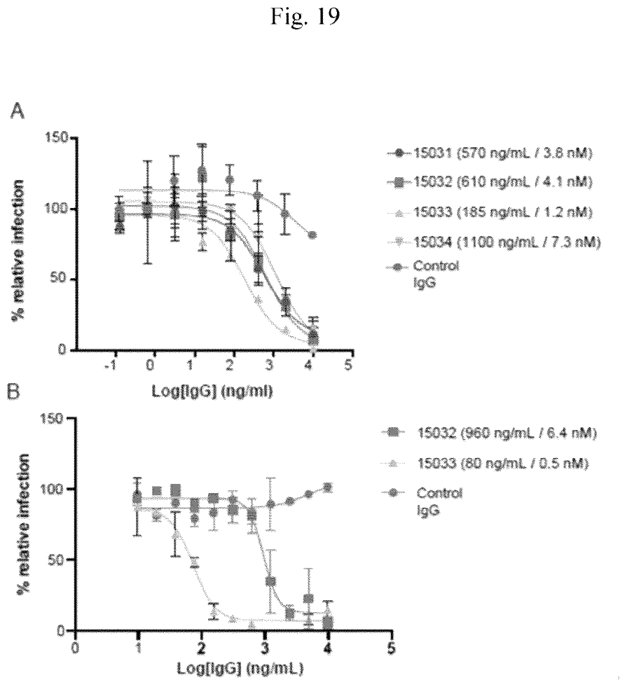

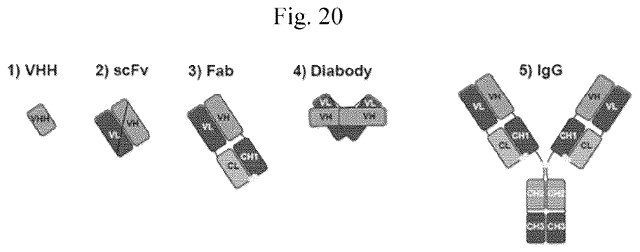

The results of these neutralization assays are depicted in FIG. 19.

Those skilled in the art will recognize, or be able to ascertain, using no more than routine experimentation, numerous equivalents to the specific procedures described herein. Such equivalents are considered to be within the scope of the inventions. Various substitutions, alterations and modifications may be made to the invention without departing from the spirit and scope of the invention. Other aspects, advantages, and modifications are within the scope of the invention.

The contents of all references, issued patents, and published patent applications cited through this application are hereby incorporated by reference. The appropriate component, process and methods of those patents, applications and other documents may be selected for the invention and embodiments thereof.

### D. Multivalent Binding Molecules

Described herein are multivalent binding molecules comprising two or more antibody binding fragments that bind a coronavirus, wherein the binding fragments are joined by a peptide linker. In an embodiment the multivalent binding molecule is at least bivalent or at least trivalent and does not comprise an Fc domain or a fragment thereof. In an embodiment of the invention at least two antibody fragments of the multivalent binding molecule are not identical.

Also described herein are multivalent binding molecules comprising an Fc domain, with or without effector function, a first and/or a second binding domain for a coronavirus polypeptide. In an embodiment of the multivalent binding molecules, the molecule comprises two binding domains, each binding domain comprises at least two coronavirus binding sites and the binding domains are attached to opposite termini of the Fc domain such that the two binding domains are not directly linked rather they are separated by the Fc domain.

Also described herein are multivalent binding molecules comprising an Fc domain, with or without effector function, and a binding domain for a coronavirus polypeptide comprising 2 or more, 3 or more, or 4 or more binding sites, wherein the binding domain is attached to the N-terminus of the Fc domain.

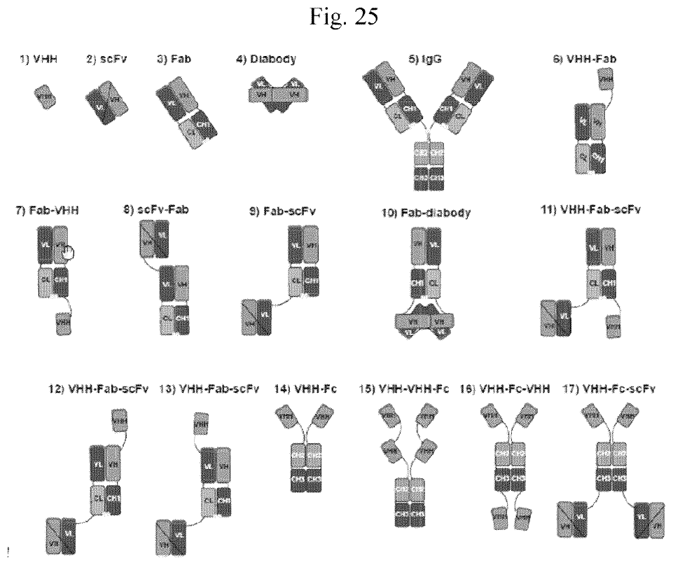

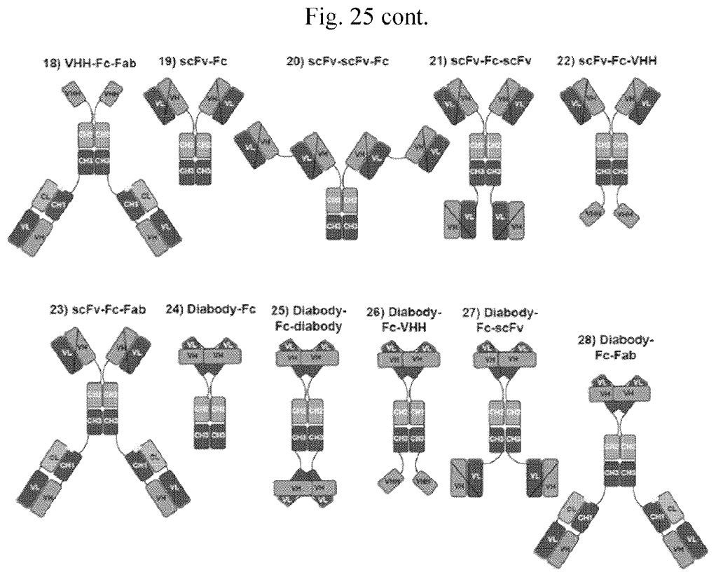

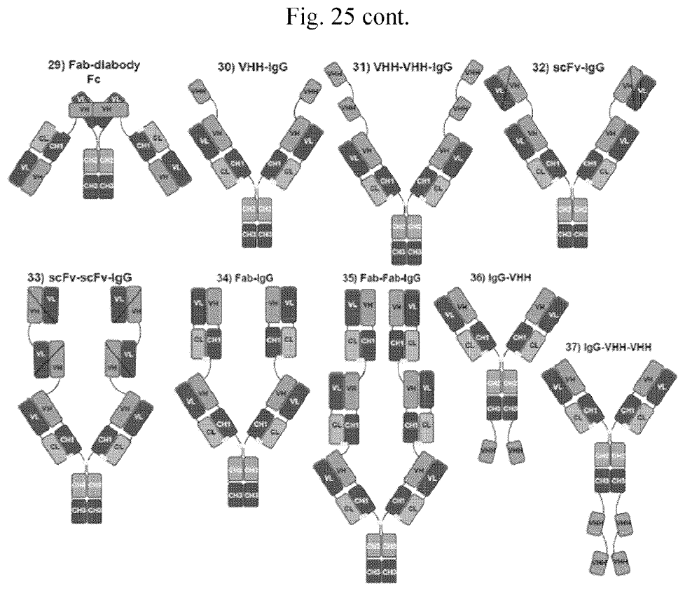

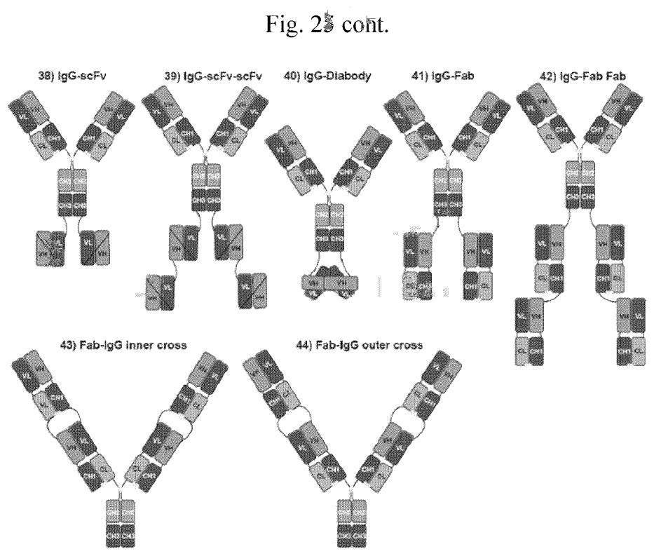

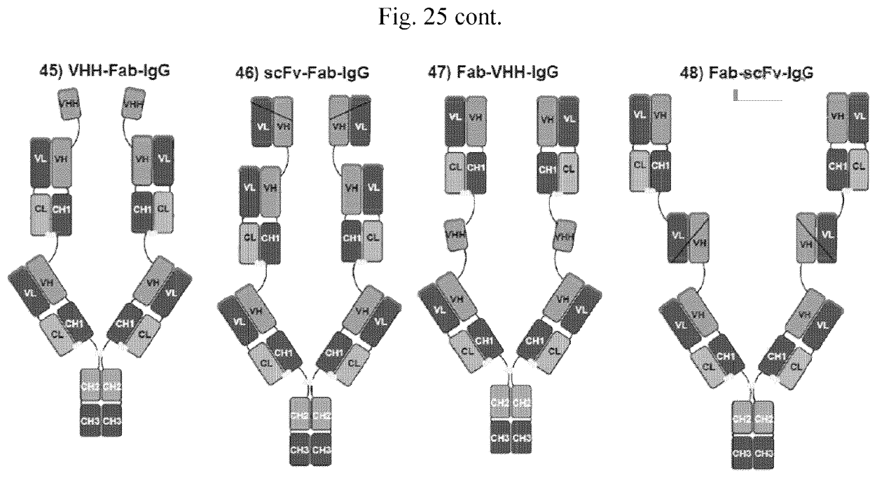

A subset of exemplary formats of the multivalent binding molecules of this invention is depicted in FIG. 25, see e.g., formats 6 to 48.

The Fc domain in the multivalent binding molecules herein may alternatively be a fragment of the Fc domain comprising the constant heavy chain domain 3 (CH3 domain). In an embodiment of the invention the Fc domain of the multivalent molecule is engineered such that it does not target the cell that binds the multivalent molecule for ADCC or CDC-dependent death. In an embodiment of the invention the Fc domain of the multivalent binding molecule is a peptide dimer in a knob-in-hole configuration. The peptide dimer may be a heterodimer.

The binding domains may be attached directly to the Fc domain or attached to the Fc domain via a linker. The binding domains may be monospecific and bind to the same epitope on a covonavirus polypeptide, or to overlapping epitopes on a coronavirus polypeptide, or may be multispecific, binding to different epitopes on one or more coronavirus polypeptide. The coronavirus polypeptide may be, e.g., a coronavirus S protein, including the S1 and S2 subunits, or the receptor binding domain (RBD) of the S protein.

The coronavirus bound by the multivalent binding molecules may be an alpha or beta coronaviraus. In an embodiment, the coronavirus may be, e.g., HCoV-229E and HCoV-NL63, HCoV-HKU1, HCoV-OC43, MERS-CoV, SARS-CoV, or SARS-CoV-2, and the polypeptide may be e.g., a spike (S) protein, a membrane (M) protein, a nucleocapsid (N) protein, a hemagglutinin-esterase glycoprotein (HE) or small envelope (e) protein of the coronavirus. In a particular embodiment the polypeptide is a protein of SARS-CoV-2, e.g. a spike (S) protein and fragments thereof.

In an embodiment of the invention one or both binding domains comprise an antibody binding fragment, e.g., a scfv, a Fab, a VHH, or a diabody or combinations thereof that bind a coronavirus polypeptide. In an embodiment of the one binding domain is a diabody or two scFvs and the other binding domain comprises two Fabs.

An embodiment of this invention is a Diabody-Fc, a tetravalent binding molecule having (i) an Fc domain, (ii) one coronavirus binding domain attached to the N-terminus of the Fc domain that comprises a coronavirus-binding diabody.

An embodiment of this invention is a Diabody-Fc-Fab, a tetravalent binding molecule having (i) an Fc domain, (ii) one coronavirus binding domain, attached to the N-terminus of the Fc domain, that comprises a diabody that binds a coronavirus polypeptide, and (iii) a second coronavirus binding domain, attached to the carboxy terminus of the Fc domain, comprising two Fab fragments each binding an epitope on a coronavirus polypeptide.

An embodiment of this invention is a Diabody-Fc-scFv, a tetravalent binding molecule having (i) an Fc domain, (ii) one binding domain that comprises a coronavirus-binding diabody attached to the N-terminus of the Fc domain, and (iii) a second binding domain, attached to the carboxy terminus of the Fc domain comprising two coronavirus-binding scFv each binding an epitope on a coronavirus polypeptide.

An embodiment of this invention is an IgG-scFv, a tetravalent binding molecule having (i) an Fc domain, (ii) one coronavirus binding domain that comprises of two Fab fragments attached to the N-terminus of the Fc domain, each Fab binding a coronavirus polypeptide and (iii) one coronavirus binding domain composed of two scFvs that each bind a site on a coronavirus, wherein the scFvs are attached to the C-terminus of the Fe domain.

An embodiment of this invention is a an Fab-IgG, a tetravalent binding molecule having (i) an Fc domain, (ii) a first coronavirus binding domain that is composed of a first set of two Fabs that bind a coronavirus polypeptide, wherein the Fabs are linked in tandem, and a second set of two Fabs that bind a coronavirus polypeptide, wherein the Fabs are linked in tandem and wherein both sets of Fabs are attached to the same terminus of the Fc domain. For example, the Fab-IgG tetravalent binding molecule may comprise,
(a) a dimer of a first and second heavy chain monomer, each monomer a single chain polypeptide comprising from N terminus to C-terminus,
(i) a first peptide comprising a first heavy chain variable domain (VH) linked to a first heavy chain constant domain (CH1),
(ii) a second peptide comprising a second heavy chain variable domain (VH) linked to a second heavy chain constant domain (CH1),
(ii) an Fc region, and
(b) a third peptide comprising a light chain variable domain (VL) and a light chain constant domain (CL),
wherein the heavy chain monomers dimerize via their Fc region and wherein the first VH and CH1 of each monomer pairs with the VL and CL of a third peptide, and the second VH and CH1 of each monomer pairs with the VL and CL of a third peptide, thereby forming the binding domain two sets of two Fabs linked in tandem, wherein each set is attached to the N-terminal of the Fc domain, see e.g., FIG. 25.

An embodiment of this invention is an IgG-Fab, a tetravalent binding molecule having (i) an Fe domain, (ii) a first coronavirus binding domain that comprises a two Fabs that bind a coronavirus polypeptide, and (iii) a second coronavirus binding domain that comprises a two Fabs that binds a coronavirus polypeptide, wherein the two binding domains are attached to opposite ends of the Fc domain. For example, the IgG-Fab may comprise,
(a) a dimer of a first and second heavy chain monomer, each monomer a single chain polypeptide comprising from N terminus to C-terminus,
(i) a first peptide comprising a first heavy chain variable domain (VH) linked to a first heavy chain constant domain (CH1),
(ii) Fc region,
(iii) a second peptide comprising a second VH linked to a second CH1, and
(b) a third peptide comprising a light chain variable domain (VL) and a light chain constant domain (CL),
wherein the monomers dimerize via their Fc regions to form the Fc domain, and wherein the first binding domain comprises two Fabs formed by the pairing of the first VH and CH1 of each monomer pair with the VL and CL of the third peptide, and
wherein the second binding domain comprises two Fabs formed by the pairing of the second VH and CH1 of each monomer with the VL and CL of the third peptide, whereby the first binding domain is attached to the N-terminus of the Fc domain and the second binding domain is attached to the C-terminus of the Fc domain. See FIG. 25.

Another embodiment of the multivalent binding molecule is an immunoglobulin-diabody, e.g., an IgG-diabody, a tetravalent binding molecule comprising a coronavirus-binding immunoglobulin, having a coronavirus-binding diabody attached to the carboxy terminal of the immunoglobulin Fe domain. For example, an IgG-diabody is a tetravalent binding molecule comprising (i) an Fc domain, (ii) an N-terminal coronavirus binding domain, comprising two coronavirus-binding Fabs and (ii) a C-terminal coronavirus binding domain comprising a coronavirus-binding diabody. The molecule comprises (1) a first set of a first and second monomer, wherein each monomer comprises a single-chain polypeptide comprising, from N-terminus to C-terminus: (a) a first heavy chain variable domain (VH) that binds a coronavirus polypeptide, (b) a heavy chain constant region domain 1 (CH1 domain) (c) an Fc region (or fragment thereof comprising a constant heavy chain domain 3 (CH3 domain)), (d) a peptide comprising a second VH that binds a coronavirus polypeptide, and a first light chain variable domain (VL) that binds a coronavirus polypeptide, and (2) second set of a third and fourth monomers, each comprising from N terminus to C terminus a second VL that binds a coronavirus polypeptide, and a constant light chain domain 1 (CL1 domain). The first set of monomers dimerize via their Fc regions, or fragments thereof, and the coronavirus polypeptide binding diabody is formed by the pairing of the second VH and first VL of one monomer with the first VL and second VH of the other monomer. The coronavirus polypeptide-binding Fabs are formed by the pairing of the first VH and CH1 of the first and second monomers, with the second VL and CL1 of the third and fourth monomers. In this format, the diabody forms the coronavirus polypeptide-binding domain on the C-terminus of the Fc domain and the Fabs form the coronavirus polypeptide-binding domain on the N-terminus of the Fc domain.

An embodiment of this invention is a tetravalent binding molecule in a Diabody-Fc-Fab format that binds a coronavirus polypeptide. This multivalent binding molecule comprises (1) a first set of a first and second monomer, wherein each monomer comprises a single-chain polypeptide comprising, from N-terminus to C-terminus: (a) a peptide comprising a first heavy chain variable (VH) domain, which binds coronavirus, and a first light chain variable (VL) domain, which binds coronavirus, (b) an Fc region (or fragment thereof comprising a constant heavy chain domain 3 (CH3 domain)), (c) a second VH domain, which binds coronavirus, and (d) a CH1 domain, and (2) a second set of a third and fourth monomer each monomer comprising from N-terminus to C-terminus a second VL domain, which binds coronavirus, and a constant light chain domain 1 (CL1). The first set of monomers dimerize via the Fc regions or fragments thereof and a bivalent coronavirus-binding diabody is formed by the pairing of the first VH domain and first VL domain of one monomer with the first VL domain and first VH domain of the other monomer, forming the first binding domain. The two coronavirus-binding Fabs are formed by the pairing of the second VL and CL1 with the second VH and CH1 thereby forming the second coronavirus binding domain. In this format, the diabody forms the first coronavirus binding domain on the amino terminus of the tetravalent molecule and the Fabs forms the second coronavirus binding domain on the C-terminus of the tetravalent binding molecule.

In an embodiment of this invention the binding moiety of the coronavirus binding domain is derived from an antibody that is monospecific binding specifically to one coronavirus polypeptide or is multispecific binding to more than one coronavirus polypeptide. In an embodiment of the invention the COVID-binding antibody binds to a RBD on the coronavirus S protein protein S1 subunit.

In an embodiment of this invention the multivalent binding molecule is monospecific binding to a single epitope on coronavirus. In an embodiment of this invention the multivalent binding molecule is multispecific binding to two or more epitopes on coronavirus.

In the multivalent binding molecules of this invention one or both of the binding domains may be multivalent, e.g., bi, tri, tetra or hexavalent and one or both of the multivalent binding domains may be multispecific, e.g., bi, tri, tetra or hexa-specific for the respective coronavirus polypeptide target. For example, the multivalent binding molecule may comprise a first coronavirus binding domain that is multivalent and monospecific (each binding site binding to the same epitopes) and a second coronavirus binding domain that is multivalent and multispecific, binding to different epitopes. In an embodiment of this invention the multivalent binding molecule comprises two binding domains that are bivalent and bispecific, each binding domain binding to two different epitopes on their respective target coronavirus polypeptides.

The VH and VL domains of the coronavirus binding domains may comprise the 3 light chain CDRs and 3 heavy chain CDRs of a coronavirus source antibody, or the three light chain CDRs and three heavy chain CDRs are at least 50%, at least 55%, at least 60%, at least 75%, at least 80%, at least 85%, at least 90%, at least 95%, at least 96%, at least 97%, at least 98% or at least 99% identical to the CDRs of a coronavirus source antibody.

In an embodiment of this invention one or both coronavirus binding domain of the multivalent binding molecules comprises 3 light chain CDRs and 3 heavy chain CDRs of the antibodies of Table 2, or CDRs that are at least 50%, at least 55%, at least 60%, at least 75%, at least 80%, at least 85%, at least 90%, at least 95%, at least 96%, at least 97%, at least 98% or at least 99% identical to 3 light chain CDRs and 3 heavy chain CDRs of the antibodies of Table 2. In some embodiments, the heavy chain and light chain CDRs of a VH and/or VL consist essentially of the heavy chain (hIg1) and light chain (hk) CDRs of an antibody of Table 2.

Also an embodiment of this invention are nucleic acid molecules encoding the multivalent binding molecules of this invention. An embodiment of this invention are nucleic acid molecules encoding the multivalent binding molecules described herein comprising the heavy chain and light chain CDRs set forth in Table 2. Also an embodiment of this invention are the nucleic acid molecules that encode VH and VL domains comprising respectively the heavy chain CDRs (CDR-H1 CDR-H2 and CDR-H3) and light chain CDRs (CDR-L1, CDR-L2 and CDR-L3) set forth in Table 2. In some embodiments, the nucleic acid molecule may comprise a nucleotide sequence encoding a multivalent binding molecule set forth in Table 3 or Table 4, e.g., Fab-VH, scFv-Fab, Fab-scFv, Fab-Diabody, VH(HC-CT)-Fab-scFv, VH(HC-NT)-Fab-scFv, VH(VL-NT)-Fab-scFv, VH-Fc, VH-VH-Fc, VH-Fc-VH, VH-Fc-scFv, VH-Fc-Fab, scFv-Fc, scFv-scFv-Fc, scFv-Fc-scFv, scFv-Fc-VH, scFv-Fc-Fab, Diabody-Fc, Diabody-Fc-Diabody, Diabody-Fc-VH, Diabody-Fc-scFv, Diabody-Fc-Fab, Fab-diabody-Fc, VH-hIgG1, VH-VH-hIgG1, scFv-hIgG1, scFv-scFv-hIgG1, Fab-hIgG1, Fab-Fab-hIgG1. In some embodiments, the nucleic acid molecule comprises a nucleotide sequence set forth in Table 3 or Table 4.

The nucleic acid molecules can be inserted into a vector and expressed in an appropriate host cell and then the multivalent binding molecules may be isolated from the cells using methods well known in the art. As such, also an aspect of this invention are expression cassettes and vectors comprising the nucleic acid molecules that encode the polypeptides of the multivalent binding molecules described herein, as well as the VL and VH domains, the Fabs, scFvs and the diabodies comprising the CDRs of an antibody set forth in Table 2 and Table 4, and the Fc domains described herein. An aspect of this invention are the host cells expressing these expression cassettes and vectors.

The degeneracy of the genetic code allows for different nucleic acids to encode the same amino acid sequence as set forth herein. Accordingly, also included are nucleotide sequences that encode a multivalent binding molecule of this invention that binds coronavirus, e.g., a coronavirus S polypeptide.

Also included in another embodiment are codon degenerate or optimized sequences. In another embodiment, the nucleic acid sequences are at least 70%, at least 75%, at least 80%, at least 90% and at least 95% identical to nucleic acid sequences encoding the CDRs and the multivalent binding molecules set forth in Table 2 and Table 4.

In an embodiment, the nucleic acid is an isolated nucleic acid.

Another aspect is a vector comprising the nucleic acid herein disclosed. In an embodiment, the vector is an isolated vector.

The vector can be any vector suitable for producing an antibody and/or binding fragment thereof, or a coronavirus-binding multivalent binding molecule of this invention, including for example vectors described herein. Possible expression vectors include but are not limited to cosmids, plasmids, or modified viruses (e.g. replication defective retroviruses, adenoviruses and adeno-associated viruses).

A further aspect is a recombinant cell producing a coronavirus-binding multivalent binding molecule of this invention or the vector herein disclosed.

The recombinant cell can be generated using any cell suitable for producing a polypeptide, for example suitable for producing an antibody and/or binding fragment thereof.

Suitable host cells include a wide variety of prokaryotic and eukaryotic host cells. For example, the proteins of the invention may be expressed in bacterial cells such as E. coli, insect cells (using baculovirus), yeast cells or mammalian cells.

More particularly, bacterial host cells suitable for producing recombinant antibody producing cells include E. coli, B. subtilis, Salmonella typhimurium, and various species within the genus Pseudomonas, Streptomyces, and Staphylococcus, as well as many other bacterial species well known to one of ordinary skill in the art. Suitable bacterial expression vectors preferably comprise a promoter which functions in the host cell, one or more selectable phenotypic markers, and a bacterial origin of replication. Representative promoters include the R-lactamase (penicillinase) and lactose promoter system, the trp promoter and the tac promoter. Representative selectable markers include various antibiotic resistance markers such as the kanamycin or ampicillin resistance genes. Suitable expression vectors include but are not limited to bacteriophages such as lambda derivatives or plasmids such as pBR322, the pUC plasmids pUC18, pUC19, pUC118, pUC119, and pNH8A, pNH16a, pNH18a, and Bluescript M13 (Stratagene, La Jolla, Calif.).

Suitable yeast and fungi host cells include, but are not limited to Saccharomyces cerevisiae, Schizosaccharomyces pombe, the genera Pichia or Kluyveromyces and various species of the genus Aspergillus. Examples of vectors for expression in yeast S. cerevisiae include pYepSecl, pMFa, pJRY88, and pYES2 (Invitrogen Corporation, San Diego, Calif.). Protocols for the transformation of yeast and fungi are well known to those of ordinary skill in the art.

Suitable mammalian cells include, among others: COS (e.g., ATCC No. CRL 1650 or 1651), BHK (e.g. ATCC No. CRL 6281), CHO (ATCC No. CCL 61), HeLa (e.g., ATCC No. CCL 2), 293 (ATCC No. 1573), NS-1 cells and any derivatives of these lines.

In an embodiment, the mammalian cells used to produce a recombinant antibody or a multivalent binding molecule of this invention are selected from CHO, HEK293 cells or FREESTYLE™ 293-F cells (Life technologies). FREESTYLE™ 293-F cell line is derived from the 293 cell line and can be used with the FREESTYLE™ MAX 293 Expression System, FREESTYLE™ 293 Expression System or other expression systems.

Suitable expression vectors for directing expression in mammalian cells generally include a promoter (e.g., derived from viral material such as polyoma, Adenovirus 2, cytomegalovirus and Simian Virus 40), as well as other transcriptional and translational control sequences.

In an embodiment, the vector is designed for production of light chain or heavy chain of the multivalent binding molecule.

Suitable insect cells include cells and cell lines from Bombyx or Spodotera species. Baculovirus vectors available for expression of proteins in cultured insect cells (SF 9 cells) include the pAc series and the pVL series.

The recombinant expression vectors may also contain genes which encode a fusion moiety (i.e. a “fusion protein”) which provides increased expression or stability of the recombinant peptide; increased solubility of the recombinant peptide; and aid in the purification of the target recombinant peptide by acting as a ligand in affinity purification, including for example tags and labels described herein. Further, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

In an embodiment, expression of the multivalent binding molecules, or components of the multivalent binding molecules, is under the control of an inducible promoter. Examples of inducible non-fusion expression vectors include pTrc (28) and pET 11d.

The recombinant expression vectors may also contain a marker gene which facilitates the selection of host cells transformed or transfected with a recombinant molecule of the invention. Examples of selectable marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. Transcription of the selectable marker gene is monitored by changes in the concentration of the selectable marker protein suchas—galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of recombinant expression vectors of the invention and in particular to determine the effect of a mutation on expression and phenotype. It will be appreciated that selectable markers can be introduced on a separate vector from the nucleic acid of interest. Other selectable markers include fluorescent proteins such as GFP which may be cotransduced with the nucleic acid of interest.

An embodiment of this invention is a pharmaceutical composition comprising a multivalent binding molecule or the nucleic acid molecules, expression cassettes and vectors encoding a multivalent binding molecule described herein and a pharmaceutically acceptable carrier, diluent or excipient. The pharmaceutical composition may further comprise an additional therapeutic agent, e.g., therapeutic small molecules, or a nucleic acid molecule, expression cassettes and vectors that encode the agent. The pharmaceutical composition may consist or consist essentially of a multivalent binding molecule of this invention, or the nucleic acid molecules expression cassettes and vectors encoding the multivalent binding molecule of this invention, and a pharmaceutically acceptable diluent, carrier or excipient. Suitable carriers, diluents and excipients, and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the multivalent binding molecule, which matrices are in the form of shaped articles, e.g., films, liposomes or microparticles. Delivery systems for administering therapeutic agents are known in the art, see e.g., Wu and Wu, J. Biol. Chem. 262:4429-4432 (1987)), Langer, 1990, Science 249:1527-1533; Sefton, 1987, CRC Crit. Ref. Biomed. Eng. 14:20; Buchwald et al., 1980, Surgery 88:507; Saudek et al., 1989, N. Engl. J. Med. 321:574; Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; PCT Publication No. WO 99/15154; and PCT Publication No. WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides, poly(lactide-co-glycolides) (PLGA), and polyorthoesters. In one embodiment, the polymer used in a sustained release formulation is inert, free of leachable impurities, stable on storage, sterile, and biodegradable. It will be apparent to those persons skilled in the art that certain carriers may be more preferable depending upon, for instance, the route of administration and concentration of the multivalent binding molecule being administered.

The composition can be a lyophilized powder or aqueous or non-aqueous solution or suspensions, which may further contain antioxidants, buffers, bacteriostats and solutes. Other components that may be present in such compositions include water, surfactants (such as polysorbates, such as TWEEN™), alcohols, polyols, glycerin and vegetable oils, for example.

Suitable diluents for nucleic acids include but are not limited to water, saline solutions and ethanol. Suitable diluents for polypeptides, including antibodies or fragments thereof and/or cells include but are not limited to saline solutions, pH buffered solutions and glycerol solutions or other solutions suitable for freezing polypeptides and/or cells.

The composition can further comprise stabilizing agents, for example reducing agents, hydrophobic additives, and protease inhibitors which are added to physiological buffers.

### Methods

Additional aspects of the invention are methods for using, producing and/or isolating the multivalent binding molecule described herein.

It has been reported the symptoms of coronavirus, e.g., COVID-19, infections include one or more of, e.g., fever, cough, shortness of breath, chills, repeated shaking with chills, muscle pain, headache, sore throat, loss of taste or smell, and can lead to physical complications, e.g., pneumonia and trouble breathing, organ failure in several organs, heart problems, severe lung condition, e.g., acute respiratory distress syndrome, blood clots, acute kidney injury and additional viral and bacterial infections. An embodiment of the invention relates to a method for treating, preventing, mitigating, or delaying the progression of a coronavirus, e.g., COVID-19, infection, or ameliorating a symptom of a coronavirus infection, comprising administering an effective amount of a multivalent binding molecule of this invention to a subject having or suspected of having a coronavirus infection. A method for treating, preventing, mitigating, or delaying the progression of a coronavirus infection includes reducing viral load in a subject in need thereof. The multivalent binding molecule of the present invention may be administered systemically or locally, e.g., by injection (e.g. subcutaneous, intravenous, intraperitoneal, intrathecal, intraocular, etc.), inhalation, implantation, topically, or orally. Depending on the route of administration, the multivalent binding molecule may be coated in a material to protect the molecules from the action of acids and other natural conditions which may inactivate them. The multivalent binding molecules described herein may be dissolved or suspended in a pharmaceutically acceptable, preferably aqueous carrier. In addition, the composition comprising the multivalent binding molecule can contain excipients, such as buffers, binding agents, blasting agents, diluents, flavors, lubricants, etc. An extensive listing of excipients that can be used in such a composition, can be found in, for example, A. Kibbe, Handbook of Pharmaceutical Excipients (Kibbe, 2000). The multivalent binding molecules can also be administered in conjunction with other therapeutic regimens or agents for treatment of the infection and the associated symptoms and complications, e.g., convalescent serum therapy, small molecules, neutralizing monoclonal antibodies, recombinant antibodies, cytokines, or antivirals, e.g., LY-CoV555 (Eli Lilly), e.g., recombinant anti-CD47/PD-L1 bispecific antibody (IBI322)(Innovent Biologics, Inc.), protease inhibitors, e.g., nelfinavir mesylate or camostat mesylate, furin inhibitors, remdesivir, Actemra, Oluminant (baricitinib), merimepodib, dexamethaone (glucocorticoid), Aviptadil (RLF-100), hydroxycloropuine, Avigan (favipiravir, Avifavir), Bucillamine, niclosamide, aviptadil, PlKfyve kinase inhibitor, tyrosine kinase inhibitor efineptakin alfa, interferon beta, or SLV213 (Selva Therapeutics).

Effective dosages and schedules for administering the multivalent binding molecule of this invention and nucleic acids that encode them described herein may be determined empirically, and making such determinations is within the skill in the art. Those skilled in the art will understand that the dosage of such multivalent binding molecule that must be administered will vary depending on, for example, the subject that will receive the multivalent binding molecule, the route of administration, the particular type of multivalent binding molecule used and other drugs being administered. Guidance in selecting appropriate doses for multivalent binding molecules of this invention is found in the literature on therapeutic uses of antibodies, e.g., Handbook of Monoclonal Antibodies, Ferrone, eds., Noges Publications, Park Ridge, N.J., (1985) ch. 22 and pp. 303-357; Smith, Antibodies in Human Diagnosis and Therapy, Haber, eds., Raven Press, New York (1977) pp. 365-389. The dosage ranges for the administration of the compositions are those large enough to produce the desired effect. The dosage should not be so large as to cause adverse side effects, such as unwanted cross-reactions, anaphylactic reactions, and the like. Generally, the dosage will vary with the age, condition, sex and extent of the inflammation in the patient and can be determined by one of skill in the art. The dosage can be adjusted by the individual physician in the event of any contraindications. Dosage can vary, and can be administered in one or more dose administrations daily, for one or several days. While individual needs vary, determination of optimal ranges of effective amounts of the multivalent binding molecules, nucleic acids and vectors is within the skill of the art.

An embodiment of the invention relates to a method for preventing or delaying the cleavage of a coronavirus S protein S2 subunit, or the fusion of the coronavirus, e.g., COVID-19, with a cell and internalization of the coronavirus by the cell, e.g., a cell having a receptor for the coronavirus, e.g. an ACE2 receptor, by contacting the cell with an effective amount of a multivalent binding molecule of this invention.

Yet another aspect relates to a method for screening or for diagnosing a subject having or suspected of having a coronavirus infection, the method comprising measuring the level of a coronavirus polypeptide or fragment thereof in a sample from a subject using a multivalent binding molecule or assay herein disclosed, wherein detecting a level of a coronavirus polypeptide or fragment thereof is indicative that the subject is infected with the coronavirus. The method may further comprise the step of comparing the level of a coronavirus polypeptide or fragment thereof in the sample bound by a multivalent binding molecule of this invention with a negative or positive control. An increased level of a coronavirus polypeptide or fragment thereof in the sample bound by the multivalent binding molecule as compared to a negative control, e.g., a value derived from a group of subjects without coronavirus infection, or convalescing from a coronavirus infection, is indicative that the subject is infected with coronavirus. A level of coronavirus similar or exceeding the level of coronavirus of the positive control, e.g., a value derived from a group of subjects known to have a coronavirus infection, is indicative that the subject is infected with coronavirus. The level of coronavirus polypeptide or fragment thereof bound by a multivalent binding molecule may be determined via an immunoassay comprising or using one or more multivalent binding molecule described herein. In an embodiment, the immunoassay is an enzyme linked immunosorbent assay (ELISA). In an embodiment the ELISA is a sandwich ELISA. The coronavirus polypeptide may be a coronavirus S protein or an S1 or S2 subunit or RBD of the S protein. If it is determined that the subject is infected with a coronavirus, therapeutic measures are administered to the subject.

An additional aspect of the invention is a method for prognosing coronavirus progression or lack thereof (e.g. recovery or worsening of disease). Accordingly an aspect is a method of prognosing a likelihood of recovery after coronavirus infection, the method comprising: a) measuring a level of a coronavirus polypeptide or fragment thereof in a sample from a subject by contacting the sample with the multivalent binding molecules of this invention; and b) comparing the level of a coronavirus polypeptide or fragment thereof bound by the multivalent binding molecules in the sample with a control, for example a control value derived from a group of subjects who recovered from or in whom the coronavirus disease symptoms regressed. A decreased level of coronavirus in the sample bound by the multivalent binding molecules compared to the control is indicative that the subject has an increased likelihood of recovery after coronavirus infection. An increased level of coronavirus in the sample bound by the multivalent binding molecules compared to the control is indicative that the subject has a reduced likelihood of recovery after coronavirus infection. The level of coronavirus may be measured by, e.g., measuring the amount of a coronavirus polypeptide or fragment thereof bound to the multivalent binding molecules of this invention by e.g., mass spectrometry, HPLC, immunoprecipitation or via an immunoassay, e.g. ELISA. The coronavirus polypeptide may be a COVID-19 S protein or an S1 or S2 subunit or RBD of the S protein. If it is determined that the subject has a decreased likelihood of recovery after coronavirus infection, therapeutic measures are administered to the subject.

A further embodiment of the invention is a method for prognosing the likelihood of progression of a coronavirus infection and associated adverse effects, the method comprising: a) measuring a level of a coronavirus polypeptide, or fragment thereof in a sample from a subject by detecting the level coronavirus polypeptide or fragment thereof with a multivalent binding molecule of this invention; and b) comparing the level of the coronavirus polypeptide or fragment thereof in the sample with a control, wherein an increased level of the coronavirus polypeptide or fragment thereof in the sample compared to the control is indicative of the level of coronavirus. In an embodiment where the control is a control value derived from a group of subjects who did not recover or progressed an increased level of the coronavirus polypeptide or fragment thereof is indicative that the subject has a decreased likelihood of recovering from a coronavirus infection. In an embodiment, the control is a control value derived from a group of subjects who recovered from coronavirus infection and an increased level of a coronavirus polypeptide or fragment thereof in the subject's sample bound by a multivalent binding molecule of this invention compared to the control is indicative that the subject has a decreased likelihood of recovery after coronavirus infection and/or an increased likelihood of coronavirus infection and associated adverse effects progressing. The level of coronavirus may be determined by immunoprecipitation of coronavirus or a coronavirus polypeptide or fragment thereof with the multivalent binding molecules of this invention, or via an immunoassay, e.g. ELISA, immunoblot, mass spectrometry, or HPLC, using the multivalent binding molecules of this invention as the capture or detection antibody. The coronavirus polypeptide may be a COVID-19 S protein or an S1 or S2 subunit or RBD of the S protein.

The sample may be taken from a subject after receiving a treatment for a coronavirus infection and compared to a pre-treatment sample. Alternatively, the patient can be monitored after a repeating interval to assess whether a treatment with the multivalent molecules of this invention or other intervention is necessary. In an embodiment, the test is repeated and plotted to assess the subject's progression.

In an embodiment, the sample used in the methods described herein is a tissue sample, e.g. lung tissue, or a biological fluid such as blood, or a fraction thereof such as plasma or serum, or a lung secretion or a pulmonary lavage, or a nasal secretion. In an embodiment the biological fluid is saliva, a lung secretion, or a nasal secretion and can be obtained as, e.g., oropharyngeal (throat) swab, nasopharyngeal swab, anterior nasal swab, and mid-turbinate nasal swab.

In another embodiment, the sample used in the methods described herein is selected from a fresh sample such as a fresh biological fluid sample or tissue sample (e.g. including not frozen), one-time frozen biological fluid sample or tissue sample (e.g. frozen a single time at the time of obtaining the sample)) or a repeat frozen sample (e.g. frozen and thawed and frozen biological fluid sample or repeat frozen tissue sample). In an embodiment, the sample comprises native or natively folded coronavirus polypeptide. In an embodiment, the sample is a fixed sample such as a mildly fixed sample wherein the fixation induces limited denaturation and/or unfolding of a coronavirus polypeptide.

Also an aspect of this invention is a method for making the multivalent binding molecules described herein. The amino acid sequences of coronaviruses, e.g., COVID-19, and its polypeptides, e.g. the S protein, are known, and methods to produce libraries of antibodies that bind to a selected protein or nucleic acid, or fragments thereof, are well known in the art (see e.g., U.S. publication no. 2015/0232554, inventors Gurney et al. and US publication no. 2016/0194394, inventors Sidhu et al. and US 20190040144, inventors Pan et al.; U.S. publication no. 2017/0166636, inventors Wu et al.; U.S. publication no. 2016/0208018, inventors Chen et al.; U.S. publication no. 2016/0053022, inventors Macheda et al.). And a variety of methods are known in the art for generating and screening phage display libraries for antibodies, and antibody fragments, scFv, Fab, VL, and VH possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004), all incorporated herein by reference. In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360, all incorporated herein by reference. The multivalent binding molecules/synthetic antibodies of this invention may be made recombinantly, e.g., by Gibson assembly (see Gibson et al. (2009) Nature Methods. 6 (5): 343-345 and Gibson DG. (2011) Methods in Enzymology. 498: 349-361), or the molecules may be made synthetically e.g., using a commercial synthetic apparatus, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like. If desired, various groups may be introduced into the peptide during synthesis or during expression, which allow for linking to other molecules or to a surface. As such libraries of antibodies and antibody fragments and multivalent binding molecules that bind a selected coronavirus polypeptide can be readily generated using methods known in the art.

In another embodiment, a nucleic acid encoding a multivalent binding molecule described herein is expressed in a host cell to make the multivalent binding molecule. In an embodiment, the method comprises: a) expressing in a host cell a nucleic acid encoding a multivalent binding molecule thereof herein disclosed; b) culturing the host cell to produce the multivalent binding molecule; and c) isolating and/or purifying the multivalent binding molecule from the host cell.

In some embodiments, a nucleic acid encoding a heavy chain or light chain of the multivalent binding molecule is expressed. In other embodiments, multiple nucleic acids are expressed, for example a nucleic acid encoding a heavy chain and a nucleic acid encoding a light chain of the multivalent binding molecule.

Suitable host cells and vectors are described above. Vectors and nucleic acids encoding a multivalent binding molecule described herein may be introduced into cells, e.g., mammalian cells via conventional techniques such as calcium phosphate or calcium chloride co-precipitation, DEAE-dextran mediated transfection, lipofection and other liposome-based transfection agents, electroporation or microinjection.

Nucleic acid encoding a multivalent binding molecule described herein may be directly introduced into mammalian cells using delivery vehicles such as retroviral vectors, adenoviral vectors and DNA virus vectors.

In an embodiment of this invention, a multivalent binding molecule comprising an Fc domain and one coronavirus binding domain attached to the N-terminus of the Fc domain comprised of a diabody is generated by,
a) selecting an Fc domain, or fragment thereof comprising CH3, having a C-terminus and an N-terminus
b) identifying an antibody that binds to a coronavirus polypeptide (the source antibody), and
c) generating a nucleic acid molecule comprising a nucleotide sequence that encodes a polypeptide monomer comprising
(i) a first peptide comprising the heavy chain and light chain CDRs, or a VL and/or a VH domains, of an antibody of step b, or comprising heavy chain and light chain CDRs or VL and/or a VH domains derived from, the antibody of step b that still bind the coronavirus polypeptide, linked to
(ii) the Fc domain, or fragment thereof comprising CH3,
d) expressing the nucleic acid molecule of step c to produce the polypeptide and then dimerizing the polypeptide,
wherein the VH and VL of one monomer pair with the VH and VL of the other monomer forming a coronavirus-binding diabody, and
wherein the polypeptide monomer dimerizes via the Fc regions to form a multivalent binding molecule comprising an Fc domain, a coronavirus-binding domain comprised of the diabody attached to the N-terminus of the Fc domain.

In an embodiment of this invention a multivalent binding molecule of this invention, comprising a first coronavirus binding domain comprised of a diabody and a second coronavirus binding domain comprised of two coronavirus-binding Fabs, is generated by,
a) selecting an Fc domain, or fragment thereof comprising CH3, having a C-terminus and an N-terminus
b) identifying an antibody that binds to a coronavirus polypeptide (the “source antibody”), and
c) generating a nucleic acid molecule comprising a nucleotide sequence that encodes a “heavy chain” polypeptide comprising
(i) a first peptide comprising the heavy chain and light chain CDRs, or a VL and/or a VH domains, of an antibody of step b, or comprising heavy chain and light chain CDRs or VL and/or a VH domains derived from the antibody of step b that still bind the coronavirus polypeptide, linked to
(ii) the Fc domain, or fragment thereof comprising CH3, of step a, linked to
(iii) a second peptide comprising VL and/or VH domains comprising the heavy chain CDRs of an antibody of step b, or comprising heavy chain CDRs derived from the antibody of step b and that still bind the coronavirus polypeptide, and a constant heavy region 1 (CH1),
d) generating a nucleic acid molecule comprising a nucleotide sequence that encodes a “light chain” polypeptide comprising a constant light region 1 (CL1) linked to a VL domain wherein the VL domain comprises the light chain CDRs of the antibody in step b,
e) expressing the nucleic acid molecules of steps c and to produce the polypeptides,
wherein two heavy chain polypeptides dimerize via their Fc regions, and the VH and VL of the first peptide of one heavy chain polypeptide pairs with the VH and VL of the first peptide of another heavy chain polypeptide to form a coronavirus-binding diabody, and the VH and CH1 domains of the second peptide of each heavy chain polypeptide bind the VL and CL1 of a light chain polypeptide forming two coronavirus-binding Fabs.

In an embodiment of this invention the multivalent binding molecule having two coronavirus binding Fabs linked to one terminus of the Fc domain and two coronavirus-binding scFvs or a coronavirus-binding diabody linked to the other terminus of the Fc domain is generated by,
a) identifying the light chain complementary determining regions (CDR-L1, CDR-L2, and CDR-L3) and heavy chains complementary determining regions (CDR-H1, CDR-H2, and CDR-H3) of an antibody that binds to a coronavirus polypeptide (the “source antibody”), and
b) generating a nucleic acid molecule encoding a “heavy chain” polypeptide comprising
(i) a first peptide comprising an immunoglobulin constant heavy chain region 1 (CH1 domain) and a heavy chain variable domain (VH domain) comprising the CDR-H1, H2 and H3 of the antibody of step a, or CDR-H1, H2 and H3 derived from the antibody of step a that still binds a coronavirus polypeptide, linked to
(ii) an Fe region, linked to
(iii) a second peptide comprising a variable light chain domain (VL domain) comprising the CDR-L1, L2 and L3 of an antibody of step b and a VH domain comprising the CDR-H1, H2 and H3 of an antibody of step b, or CDR-H1, H2 and H3 derived from the antibody of step b that binds to a coronavirus polypeptide,
c) generating a nucleic acid molecule comprising a nucleic acid sequence that encodes a “light chain” polypeptide comprising a constant light region 1 (CH1) linked to a VL domain wherein the VL domain comprises the light chain CDRs, CDR-L1, L2 and L3 of the antibody in step a,
d) expressing the nucleic acid molecules of (b) and (c) to produce a heavy chain polypeptide and a light chain polypeptide,
wherein two heavy chain polypeptides dimerize via their Fc regions and the VH and CH1 domains of the heavy chain polypeptide pair with the VL and CL1 domains of the light chain polypeptide forming two coronavirus-binding Fab and
wherein the VH and VL of the second peptide in each heavy chain polypeptide pair to form an scFv that binds the coronavirus polypeptide, or the VH and VL of the second peptide of one heavy chain polypeptide in the dimer pair with the VL and VH of the second peptide of the other heavy chain polypeptide in the dimer to form a diabody, thereby forming the multivalent binding molecule comprising an Fc domain, two coronavirus-binding Fabs linked to one terminus of the Fc domain and two coronavirus-binding scFvs or a coronavirus-binding diabody linked to the other terminus of the Fc domain.

The coronavirus-binding source antibody in the methods described herein may be an antibody that binds an epitope of any coronavirus polypeptide, but preferably to an epitope of a coronavirus S protein, e.g., the RBD or N-terminal domain (NTD) of the S1 subunit or the fusion peptide (FP), heptapeptide repeat sequences HR1 and HR2, TM domain, and cytoplasm domain of the S2 domain.

The coronavirus-binding source antibody may be an antibody that is monospecific binding specifically to one coronavirus polypeptide or multispecific binding to more than one coronavirus polypeptides.

The source antibody may be a full-length antibody or an antibody fragment that binds a coronavirus polypeptide, e.g., a Fab, a VL or VH. The light chain and heavy chain CDRs, the VH and/or VL in one or both of the coronavirus binding domains of the multivalent binding molecule may be identical to the CDRs, the VH and/or VL of the coronavirus source antibody or may be at least 50%, at least 55%, at least 60%, at least 75%, at least 80%, at least 85%, at least 90%, at least 95%, at least 96%, at least 97%, at least 98% or at least 99% identical to the CDRs, VH or VL of the source antibody and still retain binding to the coronavirus polypeptide. The CDRs, the VH and/or VL in the coronavirus-binding domain of the multivalent binding molecule may be identical to the CDRs, the VH and/or VL of a COVID-19-binding antibody of Table 2 or Table 4, or may be at least 50%, at least 55%, at least 60%, at least 75%, at least 80%, at least 85%, at least 90%, at least 95%, at least 96%, at least 97%, at least 98% or at least 99% identical to the CDRs, VH or VL of a COVID-19-binding antibody of Table 2 or Table 4 and still retain binding to the COVID-19 polypeptide. In some embodiments, the amino acid sequence of the CDRs, the VH and/or VL in the coronavirus-binding domain of the multivalent binding molecule consist essentially of the amino acid sequence of a CDR, VH, and/or VL of Table 2 or Table 4.

In an embodiment of this invention, two heavy chain polypeptide monomers of the multivalent binding molecule dimerize via knob in hole configuration of their Fc sequences. The multivalent molecules of this invention may be generated by dimerizing two polypeptides in a “knob-in-hole” configuration. The knob-in-hole configuration increases the modularity of this invention by facilitating the association of peptides that comprise binding moieties that bind different epitopes on a coronavirus polypeptide. Methods for engineering Fc molecules via the knobs into holes design are well known in the art, see e.g., WO2018/026942, inventors Van Dyk et al., Carter P. (2001) J. Immunol. Methods 248, 7-15; Ridgway et al. (1996) Protein Eng. 9, 617-621; Merchant, et al. (1998) Nat. Biotechnol. 16, 677-681, and; Atwell et al., (1997) J. Mol. Biol. 270, 26-35.

The binding domains of the multivalent binding molecules may be linked to the Fc domain via a linker. In some embodiments, adjacent VH and VL domains may be attached to each other via a peptide linker. In some embodiments, adjacent constant domains and variable domains are attached via a linker. The linker may be, e.g. a polypeptide linker, or a non-peptidic linker. In some embodiments the constant domains and variable domains of the multivalent binding molecules are attached to the Fc domain via a peptide linker. Suitable linkers are well known in the art, e.g., an XTEN linker (see WO2013120683, inventors Schellenberger et al.) In some embodiments, the peptide linker comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or at least 100 amino acids. In some embodiments, the peptide linker is between 5 to 75, 5 to 50, 5 to 25, 5 to 20, 5 to 17, 5 to 16, 5 to 15, or 5 to 10, 1-10, 1-5, or 2-4 amino acids in length. Examples of peptide linkers are set forth in Table 4.

The modular aspects of this invention allow for combining antigen binding fragments derived from antibodies that bind to the same or different coronavirus epitopes or coronavirus polypeptides, to generate various binding domains and in some embodiments attaching different binding domains on the opposite termini of the Fe domain, to generate a multivalent binding molecule that binds selected coronavirus polypeptides. The modular aspects of this invention also allow for attaching a scFv by its VL or VH domain to the C-terminus or N-terminus of the Fc domain or the C-terminus or N-terminus another antibody fragment. As such the scFv orientation in the multivalent molecules from its amino to carboxy terminus may be either VL-linker-VH or VH-linker-VL.

The Fc domain of the multivalent binding molecules, with or without the linker, is greater than 100 amino acids, greater than 125 amino acids greater than 150 amino acids, greater than 175 amino acids or greater than 200 amino acids. The Fc domain may be about 100 amino acids to about 250 amino acids in length, about 125 amino acids to about 250 amino acids in length or about 150 amino acids to about 220 amino acids in length.

### Assays

The multivalent binding molecules of this invention can be used in a variety of assays for binding, detecting and measuring coronavirus or a coronavirus polypeptide in a sample. For example, the multivalent binding molecules can be used in an ELISA, as well as immunoprecipitation, immunoblot, immunohistochemistry or immunocytochemistry, proximity ligation assay (PLA)(Muhammad S. Alam, November 2018) Current Protocols in Immunology Vol 123, Issue 1.), mass spectroscopy-based techniques, particle-based flow cytometric assay, fluorescence-activated cell sorting (FACS) and ELISA.

Immunodetection methods as described herein generally involve the detection or measuring complexes of multivalent binding molecules and a coronavirus polypeptide, e.g. an S1 of S2 subunit or RBD, using multivalent binding molecules disclosed herein. The detection of such complexes may be achieved through different methods, for example by using a detectable label or marker, such as a radioactive, fluorescent or enzymatic tag. Detection of these complexes may also involve the use of a ligand such as a secondary antibody and/or binding fragment thereof specific for a coronavirus polypeptide or for the multivalent binding molecules:coronavirus polypeptide complex.

They can also be used to make detection assays, for example a sandwich ELISA, as an antibody or the multivalent binding molecules can be used as a capture reagent to isolate a coronavirus or a coronavirus polypeptide while another antibody or multivalent binding molecule binding a distinct epitope can be used as a detection reagent.

Accordingly, another aspect is an immunoassay comprising one or more multivalent binding molecules described herein.

In an embodiment, the immunoassay is an enzyme linked immunosorbent assay (ELISA). The multivalent binding molecules may be used in the context of detection assays such as ELISAs, for example sandwich ELISAs. The multivalent binding molecules of this invention may be used as capture and detection antibodies for the detection of coronavirus or a coronavirus polypeptide in solution. For example, full length IgGs recognizing the coronavirus protein, e.g., the S1 subunit, are immobilized and incubated with coronavirus or a coronavirus polypeptide or subunit. The samples are then incubated with biotinylated IgGs recognizing epitopes on the virus, polypeptide, or subunit.

In an embodiment, the ELISA is a sandwich ELISA comprising a capture agent and a detection agent, wherein either the capture agent or the detection agent is a multivalent binding molecule that has CDRs identified herein and which specifically binds a COVID-19 polypeptide, for example which specifically binds S1 subunit, e.g., the RBD, wherein the capture agent and the detection agent bind different epitopes. In another embodiment, only one of the capture and detection antibodies is a multivalent binding molecule of this invention and the other capture or detection agent is an antibody and/or binding fragment thereof that binds a COVID-19 polypeptide, but is not a multivalent binding molecule of this invention.

In one embodiment, the CDRs of the capture agent and/or detection agent are the CDRs of an antibody in Table 2.

In one embodiment, the immunoassay is for the detection and/or measuring of a coronavirus or coronavirus polypeptide, e.g., the S1 or S2 subunit of the COVID-19 S polypeptide, in a sample, wherein the method of making the immunoassay comprises: a) coating a solid support with the capture agent; b) contacting the capture agent with the sample under conditions to form a capture agent:coronavirus complex and/or a capture agent:coronavirus polypeptide complex; c) removing unbound sample; d) contacting the complex with the detection agent; e) removing unbound detection agent; and f) detecting and/or measuring the amount of complex, wherein the amount of complex is indicative of the amount of coronavirus or coronavirus polypeptide in the sample.

In an embodiment, the ELISA is a competitive ELISA. In an embodiment, the ELISA is a direct ELISA. In an embodiment, the ELISA is an indirect ELISA.

As used herein, “solid supports” include any material to which coronavirus or a coronavirus polypeptide and multivalent binding molecules are capable of binding to. For example, the solid support can include plastic, glass, polystyrene, nylon, polypropylene, nylon, polyethylene, dextran, amylases, natural and modified celluloses and polyacrylamides. For example, the solid support is a microtiter plate, magnetic beads, latex beads or array surfaces.

In embodiments of the immunoassays described herein, the sample may be contacted with an multivalent binding molecules under appropriate conditions, for example, at a given temperature and for a sufficient period of time, to allow effective binding of a coronavirus polypeptide to the multivalent binding molecules, thus forming a multivalent binding molecules:coronavirus polypeptide complex, such as a capture multivalent binding molecules:coronavirus polypeptide complex. For example, the contacting step is carried out at room temperature for about 30 minutes, about 60 minutes, about 2 hours or about 4 hours. For example, the contacting step is carried out at about 4° C. overnight.

The multivalent binding molecules as described herein may be, e.g., complexed with a coronavirus polypeptide in a suitable buffer. For example, the buffer has a pH of about 5.0 to about 10.0. For example, the buffer has a pH of 4.5, 6.5 or 7.4. For example, the buffer is a HBS-EP buffer, a KRH buffer or Tris-buffered saline. For example, the buffer comprises BSA and/or TWEEN™ 20.

Any unbound material in a sample may be removed by washing so that only the formed multivalent binding molecules:coronavirus polypeptide complex remains on the solid support. For example, the unbound sample is washed with phosphate-buffered saline, optionally comprising bovine serum albumin (BSA).

In an embodiment, the detection agent is labelled and/or conjugated to a tag.

For example, the detection agent is directly labelled and/or conjugated. For example, the detection agent is indirectly labelled and/or conjugated. Indirect labels include for example fluorescent or chemiluminescent tags, metals, dyes or radionuclides attached to the antibody. Indirect labels include for example horseradish peroxidase, alkaline phosphatase (AP), beta-galactosidase and urease. For example, HRP can be used with a chromogenic substrate, for example tetramethybenzidine, which produces a soluble product in the presence of hydrogen peroxide that is detectable at 450 nm.

An embodiment of the invention is an assay for detecting and/or measuring level of coronavirus or a coronavirus polypeptide in a sample, the assay comprising: a) contacting a sample with the multivalent binding molecules of this invention under conditions to form a multivalent binding molecule:coronavirus or a coronavirus polypeptide complex; and b) detecting and/or measuring the multivalent binding molecule:coronavirus or a coronavirus polypeptide complex. A further embodiment is an assay for detecting and/or measuring coronavirus or a coronavirus polypeptide the method comprising: a) contacting a sample, the sample being a biological fluid, with the multivalent binding molecules of this invention under conditions to form a multivalent binding molecule:coronavirus or a coronavirus polypeptide complex; and b) detecting and/or measuring the multivalent binding molecules:coronavirus or a coronavirus polypeptide complex.

In an embodiment, the assay is for detecting coronavirus or a coronavirus polypeptide and the assay is performed under non-denaturing or mildly denaturing conditions.

In an embodiment, the multivalent binding molecule:coronavirus or a coronavirus polypeptide complex is detected directly for example wherein the multivalent binding molecule is labeled with a detectable tag or fusion moiety. In an embodiment, the complex is detected indirectly using a secondary multivalent binding molecules or antibody specific for the multivalent binding molecule:coronavirus or a coronavirus polypeptide complex.

In an embodiment, the assay for detecting the multivalent binding molecule in complex with a coronavirus or coronavirus polypeptide is an immunoprecipitation, immunoblot, immunohistochemistry or immunocytochemistry, proximity ligation assay (PLA), mass spectroscopy-based techniques (CyTof) and fluorescence-activated cell sorting (FACS), and mass spectroscopy-based technique.

Detecting can be performed using methods that are qualitative or measured using quantitative methods, for example by comparing to a standard or standard curve.

In an embodiment, sample may be a biological sample from a subject, e.g., a human or other primate, or the sample may be from another source. The biological sample from a subject may be a tissue or a fluid, e.g., blood and its component parts, serum or plasma, saliva, a lung secretion or a lung lavage, nasal secretion, or urine etc. Other samples may be, e.g., wastewater or a body of water where wastewater drains, e.g., a river, canal, pond, bay etc.

### Kits

A further aspect relates to a kit comprising i) a multivalent binding molecule of this invention, ii) a nucleic acid, iii) a composition or iv) a recombinant cell of this invention, comprised in a vial such as a sterile vial or other housing and optionally a reference agent and/or instructions for use thereof.

In an embodiment, the kit further comprises an additional binding agent, e.g., another multivalent binding molecule of this invention comprising the light chain CDRS having amino acid sequences of the hk CDR1, CDR2 and CDR3 of an antibody of Table 2 and heavy chain CDRs of the hIg1 CDR1, CDR2 and CDR3 of an antibody in Table 2, or of an antibody that binds a coronavirus polypeptide.

In an embodiment, the kit comprises components and/or is for use in performing an assay described herein.

For example, the kit is an ELISA kit and can comprise a capture agent that binds coronavirus, e.g., an antibody or multivalent antibody of this invention, for example attached to a solid support, and a second detection agent, e.g., an antibody or multivalent binding molecule of this invention that binds to (i) coronavirus or a coronavirus polypeptide, and/or (ii) a complex of the capture agent:coronavirus or a coronavirus polypeptide, and that is conjugated to a detectable label. Any combination of antibodies or antibody fragments or multivalent binding molecules described herein can be used with the provision that at least one of the capture or detection agent is a coronavirus binding multivalent binding molecule of this invention.

In an embodiment, the kit is a diagnostic kit and the instructions are directed to a method described herein.

General methods in molecular and cellular biochemistry can be found in such standard textbooks as Molecular Cloning: A Laboratory Manual, 3rd Ed. (Sambrook et al., CSH Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998).

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

### EXAMPLES

### Cell Culture

Vero E6 (CRL-1586, American Type Culture (ATCC)), HEK293T (CRL-3216 ATCC) and HEK293T cells stably overexpressing ACE2 were maintained at 37° C. in 5% CO₂ in DMEM containing 10% (vol/vol) FBS. Expi293F cells (Thermo Fisher Scientific, A1452) were maintained at 37° C. in 8% CO₂ in Expi293F Expression Medium (Thermo Fisher Scientific, A1435102).

### Protein Production

The previously reported piggyBac transposase-based expression plasmid PB-T-PAF (PMID: 23476064) containing a CMV promotor (PB-CMV) and a woodchuck hepatitis virus posttranscriptional regulatory element (WPRE) was used for large-scale transient expression. cDNA encoding the SARS-CoV-2 S protein ectodomain trimer (residues 1-Attorney Docket No. 17344P-000035-US-NP 1211), followed by a foldon trimerization motif (PMID: 9261070), a 6×His tag and an AviTag biotinylation motif (PMID: 25560075) was cloned in to the PB-CMV vector using standard molecular biology techniques and residues 682-685 (RRAR) and 986-987 (KV) were mutated to SSAS and two proline residues to remove the furin cleavage site on the SARS-CoV-2 S protein and stabilize the pre-fusion form (PMID: 28807998), respectively. The SARS-CoV-2 receptor binding domain (RBD, residues 328-528), the soluble human ACE2 construct (residues 19-615), and the SARS-CoV RBD (residues 315-514), each followed by a 6×His tag and an AviTag, were similarly cloned in to the same vector. For expression, PB-CMV expression constructs were mixed with Opti-MEM medium containing 293fectin reagent (Thermo Fisher) and the mixture incubated for 5 minutes before addition to the shaker flask containing 10⁶ Freestyle 293-F cells/mL grown in suspension in Freestyle 293 expression medium (Thermo Fisher). Expression was allowed to continue for 6 days before purification.

Protein purification and in vitro biotinylation

Expressed proteins were harvested from expression medium by binding to Ni-NTA affinity resin, eluted with IX PBS containing 300 mM imidazole and 0.1% (v/v) protease inhibitor cocktail (Sigma, P-8849), then further purified by size-exclusion chromatography. For the RBDs and ACE2, a Superdex 200 Increase (GE healthcare) column was used. For the S protein ectodomain, a Superose 6 Increase (GE healthcare) column was used. Purified proteins were site-specifically biotinylated in a reaction with 200 μM biotin, 500 μM ATP, 500 μM MgCl2, 30 μg/mL BirA, 0.1% (v/v) protease inhibitor cocktail and not more than 100 μM of the protein-AviTag substrate. The reactions were incubated at 30° C. for 2 hours and biotinylated proteins then purified by size-exclusion chromatography.

### Phage Panning

A highly validated, synthetic, phage-displayed antibody library (Persson et al. CDR-H₃ diversity is not required for antigen recognition by synthetic antibodies. J Mol Biol 2013; 425:803-11) was panned on SARS-CoV-2 RBD in solution. In each round, phage library was first depleted on neutravidin immobilized in wells of a 96 Maxisorp plate from a 2 μg/mL solution incubated with shaking overnight at 4° C., then incubated with 50 nM biotinylated RBD in solution for two hours at room temperature. Protein-phage complexes were captured in wells coated with neutravidin as above for 15 min at RT, then washed with IX PBS pH7.4 containing 0.05% TWEEN™ and phage eluted for 5 min with 0.1 M HCl before neutralizing with IM Tris pH 8.0. Eluted phage were amplified, purified, then panned against target for a total of 5 rounds, before isolation, amplification and sequencing of individual phage clones as previously described (Persson).

### Enzyme Linked Immunosorbent Assays

For ELISAs, plates were coated with neutravidin, as above, then blocked with 0.2% BSA in PBS for 1 hr. Biotinylated target protein was captured from solution by incubation in neutravidin-coated and BSA-blocked wells for 15 min with shaking at RT before addition and 30 min incubation of binding phage or antibody. Plates were washed then incubated with an appropriate secondary antibody before development with TB substrate as described (Sidhu et al., Journal of Molecular Biology Vol. 338(2): 299-310 (2004)).

### Tetravalent Antibody Construction

DNA fragments encoding heavy chain FAb regions (VH-CH1; terminating at threonine 238, Kabat numbering) were amplified by PCR from the IgG expression constructs. Tetravalent antibody constructs were generated by fusing these fragments with their respective IgG heavy chain in the pSCSTa mammalian expression vector using Gibson assembly (NEBuilder HiFi DNA Assembly Cloning Kit; NEB. Fab-IgG constructs were arranged by fusing a heavy chain FAb domain to the N-terminus of the IgG via a S(G4S)₃ linker. IgG-Fab constructs were arranged by fusing a heavy chain FAb domain to the C-terminus of using a G(G4S)₂GGGTG linker. For both formats, the Fc region terminated at glycine 447 (Kabat numbering).

### Antibody Production

IgG and tetravalent antibodies were produced in Expi293F (ThermoFisher) by transient transfection, as described (Miersch, BioRxiv, 2020 https://doi.org/10.1101/2020.06.05.137349). For IgG expression, equivalent amounts of plasmids encoding heavy chain and light chains were transfected, whereas for tetravalent modalities, a ratio of 2:1 light chain to heavy chain plasmids was used. After a 5-day expression period, antibodies were purified using rProtein A Sepharose (GE Healthcare), then buffer exchanged and concentrated using Amicon Ultra-15 Centrifugal Filter devices (Millipore). IgGs were stored in PBS (Gibco), while tetravalent antibodies were stored in a buffer consisting of 10 mM L-Histidine, 0.9% sucrose, 140 mM NaCl, pH 6.0.

### Size-Exclusion Chromatography

Fifty micrograms of protein were injected onto a TSKgel BioAssist G3SWxl (Tosoh) fitted with a guard column using an NGC chromatography system and a C96 autosampler (Biorad). The column was preequilibrated in a PBS mobile phase and protein retention was monitored by absorbance at 215 nm during a 1.5 CV isocratic elution in PBS.

### Biolayer Interferometry

The binding kinetics and estimation of apparent affinity (K_{D}) of antibodies against SARS-CoV-2 RBD we determined by biolayer interferometry experiments performed on an Octet HTX instrument (ForteBio) at 1000 rpm and 25° C. Biotinylated CoV2 S protein was first captured on streptavidin biosensors from a 2 μg/mL solution to achieve a binding response of 0.4-0.6 nm and unoccupied sites quenched with 100 μg/mL biotin. Antibodies were diluted with assay buffer (PBS, 1% BSA, 0.05% TWEEN™ 20) and 67 nM of an unrelated biotinylated protein of similar size was used as negative control. Following equilibration with assay buffer, loaded biosensors were dipped for 600 s into wells containing 3-fold serial dilutions of each antibody from 67 nM and subsequently were transferred for 600 s back into assay buffer. Binding response data were reference subtracted and were fitted with 1:1 binding model using ForteBio's Data Analysis software 9.0.

### Differential Scanning Fluorimetry

Thermostability of expressed antibodies were determined by differential scanning fluorimetry (DSF) using Sypro Orange as described (Majka, J Biomol Screen, 2015) instead using a 1 μM solution of antibody and temperature range from 25-100° C. in 0.5° C. increments.

### Generation of SARS-CoV-2 Pseudovirus

To generate SARS-Cov-2 pseudovirus, human embryonic kidney 293 (HEK 293, ATCC® CRL-1573™) cells were seeded at 0.3×10⁶ cells/well in DMEM (ThermoFisher Scientific, Cat. No. 11995073) supplemented with 10% FBS and 1% penicillin-streptomycin (Gibco, Cat. No. 15070063) and grown overnight at 37° C., 5% CO₂. HEK 293 cells were then co-transfected with 1 μg of pNL4-3.luc.R-E-plasmid (luciferase expressing HIV-1 with defective envelop protein) (NIH AIDS Reagent Program, Cat. No. 3418) and 0.06 μg of CMV-promoter driven plasmid encoding SARS-CoV-2 wild type or mutant S protein variants using Lipofectamine™ 2000 transfection reagent (ThermoFisher Scientific, Cat. No. 11668027). Pseudovirus was harvested by collecting supernatant 48 h after transfection and filter sterilized (0.44 μm, Millipore Sigma, Cat. No. SLHA033SS).

### Pseudovirus Entry Assay

HEK 293 cells (ATCC® CRL-1573) stably over-expressing full-length human ACE2 protein were seeded in 96 well white polystyrene microplates (Corning, Cat. No. CLS3610) at 0.03×10⁶ cells/well in DMEM (10% FBS and 1% Pen-Strep), and grown overnight at 37° C., 5% CO₂. To test the inhibition of pseudovirus entry of antibodies, the desired concentrations of antibodies were mixed with pseudoviruses, incubated at room temperature for 10 m, and were used to infect cells. The cells were incubated at 37° C., 5% CO₂ for 6 hours, then the medium was replaced with fresh DMEM (10% FBS and 1% Pen-Strep), and again every 24 hours up to 72 hours. To measure the luciferase signal (virus uptake), DMEM was removed and cells were replaced in DPBS (ThermoFisher, Cat. No. 14190250) and mixed with an equal volume of ONE-Glo™ EX Luciferase Assay System (Promega, Cat. No. 8130). Relative luciferase units were measured using a BioTek Synergy Neo plate reader (BioTek Instruments Inc.). The data was then analyzed by GraphPad Prism Version 8.4.3 (GraphPad Software, LLC.).

### SARS-CoV-2 Neutralization Assay

SARS-CoV-2 strain 2019 n-CoV/USA_WA1/2020 was obtained from the Centers for Disease Control and Prevention (gift of Natalie Thornburg). Virus stocks were produced in Vero CCL81 cells (ATCC) and titrated by focus-forming assay on Vero E6 cells. Serial dilutions of mAbs were incubated with 10² focus-forming units (FFU) of SARS-CoV-2 for 1 h at 37° C. MAb-virus complexes were added to Vero E6 cell monolayers in 96-well plates and incubated at 37° C. for 1 h. Subsequently, cells were overlaid with 1% (w/v) methylcellulose in MEM supplemented with 2% FBS. Plates were harvested 30 h later by removing overlays and fixed with 4% PFA in PBS for 20 min at room temperature. Plates were washed and sequentially incubated with 1 μg/mL of CR3022 (Yuan et al, Science, 2020,) anti-S antibody and HRP-conjugated goat anti-human IgG in PBS supplemented with 0.1% saponin and 0.1% BSA. SARS-CoV-2-infected cell foci were visualized using TrueBlue peroxidase substrate (KPL) and quantitated on an ImmunoSpot microanalyzer (Cellular Technologies). Data were processed using Prism software (GraphPad Prism 8.0).

### Results

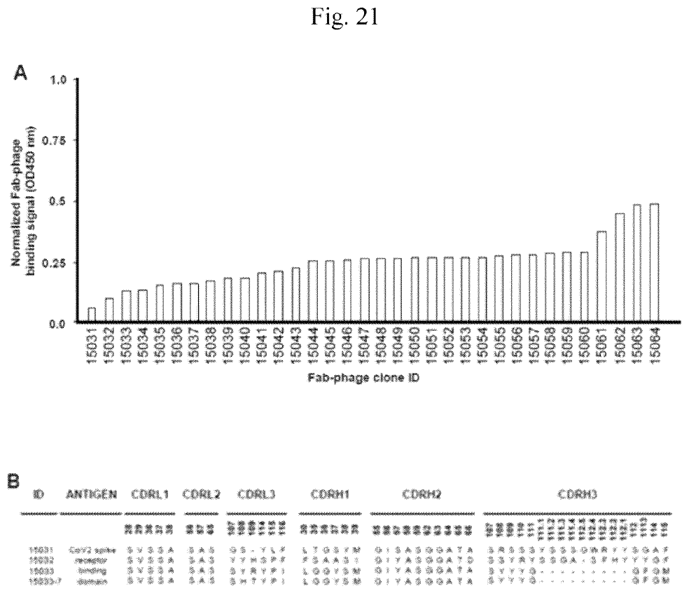

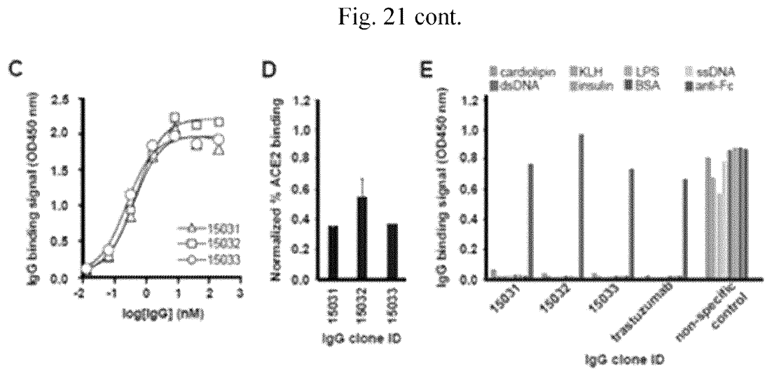

Using a phage-displayed human antigen-binding fragment (Fab) library similar to the highly validated library F (Persson et al., J Mol Biol 2013; 425:803-11), we performed four rounds of selection for binding to the biotinylated RBD of SARS-CoV-2 immobilized on streptavidin-coated plates. Screening of 384 clones for binding to CoV-2 RBD, revealed 358 clones that bound to the RBD but not to streptavidin, and for 66 of these clones, binding to the RBD was blocked by ACE2 (FIG. 21A). Sequencing of their antibody variable regions revealed 38 unique Fab-phage, which were then converted into the full-length synthetic human IgG1 format for purification and functional characterization.

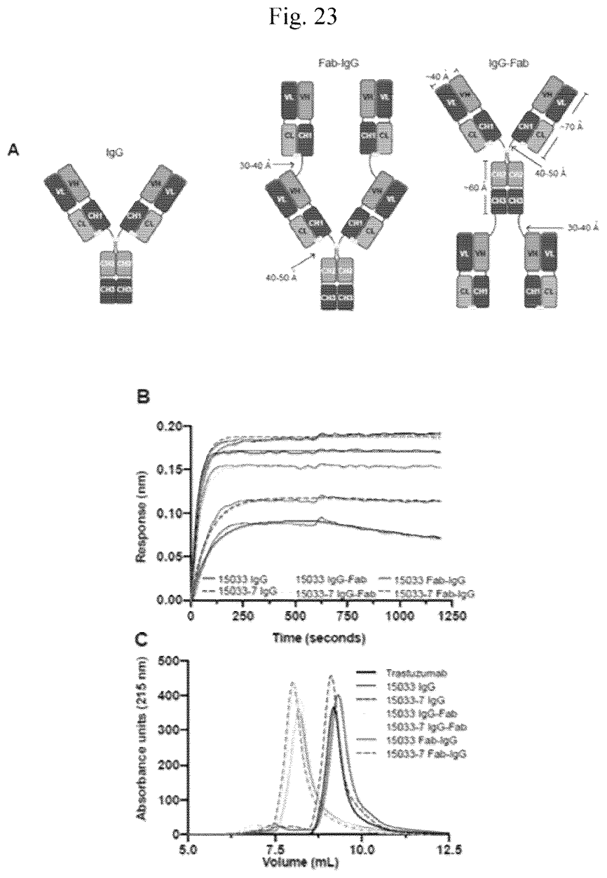

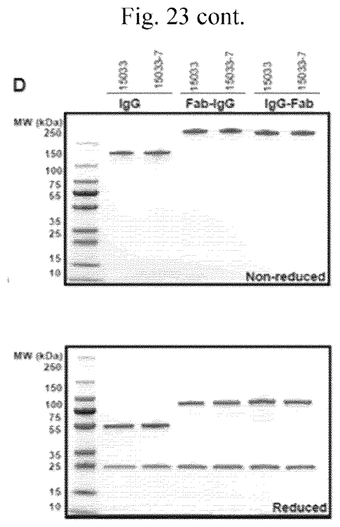

To estimate affinities, ELISAs were performed with serial dilutions of IgG protein binding to biotinylated S protein trimer captured with immobilized streptavidin, and these assays showed that three synthetic IgGs 15031, 15032, and 15033 bound with EC₅₀ values in the sub-nanomolar range (FIG. 21C, Table 5). ELISAs also confirmed that a saturating concentration of each IgG was able to at least partially block the binding of biotinylated ACE2 to immobilized S protein (FIG. 21D). Moreover, similar to the highly specific IgG trastuzumab, ELISAs showed that the three IgGs did not bind to seven immobilized proteins that are known to exhibit high non-specific binding to some IgGs (Mouquet et al. Polyreactivity increases the apparent affinity of anti-HIV antibodies by heteroligation. Nature 2010; 467:591-5; Jain et al. Biophysical properties of the clinical-stage antibody landscape. Proc Natl Acad Sci USA 2017; 114:944-9) and be predictive of pharmacokinetics (FIG. 21E). We also used biolayer interferometry (BLI) to measure binding kinetics and to determine affinities more accurately, and all three antibodies exhibited sub-nanomolar dissociation constants (Table 5, FIG. 23), in close accord with the estimated affinities determined by ELISA. IgG 15033 exhibited the highest affinity, which was mainly due to a two- or seven-fold faster on-rate than IgG 15031 or 15032, respectively, and thus, we focused further efforts on this antibody.

TABLE 5

| | | | | | | Apparent | Viral | Viral | Spike | SEC Elution | Mono- | |
|---|---|---|---|---|---|---|---|---|---|---|---|---|
| | | Yield | EC₅₀ | kₒₙ | k_{off} | K_{D} | IC₅₀ | IC₅₀ | VLP EC₅₀ | volume | dispersity | Tm2 |
| ID | Format | (mg · L⁻¹) | (nM) | (10⁵ M⁻¹s⁻¹) | (10⁻⁸s⁻¹) | (pM) | (ng · mL⁻¹) | (pM) | (nM) | (mL) | (%) | (° C.) |
| trastuzumab | IgG | 303 | NA | NA | NA | NA | NA | NA | NA | 9.20 | >95 | 79.5 |
| 15033 | IgG | 207 | 0.24 | 17 | 50 | 300 | 523 | 3540 | 0.44 | 9.34 | 91 | 81 |
| 15033-7 | IgG | 165 | 0.09 | 18 | 7 | 39 | 118 | 790 | 0.37 | 9.13 | 95 | 81 |
| 15033 | Fab-IgG | 214 | 0 18 | 46 | 1.5 | 3 | 43 | 320 | 0.27 | 8.22 | >95 | 87 |
| 15033-7 | Fab-IgG | 194 | 0.14 | 40 | 0.8 | 2 | 15 | 100 | 0.35 | 8.02 | 93 | 86.6 |
| 15033 | IgG-Fab | 166 | 0.11 | 36 | 1.9 | 5 | 51 | 340 | 0.27 | 8.22 | >95 | 87 |
| 15033-7 | IgG-Fab | 118 | 0.15 | 30 | <0.1 | <1 | 9.1 | 61 | 0.47 | 8.00 | 93 | 85 |

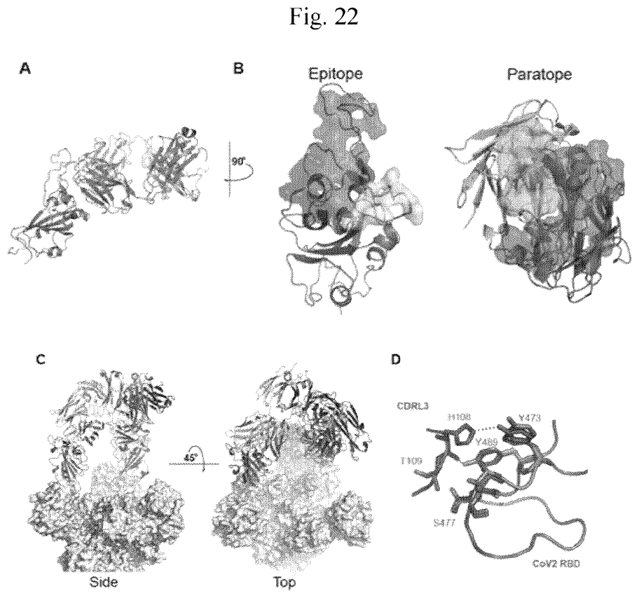

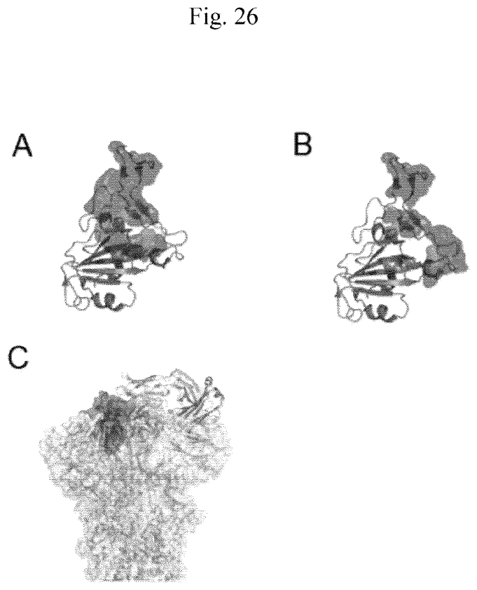

To understand the molecular basis for high affinity binding and antagonism of ACE2 binding, we solved the crystal structure of an affinity matured variant of Fab 15033 in complex with the SARS-CoV2 RBD at 3.0 Å (FIG. 22A). Binding of the Fab to the RBD results in an extensive interface, with 1130 or 1112 Å² of surface area buried on the epitope (left panel) or paratope (right panel), respectively (FIG. 22B). On the paratope side, the heavy and light chains contribute 41% and 59% of the buried surface area. On the epitope side, we compared the surface buried by ACE2 versus that buried by Fab 15033-7 and found extensive overlap in their binding sites such that 79% of Fab epitope and 69% of the ACE2 epitope overlap. The extent of overlap is illustrated in FIG. 22B (left panel—dark grey). The regions of the Fab and ACE2 epitopes that do not overlap are also shown in FIG. 22B (left panel—light grey). In the full-length S protein, the RBD can adapt two distinct conformations: the “down” and “up” conformations in which the ACE2-binding site is partially occluded or fully exposed. Interestingly, most of both the Fab (790 Å²) and ACE2 (750 Å²) epitopes are available in the down conformation with only 21 and 13% occluded from solvent (FIG. 26AB). Nevertheless, when superimposed, steric clash with the adjacent RBDs clearly prevents binding of either (FIG. 26C), whereas the entire epitope is exposed in the up conformation suggesting that they can only bind when in the “up” position. Thus, the Fab 15033 mimics ACE2, as the two molecules have similar epitopes and both most likely bind to the RBD in the up conformation in the S protein. Moreover, superposition of the Fab/RBD complex with the structure of the S protein of SARS-CoV2 (PDB ID:6X2B) showed that simultaneous binding of two Fabs to two RBDs in the up conformation can be readily accommodated (FIG. 22C).

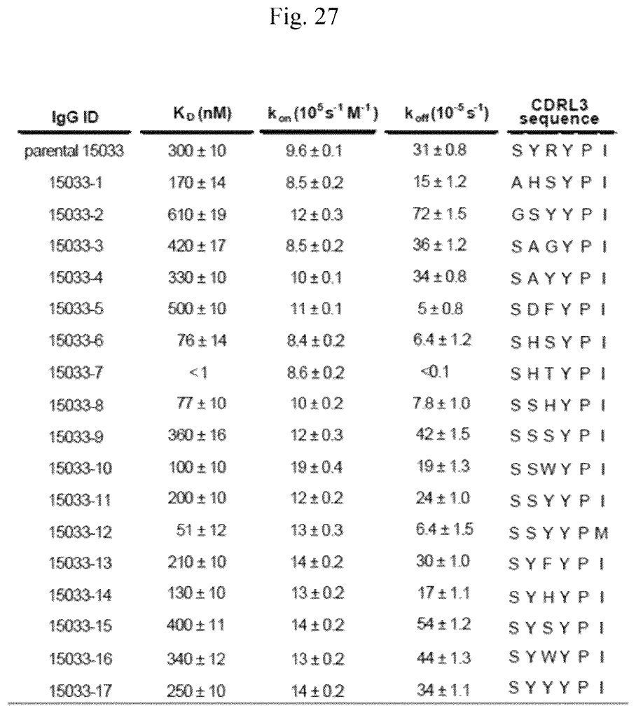

Based on our structural analysis and the rapid combinatorial protein engineering and our evaluation of binding kinetics described herein we improved the affinity of IgG 15033 and contemplate that the multivalent binding molecules described herein based on this IgG will likewise show improved affinity. The synthetic library was designed with tailored diversification of key positions in all three heavy chain complementarity-determining regions (CDRs) and the third CDR of the light chain (CDR-L3). Notably, CDR-L3 is substantially buried at the interface with the RBD (FIG. 22B—right panel), and given that sequence diversity was incompletely sampled in the naïve library, we reasoned that the already high affinity of IgG 15033 could be further improved by recombining the heavy chain with a library of light chains with diverse CDR-L3 sequences. Following selection for binding to the RBD, the light chain library yielded numerous variants, 17 of which were purified in the IgG format and compared to IgG 15033 by BLI (FIG. 27). This analysis revealed that most of the 17 variant light chains resulted in IgGs with improved affinities compared with IgG 15033, and in particular, IgG 15033-7 exhibited significantly improved affinity (K_{D}<1 pM) due to an off-rate that was >100-fold slower than that of IgG 15033 (FIG. 23B, Table 5, FIG. 27). The 15033-7 paratope contained two substitutions in CDRL3 relative to 15033. The Tyr->His mutation at position 108 introduced a novel hydrogen bond with RBD Tyr473 that appears to pull electron density toward CDRL3 (FIG. 22D). Alternately, the role of R109T mutation, while not entirely clear can be deduced, in part, by comparison to clone 15033-6. While clone 15033-6 bears only a single residue mutated from 15033-7, this possesses an apparent affinity at least one order of magnitude lesser, despite conservation of charge and polarity. Thus, in addition to the clear role of the Y108H mutation, T109 may also make a substantial contribution to the improved binding.

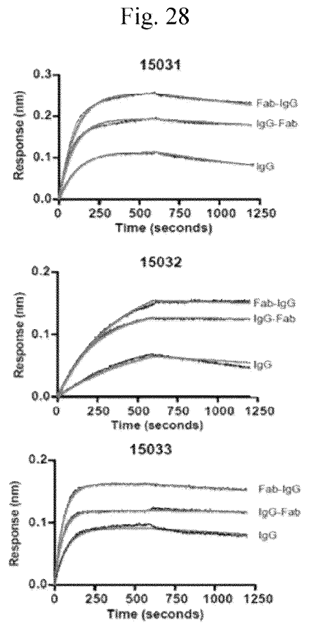

We further enhanced the binding and neutralization of our best Abs by generating tetravalent antibody formats that do not exist in nature. Each SARS-CoV2 particle displays multiple S protein trimers, suggesting that multivalent Fab binding could significantly enhance apparent affinity, especially since at least two Fab 15033 molecules could potentially bind to even a single S protein trimer (FIG. 22C). Based on our analysis, our multivalent binding molecules enhanced affinity beyond what could be achieved with a natural bivalent IgG. We constructed tetravalent versions of each of the four synthetic IgGs—clones 15031, 15032, 15033 and 15033-7, by fusing additional Fabs comprising the heavy chain and light chain CDRs of the clones to either the N- or C-terminus of the IgG heavy chain to construct molecules termed Fab-IgG or IgG-Fab, respectively (FIG. 23A). All our tetravalent molecules exhibited greatly reduced off rates compared with their bivalent counterparts, and consequently, significantly higher apparent affinities with dissociation constants in the low single-digit picomolar range (FIG. 23B, Table 5, FIG. 28).

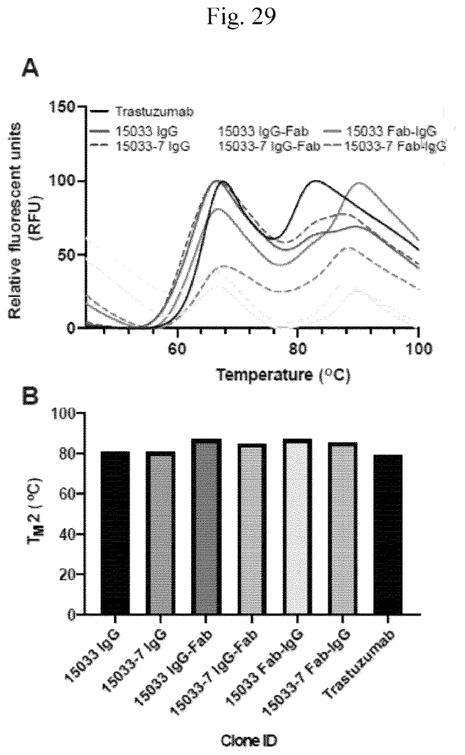

Our ultimate aim was to produce therapeutic antibodies that could be used to treat COVID-19 in patients, and aside from high affinity and specificity, effective antibody drugs must also possess favorable biophysical properties including high yields from recombinant expression in mammalian cells, high thermodynamic stability, and lack of aggregation and excessive hydrophobic surface area. All synthetic IgGs and Fab-IgG and IgG-Fabs comprising the CDRs of 15031, 15032, 15033 and 15033-7 were produced in very high yields by transient expression in 293T cells (160-200 mg/L, Table 5). All proteins were also highly thermostable with melting temperatures (Tm2) of the CH3/Fab domain (Ionescu et al. Contribution of Variable Domains to the Stability of Humanized IgG1 Monoclonal Antibodies. J. Pharm. Sci. 2008, 97, 1414-1426. 10. Garber, E.; Demarest, S. J. A Broad Range of Fab Stabilities within a Host of Therapeutic IgGs. Biochem. Biophys. Res. Commun. 2007, 355, 751-757.) ranging from 81-87° C., which was comparable to the melting temperature of the trastuzumab Fab (79.5° C., Table 5, FIG. 29). Size exclusion chromatography revealed that all IgGs eluted as a predominant monodisperse single peak with retention times nearly identical to that of trastuzumab (FIG. 23C and Table 5), and the monomeric fraction was calculated to be 91 to >95% (Table 5). Notably, the tetravalent molecules in either the Fab-IgG or IgG-Fab form also eluted as single peaks with retention times slightly in advance relative to trastuzumab, consistent with their larger molecular weights and could be purified to near homogeneity by protein-A affinity chromatography, as evidenced by SDS-PAGE (FIG. 23D). Taken together, these analyses demonstrate that the IgGs and their tetravalent derivatives possess good drug-like biophysical properties which we contemplate will enable drug development at large scale.

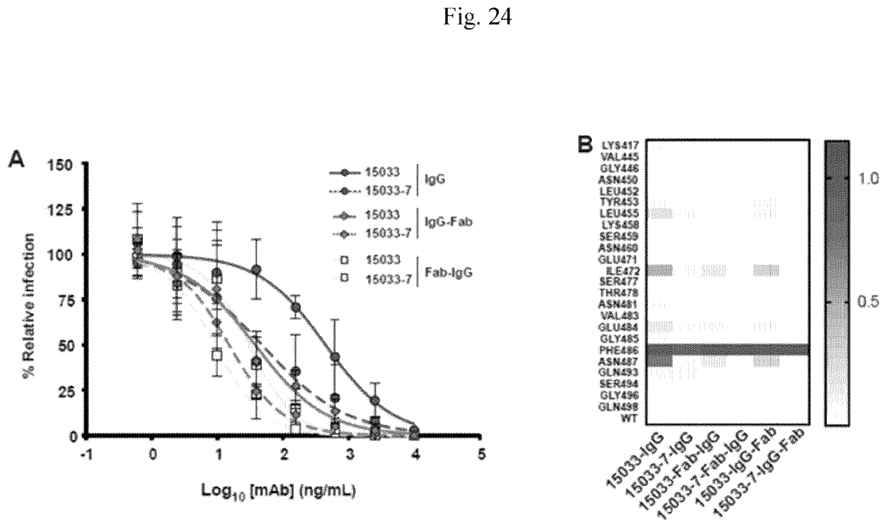

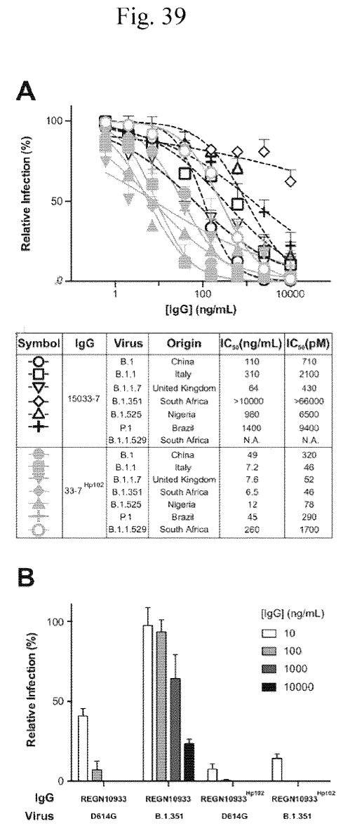

We assessed the effects of the synthetic IgGs on virus infection in a focus reduction neutralization test that measured the infection of ACE2-expressing VeroE6 cells with the clinically isolated SARS-CoV-2 strain 2019 n-CoV/USA_WA1/2020. All three synthetic IgGs from the naïve library exhibited dose-dependent neutralization of viral infection, confirming their neutralization capacity and the absence of cytopathic effects (FIG. 24, Table 5). Consistent with affinities, IgG 15033 was most potent with an IC₅₀ of 3.5 nM and its potency was confirmed with similar inhibition assays against a second strain of SARS-CoV-2 (2019-nCoV/Italy-INMI) (IC₅₀=0.5 nM, FIG. 39). Moreover, the tetravalent Fab-IgG and IgG-Fab versions of 15033 exhibited significantly improved potencies with IC₅₀ values of 320 or 320 μM, respectively. Also consistent with affinity, the optimized IgG 15033-7 exhibited very high potency with an IC₅₀ of 790 μM and its tetravalent Fab-IgG and IgG-Fab versions exhibited the best potencies of all molecules tested with IC₅₀ values of 100 and 61 μM, respectively. Taken together, these results show that naïve synthetic antibody libraries can yield highly potent neutralizing antibodies (nAbs) with excellent drug-like properties, and further modification and reformatting to tetravalent formats can produce drug-like molecules with ultra-high potencies beyond those of bivalent IgGs.

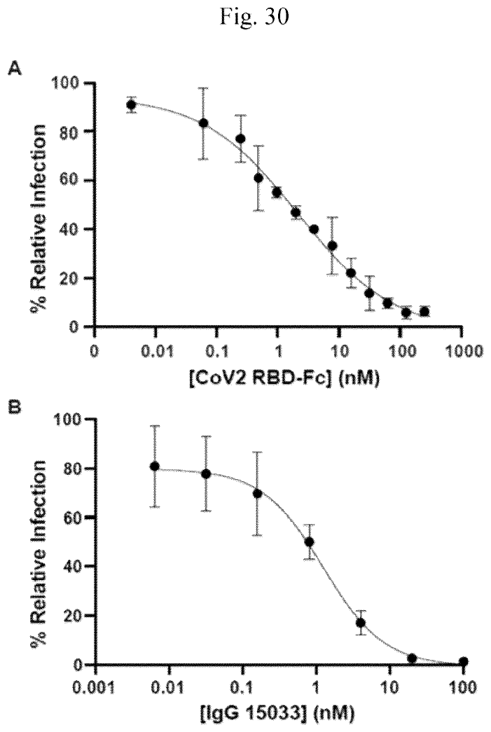

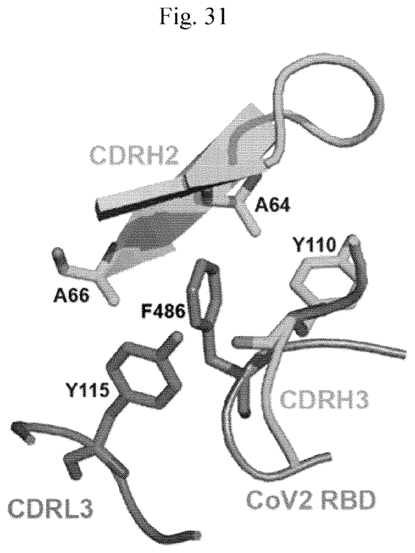

To explore antibody-mediated neutralization of potential escape mutants, we generated an HIV-gag-based virus-like particle pseudo-typed with SARS-CoV-2 S protein and assessed uptake in HEK-293 cells stably over-expressing ACE2 cells. After confirming ACE2-dependent uptake, RBD-mediated inhibition (FIG. 30A) and susceptibility to antibody-mediated inhibition of uptake (FIG. 30B), we generated a panel of 44 alanine-scanning mutants in SARS-Cov2 S protein pseudo-typed VLPs that spanned every residue within and in close proximity to the surface of the RBD buried by ACE2 as a model to investigate potential escape mutants. Of these, 20 mutations reduced internalization of the pseudo-typed VLP to <25% of the CoV2 parent VLP and were omitted from further analysis. For the remaining mutants, IgG 15033 was capable of fully inhibiting uptake of 14 of the 24 mutants at 50 nM antibody, while the other mutants exhibited either complete resistance (F486) or partial resistance ranging from <10% to ˜70% (FIG. 24B). Treatment with affinity matured 15033-7, however, reduced uptake of all but one mutant (F486) and fully neutralized 5 of the 10 that were resistant to IgG 15033. Similarly, conversion of IgG 15033 to either tetravalent antibody format reduced resistance to the parent IgG and more importantly, the matured 15033-7 variant, as either tetravalent format, eliminated remaining resistance in all but a single clone (F486). Inspection of the 15033-7/RBD complex reveals that this residue sits in the center of the epitope making contacts with numerous residues in CDRs L3, H2 and H3 (FIG. 31) and is a clear potential vulnerability. However, inspection of the GISAID database detailing the sequences of clinical isolates have detected no such mutation circulating to date.

### E. Tetravalent IgG-Like Molecules with Enhanced Potency and Effectiveness Against Variants of Concern (VOC)

Most natural neutralizing antibodies (nAbs) target the S1 subunit, and bind to the RBD and compete with ACE2. A distinct subset of nAbs, however, have been shown to target a neutralizing epitope on the NTD (Liu, L., et al., (2020) Potent neutralizing antibodies against multiple epitopes on SARS-CoV-2 S protein. Nature 584, 450-456.). Several natural nAbs have been produced recombinantly and engineered further to develop drugs that have been successful for inhibiting SARS-CoV-2 infection in patients (Taylor, P. C., et al., (2021) Neutralizing monoclonal antibodies for treatment of COVID-19. Nat Rev Immunol 21, 382-393.). However, current approved antibody drugs must be administered at very high doses and have proven to be ineffective against many variants of concern (VOCs) that have arisen since the original COVID-19 outbreak. Indeed, most VOCs that resist current therapeutic nAbs contain mutations within the RBD that disrupt binding to the nAbs (Wang, P., et al., (2021) Antibody resistance of SARS-CoV-2 variants B.1.351 and B.1.1.7. Nature 593, 130-135.) but not to ACE2 (Zhu, X., et al., (2021) Cryo-electron microscopy structures of the N501Y SARS-CoV-2 S protein in complex with ACE2 and 2 potent neutralizing antibodies. PLoS Biol 19, e3001237.).

To address these limitations on the potency and breadth of coverage of current bivalent IgG therapies, several groups have developed higher valence protein-based inhibitors (De Gasparo, R., et al., (2021) Cryo-electron microscopy structures of the N501Y SARS-CoV-2 S protein in complex with ACE2 and 2 potent neutralizing antibodies. PLoS Biol 19, e3001237.). These include small modular Ab domains or non-antibody scaffolds that can be assembled as multimers with enhanced potency due to simultaneous engagement with all three RBDs on an S protein trimer (Rujas, E., et al., (2021) Multivalency transforms SARS-CoV-2 antibodies into ultrapotent neutralizers. Nat Commun 12, 3661-12.) (Cao, L., et al., (2020) De novo design of picomolar SARS-CoV-2 miniprotein inhibitors. Science 370, 426-431.). Alternatively, we have shown that the fusion of additional Fab arms to either the N- or C-terminus of an IgG heavy chain results in tetravalent IgG-like molecules with enhanced potency and effectiveness against VOCs that resist bivalent IgGs (Miersch, et al., (2021) Tetravalent SARS-CoV-2 Neutralizing Antibodies Show Enhanced Potency and Resistance to Escape Mutations. Journal of Molecular Biology 433, 167177.).

Described herein are synthetic peptides that bind to the S protein to augment neutralization potency of IgGs in the form of tetravalent peptide-IgG fusions comprising binding sites of potent nAbs combined with the small, modular binding sites of peptides using naïve phage-displayed peptide libraries to derive synthetic peptides that bind to neutralizing epitopes on the RBD or the NTD. We showed that these small peptides could be fused to a moderate affinity nAb to develop peptide-IgG fusions with affinities enhanced by over two orders of magnitude. Most importantly, one such peptide fusion was able to greatly enhance the neutralization potency against SARS-CoV-2 and VOCs for a high affinity nAb we had engineered earlier (Miersch, et al., (2021) Tetravalent SARS-CoV-2 Neutralizing Antibodies Show Enhanced Potency and Resistance to Escape Mutations. Journal of Molecular Biology 433, 167177.), and also, for a clinically approved therapeutic nAb developed by others (Hansen, J., et al. (2020) Studies in humanized mice and convalescent humans yield a SARS-CoV-2 antibody cocktail. Science 369, 1010-1014.).

The trimeric structure of the SARS-CoV-2 S protein can be exploited to engineer Ab-based inhibitors with enhanced neutralization, by engagement of three neutralizing epitopes on a single trimer. Structural studies from us and others have shown that potent neutralizing IgGs engage two RBDs on an S protein trimer (Yan, R., et al., (2021) Structural basis for bivalent binding and inhibition of SARS-CoV-2 infection by human potent neutralizing antibodies. Cell Res. 31, 517-525.), and we have shown that the addition of Fab arms to either end of an IgG can further enhance neutralization by enabling engagement to the third RBD (Miersch, S., et al., (2021) Tetravalent SARS-CoV-2 Neutralizing Antibodies Show Enhanced Potency and Resistance to Escape Mutations. Journal of Molecular Biology 433, 167177.).

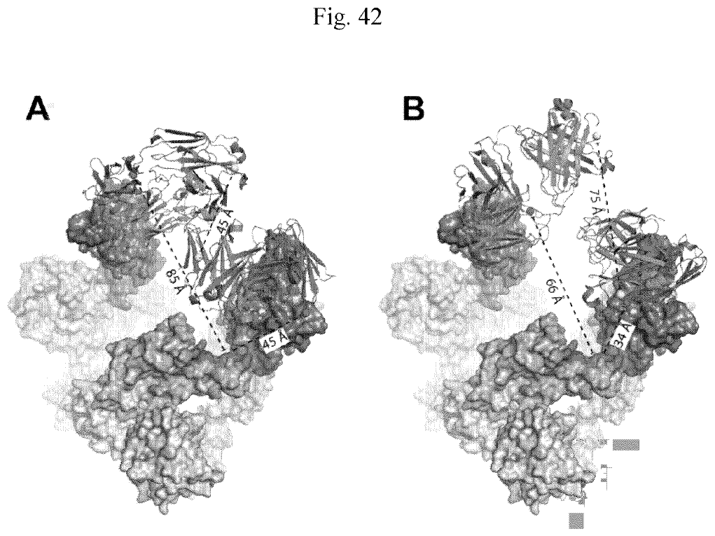

Described herein are small peptides that greatly enhance the affinities and potencies of tetravalent peptide-IgG fusions compared with our bivalent IgG 15033-7. Most importantly, peptide-IgG 33-7^{Hp102} acted as a potent inhibitor of virus variants that resisted IgG 15033-7. Indeed, we showed further that a peptide-IgG version of the approved drug RGN10933 was able to potently neutralize a virus variant against which REGN10933 was completely ineffective. To gain insight into the structural basis for how a peptide within a peptide-IgG fusion could enhance affinity, we examined our published model of two 15033-7 Fabs bound to the S protein trimer, reasoning that this likely provides an accurate view of how the two Fab arms of a bivalent IgG would bind (FIG. 42A). In this model, the RBDs bound to Fabs are in an “up” conformation, whereas the unbound RBD is in a “down” conformation. The C-termini of the two Fab HCs are separated by 45 Å and are well-positioned to be linked to an Fc in an IgG molecule, whereas their N-termini are fairly close to the unbound RBD. In particular, the N-terminus of one HC is 45 Å from the center of the exposed region of the neutralizing epitope for Fab 15033-7 on the unbound RBD. Peptide p102 likely binds to a site that overlaps with this epitope, given that peptide p102 and IgG 15033-7 compete for binding to the S protein (FIG. 3C). Consequently, in peptide-IgG 33-7^{Hp102} with two Fabs bound to an S protein timer, it is reasonable to assume that one of the p102 peptides could bind simultaneously to this third epitope, and the estimated length (˜70 Å) of the extended 20-residue linker that connects the peptide to the IgG is consistent with this model.

Next, an analogous structural model of two RGN10933 Fabs bound to an S protein trimer (FIG. 42B) was examined. The epitopes for REGN10933 and 15033-7 Fabs share significant overlap, but the REGN10933 Fabs are oriented such that the N-termini of the two HCs are 34 Å or 66 Å from the 15033-7 epitope on the unbound RBD. Consequently, the ˜70 Å linker that connects each peptide to the IgG would be sufficient to enable either of the two fused peptides within peptide-IgG10933^{Hp102} to bind a site close to this epitope.

We also developed peptides that bound to the NTD, likely at a site that overlaps with epitopes for other neutralizing nAbs (FIG. 3C), and we showed that peptide p16 greatly enhanced the affinity of peptide-IgG 33^{LN1}. While not yet explored, it is likely that the enhanced affinity of peptide-IgG 33^{LN1} should also be reflected in enhanced potency of virus neutralization. Notably, peptides p102 and p16 appear to work best as fusions to the HC or LC, respectively, and given that they recognize completely distinct epitopes, it will be interesting to explore whether dual fusions of the two peptides could enhance the potencies of peptide-IgG fusions even further, and these studies are ongoing.

The ultimate value of peptide-IgG fusions targeting SARS-CoV-2 and its variants resides in the potential for the development of superior therapeutics for the treatment of COVID-19 and other viral diseases. Currently the biophysical properties of the peptide-IgG fusions are being assessed and optimized with the aim of developing drug-grade biologics. However, there is already strong precedent for peptide-IgG fusions as clinical drugs in the form of biologics that have reached phase 2 clinical trials as cancer therapeutics (Autio, K. A., et al., (2020) Probody Therapeutics: An Emerging Class of Therapies Designed to Enhance On-Target Effects with Reduced Off-Tumor Toxicity for Use in Immuno-Oncology. Clin Cancer Res 26, 984-989.). These anti-cancer biologics are very similar in format to the anti-viral peptide-IgG fusions we report here (Polu, K. R., and Lowman, H. B. (2014) Probody therapeutics for targeting antibodies to diseased tissue. Expert Opin Biol Ther 14, 1049-1053.). Additionally, anti-cancer peptide-IgG fusions have exhibited good pharmacokinetic profiles (Stroh, M., et al., (2019) Quantitative Systems Pharmacology Model of a Masked, Tumor-Activated Antibody. CPT Pharmacometrics Syst Pharmacol 8, 676-684.) and tissue penetration (Desnoyers, L. R., et al., (2013) Tumor-specific activation of an EGFR-targeting probody enhances therapeutic index. Sci Transl Med 5, 207ra144-207ra144.), and they have been well tolerated in the clinic (Sanbom, R. E., et al., (2021) CX-072 (pacmilimab), a Probody PD-L1 inhibitor, in combination with ipilimumab in patients with advanced solid tumors (PROCLAIM-CX-072): a first-in-human, dose-finding study. J Immunother Cancer 9, e002446.). Taken together, our results establish peptide-IgG fusions as a powerful means for greatly enhancing the potency and coverage of next-generation biologics for the treatment of disease caused by SARS-CoV-2 and its variants.

In a first embodiment of the invention, at least one peptide that binds the SARS-CoV-2-S protein is fused to an anti-SARS-CoV-2 antibody. In another embodiment, the fusion includes at least one peptide fused to the N-terminus of at least one of the light chains of the anti-SARS-CoV-2 antibody. In another embodiment, the fusion includes at least one peptide fused to the N-terminus of at least one of the heavy chains of the anti-SARS-CoV-2 antibody. In another embodiment, the fusion molecule comprises at least one peptide fused to the N-terminus of anti-SARS-CoV-2 antibody IgG 33-7. In another embodiment, the fusion molecule comprises at least one peptide fused to the N-terminus of anti-SARS-CoV-2 antibody that engages at least one neutralizing epitopes on a single S protein trimer. In another embodiment, the fusion molecule comprises at least one peptide fused to the N-terminus of anti-SARS-CoV-2 antibody that engages three neutralizing epitopes on a single S protein trimer.

In another embodiment, the fusion molecule comprises peptide p102 and/or p16 fused to the N-terminus of anti-SARS-CoV-2 antibody IgG 33-7. In another embodiment, the fusion molecule comprises peptide p102 and/or p16 fused to the N-terminus of anti-SARS-CoV-2 antibody IgG 33-7.

In another embodiment, the fusion includes at least one peptide fused to the N-terminus of each of the heavy chains of the anti-SARS-CoV-2 antibody. In another embodiment, the fusion includes at least one peptide fused to the N-terminus of each of the light chains of the anti-SARS-CoV-2 antibody. In another embodiment, the fusion molecule comprises an anti-SARS-CoV-2 antibody and peptides p102 and/or p16. In another embodiment, the fusion molecule comprises an anti-SARS-CoV-2 antibody and at least one peptide fused on each of the heavy chain and the light chains, where the peptide fused to the heavy chain can be the same or different than the peptide fused to the light chain. In another embodiment, the fusion molecule comprises an anti-SARS-CoV-2 antibody IgG 33-7 and at least one peptide fused on each of the heavy chain and the light chains, where the peptide fused to the heavy chain can be the same or different than the peptide fused to the light chain. In another embodiment, the fusion molecule comprises an anti-SARS-CoV-2 antibody IgG 33-7 and peptide p102 and/or p16 fused on each of the heavy chain and the light chains, where the peptide fused to the heavy chain can be the same or different than the peptide fused to the light chain.

### EXAMPLES

### Protein Production

The SARS-CoV-2 S protein ectodomain and RBD were produced and purified as described (Miersch, S., et al., (2021) Tetravalent SARS-CoV-2 Neutralizing Antibodies Show Enhanced Potency and Resistance to Escape Mutations. Journal of Molecular Biology 433, 167177.) Purified proteins were site-specifically biotinylated in a reaction with 200 μM biotin, 500 μM ATP, 500 μM MgCl2, 30 μg/mL BirA, 0.1% (v/v) protease inhibitor cocktail and not more than 100 μM of the protein-AviTag substrate. The reactions were incubated at 30° C. for 2 hours and biotinylated proteins were purified by size-exclusion chromatography.

### Antibody Production

IgG and peptide-IgG fusion proteins were produced as described (Miersch, S., et al., (2021) Tetravalent SARS-CoV-2 Neutralizing Antibodies Show Enhanced Potency and Resistance to Escape Mutations. Journal of Molecular Biology 433, 167177.). S protein-binding peptides were fused to the N-terminus of the heavy or light chain through a 20-residue linker (sequence: GGGGSGGGGSGGGGSGGGGS) using standard molecular biology techniques.

### Phage Display Selections

Naïve libraries were constructed as described (Arita, Y., et al., (2016) Rapid isolation of peptidic inhibitors of the solute carrier family transporters OATP1B1 and OATP1B3 by cell-based phage display selections. Biochem. Biophys. Res. Commun. 473, 370-376.). For libraries for peptide optimization, oligonucleotides were synthesized using degenerate codons encoding for the amino acids at each position indicated in FIG. 34. Oligonucleotide-directed mutagenesis was performed to introduce randomized sequences fused to the gene-8 major coat protein of M-13 bacteriophage as described (Tonikian, R., et al., (2007) Identifying specificity profiles for peptide recognition modules from phage-displayed peptide libraries. Nat Protoc 2, 1368-1386.). Each phage-displayed peptide library was selected for binding to immobilized S protein ECD or RBD, as described (Tonikian, R., et al., (2007) Identifying specificity profiles for peptide recognition modules from phage-displayed peptide libraries. Nat Protoc 2, 1368-1386.), but with the following modifications. Biotinylated S protein ECD or RBD was captured on wells coated with neutravidin (NAV), followed by incubation with the phage-displayed peptide library. After five rounds of binding selections, individual clones were picked and DNA was sequenced. Clones that showed a significant binding signal to S protein ECD and/or RBD, but not to BSA or NAV, were selected for further analysis.

### ELISAs

Phage ELISAs were performed, as described (Miersch, S., et al., (2021) Tetravalent SARS-CoV-2 Neutralizing Antibodies Show Enhanced Potency and Resistance to Escape Mutations. Journal of Molecular Biology 433, 167177.), but with the following modifications; 384-well maxisorp plates (Sigma) were coated with NAV, or left uncoated as negative control, and blocked with PBS, 0.5% Bovine serum albumin (BSA). Biotinylated target protein was captured by incubation in NAV-coated and BSA-blocked wells, or with buffer solution alone as negative control, at room temperature. For competition ELISAs, blocking IgG was incubated with coated and blocked wells for 1 hour at room temperature. Wells were incubated with peptide-phage in PBS, 0.5% BSA for 1 hour. Plates were washed, incubated with anti-M13-HRP antibody (Sino Biological, catalog number 11973-MM05T-H) and developed with TMB substrate (Mandel, catalog number KP-50-76-03).

### Biolayer Interferometry

For IgGs and peptide-IgG fusions, binding kinetics for the S protein were determined by BLI with an Octet HTX instrument (ForteBio), as described (Miersch, S., et al., (2021) Tetravalent SARS-CoV-2 Neutralizing Antibodies Show Enhanced Potency and Resistance to Escape Mutations. Journal of Molecular Biology 433, 167177.). For peptides, biotinylated peptides (Table 6) were immobilized on streptavidin-coated sensors that were subsequently blocked with excess biotin. Following equilibration with assay buffer, loaded biosensors were dipped for 600 seconds into wells containing 3-fold serial dilutions of S protein ECD, and subsequently, were transferred back into assay buffer for 600 seconds. Binding response data were corrected by subtraction of response from a reference and were fitted with a 1:1 binding model using the ForteBio Octet Systems software 9.0. We determined KD values at various peptide loading densities to ensure that high densities were not impacting kinetic measurements at the sensor.

### Sequence Alignment and Analysis

Sequences of binding peptides were imported into Geneious R9 software (Biomatters Ltd.). Peptides were sorted based on the library from which they originated and aligned separately using the MUSCLE algorithm. In the case of peptides lacking cysteines, a strict penalty was imposed on the formation of gaps during alignment. Sequence logos were created using the weblogo server (https://weblogo.berkeley.edu/logo.cgi).

### Virus Neutralization Assay

Serial dilutions of IgGs were incubated with the virus at 37° C. for 1 hour. The IgG-virus mixture was transferred to 96-well tissue culture plates containing sub-confluent monolayers of Vero E6 cells in duplicate and incubated at 37° C. and 5% CO₂. Infection was monitored in one of two ways. The first used a crystal violet-based method as follows. Supernatants were carefully discarded and 100 μl Crystal Violet solution containing 4% formaldehyde was added to each well. Cells were washed and 100 μl containing 50 parts of absolute ethanol (Sigma, St. Louis, MO), 49 parts of MilliQ water and 1 part of glacial acetic acid (Sigma catalog number 64-19-7) were added to each well. Plates were incubated for 15 minutes at room temperature and read by a spectrophotometer at 590 nm. Alternatively, infection was monitored using an ELISA-based method with the anti-S protein antibody CR3022, followed by incubation with HRP-conjugated goat anti-Fc antibody (Invitrogen, catalog number A18817). Stained cells were visualized using KPL TrueBlue peroxidase (SeraCare, catalog number 5510) and quantified on an ImmunoSpot microanalyzer (Cellular Technologies). All data were analyzed using Prism software (GraphPad Prism 8.0).

### Structural Analysis

The model of two 15033-7 Fabs bound to the SARS-CoV-2 S protein (PDB entry 7KMK) was imported into PyMol (DeLano Scientific, LLC). Distances between the HC N-termini and the 15033-7 epitope on the unbound RBD were measured as the distance between the Ca of the first residue of the HC and the CP of Tyr489 of the S protein. To build the model of two RGN10933 Fabs bound to the S protein, the model of RGN10933 and RGN10987 bound to the RBD (PDB entry 6XDG) was imported into PyMol along with the data from PDB entry 7KMK. The data from PDB entry 6XDG were duplicated and the RBDs of the model were superposed with the two RBDs in the “up” position in the model from PDB entry 7KMK. The RBDs in the model from PDB entry 6XDG, RGN10987 Fabs, and 15033-7 Fabs were then eliminated from the model, leaving only the two RNG10933 from PDB entry 6XDG bound to the S protein from PDB entry 7KMK. Distances were measured the same way as for 15033-7.

### Results

### Isolation and Characterization of S Protein-Binding Peptides

In order to isolate novel peptides that bound to the S protein of SARS-CoV-2, we used phage-displayed libraries of 16-residue peptides. Next, phage representing a panel of 10 peptide libraries in which diversified positions were encoded by an equimolar mixture of 19 codons representing all genetically-encoded amino acids except cysteine, and an “unconstrained” library (X16) containing 16 diversified positions with no fixed positions (Arita, Y., et al., (2016) Rapid isolation of peptidic inhibitors of the solute carrier family transporters OATP1B1 and OATP1B3 by cell-based phage display selections. Biochem. Biophys. Res. Commun. 473, 370-376.) were pooled together. Other libraries contained 14 diversified positions and two fixed cysteine residues separated by 4-12 diversified positions. These “constrained” libraries were designed to display peptides containing disulfide-bonded loops, which have been found to promote tertiary structures that can enhance binding to proteins (Bozovicar, K., and Bratkovie, T. (2021) Small and Simple, yet Sturdy: Conformationally Constrained Peptides with Remarkable Properties. Int J Mol Sci 22, 1611.).

Phage representing the library pool were cycled through five rounds of binding selections with immobilized S protein ECD or RBD, and several hundred clones were analyzed for binding to the ECD. Clones that exhibited strong binding signals in phage ELISAs with the ECD and negligible signals with bovine serum albumin (BSA) and neutravidin (NAV) were subjected to DNA sequence analysis. This process yielded 160 and 128 unique peptide sequences from the ECD and RBD selections, respectively. Alignment of the sequences revealed that most (85%) of the ECD-selected peptides were derived from two libraries: 63% and 22% were from the X16 or C-X4-C library, respectively (FIG. 32A). In contrast, most (69%) of the RBD-selected peptides were from two different libraries: 42% and 27% were from the C-X10-C or C-X12-C library, respectively (FIG. 32B). Inspection of the aligned sequences revealed significant homology within each peptide family, which enabled us to derive consensus motifs (FIG. 32). Notably, the peptides derived from the C-X10-C and C-X12-C libraries exhibited similar consensus sequences, suggesting that they bind to the S protein in a similar manner.

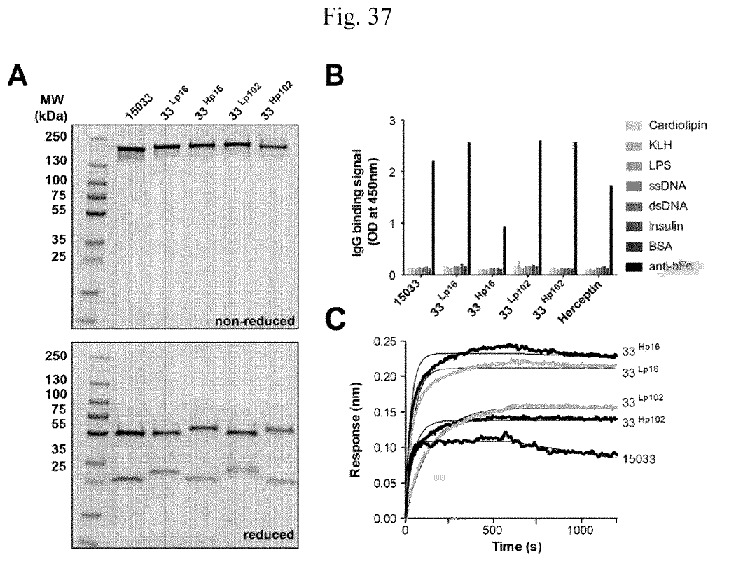

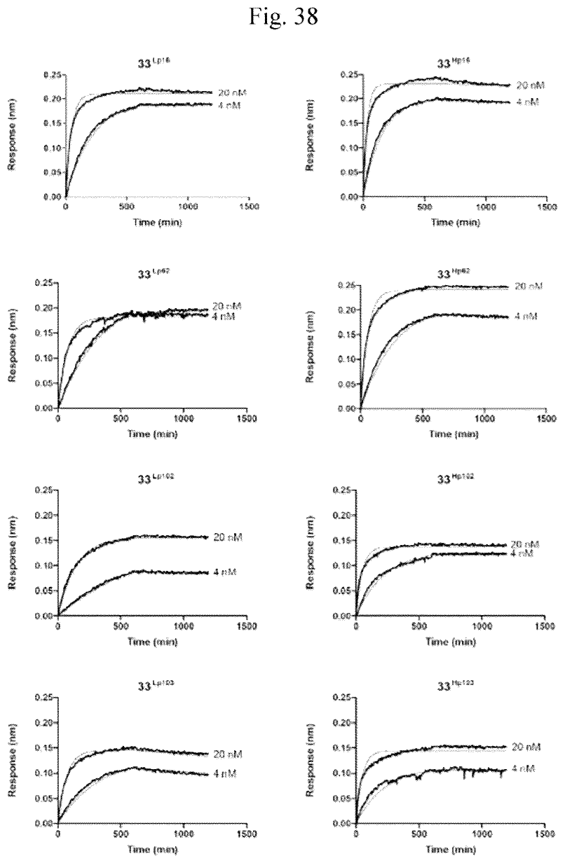

For more detailed characterization, we chose four peptides that closely matched the consensus motifs for their respective families. These included a peptide from the X16 library (p16) and a peptide from the C-X4-C library (p62), which were selected for binding to the S protein ectodomain, and two peptides (p102 and p103) from the C-X12-C library, which were selected for binding to the RBD (FIG. 2). As expected, phage ELISAs showed that all four peptide-phage bound to immobilized S protein ectodomain, but only peptides p102 and p103 bound to immobilized RBD (FIG. 3A). To further define where each peptide bound, we assessed whether binding of peptide-phage to the ECD could be blocked by nAbs that recognized known epitopes on either the N-terminal domain (NTD) (IgGs 5-24 and 4-8) (Liu, L., et al., (2020) Potent neutralizing antibodies against multiple epitopes on SARS-CoV-2 S protein. Nature 584, 450-456.) or the RBD (IgGs 15033-7 and REGN10933) (Hansen, J., et al., (2020) Studies in humanized mice and convalescent humans yield a SARS-CoV-2 antibody cocktail. Science 369, 1010-1014.)(Miersch, S., et al., (2021) Tetravalent SARS-CoV-2 Neutralizing Antibodies Show Enhanced Potency and Resistance to Escape Mutations. Journal of Molecular Biology 433, 167177.). Binding of peptide-phage p16 and p62, but not p102 and p103, was blocked by the two antibodies that bound to a neutralizing epitope on the NTD but not by those that bound to the ACE2-binding site of the RBD. Conversely, binding of peptide-phage p102 and p103, but not p16 and p62, was blocked by the two antibodies that bound to the RBD but not by those that bound to the NTD (FIG. 37B). Taken together, these results showed that peptides p16 and p62 likely bind to the NTD whereas peptides p102 and p103 bind to the RBD, and all peptides likely bind to sites that overlap with epitopes of nAbs.

### Optimization of S Protein-Binding Peptides

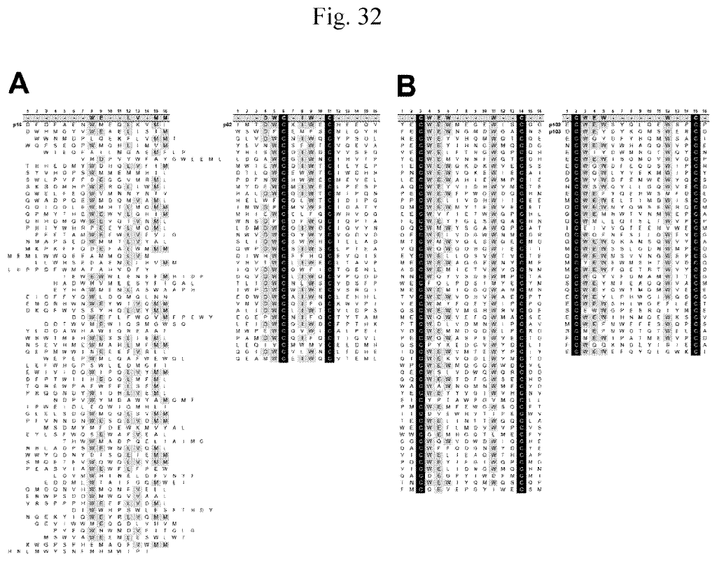

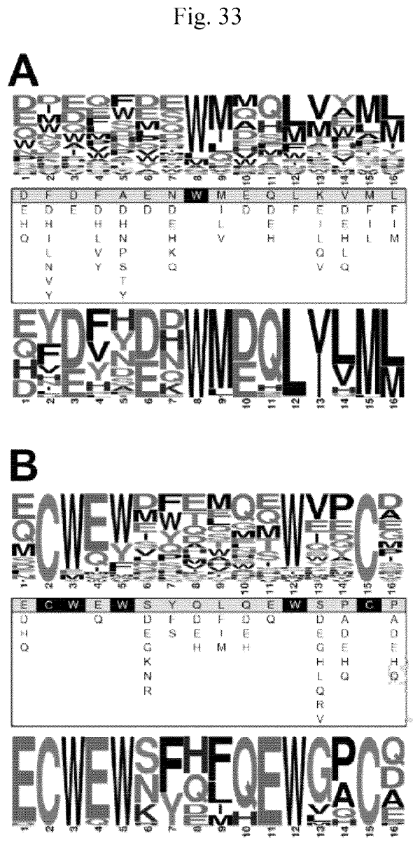

Biased peptide-phage libraries were designed to further optimize peptides p16 and p102 (FIG. 33). For each peptide, we used the sequence logo of its family members to identify highly conserved positions that we fixed as the wt, and variable positions for which we used degenerate codons that encoded for the wt sequence and other sequences that were prevalent in the sequence logo. Phage pools representing each biased library were cycled through five rounds of selection for binding to the S protein ECD, positive clones were identified by phage ELISA, and DNA sequencing revealed 86 and 25 unique sequences from the p16 and p102 libraries (Table 9), respectively for which a subset is shown in FIG. 34. We used the unique sequences to derive a sequence logo for each selection pool (FIG. 33). We chose unique peptide sequences that closely matched the logo for further characterization, reasoning that these likely represented high affinity binders.

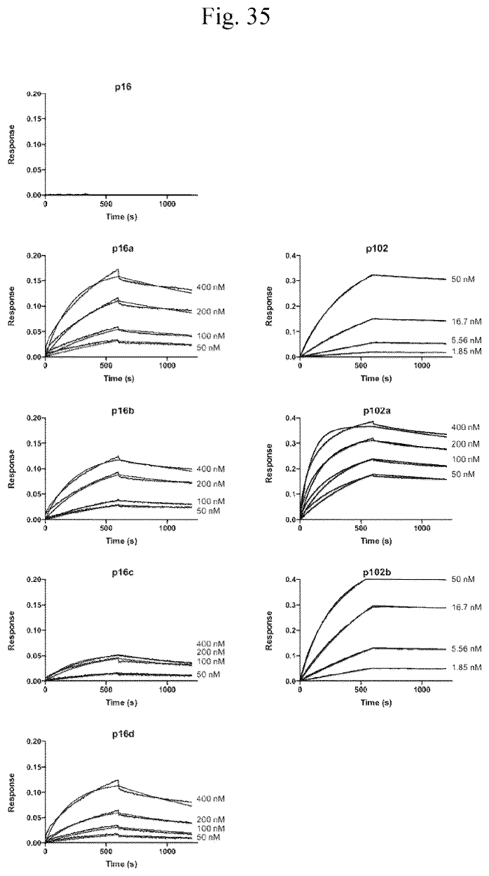

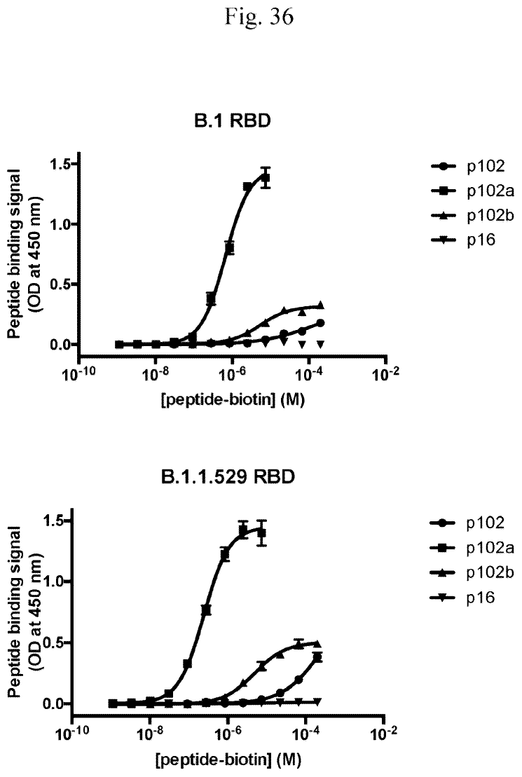

Based on this process, we identified four variants of peptide p16 (p16a, p16b, p16c, p16d) and two variants of peptide p102 (p102a, p102b) for chemical synthesis (FIG. 34), and these were synthesized as 22-residue peptides consisting of the following: a Gly residue, followed by the selected 16-residue sequence, followed by a 5-residue hydrophilic linker (Gly-Gly-Lys-Gly-Lys), followed by biotin. We used biolayer interferometry (BLI) assays to determine binding kinetics and affinities (FIG. 35). Biotinylated peptides were immobilized on sensor chips that were incubated with solutions of S protein ectodomain at various concentrations (FIG. 35). Peptide p16 did not exhibit detectable binding, presumably due to low affinity, but its variants bound with moderate apparent affinities (apparent KD=26-68 nM). Peptide p102 and its variants exhibited high apparent affinities in the single-digit nanomolar range (apparent KD=0.8-5.6 nM) (Table 6). Moreover, peptide p102 showed equivalent binding efficiency to omicron RBD, as determined by ELISA (FIG. 36), suggesting this peptide binds to a site that has not mutated in emerged variants. We note that polyvalent avidity effects likely enhance these apparent affinities, and the intrinsic monovalent affinities are likely lower. Nonetheless, this format does mimic the polyvalent nature of the virus-neutralizing molecules we ultimately intended to produce, as described below.

### Production and Characterization of Peptide-IgG Fusions

We next explored whether peptide fusions could enhance the affinity of a neutralizing IgG targeting the ACE2-binding site of the RBD. For this purpose, we used the moderate affinity IgG 15033 that we had selected from a naïve phage-displayed synthetic antibody library (Miersch, S., et al., (2021) Tetravalent SARS-CoV-2 Neutralizing Antibodies Show Enhanced Potency and Resistance to Escape Mutations. Journal of Molecular Biology 433, 167177.), in order to accurately discern affinity differences. Peptide p16 or p102 was fused to the N-terminus of either the light chain (LC) or heavy chain (HC) of IgG 15033 with an intervening 20-residue Gly/Ser linker. The resulting peptide-IgG fusion proteins and IgG 15033 were purified by transient transfection in mammalian Expi293F cells (Miersch, S., et al., (2021) Tetravalent SARS-CoV-2 Neutralizing Antibodies Show Enhanced Potency and Resistance to Escape Mutations. Journal of Molecular Biology 433, 167177.). Peptide-IgG fusions can be purified to homogeneity with a single Protein A affinity chromatography step as evidenced by Coomassie-stained SDS-PAGE gels, that resolve differences in molecular weight between peptide-fused heavy or light chain under reducing conditions (FIG. 37A). Evaluation of peptide-fused IgGs by non-specificity ELISA show no evidence of binding to a panel of non-specific biomolecules (FIG. 37B), suggesting an absence of non-specific binding that can undermine developability, pharmacokinetics and efficacy. Lastly, kinetic binding analysis by BLI showed that the tetravalent peptide-IgG fusions exhibited greatly reduced off-rates compared with IgG 15033 (FIG. 37C), and consequently, affinities were greatly improved (Table 6). These results were as expected, since the addition of a peptide ligand to an RBD-binding IgG should reduce the overall free energy of binding, and thus increase binding efficiency. In particular, the peptide-IgG with peptide p102 fused to its HC (33^{Hp102}) and the peptide-IgG with peptide p16 fused to its LC (33^{Lp16}) exhibited extremely slow off-rates that were beyond the sensitivity of the instrument, and consequently, the apparent dissociation constants were estimated to be sub-picomolar (apparent K_{D}<1 μM), which was >100-fold improved relative to IgG 15033 (apparent K_{D}=100 μM).

TABLE 7

| IgG^{a} | kₒₙ (10⁴ M⁻¹ s⁻¹) | k_{off} (10⁻⁶ s⁻¹) | K_{D} (pM)^{b} |
|---|---|---|---|
| 15033 | 170 | 500 | 300 |
| 33^{HN1} | 160 | 38 | 24 |
| 33^{LN1} | 110 | <0.1 | <1 |
| 33^{HR1} | 120 | <0.1 | <1 |
| 33^{LR1} | 35 | 2.2 | 6.1 |

*as noted herein, 33 is shorthand for 15033; H or L indicates that the peptide is attached to either heavy chain or light chain; and N1 corresponds to peptide p16 and R1 corresponds to peptide p102.

### Inhibition of SARS-CoV-2 Infection in Cell-Based Assays

We explored the critical question of whether peptide fusions could enhance the potency of nAbs in cell-based assays of SARS-CoV-2 infection. For this purpose, we fused peptide p102 to the N-terminus of the HC of IgG 15033-7, a more potent variant of IgG 15033 with an optimized LC²⁰. The resulting peptide-IgG fusion protein (33-7^{Hp102}) was compared to IgG 15033-7 in assays that measured the infection of ACE2-expressing Vero E6 cells with a panel of six authentic SARS-CoV-2 variants including the original Wuhan virus (B.1) and later emerging VoC, including variants isolated in Italy (B.1.1), the United Kingdom (B.1.1.7), South Africa (B.1.351 and B.1.529), Nigeria (B.1.525), and Brazil (P.1). In every case, the potency of the peptide-IgG fusion greatly exceeded that of the IgG (FIG. 39A). Indeed, peptide-IgG 33-7^{Hp102} neutralized three VoC in the single-digit ng/mL range (IC₅₀=6.5-7.6 ng/mL) and it neutralized the other three viruses in the double-digit ng/mL range (IC₅₀=12-49 ng/mL). Intriguingly, 33-7^{Hp102} was even able to neutralize the highly mutated B.1.1.529 (omicron), albeit at elevated IC₅₀ (260 ng/mL). In contrast, IgG 15033-7 was ineffective against three VoC (IC₅₀>900 ng/mL), much less effective than IgG 33-7^{Hp102} against the other three VoC (IC₅₀=64-310 ng/mL).

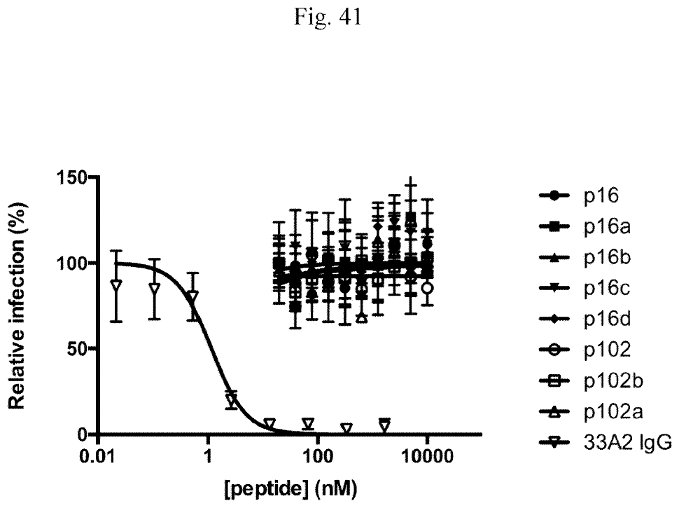

We also explored whether the modularity of the peptides could be exploited to enhance the potency of a clinically approved therapeutic nAb (REGN10933, FIG. 12B). We constructed a variant of REGN10933 by fusing peptide p102 to the N-terminus of the HC. The resulting peptide-IgG fusion (10933^{Hp102}) proved to be more potent than REGN10933 in neutralization assays against the SARS-CoV-2 variant D614G (IC50<10 or ˜10 ng/mL, respectively). Most strikingly, peptide-IgG 10933 H^{p102} was also extremely potent against the South African variant B.1.351 (IC₅₀<10 ng/mL), against which REGN10933 was completely ineffective (IC50>1000 ng/mL), consistent with previous reports. Finally, since peptide p102 binds to the RBD in competition with the 15033 epitope, which overlaps substantially with the ACE2 binding site, we sought to determine whether these peptides alone are able to neutralize SARS-CoV-2. At concentrations up to 10 μM, no infection neutralization was observed in a pseudovirus assay (FIG. 41). Taken together, these results show that peptide fusions greatly enhanced the potency and breadth of coverage of neutralizing IgGs against SARS-CoV-2 and its VOCs, and that these effects are entirely due to enhanced affinity obtained by fusion of S protein binding peptides to the n-terminus of nAbs.

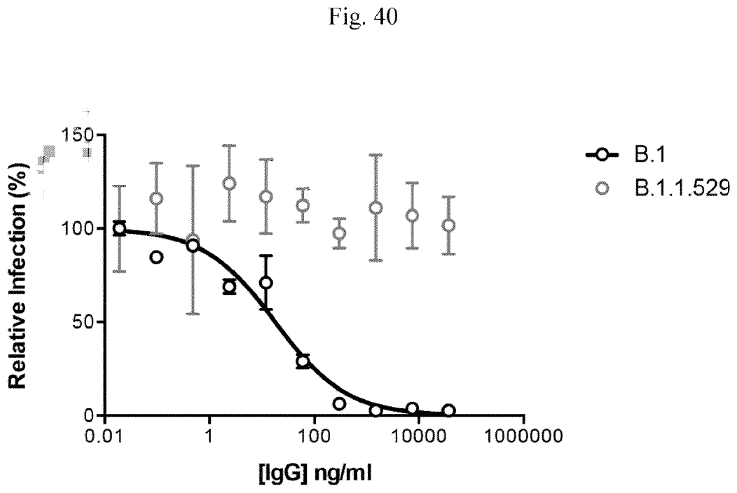

In a pseudovirus infection assay, IgG 15033-7 was evaluated in parallel against both the B.1 and B.1.1.529 pseudovariants and while it potently neutralized the B.1 variant (IC₅₀<100 ng/mL), it was inactive against B.1.1.529 up to the maximum antibody concentration tested (250 nM) (FIG. 40), confirming the mutational escape from the parental IgG 15033-7.

Those skilled in the art will recognize, or be able to ascertain, using no more than routine experimentation, numerous equivalents to the specific procedures described herein. Such equivalents are considered to be within the scope of the inventions. Various substitutions, alterations and modifications may be made to the invention without departing from the spirit and scope of the invention. Other aspects, advantages, and modifications are within the scope of the invention.

The contents of all references, issued patents, and published patent applications cited through this application are hereby incorporated by reference. The appropriate component, process and methods of those patents, applications and other documents may be selected for the invention and embodiments thereof.

### CDR Overview

TABLE 2
Lead antibody clones-CDR amino acid sequences

| | | CDR1 | CDR2 | CDR3 |
|---|---|---|---|---|
| 15031 | hIg1 | LTGSYM | GISASGGATA | SRSSSYSSSGWRYYSGAF |
| | hk | SVSSA | SAS | GSYLF |
| 15032 | hIg1 | FSAASI | GIYASGGATD | SSYRYSSGASFHYYYGF |
| | hk | SVSSA | SAS | YYHSPF |
| 15033 | hIg1 | LGGYSM | GIYASGGATA | SYYYGGFGM |
| | hk | SVSSA | SAS | SYRYPI |
| 15033-7 | hIg1 | LGGYSM | GIYASGGATA | SYYYGGFGM |
| | hk | SVSSA | SAS | SHTYPI |
| 15033 A2 | hIg1 | LGGYSM | GIYASGGATA | SYYYGGFGM |
| | hk | DVAYA | SAD | SHTYPI |
| 15034 | hIg1 | IGGDSI | GIYASGGSTA | GGWGPYSYYYHYGL |
| | hk | SVSSA | SAS | YSYYYDLI |
| 15036 | hIg1 | FTAYDI | GISASGGYTA | DGFHSYAL |
| | hk | SVSSA | SAS | YSYDSLI |
| 15046 | hIg1 | FGSYAM | GIYASGGYTD | GWSYYWGF |
| | hk | SVSSA | SAS | YDYGLF |
| 15046 A1 | hIg1 | FGSYAM | GIYASGGYTD | GWSYYWGF |
| | hk | SVSSA | SAS | SSPSDLF |
| 15049 | hIg1 | FSSSDI | RIYASGGYTD | WGHGYGYFYYGF |
| | hk | SVSSA | SAS | SSGDLI |

TABLE 3
Lead antibody clones-CDR nucleotide sequences

| | | CDR1 | CDR2 | CDR3 |
|---|---|---|---|---|
| 15031 | hIg1 | CTCACTGGTTCTTACATG | GGTATTTCCGCTTCTG | TCTCGTTCTTCTTCTTACTC |
| | | | GAGGCGCTACTGCT | TTCTTCTGGTTGGCGTTACT |
| | | | | ACTCTGGTGCTTTT |
| | hk | TCCGTGTCCAGCGCT | TCGGCATCC | GGTTCTTACCTGTTC |
| 15032 | hIg1 | TTCAGTGCTGCTTCGATC | GGTATTTACGCTTCTG | TCTTCTTACCGTTACTC |
| | | | GAGGCGCTACTGAT | TTCTGGTGCTTCTTTCCATTACT |
| | | | | ACTACGGT |
| | hk | TCCGTGTCCAGCGCT | TCGGCATCC | TACTACCATTCTCCGTTC |
| 15033 | hIg1 | CTCGGTGGTTATTCGATG | GGTATTTACGCTTCTG | TCTTACTACTACGG |
| | | | GAGGCGCTACTGCT | TGGTTTCGGTATG |
| | hk | TCCGTGTCCAGCGCT | TCGGCATCC | TCTTACCGTTACCCGATC |
| 15033- | hIg1 | CTCGGTGGTTATTCGATG | GGTATTTACGCTTCTG | TCTTACTACTACGG |
| 7 | | | GAGGCGCTACTGCT | TGGTTTCGGTATG |
| | hk | TCCGTGTCCAGCGCT | TCGGCATCC | TCTCATACTTACCCGATC |
| 15033 | hIg1 | CTCGGTGGTTATTCGATG | GGTATTTACGCTTCTG | TCTTACTACTACGGTGGTTTCGGTATG |
| A2 | | | GAGGCGCTACTGCT | |
| | hk | GATGTGGCTTATGCT | TCTGCAGAT | TCTCATACTTACCCGATC |
| 15034 | hIg1 | ATCGGTGGTGATTCGATC | GGTATTTACGCTTCTG | GGTGGTTGGGGTCCGT |
| | | | GAGGCTCTACTGCT | ACTCTTACTACTAC |
| | | | | CATTACGGTTTG |
| | hk | TCCGTGTCCAGCGCT | TCGGCATCC | TACTCTTACTACTACGACCTGATC |
| 15036 | hIg1 | TTCACTGCTTATGACATC | GGTATTTCCGCTTCTG | GACGGTTTCCATTCTTACGCTTTG |
| | | | GAGGCTATACTGCT | |
| | hk | TCCGTGTCCAGCGCT | TCGGCATCC | TACTCTTACGACTCTCTGATC |
| 15046 | hIg1 | TTCGGTAGTTATGCCATG | GGTATTTACGCTTCTG | GGTTGGTCTTACTACTGGGGTTTT |
| | | | GAGGCTATACTGAT | |
| | hk | TCCGTGTCCAGCGCT | TCGGCATCC | TACGACTACGGTCTGTTC |
| 15046 | hIg1 | TTCGGTAGTTATGCCATG | GGTATTTACGCTTCTG | GGTTGGTCTTACTACTGGGGTTTT |
| A1 | | | GAGGCTATACTGAT | |
| | hk | TCCGTGTCCAGCGCT | TCGGCATCC | TCTTCTCCATCTGATCTGTTC |
| 15049 | hIg1 | TTCAGTAGTTCTGACATC | CGTATTTACGCTTCTG | TGGGGTCATGGTTACG |
| | | | GAGGCTATACTGAT | GTTACTTCTACTACGGTTTT |
| | hk | TCCGTGTCCAGCGCT | TCGGCATCC | TCTTCTGGTGACCTGATC |

TABLE 4
Notes-
CDRs in the nucleotide and amino acid sequences are shown as underlined
Linkers in the nucleotide and amino acid sequences are shown as underlined, italicized and BOLD
Although all the CDR sequences in the various formats are those of clone 15031, the CDRs in
these various formats may be replaced with the CDRs of the clones shown in Tables 2 or 3.

| 1. LEAD VHH clone example | |
|---|---|
| 15031 VHH | |
| VHH NT | GAGGTTCAGCTGGTGGAGTCTGGCGGTGGCCTGGTGCAGCCAGGGCGTTCACTCCGTTTGTCCTGTGCAGCTTCTG |
| | GCTTCGACCTCACTGGTTCTTACATGCACTGGGTGCGTCGTGCCCCGGGTAAGGGCGAGGAACTGGTTGCAGGTATT |
| | TCCGCTTCTGGAGGCGCTACTGCTTATGCCGATAGCGTCAAGGGCCGTTTCACTATAAGCGCAGACACATCCAAAAA |
| | CACAGCCTACCTACAAATGAACAGCTTAAGAGCTGAGGACACTGCCGTCTATTATTGTGCTCGCTCTCGTTCTTCTTCT |
| | TACTCTTCTTCTGGTTGGCGTTACTACTCTGGTGCTTTTTGACTACTGGGGTCAAGGAACCCTGGTCACCGTCTCCTCG |
| VHH AA | EVQLVESGGGLVQPGRSLRLSCAASGFDLTGSYMHWVRRAPGKGEELVAGISASGGATAYADSVKGRFTISADTSKNTAY |
| | LQMNSLRAEDTAVYYCARSRSSSYSSSGWRYYSGAFDYWGQGTLVTVSS |
| 2. LEAD scFv clone example | |
| 15031 scPv | |
| VL-VH NT | GATATCCAGATGACCCAGTCCCCGAGCTCCCTGTCCGCCTCTGTGGGCGATAGGGTCACCATCACCTGCCGTGCCAG |
| | TCAGTCCGTGTCCAGCGCTGTAGCCTGGTATCAACAGAAACCAGGAAAAGCTCCGAAGCTTCTGATTTACTCGGCAT |
| | CCGATCTCTACTCTGGAGTCCCTTCTCGCTTCTCTGGTAGCCGTTCCGGGACGGATTTCACTCTGACCATCAGCAGTCT |
| | GCAGCCGGAAGACTTCGCAACTTATTACTGTCAGCAAGGTTCTTACCTGTTCACGTTCGGACAGGGTACCAAGGTGG |
| | AGATCAAAGGTACTACTGCCGCTAGTGGTAGTAGTGGTGGCAGTAGCAGTGGTGCCGAGGTTCAGCTGGTGGAGT |
| | CTGGCGGTGGCCTGGTGCAGCCAGGGGGCTCACTCCGTTTGTCCTGTGCAGCTTCTGGCTTCGACCTCACTGGTTCTT |
| | ACATGCACTGGGTGCGTCAGGCCCCGGGTAAGGGCCTGGAATGGGTTGCAGGTATTTCCGCTTCTGGAGGCGCTAC |
| | TGCTTATGCCGATAGCGTCAAGGGCCGTTTCACTATAAGCGCAGACACATCCAAAAACACAGCCTACCTACAAATGA |
| | ACAGCTTAAGAGCTGAGGACACTGCCGTCTATTATTGTGCTCGCTCTCGTTCTTCTTCTTACTCTTCTTCTGGTTGGCG |
| | TTACTACTCTGGTGCTTTTGACTACTGGGGTCAAGGAACCCTGGTCACCGTCTCCTCG |
| VL-VH AA | DIQMTQSPSSLSASVGDRVTITCRASQSVSSAVAWYQQKPGKAPKLLIYSASDLYSGVPSRFSGSRSGTDFTLTISSLQPEDF |
| | ATYYCQQGSYLFTFGQGTKVEIKGTTAASGSSGGSSSGAEVQLVESGGGLVQPGGSLALSCAASGFDLTGSYMHWVRQA |
| | PGKGLEWVAGISASGGATAYADSVKGRFTISADISKNTAYLQMNSLRAEDTAVYYCARSRSSSYSSSGWRYYSGAFDYW |
| | GQGTLVTVSS |
| 3. LEAD Fab clone example | |
| 15031 Fab | |
| VL-CL NT | GATATCCAGATGACCCAGTCCCCGAGCTCCCTGTCCGCCTCTGTGGGCGATAGGGTCACCATCACCTGCCGTGCCAG |
| | TCAGTCCGTGTCCAGCGCTGTAGCCTGGTATCAACAGAAACCAGGAAAAGCTCCGAAGCTTCTGATTTACTCGGCAT |
| | CCGATCTCTACTCTGGAGTCCCTTCTCGCTTCTCTGGTAGCCGTTCCGGGACGGATTTCACTCTGACCATCAGCAGTCT |
| | GCAGCCGGAAGACTTCGCAACTTATTACTGTCAGCAAGGTTCTTACCTGTTCACGTTCGGACAGGGTACCAAGGTGG |
| | AGATCAAACGTACGGTGGCTGCACCATCTGTCTTCATCTTCCCGCCATCTGATGAGCAGTTGAAATCTGGAACTGCCT |
| | CTGTTGTGTGCCTGCTGAATAACTTCTATCCCAGAGAGGCCAAAGTACAGTGGAAGGTGGATAACGCCCTCCAATCG |
| | GGTAACTCCCAGGAGAGTGTCACAGAGCAGGACAGCAAGGACAGCACCTACAGCCTCAGCAGCACCCTGACGCTGA |
| | GCAAAGCAGACTACGAAAAACATAAAGTCTACGCCTGCGAAGTCACCCATCAGGGCCTGAGCTCGCCCGTCACAAA |
| | GAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTLTISSLQPEDF |
| | ATYYCQQGSYLFTFGQGTKVEIKRTVAAPSVFIFPPSDEQLKSGTASVVCLLNNFYPREAKVQWKVDNALQSGNSQESVTE |
| | QQSKQSTYSLSSTLTLSKADYEKHKVYACEVTHQGLSSPVTKSFNRGEC |
| VH-CH1 NT | GAGGTTCAGCTGGTGGAGTCTGGCGGTGGCCTGGTGCAGCCAGGGGGCTCACTCCGTTTGTCCTGTGCAGCTTCTG |
| | GCTTCGATCTCACTGGTTCTTACATGCACTGGGTGCGTCAGGCCCCGGGTAAGGGCCTGGAATGGGTTGCAGGTATT |
| | TCCGCTTCTGGAGGCGCTACTGCTTATGCCGATAGCGTCAAGGGCCGTTTCACTATAAGCGCAGACACATCCAAAAA |
| | CACAGCCTACCTACAAATGAACAGCTTAAGAGCTGAGGACACTGCCGTCTATTATTGTGCTCGCTCTCGTTCTTCTTCT |
| | TACTCTTCTTCTGGTTGGCGTTACTACTCTGGTGCTTTTGACTACTGGGGTCAAGGAACCCTGGTCACCGTCTCCTCGG |
| | CTAGCACCAAGGGCCCATCGGTCTTCCCCCTGGCACCCTCCTCCAAGAGCACCTCTGGGGGCACAGCGGCCCTGGGC |
| | TGCCTGGTCAAGGACTACTTCCCCGAACCGGTGACGGTGTCGTGGAACTCAGGCGCCCTGACCAGCGGCGTGCACA |
| | CCTTCCCGGCTGTCCTACAGTCCTCAGGACTCTACTCCCTCAGCAGCGTGGTGACCGTGCCCTCCAGCAGCTTGGGCA |
| | CCCAGACCTACATCTGCAACGTGAATCACAAGCCCAGCAACACCAAGGTGGACAAGAAAGTTGAGCCCAAATCTTGT |
| | GACAAAACTCACACA |
| VH-CH1 AA | EVQLVESGGGLVQPGGSLRLSCAASGFDLTGSYMHWVRQAPGKGLEWVAGISASGGATAYADSVKGRFTISADTSKNT |
| | AYLQMNSLRAEDTAVYYCARSRSSSYSSSGWRYYSGAFDYWGQGTLVTVSSASTKGPSVFPLAPSSKSTSGGTAALGCLV |
| | KDYFPEPVTVSWNSGALTSGVHTFPAVLQSSGLYSLSSVVTVPSSSLGTQTYICNVNHKPSNTKVDKKVEPKSCDKTHT |
| 4. LEAD Diabody clone example | |
| 15031 Diabody | |
| VH-VL NT | GAGGTTCAGCTGGTGGAGTCTGGCGGTGGCCTGGTGCAGCCAGGGGGCTCACTCCGTTTGTCCTGTGCAGCTTCTG |
| | GCTTCGACCTCACTGGTTCTTACATGCACTGGGTGCGTCAGGCCCCGGGTAAGGGCCTGGAATGGGTTGCAGGTATT |
| | TCCGCTTCTGGAGGCGCTACTGCTTATGCCGATAGCGTCAAGGGCCGTTTCACTATAAGCGCAGACACATCCAAAAA |
| | CACAGCCTACCTACAAATGAACAGCTTAAGAGCTGAGGACACTGCCGTCTATTATTGTGCTCGCTCTCGTTCTTCTTCT |
| | TACTCTTCTTCTGGTTGGCGTTACTACTCTGGTGCTTTTGACTACTGGGGTCAAGGAACCCTGGTCACCGTCTCCTCGG |
| | GTGGAGGTGGCAGTGATATCCAGATGACCCAGTCCCCGAGCTCCCTGTCCGCCTCTGTGGGCGATAGGGTCACCAT |
| | CACCTGCCGTGCCAGTCAGTCCGTGTCCAGCGCTGTAGCCTGGTATCAACAGAAACCAGGAAAAGCTCCGAAGCTTC |
| | TGATTTACTCGGCATCCGATCTCTACTCTGGAGTCCCTTCTCGCTTCTCTGGTAGCCGTTCCGGGACGGATTTCACTCT |
| | GACCATCAGCAGTCTGCAGCCGGAAGACTTCGCAACTTATTACTGTCAGCAAGGTTCTTACCTGTTCACGTTCGGACA |
| | GGGTACCAAGGTGGAGATCAAA |
| VH-VL AA | EVQLVESGGGLVQPGGSLRLSCAASGFDLTGSYMHWVRQAPGKGLEWVAGISASGGATAYADSVKGRFTISADTSKNT |
| | AYLQMNSLRAEDTAVYYCARSRSSSYSSSGWRYYSGAFDYWGQGTLVTVSSGGGGSDIQMTQSPSSLSASVGDRVTITC |
| | RASQSVSSAVAWYQQKPGKAPKLLIYSASDLYSGVPSRFSGSRSGTDFTLTISSLQPEDFATYYCQQGSYLFTFGQGTKVEIK |
| 5. LEAD higG1 clone example | |
| 15031 higG1 | |
| VL-CL NT | GATATCCAGATGACCCAGTCCCCGAGCTCCCTGTCCGCCTCTGTGGGCGATAGGGTCACCATCACCTGCCGTGCCAG |
| | TCAGTCCGTGTCCAGCGCTGTAGCCTGGTATCAACAGAAACCAGGAAAAGCTCCGAAGCTTCTGATTTACTCGGCAT |
| | CCGATCTCTACTCTGGAGTCCCTTCTCGCTTCTCTGGTAGCCGTTCCGGGACGGATTTCACTCTGACCATCAGCAGTCT |
| | GCAGCCGGAAGACTTCGCAACTTATTACTGTCAGCAAGGTTCTTACCTGTTCACGTTCGGACAGGGTACCAAGGTGG |
| | AGATCAAACGTACGGTGGCTGCACCATCTGTCTTCATCTTCCCGCCATCTGATGAGCAGTTGAAATCTGGAACTGCCT |
| | CTGTTGTGTGCCTGCTGAATAACTTCTATCCCAGAGAGGCCAAAGTACAGTGGAAGGTGGATAACGCCCTCCAATCG |
| | GGTAACTCCCAGGAGAGTGTCACAGAGCAGGACAGCAAGGACAGCACCTACAGCCTCAGCAGCACCCTGACGCTGA |
| | GCAAAGCAGACTACGAAAAACATAAAGTCTACGCCTGCGAAGTCACCCATCAGGGCCTGAGCTCGCCCGTCACAAA |
| | GAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTLTISSLQPEDF |
| | ATYYCQQGSYLFTFGQGTKVEIKRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| hIgG1 NT | GAGGTTCAGCTGGTGGAGTCTGGCGGTGGCCTGGTGCAGCCAGGGGGCTCACTCCGTTTGTCCTGTGCAGCTTCTG |
| | GCTTCGATCTCACTGGTTCTTACATGCACTGGGTGCGTCAGGCCCCGGGTAAGGGCCTGGAATGGGTTGCAGGTATT |
| | TCCGCTTCTGGAGGCGCTACTGCTTATGCCGATAGCGTCAAGGGCCGTTTCACTATAAGCGCAGACACATCCAAAAA |
| | CACAGCCTACCTACAAATGAACAGCTTAAGAGCTGAGGACACTGCCGTCTATTATTGTGCTCGCTCTCGTTCTTCTTCT |
| | TACTCTTCTTCTGGTTGGCGTTACTACTCTGGTGCTTTTGACTACTGGGGTCAAGGAACCCTGGTCACCGTCTCCTCGG |
| | CTAGCACCAAGGGCCCATCGGTCTTCCCCCTGGCACCCTCCTCCAAGAGCACCTCTGGGGGCACAGCGGCCCTGGGC |
| | TGCCTGGTCAAGGACTACTTCCCCGAACCGGTGACGGTGTCGTGGAACTCAGGCGCCCTGACCAGCGGCGTGCACA |
| | CCTTCCCGGCTGTCCTACAGTCCTCAGGACTCTACTCCCTCAGCAGCGTGGTGACCGTGCCCTCCAGCAGCTTGGGCA |
| | CCCAGACCTACATCTGCAACGTGAATCACAAGCCCAGCAACACCAAGGTGGACAAGAAAGTTGAGCCCAAATCTTGT |
| | GACAAAACTCACACATGCCCACCGTGCCCAGCACCTGAACTCCTGGGGGGACCGTCAGTCTTCCTCTTCCCCCCAAAA |
| | CCCAAGGACACCCTCATGATCTCCCGGACCCCTGAGGTCACATGCGTGGTGGTGGACGTGAGCCACGAAGACCCTG |
| | AGGTCAAGTTCAACTGGTACGTGGACGGCGTGGAGGTGCATAATGCCAAGACAAAGCCGCGGGAGGAGCAGTACA |
| | ACAGCACGTACCGTGTGGTCAGCGTCCTCACCGTCCTGCACCAGGACTGGCTGAATGGCAAGGAGTACAAGTGCAA |
| | GGTCTCCAACAAAGCCCTCCCAGCCCCCATCGAGAAAACCATCTCCAAAGCCAAAGGGCAGCCCCGAGAACCACAG |
| | GTGTACACCCTGCCCCCATCCCGGGAGGAGATGACCAAGAACCAGGTCAGCCTGACCTGCCTGGTCAAAGGCTTCTA |
| | TCCCAGCGACATCGCCGTGGAGTGGGAGAGCAATGGGCAGCCGGAGAACAACTACAAGACCACGCCTCCCGTGCTG |
| | GACTCCGACGGCTOCTTCTTCCTCTACAGCAAGCTCACCGTGGACAAGAGCAGGTGGCAGCAGGGGAACGTCTTCTC |
| | ATGCTCCGTGATGCATGAGGCTCTGCACAACCACTACACGCAGAAGAGCCTCTCCCTGTCTCCGGGTAAATGA |
| hIgG1 AA | EVQLVESGGGLVQPGGSLRLSCAASGFDLTGSYMHWVRQAPGKGLEWVAGISASGGATAYADSVKGRFTISADTSKNT |
| | AYLQMNSLRAEDTAVYYCARSRSSSYSSSGWRYYSGAFDYWGQGTLVTVSSASTKGPSVFPLAPSSKSTSGGTAALGCLV |
| | KDYFPEPVTVSWNSGALTSGVHTFPAVLQSSGLYSLSSVVTVPSSSLGTQTYICNVNHKPSNTKVDKKVEPKSCDKTHTCP |
| | PCPAPELLGGPSVFLFPPKPKDTLMISRTPEVTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSREEMTKNQVSLTCLVKGFYPSDIAVEWESNGQP |
| | ENNYKTTPPVLDSDGSFFLYSKLTVDKSRWQQGNVFSCSVMHEALHNHYTQKSLSLSPGK |
| 6. LEAD VHH(HC-NT)-Fab clone example | |
| 15031 VHH(HC-NT)-Fab | |
| VL-CL NT | GATATCCAGATGACCCAGTCCCCGAGCTCCCTGTCCGCCTCTGTGGGCGATAGGGTCACCATCACCTGCCGTGCCAG |
| | TCAGTCCGTGTCCAGCGCTGTAGCCTGGTATCAACAGAAACCAGGAAAAGCTCCGAAGCTTCTGATTTACTCGGCAT |
| | CCGATCTCTACTCTGGAGTCCCTTCTCGCTTCTCTGGTAGCCGTTCCGGGACGGATTTCACTCTGACCATCAGCAGTCT |
| | GCAGCCGGAAGACTTCGCAACTTATTACTGTCAGCAAGGTTCTTACCTGTTCACGTTCGGACAGGGTACCAAGGTGG |
| | AGATCAAACGTACGGTGGCTGCACCATCTGTCTTCATCTTCCCGCCATCTGATGAGCAGTTGAAATCTGGAACTGCCT |
| | CTGTTGTGTGCCTGCTGAATAACTTCTATCCCAGAGAGGCCAAAGTACAGTGGAAGGTGGATAACGCCCTCCAATCG |
| | GGTAACTCCCAGGAGAGTGTCACAGAGCAGGACAGCAAGGACAGCACCTACAGCCTCAGCAGCACCCTGACGCTGA |
| | GCAAAGCAGACTACGAAAAACATAAAGTCTACGCCTGCGAAGTCACCCATCAGGGCCTGAGCTCGCCCGTCACAAA |
| | GAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTLTISSLQPEDF |
| | ATYYCQQGSYLFTFGQGTKVEIKRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| VHH-VH-CH1 | GAGGTTCAGCTGGTGGAGTCTGGCGGTGGCCTGGTGCAGCCAGGGCGTTCACTCCGTTTGTCCTGTGCAGCTTCTG |
| NT | GCTTCGACCTCACTGGTTCTTACATGCACTGGGTGCGTCGTGCCCCGGGTAAGGGCGAGGAACTGGTTGCAGGTATT |
| | TCCGCTTCTGGAGGCGCTACTGCTTATGCCGATAGCGTCAAGGGCCGTTTCACTATAAGCGCAGACACATCCAAAAA |
| | CACAGCCTACCTACAAATGAACAGCTTAAGAGCTGAGGACACTGCCGTCTATTATTGTGCTCGCTCTCGTTCTTCTTCT |
| | TACTCTTCTTCTGGTTGGCGTTACTACTCTGGTGCTTTTGACTACTGGGGTCAAGGAACCCTGGTCACCGTCTCCTCGG |
| | GTGGAGGTGGCTCCGGAGGAGGTGGCAGTGAGGTTCAGCTGGTGGAGTCTGGCGGTGGCCTGGTGCAGCCAGGG |
| | GGCTCACTCCGTTTGTCCTGTGCAGCTTCTGGCTTCGATCTCACTGGTTCTTACATGCACTGGGTGCGTCAGGCCCCG |
| | GGTAAGGGCCTGGAATGGGTTGCAGGTATTTCCGCTTCTGGAGGCGCTACTGCTTATGCCGATAGCGTCAAGGGCC |
| | GTTTCACTATAAGCGCAGACACATCCAAAAACACAGCCTACCTACAAATGAACAGCTTAAGAGCTGAGGACACTGCC |
| | GTCTATTATTGTGCTCGCTCTCGTTCTTCTTCTTACTCTTCTTCTGGTTGGCGTTACTACTCTGGTGCTTTTGACTACTG |
| | GGGTCAAGGAACCCTGGTCACCGTCTCCTCGGCTAGCACCAAGGGCCCATCGGTCTTCCCCCTGGCACCCTCCTCCAA |
| | GAGCACCTCTGGGGGCACAGCGGCCCTGGGCTGCCTGGTCAAGGACTACTTCCCCGAACCGGTGACGGTGTCGTGG |
| | AACTCAGGCGCCCTGACCAGCGGCGTGCACACCTTCCCGGCTGTCCTACAGTCCTCAGGACTCTACTOCCTCAGCAGC |
| | GTGGTGACCGTGCCCTCCAGCAGCTTGGGCACCCAGACCTACATCTGCAACGTGAATCACAAGCCCAGCAACACCAA |
| | GGTGGACAAGAAAGTTGAGCCCAAATCTTGTGACAAAACTCACACA |
| VHH-VH-CH1 | EVQLVESGGGLVQPGRSLRLSCAASGFDLTGSYMHWVRRAPGKGEELVAGISASGGATAYADSVKGRFTISADTSKNTAY |
| AA | LQMNSERAEDTAVYYCARSRSSSYSSSGWRYYSGAFDYWGQGTLVTVSSGGGGSGGGGSEVQLVESGGGLVQPGGSLR |
| | LSCAASGFDLTGSYMHWVRQAPGKGLEWVAGISASGGATAYADSVKGRFTISADTSKNTAYLQMNSLRAEDTAVYYCA |
| | RSRSSSYSSSGWRYYSGAFDYWGQGTLVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKKVEPKSCDKTHT |
| 7. LEAD Fab-VHH(HC-CT) clone example | |
| 15031 Fab-VHH(HC-CT) | |
| VL-CL NT | GATATCCAGATGACCCAGTCCCCGAGCTCCCTGTCCGCCTCTGTGGGCGATAGGGTCACCATCACCTGCCGTGCCAG |
| | TCAGTCCGTGTCCAGCGCTGTAGCCTGGTATCAACAGAAACCAGGAAAAGCTCCGAAGCTTCTGATTTACTCGGCAT |
| | CCGATCTCTACTCTGGAGTCCCTTCTCGCTTCTCTGGTAGCCGTTCCGGGACGGATTTCACTCTGACCATCAGCAGTCT |
| | GCAGCCGGAAGACTTCGCAACTTATTACTGTCAGCAAGGTTCTTACCTGTTCACGTTCGGACAGGGTACCAAGGTGG |
| | AGATCAAACGTACGGTGGCTGCACCATCTGTCTTCATCTTCCCGCCATCTGATGAGCAGTTGAAATCTGGAACTGCCT |
| | CTGTTGTGTGCCTGCTGAATAACTTCTATCCCAGAGAGGCCAAAGTACAGTGGAAGGTGGATAACGCCCTCCAATCG |
| | GGTAACTCCCAGGAGAGTGTCACAGAGCAGGACAGCAAGGACAGCACCTACAGCCTCAGCAGCACCCTGACGCTGA |
| | GCAAAGCAGACTACGAAAAACATAAAGTCTACGCCTGCGAAGTCACCCATCAGGGCCTGAGCTCGCCCGTCACAAA |
| | GAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTLTISSLQPEDF |
| | ATYYCQQGSYLFTFGQGTKVEIKRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| VH-CH1-VHH | GAGGTTCAGCTGGTGGAGTCTGGCGGTGGCCTGGTGCAGCCAGGGGGCTCACTCCGTTTGTCCTGTGCAGCTTCTG |
| NT | GCTTCGATCTCACTGGTTCTTACATGCACTGGGTGCGTCAGGCCCCGGGTAAGGGCCTGGAATGGGTTGCAGGTATT |
| | TCCGCTTCTGGAGGCGCTACTGCTTATGCCGATAGCGTCAAGGGCCGTTTCACTATAAGCGCAGACACATCCAAAAA |
| | CACAGCCTACCTACAAATGAACAGCTTAAGAGCTGAGGACACTGCCGTCTATTATTGTGCTCGCTCTCGTTCTTCTTCT |
| | TACTCTTCTTCTGGTTGGCGTTACTACTCTGGTGCTTTTGACTACTGGGGTCAAGGAACCCTGGTCACCGTCTCCTCGG |
| | CTAGCACCAAGGGCCCATCGGTCTTCCCCCTGGCACCCTCCTCCAAGAGCACCTCTGGGGGCACAGCGGCCCTGGGC |
| | TGCCTGGTCAAGGACTACTTCCCCGAACCGGTGACGGTGTCGTGGAACTCAGGCGCCCTGACCAGCGGCGTGCACA |
| | CCTTCCCGGCTGTCCTACAGTCCTCAGGACTCTACTCCCTCAGCAGCGTGGTGACCGTGCCCTCCAGCAGCTTGGGCA |
| | CCCAGACCTACATCTGCAACGTGAATCACAAGCCCAGCAACACCAAGGTGGACAAGAAAGTTGAGCCCAAATCTTGT |
| | GACAAAACTCACACAGGTGGAGGTGGCTCCGGAGGAGGTGGCAGTGAGGTTCAGCTGGTGGAGTCTGGCGGTGG |
| | CCTGGTGCAGCCAGGGCGTTCACTCCGTTTGTCCTGTGCAGCTTCTGGCTTCGACCTCACTGGTTCTTACATGCACTG |
| | GGTGCGTCGTGCCCCGGGTAAGGGCGAGGAACTGGTTGCAGGTATTTCCGCTTCTGGAGGCGCTACTGCTTATGCC |
| | GATAGCGTCAAGGGCCGTTTCACTATAAGCGCAGACACATCCAAAAACACAGCCTACCTACAAATGAACAGCTTAAG |
| | AGCTGAGGACACTGCCGTCTATTATTGTGCTCGCTCTCGTTCTTCTTCTTACTCTTCTTCTGGTTGGCGTTACTACTCTG |
| | GTGCTTTTGACTACTGGGGTCAAGGAACCCTGGTCACCGTCTCCTCG |
| VH-CH1-VHH | EVQLVESGGGLVQPGGSLRISCAASGFDLTGSYMHWVROAPGKGLEWVAGISASGGATAYADSVKGRFTISADTSKNT |
| AA | AYLQMNSLRAEDTAVYYCARSRSSSYSSSGWRYYSGAFDYWGQGTLVTVSSASTKGPSVFPLAPSSKSTSGGTAALGCLV |
| | KDYFPEPVTVSWNSGALTSGVHTFPAVLQSSGLYSLSSVVTVPSSSLGTQTYICNVNHKPSNIKVDKKVEPKSCDKTHTGG |
| | GGSGGGGSEVQLVESGGGLVQPGRSLRLSCAASGFDLTGSYMHWVRRAPGKGEELVAGISASGGATAYADSVKGRATIS |
| | ADTSKNTAYLQMNSLRAEDTAVYYCARSRSSSYSSSGWRYYSGAFDYWGQGTLVTVSS |
| 8. LEAD scFV-Fab clone example | |
| 15031 scFv-Fab | |
| VL-VH-VL-CL | GATATCCAGATGACCCAGTCCCCGAGCTCCCTGTCCGCCTCTGTGGGCGATAGGGTCACCATCACCTGCCGTGCCAG |
| NT | CCAGAGCGTGTCCAGCGCCGTAGCCTGGTATCAACAGAAACCAGGAAAAGCTCCGAAGCTTCTGATTTACAGGGCCA |
| | GCGACCTGTACAGCGGAGTCCCTTCTCGCTTCTCTGGTAGCCGTTCCGGGACGGATTTCACTCTGACCATCAGCAGTC |
| | TGCAGCCGGAAGACTTCGCAACTTATTACTGTCAGCAAGGTTCTTACCTGTTCACGTTCGGACAGGGTACCAAGGTG |
| | GAGATCAAAGGTACTACTGCCGCTAGTGGTAGTAGTGGTGGCAGTAGCAGTGGTGCCGAGGTTCAGCTGGTGGAG |
| | TCTGGCGGTGGCCTGGTGCAGCCAGGGGGCTCACTCCGTTTGTCCTGTGCAGCTTCTGGCTTCGACCTCACTGGTTCT |
| | TACATGCACTGGGTGCGTCAGGCCCCGGGTAAGGGCCTGGAATGGGTTGCAGGTATTTCCGCTTCTGGAGGCGCTA |
| | CTGCTTATGCCGATAGCGTCAAGGGCCGTTTCACTATAAGCGCAGACACATCCAAAAACACAGCCTACCTACAAATG |
| | AACAGCTTAAGAGCTGAGGACACTGCCGTCTATTATTGTGCTCGCTCTCGTTCTTCTTCTTACTCTTCTTCTGGTTGGC |
| | GTTACTACTCTGGTGCTTTTGATTACTGGGGTCAAGGAACCCTGGTCACCGTCTCCTCGGGCGGCGGAGGCTCCGGC |
| | GGCGGAGGATCCGATATCCAGATGACCCAGTCCCCGAGCTCCCTGTCCGCCTCTGTGGGCGATAGGGTCACCATCAC |
| | CTGCCGTGCCAGTCAGTCCGTGTCCAGCGCTGTAGCCTGGTATCAACAGAAACCAGGAAAAGCTCCGAAGCTTCTGA |
| | TTTACTCGGCATCCGACCTTCTACTCTGGAGTCCCTTCTCGCTTCTCTGGTAGCCGTTCCGGGACGGATTTCACTCTGAC |
| | CATCAGCAGTCTGCAGCCGGAAGACTTCGCAACTTATTACTGTCAGCAAGGTTCTTACCTGTTCACGTTCGGACAGG |
| | GTACCAAGGTGGAGATCAAACGTACGGTGGCTGCACCCAGCGTGTTCATCTTCCCACCCAGCGACGAGCAGCTGAA |
| | GTCCGGCACAGCCAGCGTGGTCTGCCTGCTGAACAACTTCTACCCCCGCGAGGCCAAGGTGCAGTGGAAGGTGGAC |
| | AACGCCCTGCAGAGCGGCAACAGCCAGGAAAGCGTGACCGAGCAGGACAGCAAGGACTCCACCTACAGCCTGAGC |
| | AGCACCCTGACCCTGAGCAAGGCCGACTACGAGAAGCACAAGGTGTACGCCTGCGAAGTGACCCACCAGGGCCTGT |
| | CCAGCCCCGTGACCAAGAGCTTCAACCGGGGCGAGTGT |
| VL-VH-VL-CL | DIQMTQSPSSLSASVGDRVTITCRASQSVSSAVAWYQQKPGKAPKLLIYSASDLYSGVPSRFSGSRSGTDFTLTISSLQPEDF |
| AA | ATYYCQQGSYLFTFGQGTKVEIKGTTAASGSSGGSSSGAEVQLVESGGGLVQPGGSLRLSCAASGFDLTGSYMHWVRQA |
| | PGKGLEWVAGISASGGATAYADSVKGRFTISADTSKNTAYLQMNSLRAEDTAVYYCARSRSSSYSSSGWRYYSGAFDYW |
| | GQGTLVTVSSGGGGGGGGSDIQMTQSPSSLSASVGDRVTITCRASQSVSSAVAWYQQKPGKAPKLLIYSASDLYSGVPS |
| | RFSGSRSGTDFTLTISSLQPEDFATYYCQQGSYLFTFGQGTKVEIKRTVAAPSVFIFPPSDEQLKSGTASVVCLLNNFYPREAK |
| | VQWKVDNALQSGNSQESVTEQDSKOSTYSLSSTLTLSKADYCKIIKVYACCVTHQGLSSPVTKSFNRGEC |
| VH-CH1 NT | GAGGTTCAGCTGGTGGAGTCTGGCGGTGGCCTGGTGCAGCCAGGGGGCTCACTCCGTTTGTCCTGTGCAGCTTCTG |
| | GCTTCGATCTCACTGGTTCTTACATGCACTGGGTGCGTCAGGCCCCGGGTAAGGGCCTGGAATGGGTTGCAGGTATT |
| | TCCGCTTCTGGAGGCGCTACTGCTTATGCCGATAGCGTCAAGGGCCGTTTCACTATAAGCGCAGACACATCCAAAAA |
| | CACAGCCTACCTACAAATGAACAGCTTAAGAGCTGAGGACACTGCCGTCTATTATTGTGCTCGCTCTCGTTCTTCTTCT |
| | TACTCTTCTTCTGGTTGGCGTTACTACTCTGGTGCTTTTGACTACTGGGGTCAAGGAACCCTGGTCACCGTCTCCTCGG |
| | CTAGCACCAAGGGCCCATCGGTCTTCCCCCTGGCACCCTCCTCCAAGAGCACCTCTGGGGGCACAGCGGCCCTGGGC |
| | TGCCTGGTCAAGGACTACTTCCCCGAACCGGTGACGGTGTCGTGGAACTCAGGCGCCCTGACCAGCGGCGTGCACA |
| | CCTTCCCGGCTGTCCTACAGTCCTCAGGACTCTACTCCCTCAGCAGCGTGGTGACCGTGCCCTCCAGCAGCTTGGGCA |
| | CCCAGACCTACATCTGCAACGTGAATCACAAGCCCAGCAACACCAAGGGGACAAGAAAGTTGAGCCCAAATCTTGT |
| | GACAAAACTCACACA |
| VH-CH1 AA | EVQLVESGGGLVQPGGSLRLSCAASGFDLTGSYMHWVRQAPGKGLEWVAGISASGGATAYADSVKGRFTISADTSKNT |
| | AYLQMNSLRAEDTAVYYCARSRSSSYSSSGWRYYSGAFDYWGQGTLVTVSSASTKGPSVFPLAPSSKSTSGGTAALGCLV |
| | KDYFPEPVTVSWNSGALTSGVHTFPAVLQSSGLYSLSSVVTVPSSSLGTQTYICNVNHKPSNTKVDKKVEPKSCDKTHT |
| 9. LEAD Fab-scFV clone example | |
| 15031 Fab-scFv | |
| VL-CL-VL-VH | GATATCCAGATGACCCAGTCCCCGAGCTCCCTGTCCGCCTCTGTGGGCGATAGGGTCACCATCACCTGCCGTGCCAG |
| NT | TCAGTCCGTGTCCAGCGCTGTAGCCTGGTATCAACAGAAACCAGGAAAAGCTCCGAAGCTTCTGATTTACTCGGCAT |
| | CCGATCTCTACTCTGGAGTCCCTTCTCGCTTCTCTGGTAGCCGTTCCGGGACGGATTTCACTCTGACCATCAGCAGTCT |
| | GCAGCCGGAAGACTTCGCAACTTATTACTGTCAGCAAGGTTCTTACCTGTTCACGTTCGGACAGGGTACCAAGGTGG |
| | AGATCAAACGTACGGTGGCTGCACCATCTGTCTTCATCTTCCCGCCATCTGATGAGCAGTTGAAATCTGGAACTGCCT |
| | CTGTTGTGTGCCTGCTGAATAACTTCTATCCCAGAGAGGCCAAAGTACAGTGGAAGGTGGATAACGCCCTCCAATCG |
| | GGTAACTCCCAGGAGAGTGTCACAGAGCAGGACAGCAAGGACAGCACCTACAGCCTCAGCAGCACCCTGACGCTGA |
| | GCAAAGCAGACTACGAAAAACATAAAGTCTACGCCTGCGAAGTCACCCATCAGGGCCTGAGCTCGCCCGTCACAAA |
| | GAGCTTCAACAGGGGAGAGTGTGGCGGCGGAGGATCCGATATCCAGATGACCCAGTCCCCGAGCTCOCTGTCCGCC |
| | TCTGTGGGCGATAGGGTCACCATCACCTGCCGTGCCAGCCAGAGCGTGTCCAGCGCCGTAGCCTGGTATCAACAGA |
| | AACCAGGAAAAGCTCCGAAGCTTCTGATTTACAGCGCCAGCGACCTGTACAGCGGAGTCCCTTCTCGCTTCTCTGGT |
| | AGCCGTTCCGGGACGGATTTCACTCTGACCATCAGCAGTCTGCAGCCGGAAGACTTCGCAACTTATTACTGTCAGCA |
| | AGGTTCTTACCTGTTCACGTTCGGACAGGGTACCAAGGTGGAGATCAAAGGTACTACTGCCGCTAGTGGTAGTAGT |
| | GGTGGCAGTAGCAGTGGTGCCGAGGTTCAGCTGGTGGAGTCTGGCGGTGGCCTGGTGCAGCCAGGGGGCTCACTC |
| | CGTTTGTCCTGTGCAGCTTCTGGCTTCGACCTCACTGGTTCTTACATGCACTGGGTGCGTCAGGCCCCGGGTAAGGG |
| | CCTGGAATGGGTTGCAGGTATTTCCGCTTCTGGAGGGGCTACTGCTTATGCCGATAGCGTCAAGGGCCGTTTCACTA |
| | TAAGCGCAGACACATCCAAAAACACAGCCTACCTACAAATGAACAGCTTAAGAGCTGAGGACACTGCCGTCTATTAT |
| | TGTGCTCGCTCTCGTTCTTCTTCTTACTCTTCTTCTGGTTGGCGTTACTACTCTGGTGCTTTTGATTACTGGGGTCAAGG |
| | AACCCTGGTCACCGTCTCCTCG |
| VL-CL-VL-VH | DIQMTQSPSSLSASVGDRVTITCRASQSVSSAVAWYQQKPGKAPKLLIYSASDLYSGVPSRFSGSRSGTDFTLTISSLQPEDF |
| AA | ATYYCQQGSYLFTFGQGTKVEIKRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGECGGGGSDIQMTQSPSSLSASVGDRVTITCRASQ |
| | SVSSAVAWYQQKPGKAPKLLYSASDLYSGVPSRFSGSRSGTDFTLTISSLQPEDFATVYCQQGSYLFTFGQGTKVEIKGTTA |
| | ASGSSGGSSSGAEVQLVESGGGLVQPGGSLRISCAASGFDLTGSYMHWVRQAPGKGLEWVAGISASGGATAYADSVKG |
| | RFTISADTSKNTAYLQMNSLRAEDTAVYYCARSRSSSYSSSGWRYYSGAFDYWGQGTLVTVSS |
| VH-CH1 | GAGGTTCAGCTGGTGGAGTCTGGCGGTGGCCTGGTGCAGCCAGGGGGCTCACTCCGTTTGTCCTGTGCAGCTTCTG |
| NT | GCTTCGATCTCACTGGTTCTTACATGCACTGGGTGCGTCAGGCCCCGGGTAAGGGCCTGGAATGGGTTGCAGGTATT |
| | TCCGCTTCTGGAGGCGCTACTGCTTATGCCGATAGCGTCAAGGGCCGTTTCACTATAAGCGCAGACACATCCAAAAA |
| | CACAGCCTACCTACAAATGAACAGCTTAAGAGCTGAGGACACTGCCGTCTATTATTGTGCTCGCTCTCGTTCTTCTTCT |
| | TACTCTTCTTCTGGTTGGCGTTACTACTCTGGTGCTTTTGACTACTGGGGTCAAGGAACCCTGGTCACCGTCTCCTCGG |
| | CTAGCACCAAGGGCCCATCGGTCTTCCCCCTGGCACCCTCCTCCAAGAGCACCTCTGGGGGCACAGCGGCCCTGGGC |
| | TGCCTGGTCAAGGACTACTTCCCCGAACCGGTGACGGTGTCGTGGAACTCAGGCGCCCTGACCAGCGGCGTGCACA |
| | CCTTCCCGGCTGTCCTACAGTCCTCAGGACTCTACTCCCTCAGCAGCGTGGTGACCGTGCCCTCCAGCAGCTTGGGCA |
| | CCCAGACCTACATCTGCAACGTGAATCACAAGCCCAGCAACACCAAGGTGGACAAGAAAGTTGAGCCCAAATCTTGT |
| | GACAAAACTCACACA |
| VH-CH1 | EVQLVESGGGLVQPGGSLRLSCAASGFDLTGSYMHWVRQAPGKGLEWVAGISASGGATAYADSVKGRFTISADTSKNT |
| AA | AYLQMNSLRAEDTAVYYCARSRSSSYSSSGWRYYSGAFDYWGQGTLVTVSSASTKGPSVFPLAPSSKSTSGGTAALGCLV |
| | KDYFPEPVTVSWNSGALTSGVHTFPAVLQSSGLYSLSSVVTVPSSSLGTQTYICNVNHKPSNTKVDKKVEPKSCDKTHT |
| 10. LEAD Fab-diabody clone example | |
| 15031 Fab-diabody | |
| VL-CL-VH-VL | GATATCCAGATGACCCAGTCCCCGAGCTCCCTGTCCGCCTCTGTGGGCGATAGGGTCACCATCACCTGCCGTGCCAG |
| NT | TCAGTCCGTGTCCAGCGCTGTAGCCTGGTATCAACAGAAACCAGGAAAAGCTCCGAAGCTTCTGATTTACTCGGCAT |
| | CCGACCTCTACTCTGGAGTCCCTTCTCGCTTCTCTGGTAGCCGTTCCGGGACGGATTTCACTCTGACCATCAGCAGTCT |
| | GCAGCCGGAAGACTTCGCAACTTATTACTGTCAGCAAGGTTCTTACCTGTTCACGTTCGGACAGGGTACCAAGGTGG |
| | AGATCAAACGTACGGTGGCTGCACCCAGCGTGTTCATCTTCCCACCCAGCGACGAGCAGCTGAAGTOCGGCACAGCC |
| | AGCGTGGTCTGCCTGCTGAACAACTTCTACCCCCGCGAGGCCAAGGTGCAGTGGAAGGTGGACAACGCCCTGCAGA |
| | GCGGCAACAGCCAGGAAAGCGTGACCGAGCAGGACAGCAAGGACTCCACCTACAGCCTGAGCAGCACCCTGACCCT |
| | GAGCAAGGCCGACTACGAGAAGCACAAGGTGTACGCCTGCGAAGTGACCCACCAGGGCCTGTCCAGCCCCGTGACC |
| | AAGAGCTTCAACCGGGGCGAGTGTGGCGGCGGAGGATCCGAGGTTCAGCTGGTGGAGTCTGGCGGTGGCCTGGTG |
| | CAGCCAGGGGGCTCACTCCGTTTGTCCTGTGCAGCTTCTGGCTTCGACCTCACTGGTTCTTACATGCACTGGGTGCGT |
| | CAGGCCCCGGGTAAGGGCCTGGAATGGGTTGCAGGTATTTCCGCTTCTGGAGGCGCTACTGCTTATGCCGATAGCG |
| | TCAAGGGCCGTTTCACTATAAGCGCAGACACATCCAAAAACACAGCCTACCTACAAATGAACAGCTTAAGAGCTGAG |
| | GACACTGCCGTCTATTATTGTGCTCGCTCTCGTTCTTCTTCTTACTCTTCTTCTGGTTGGCGTTACTACTCTGGTGCTTTT |
| | GATTACTGGGGTCAAGGAACCCTGGTCACCGTCTCCTCGGGCGGCGGAGGATCCGATATCCAGATGACCCAGTCCC |
| | CGAGCTCCCTGTCCGCCTCTGTGGGCGATAGGGTCACCATCACCTGCCGTGCCAGCCAGAGCGTGTCCAGCGCCGTA |
| | GCCTGGTATCAACAGAAACCAGGAAAAGCTCCGAAGCTTCTGATTTACAGCGCCAGCGACCTGTACAGCGGAGTCCC |
| | TTCTCGCTTCTCTGGTAGCCGTTCCGGGACGGATTTCACTCTGACCATCAGCAGTCTGCAGCCGGAAGACTTCGCAAC |
| | TTATTACTGTCAGCAAGGTTCTTACCTGTTCACGTTCGGACAGGGTACCAAGGTGGAGATCAAA |
| VL-CL-VH-VL | DIQMTQSPSSLSASVGDRVTITCRASQSVSSAVAWYQQKPGKAPKLLIYSASDLYSGVPSRFSGSRSGTDFTLTISSLQPEDF |
| AA | ATYYCQQGSYLFTFGQGTKVEIKRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGECGGGGSEVQLVESGGGLVQPGGSLRLSCAASGF |
| | DLTGSYMHWVRQAPGKGLEWVAGISASGGATAYADSVKGRFTISADTSKNTAYLQMNSLRAEDTAVYYCARSRSSSYSS |
| | SGWRYYSGAFDYWGQGTLVTVSSGGGGSDIQMTQSPSSLSASVGDRVTITCRASQSVSSAVAWYQQKPGKAPKLLIYSA |
| | SDLYSGVPSRFSGSRSGTDFTLTISSLQPEDFATYYCQQGSYLFTFGQGTKVEIK |
| VH-CH1-VH-VL | GAGGTTCAGCTGGTGGAGTCTGGCGGTGGCCTGGTGCAGCCAGGGGGCTCACTCCGTTTGTCCTGTGCAGCTTCTG |
| NT | GCTTCGACCTCACTGGTTCTTACATGCACTGGGTGCGTCAGGCCCCGGGTAAGGGCCTGGAATGGGTTGCAGGTATT |
| | TCCGCTTCTGGAGGCGCTACTGCTTATGCCGATAGCGTCAAGGGCCGTTTCACTATAAGCGCAGACACATCCAAAAA |
| | CACAGCCTACCTACAAATGAACAGCTTAAGAGCTGAGGACACTGCCGTCTATTATTGTGCTCGCTCTCGTTCTTCTTCT |
| | TACTCTTCTTCTGGTTGGCGTTACTACTCTGGTGCTTTTGACTACTGGGGTCAAGGAACCCTGGTCACCGTCTCCTCGG |
| | CTAGCACCAAGGGCCCATCGGTCTTCCCCCTGGCACCCTCCTCCAAGAGCACCTCTGGGGGCACAGCGGCCCTGGGC |
| | TGCCTGGTCAAGGACTACTTCCCCGAACCGGTGACGGTGTCGTGGAACTCAGGCGCCCTGACCAGCGGCGTGCACA |
| | CCTTCCCGGCTGTCCTACAGTCCTCAGGACTCTACTCCCTCAGCAGCGTGGTGACCGTGCCCTCCAGCAGCTTGGGCA |
| | CCCAGACCTACATCTGCAACGTGAATCACAAGCCCAGCAACACCAAGGTGGACAAGAAAGTTGAGCCCAAATCTTGT |
| | GACAAAACTCACACAGGTGGATCCGGCGGCGAGGTTCAGCTGGTGGAGTCTGGCGGTGGCCTGGTGCAGCCAGGG |
| | GGCTCACTCCGTTTGTCCTGTGCAGCTTCTGGCTTCGACCTCACTGGTTCTTACATGCACTGGGTGCGTCAGGCCCCG |
| | GGTAAGGGCCTGGAATGGGTTGCAGGTATTTCCGCTTCTGGAGGCGCTACTGCTTATGCCGATAGCGTCAAGGGCC |
| | GTTTCACTATAAGCGCAGACACATCCAAAAACACAGCCTACCTACAAATGAACAGCTTAAGAGCTGAGGACACTGCC |
| | GTCTATTATTGTGCTCGCTCTCGTTCTTCTTCTTACTCTTCTTCTGGTTGGCGTTACTACTCTGGTGCTTTTATGGACTAC |
| | TGGGGTCAAGGAACCCTGGTCACCGTCTCCTCGGGTGGAGGTGGCAGTGATATCCAGATGACCCAGTCCCCGAGCT |
| | CCCTGTCCGCCTCTGTGGGCGATAGGGTCACCATCACCTGCCGTGCCAGTCAGTCCGTGTCCAGCGCTGTAGCCTGG |
| | TATCAACAGAAACCAGGAAAAGCTCCGAAGCTTCTGATTTACTCGGCATCCGACCTCTACTCTGGAGTCCCTTCTCGC |
| | TTCTCTGGTAGCCGTTCCGGGACGGATTTCACTCTGACCATCAGCAGTCTGCAGCCGGAAGACTTCGCAACTTATTAC |
| | TGTCAGCAAGGTTCTTACCTGTTCACGTTCGGACAGGGTACCAAGGTGGAGATCAAA |
| VH-CH1-VH-VL | EVQLVESGGGLVQPGGSLRLSCAASGFDLTGSYMHWVRQAPGKGLEWVAGISASGGATAYADSVKGRFTISADISKNT |
| AA | AYLQMNSLRAEDTAVYYCARSRSSSYSSSGWRYYSGAFDYWGQGTLVTVSSASTKGPSVFPLAPSSKSTSGGTAALGCLV |
| | KDYFPEPVTVSWNSGALTSGVHTFPAVLQSSGLYSLSSVVTVPSSSLGTQTYICNVNHKPSNTKVDKKVEPKSCDKTHTGG |
| | SGGEVQLVESGGGLVQPGGSLRLSCAASGFDLTGSYMHWVRQAPGKGLEWVAGISASGGATAYADSVKGRFTISADTS |
| | KNTAYLQMNSLRAEDTAVYYCARSRSSSYSSSGWRYYSGAFMDYWGQGTLVTVSSGGGGSDIQMTQSPSSLSASVGDR |
| | VTITCRASQSVSSAVAWYQQKPGKAPKLLIYSASDLYSGVPSRESGSRSGTDFTLTISSLQPEDFATYYCQQGSYLFTFGQGT |
| | KVEIK |
| 11. LEAD VHH(HC-CT)-Fab-scFV clone example | |
| 15031 VHH(HC-CT)-Fab-scFv | |
| VL-CL-VL-VH | GATATCCAGATGACCCAGTCCCCGAGCTCCCTGTCCGCCTCTGTGGGCGATAGGGTCACCATCACCTGCCGTGCCAG |
| NT | TCAGTCCGTGTCCAGCGCTGTAGCCTGGTATCAACAGAAACCAGGAAAAGCTCCGAAGCTTCTGATTTACTCGGCAT |
| | CCGATCTCTACTCTGGAGTCCCTTCTCGCTTCTCTGGTAGCCGTTCCGGGACGGATTTCACTCTGACCATCAGCAGTCT |
| | GCAGCCGGAAGACTTCGCAACTTATTACTGTCAGCAAGGTTCTTACCTGTTCACGTTCGGACAGGGTACCAAGGTGG |
| | AGATCAAACGTACGGTGGCTGCACCATCTGTCTTCATCTTCCCGCCATCTGATGAGCAGTTGAAATCTGGAACTGCCT |
| | CTGTTGTGTGCCTGCTGAATAACTTCTATCCCAGAGAGGCCAAAGTACAGTGGAAGGTGGATAACGCCCTCCAATCG |
| | GGTAACTCCCAGGAGAGTGTCACAGAGCAGGACAGCAAGGACAGCACCTACAGCCTCAGCAGCACCCTGACGCTGA |
| | GCAAAGCAGACTACGAAAAACATAAAGTCTACGCCTGCGAAGTCACCCATCAGGGCCTGAGCTCGCCCGTCACAAA |
| | GAGCTTCAACAGGGGAGAGTGTGGCGGCGGAGGATCCGATATCCAGATGACCCAGTCCCCGAGCTCCCTGTCCGCC |
| | TCTGTGGGCGATAGGGTCACCATCACCTGCCGTGCCAGCCAGAGCGTGTCCAGCGCCGTAGCCTGGTATCAACAGA |
| | AACCAGGAAAAGCTCCGAAGCTTCTGATTTACAGCGCCAGCGACCTGTACAGGGGAGTCCCTTCTOGCTTCTCTGGT |
| | AGCCGTTCCGGGACGGATTTCACTCTGACCATCAGCAGTCTGCAGCCGGAAGACTTCGCAACTTATTACTGTCAGCA |
| | AGGTTCTTACCTGTTCACGTTCGGACAGGGTACCAAGGTGGAGATCAAAGGTACTACTGCCGCTAGTGGTAGTAGT |
| | GGTGGCAGTAGCAGTGGTGCCGAGGTTCAGCTGGTGGAGTCTGGCGGTGGCCTGGTGCAGCCAGGGGGCTCACTC |
| | CGTTTGTCCTGTGCAGCTTCTGGCTTCGACCTCACTGGTTCTTACATGCACTGGGTGCGTCAGGCCCCGGGTAAGGG |
| | CCTGGAATGGGTTGCAGGTATTTCCGCTTCTGGAGGCGCTACTGCTTATGCCGATAGCGTCAAGGGCCGTTTCACTA |
| | TAAGCGCAGACACATCCAAAAACACAGCCTACCTACAAATGAACAGCTTAAGAGCTGAGGACACTGCCGTCTATTAT |
| | TGTGCTCGCTCTCGTTCTTCTTCTTACTCTTCTTCTGGTTGGCGTTACTACTCTGGTGCTTTTGATTACTGGGGTCAAGG |
| | AACCCTGGTCACCGTCTCCTCG |
| VL-CL-VL-VH | DIQMTQSPSSLSASVGDRVTITCRASQSVSSAVAWYQQKPGKAPKLLIYSASDLYSGVPSRFSGSRSGTDFTLTISSLQPEDF |
| AA | ATYYCQQGSYLFTFGQGTKVEIKRTVAAPSVFIFPPSDEQLKSGTASVVCLLNNFYPREAKVQWKVDNALQSGNSQESVTE |
| | QDSKDSTYSLSSTLILSKADYEKHKVYACEVTHQGLSSPVTKSFNRGECGGGGSDIQMTQSPSSLSASVGDRVTITCRASQ |
| | SVSSAVAWYQQKPGKAPKLLIYSASDLYSGVPSRFSGSRSGTDFTLTISSLQPEDFATYYCQQGSYLFTFGQGTKVEIKGTTA |
| | ASGSSGGSSSGAEVQLVESGGGLVQPGGSLRLSCAASGFDLTGSYMHWVRQAPGKGLEWVAGISASGGATAYADSVKG |
| | RFTISADTSKNTAYLQMNSLRAEDTAVYYCARSRSSSYSSSGWRYYSGAFDYWGQGTLVTVSS |
| VH-CH1-VHH | GAGGTTCAGCTGGTGGAGTCTGGCGGTGGCCTGGTGCAGCCAGGGGGCTCACTCCGTTTGTCCTGTGCAGCTTCTG |
| NT | GCTTCGACCTCACTGGTTCTTACATGCACTGGGTGCGTCAGGCCCCGGGTAAGGGCCTGGAATGGGTTGCAGGTATT |
| | TCCGCTTCTGGAGGCGCTACTGCTTATGCCGATAGCGTCAAGGGCCGTTTCACTATAAGCGCAGACACATCCAAAAA |
| | CACAGCCTACCTACAAATGAACAGCTTAAGAGCTGAGGACACTGCCGTCTATTATTGTGCTCGCTCTCGTTCTTCTTCT |
| | TACTCTTCTTCTGGTTGGCGTTACTACTCTGGTGCTTTTGACTACTGGGGTCAAGGAACCCTGGTCACCGTCTCCTCGG |
| | CTAGCACCAAGGGCCCATCGGTCTTCCCCCTGGCACCCTCCTCCAAGAGCACCTCTGGGGGCACAGOGGCCCTGGGC |
| | TGCCTGGTCAAGGACTACTTCCCCGAACCGGTGACGGTGTCGTGGAACTCAGGCGCCCTGACCAGCGGCGTGCACA |
| | CCTTCCCGGCTGTCCTACAGTCCTCAGGACTCTACTCCCTCAGCAGCGTGGTGACCGTGCCCTCCAGCAGCTTGGGCA |
| | CCCAGACCTACATCTGCAACGTGAATCACAAGCCCAGCAACACCAAGGTGGACAAGAAAGTTGAGCCCAAATCTTGT |
| | GACAAAACTCACACAGGAGGAGGTGGGGGATCAGGTGGTGGGGGCTCTGGAGGCGGAACCGGTGAGGTTCAGCT |
| | GGTGGAGTCTGGCGGTGGCCTGGTGCAGCCAGGGCGTTCACTCCGTTTGTCCTGTGCAGCTTCTGGCTTCGACCTCA |
| | CTGGTTCTTACATGCACTGGGTGCGTCGTGCCCCGGGTAAGGGCGAGGAACTGGTTGCAGGTATTTCCCCTTCTGGA |
| | GGCGCTACTGCTTATGCCGATAGCGTCAAGGGCCGTTTCACTATAAGCGCAGACACATCCAAAAACACAGCCTACCT |
| | ACAAATGAACAGCTTAAGAGCTGAGGACACTGCCGTCTATTATTGTGCTCGCTCTCGTTCTTCTTCTTACTCTTCTTCT |
| | GGTTGGCGTTACTACTCTGGTGCTTTTGACTACTGGGGTCAAGGAACCCTGGTCACCGTCTCCTCG |
| VH-CH1-VHH | EVQLVESGGGLVQPGGSLRLSCAASGFDLTGSYMHWVRQAPGKGLEWVAGISASGGATAYADSVKGRATISADTSKNT |
| | AYLQMNSLRAEDTAVYYCARSRSSSYSSSGWRYYSGAFDYWGQGTLVTVSSASTKGPSVFPLAPSSKSTSGGTAALGCLV |
| | KDYFPEPVTVSWNSGALTSGVHTFPAVLQSSGLYSLSSVVTVPSSSIGTQTYICNVNHKPSNTKVDKKVEPKSCDKTHTGG |
| | GGGSGGGGSGGGTGEVQLVESGGGLVQPGRSLRLSCAASGFDLTGSYMHWVRRAPGKGEELVAGISASGGATAYADS |
| | VKGRFTISADTSKNTAYLQMNSLRAEDTAVYYCARSRSSSYSSSGWRYYSGAFDYWGQGTLVTVSS |
| 12. LEAD VHH(HC-NT)-Fab-scFV clone example | |
| 15031 VHH(HC-NT)-Fab-scFv | |
| VL-CL-VL-VH | GATATCCAGATGACCCAGTCCCCGAGCTCCCTGTCCGCCTCTGTGGGCGATAGGGTCACCATCACCTGCCGTGCCAG |
| NT | TCAGTCCGTGTCCAGCGCTGTAGCCTGGTATCAACAGAAACCAGGAAAAGCTCCGAAGCTTCTGATTTACTCGGCAT |
| | CCGATCTCTACTCTGGAGTCCCTTCTCGCTTCTCTGGTAGCCGTTCCGGGACGGATTTCACTCTGACCATCAGCAGTCT |
| | GCAGCCGGAAGACTTCGCAACTTATTACTGTCAGCAAGGTTCTTACCTGTTCACGTTCGGACAGGGTACCAAGGTGG |
| | AGATCAAACGTACGGTGGCTGCACCATCTGTCTTCATCTTCCCGCCATCTGATGAGCAGTTGAAATCTGGAACTGCCT |
| | CTGTTGTGTGCCTGCTGAATAACTTCTATCCCAGAGAGGCCAAAGTACAGTGGAAGGTGGATAACGCCCTCCAATCG |
| | GGTAACTCCCAGGAGAGTGTCACAGAGCAGGACAGCAAGGACAGCACCTACAGCCTCAGCAGCACCCTGACGCTGA |
| | GCAAAGCAGACTACGAAAAACATAAAGTCTACGCCTGCGAAGTCACCCATCAGGGCCTGAGCTCGCCCGTCACAAA |
| | GAGCTTCAACAGGGGAGAGTGTGGCGGCGGAGGATCCGATATCCAGATGACCCAGTCCCCGAGCTCCCTGTCCGCC |
| | TCTGTGGGCGATAGGGTCACCATCACCTGCCGTGCCAGCCAGAGCGTGTCCAGCGCCGTAGCCTGGTATCAACAGA |
| | AACCAGGAAAAGCTCCGAAGCTTCTGATTTACAGCGCCAGCGACCTGTACAGCGGAGTCCCTTCTCGCTTCTCTGGT |
| | AGCCGTTCCGGGACGGATTTCACTCTGACCATCAGCAGTCTGCAGCCGGAAGACTTCGCAACTTATTACTGTCAGCA |
| | AGGTTCTTACCTGTTCACGTTCGGACAGGGTACCAAGGTGGAGATCAAAGGTACTACTGCCGCTAGTGGTAGTAGT |
| | GGTGGCAGTAGCAGTGGTGCCGAGGTTCAGCTGGTGGAGTCTGGCGGTGGCCTGGTGCAGCCAGGGGGCTCACTC |
| | CGTTTGTCCTGTGCAGCTTCTGGCTTCGACCTCACTGGTTCTTACATGCACTGGGTGCGTCAGGCCCCGGGTAAGGG |
| | CCTGGAATGGGTTGCAGGTATTTCCGCTTCTGGAGGCGCTACTGCTTATGCCGATAGCGTCAAGGGCCGTTTCACTA |
| | TAAGCGCAGACACATCCAAAAACACAGCCTACCTACAAATGAACAGCTTAAGAGCTGAGGACACTGCCGTCTATTAT |
| | TGTGCTCGCTCTCGTTCTTCTTCTTACTCTTCTTCTGGTTGGCGTTACTACTCTGGTGCTTTTGATTACTGGGGTCAAGG |
| | AACCCTGGTCACCGTCTCCTCG |
| VL-CL-VL-VH | DIQMTQSPSSLSASVGDRVTITCRASQSVSSAVAWYQQKPGKAPKLLIYSASDLYSGVPSRFSGSRSGTDFTLTISSLQPEDF |
| AA | ATYYCQQGSYLFTFGQGTKVEIKRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGECGGGGSDIQMTQSPSSLSASVGDRVTITCRASQ |
| | SVSSAVAWYQQKPGKAPKLLIYSASDLYSGVPSRFSGSRSGTDFTLTISSLQPEDFATYYCQQGSYLETFGQGTKVEIKGTTA |
| | ASGSSGGSSSGAEVQLVESGGGLVQPGGSLRLSCAASGFDLTGSYMHWVRQAPGKGLEWVAGISASGGATAYADSVKG |
| | RFTISADTSKNTAYLQMNSLRAEDTAVYYCARSRSSSYSSSGWRYYSGAFDYWGQGTLVTVSS |
| VHH-VH-CH1 | GAGGTTCAGCTGGTGGAGTCTGGCGGTGGCCTGGTGCAGCCAGGGCGTTCACTCCGTTTGTCCTGTGCAGCTTCTG |
| NT | GCTTCGACCTCACTGGTTCTTACATGCACTGGGTGCGTCGTGCCCCGGGTAAGGGCGAGGAACTGGTTGCAGGTATT |
| | TCCGCTTCTGGAGGCGCTACTGCTTATGCCGATAGCGTCAAGGGCCGTTTCACTATAAGCGCAGACACATCCAAAAA |
| | CACAGCCTACCTACAAATGAACAGCTTAAGAGCTGAGGACACTGCCGTCTATTATTGTGCTCGCTCTCGTTCTTCTTCT |
| | TACTCTTCTTCTGGTTGGCGTTACTACTCTGGTGCTTTTGACTACTGGGGTCAAGGAACCCTGGTCACCGTCTCCTCGG |
| | GTGGAGGTGGCTCCGGAGGAGGTGGCAGTGAGGTTCAGCTGGTGGAGTCTGGCGGTGGCCTGGTGCAGCCAGGG |
| | GGCTCACTCCGTTTGTCCTGTGCAGCTTCTGGCTTCGACCTCACTGGTTCTTACATGCACTGGGTGCGTCAGGCCCCG |
| | GGTAAGGGCCTGGAATGGGTTGCAGGTATTTCCGCTTCTGGAGGCGCTACTGCTTATGCCGATAGCGTCAAGGGCC |
| | GTTTCACTATAAGCGCAGACACATCCAAAAACACAGCCTACCTACAAATGAACAGCTTAAGAGCTGAGGACACTGCC |
| | GTCTATTATTGTGCTCGCTCTCGTTCTTCTTCTTACTCTTCTTCTGGTTGGCGTTACTACTCTGGTGCTTTTGACTACTG |
| | GGGTCAAGGAACCCTGGTCACCGTCTCCTCGGCTAGCACCAAGGGCCCATCGGTCTTCCCCCTGGCACCCTCCTOCAA |
| | GAGCACCTCTGGGGGCACAGCGGCCCTGGGCTGCCTGGTCAAGGACTACTTCCCCGAACCGGTGACGGTGTCGTGG |
| | AACTCAGGCGCCCTGACCAGCGGCGTGCACACCTTCCCGGCTGTCCTACAGTCCTCAGGACTCTACTCCCTCAGCAGC |
| | GTGGTGACCGTGCCCTCCAGCAGCTTGGGCACCCAGACCTACATCTGCAACGTGAATCACAAGCCCAGCAACACCAA |
| | GGTGGACAAGAAAGTTGAGCCCAAATCTTGTGACAAAACTCACACA |
| VHH-VH-CH1 | EVQLVESGGGLVQPGRSLRLSCAASGFDLTGSYMHWVRRAPGKGEELVAGISASGGATAYADSVKGRFTISADTSKNTAY |
| AA | LQMNSLRAEDTAVYYCARSRSSSYSSSGWRYYSGAFDYWGQGTLVTVSSGGGGSGGGGSEVQLVESGGGLVQPGGSLR |
| | LSCAASGFDLTGSYMHWVRQAPGKGLEWVAGISASGGATAYADSVKGRFTISADTSKNTAYLQMNSLRAEDTAVYYCA |
| | RSRSSSYSSSGWRYYSGAFDYWGQGTLVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKKVEPKSCDKTHT |
| 13. LEAD VHH(LC-NT)-Fab-scFV clone example | |
| 15031 VHH(LC-NT)-Fab-scFv | |
| VHH-VL-CL-VL- | GAGGTTCAGCTGGTGGAGTCTGGCGGTGGCCTGGTGCAGCCAGGGCGTTCACTCCGTTTGTCCTGTGCAGCTTCTG |
| VH NT | GCTTCGACCTCACTGGTTCTTACATGCACTGGGTGCGTCGTGCCCCGGGTAAGGGCGAGGAACTGGTTGCAGGTATT |
| | TCCGCTTCTGGAGGCGCTACTGCTTATGCCGATAGCGTCAAGGGCCGTTTCACTATAAGCGCAGACACATCCAAAAA |
| | CACAGCCTACCTACAAATGAACAGCTTAAGAGCTGAGGACACTGCCGTCTATTATTGTGCTCGCTCTCGTTCTTCTTCT |
| | TACTCTTCTTCTGGTTGGCGTTACTACTCTGGTGCTTTTGACTACTGGGGTCAAGGAACCCTGGTCACCGTCTCCTCGG |
| | GTGGAGGTGGCTCCGGAGGAGGTGGCAGTGATATCCAGATGACCCAGTCCCCGAGCTCCCTGTCCGCCTCTGTGGG |
| | CGATAGGGTCACCATCACCTGCCGTGCCAGTCAGTCCGTGTCCAGCGCTGTAGCCTGGTATCAACAGAAACCAGGAA |
| | AAGCTCCGAAGCTTCTGATTTACTCGGCATCCGATCTCTACTCTGGAGTCCCTTCTCGCTTCTCTGGTAGCCGTTCCGG |
| | GACGGATTTCACTCTGACCATCAGCAGTCTGCAGCCGGAAGACTTCGCAACTTATTACTGTCAGCAAGGTTCTTACCT |
| | GTTCACGTTCGGACAGGGTACCAAGGTGGAGATCAAACGTACGGTGGCTGCACCATCTGTCTTCATCTTCCCGCCAT |
| | CTGATGAGCAGTTGAAATCTGGAACTGCCTCTGTTGTGTGCCTGCTGAATAACTTCTATCCCAGAGAGGCCAAAGTA |
| | CAGTGGAAGGTGGATAACGCCCTCCAATCGGGTAACTCCCAGGAGAGTGTCACAGAGCAGGACAGCAAGGACAGC |
| | ACCTACAGCCTCAGCAGCACCCTGACGCTGAGCAAAGCAGACTACGAAAAACATAAAGTCTACGCCTGCGAAGTCAC |
| | CCATCAGGGCCTGAGCTCGCCCGTCACAAAGAGCTTCAACAGGGGAGAGTGTGGCGGGGAGGATCCGATATCCA |
| | GATGACCCAGTCCCCGAGCTCCCTGTCCGCCTCTGTGGGCGATAGGGTCACCATCACCTGCCGTGCCAGCCAGAGCG |
| | TGTCCAGCGCCGTAGCCTGGTATCAACAGAAACCAGGAAAAGCTCCGAAGCTTCTGATTTACAGCGCCAGCGACCTG |
| | TACAGCGGAGTCCCTTCTCGCTTCTCTGGTAGCCGTTCCGGGACGGATTTCACTCTGACCATCAGCAGTCTGCAGCCG |
| | GAAGACTTCGCAACTTATTACTGTCAGCAAGGTTCTTACCTGTTCACGTTCGGACAGGGTACCAAGGTGGAGATCAA |
| | AGGTACTACTGCCGCTAGTGGTAGTAGTGGTGGCAGTAGCAGTGGTGCCGAGGTTCAGCTGGTGGAGTCTGGGGG |
| | TGGCCTGGTGCAGCCAGGGGGCTCACTCCGTTTGTCCTGTGCAGCTTCTGGCTTCGACCTCACTGGTTCTTACATGCA |
| | CTGGGTGCGTCAGGCCCCGGGTAAGGGCCTGGAATGGGTTGCAGGTATTTCCGCTTCTGGAGGCGCTACTGCTTAT |
| | GCCGATAGCGTCAAGGGCCGTTTCACTATAAGCGCAGACACATCCAAAAACACAGCCTACCTACAAATGAACAGCTT |
| | AAGAGCTGAGGACACTGCCGTCTATTATTGTGCTCGCTCTCGTTCTTCTTCTTACTCTTCTTCTGGTTGGCGTTACTACT |
| | CTGGTGCTTTTGATTACTGGGGTCAAGGAACCCTGGTCACCGTCTCCTCG |
| VHH-VL-CL-VL- | EVQLVESGGGLVQPGRSLRLSCAASGFDLTGSYMHWVRRAPGKGEELVAGISASGGATAYADSVKGRFTISADTSKNTAY |
| VH AA | LQMNSLRAEDTAVYYCARSRSSSYSSSGWRYYSGAFDYWGQGTLVTVSSGGGGSGGGGSDIQMTQSPSSLSASVGDRV |
| | TITCRASQSVSSAVAWYQQKPGKAPKLLIYSASDLYSGVPSRFSGSRSGTDFTLTISSLQPEDFATYYCQQGSYLFTFGQGTK |
| | VEIKRTVAAPSVFIFPPSDEQLKSGTASVVCLLNNFYPREAKVQWKVDNALQSGNSQESVTEQDSKDSTYSLSSTLTLSKAD |
| | YEKHKVYACEVTHQGLSSPVTKSFNRGECGGGGSDIQMTQSPSSLSASVGDRVTITCRASQSVSSAVAWYQQKPGKAPK |
| | LLIYSASDLYSGVPSRFSGSRSGTDFTLTISSLQPEDFATYYCQQGSYLFTFGQGTKVEIKGTTAASGSSGGSSSGAEVQLVES |
| | GGGLVQPGGSLRLSCAASGFDLTGSYMHWVRQAPGKGLEWVAGISASGGATAYADSVKGRFTISADTSKNTAYLQMNS |
| | LRAEDTAVYYCARSRSSSYSSSGWRYYSGAFDYWGQGTLVTVSS |
| VH-CH1 NT | GAGGTTCAGCTGGTGGAGTCTGGCGGTGGCCTGGTGCAGCCAGGGGGCTCACTCCGTTTGTCCTGTGCAGCTTCTG |
| | GCTTCGACCTCACTGGTTCTTACATGCACTGGGTGCGTCAGGCCCCGGGTAAGGGCCTGGAATGGGTTGCAGGTATT |
| | TCCGCTTCTGGAGGCGCTACTGCTTATGCCGATAGCGTCAAGGGCCGTTTCACTATAAGCGCAGACACATCCAAAAA |
| | CACAGCCTACCTACAAATGAACAGCTTAAGAGCTGAGGACACTGCCGTCTATTATTGTGCTCGCTCTCGTTCTTCTTCT |
| | TACTCTTCTTCTGGTTGGCGTTACTACTCTGGTGCTTTTGACTACTGGGGTCAAGGAACCCTGGTCACCGTCTCCTCGG |
| | CTAGCACCAAGGGCCCATCGGTCTTCCCCCTGGCACCCTCCTCCAAGAGCACCTCTGGGGGCACAGCGGCCCTGGGC |
| | TGCCTGGTCAAGGACTACTTCCCCGAACCGGTGACGGTGTCGTGGAACTCAGGCGCCCTGACCAGCGGCGTGCACA |
| | CCTTCCCGGCTGTCCTACAGTCCTCAGGACTCTACTCCCTCAGCAGCGTGGTGACCGTGCCCTCCAGCAGCTTGGGCA |
| | CCCAGACCTACATCTGCAACGTGAATCACAAGCCCAGCAACACCAAGGGGACAAGAAAGTTGAGCCCAAATCTTGT |
| | GACAAAACTCACACA |
| VH-CH1 AA | EVQLVESGGGLVQPGGSLRLSCAASGFDLTGSYMHWVRQAPGKGLEWVAGISASGGATAYADSVKGRFTISADTSKNT |
| | AYLQMNSLRAEDTAVYYCARSRSSSYSSSGWRYYSGAFDYWGQGTLVTVSSASTKGPSVFPLAPSSKSTSGGTAALGCLV |
| | KDYFPEPVTVSWNSGALTSGVHTFPAVLQSSGLYSLSSVVTVPSSSLGTQTYICNVNHKPSNTKVDKKVEPKSCDKTHT |
| 14. LEAD VHH-Fc clone example | |
| 15031 VHH-FC | |
| VHH-CH2-CH3 | GAGGTTCAGCTGGTGGAGTCTGGCGGTGGCCTGGTGCAGCCAGGGCGTTCACTCCGTTTGTCCTGTGCAGCTTCTG |
| NT | GCTTCGACCTCACTGGTTCTTACATGTGGGTGCGTCGTGCCCCGGGTAAGGGCGAGGAACTGGTTGCAGGTATTTCC |
| | GCTTCTGGAGGCGCTACTGCTTATGCCGATAGCGTCAAGGGCCGTTTCACTATAAGCGCAGACACATCCAAAAACAC |
| | AGCCTACCTACAAATGAACAGCTTAAGAGCTGAGGACACTGCCGTCTATTATTGTGCTCGCTCTCGTTCTTCTTCTTAC |
| | TCTTCTTCTGGTTGGCGTTACTACTCTGGTGCTTTTGACTACTGGGGTCAAGGAACCCTGGTCACCGTCTCCTCGGGT |
| | GGAGGTGGCTCCGGAGGAGGTGGCAGTGACAAAACTCACACATGCCCACCGTGCCCAGCACCTGAACTCCTGGGG |
| | GGACCGTCAGTCTTCCTCTTCCCCCCAAAACCCAAGGACACCCTCATGATCTCCCGGACCCCTGAGGTCACATGCGTG |
| | GTGGTGGACGTGAGCCACGAAGACCCTGAGGTCAAGTTCAACTGGTACGTGGACGGCGTGGAGGTGCATAATGCC |
| | AAGACAAAGCCGCGGGAGGAGCAGTACAACAGCACGTACCGTGTGGTCAGCGTCCTCACCGTCCTGCACCAGGACT |
| | GGCTGAATGGCAAGGAGTACAAGTGCAAGGTCTCCAACAAAGCCCTCCCAGCCCCCATCGAGAAAACCATCTCCAA |
| | AGCCAAAGGGCAGCCCCGAGAACCACAGGTGTACACCCTGCCCCCATCCCGGGAGGAGATGACCAAGAACCAGGTC |
| | AGCCTGACCTGCCTGGTCAAAGGCTTCTATCCCAGCGACATCGCCGTGGAGTGGGAGAGCAATGGGCAGCCGGAGA |
| | ACAACTACAAGACCACGCCTCCCGTGCTGGACTCCGACGGCTCCTTCTTCCTCTACAGCAAGCTCACCGTGGACAAGA |
| | GCAGGTGGCAGCAGGGGAACGTCTTCTCATGCTCCGTGATGCATGAGGCTCTGCACAACCACTACACGCAGAAGAG |
| | CCTCTCCCTGTCTCCGGGTAAA |
| VHH-CH2-CH3 | EVQLVESGGGLVQPGRSLRLSCAASGFDLTGSYMWVRRAPGKGEELVAGISASGGATAYADSVKGRFTISADTSKNTAYL |
| AA | QMNSLRAEDTAVYYCARSRSSSYSSSGWRYYSGAFDYWGQGTLVTVSSGGGGGGGGSDKTHTCPPCPAPELLGGPSV |
| | FLFPPKPKDTLMISRTPEVTCVVVDVSHEDPEVKFNWYVDGVEVHNAKTKPREEQYNSTYRVVSVLTVLHQDWLNGKEY |
| | KCKVSNKALPAPIEKTISKAKGQPREPQVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 15. LEAD VHH-VHH-Fc clone example | |
| 15031 VHH-VHH-Fc | |
| VHH-VHH-CH2- | GAGGTTCAGCTGGTGGAGTCTGGCGGTGGCCTGGTGCAGCCAGGGCGTTCACTCCGTTTGTCCTGTGCAGCTTCTG |
| CH3 NT | GCTTCGACCTCACTGGTTCTTACATGTGGGTGCGTCGTGCCCCGGGTAAGGGCGAGGAACTGGTTGCAGGTATTTCC |
| | GCTTCTGGAGGCGCTACTGCTTATGCCGATAGCGTCAAGGGCCGTTTCACTATAAGCGCAGACACATCCAAAAACAC |
| | AGCCTACCTACAAATGAACAGCTTAAGAGCTGAGGACACTGCCGTCTATTATTGTGCTCGCTCTCGTTCTTCTTCTTAC |
| | TCTTCTTCTGGTTGGCGTTACTACTCTGGTGCTTTTGACTACTGGGGTCAAGGAACCCTGGTCACCGTCTCCTCGGGT |
| | GGAGGTGGCTCCGGAGGAGGTGGCAGTGAGGTTCAGCTGGTGGAGTCTGGCGGTGGCCTGGTGCAGCCAGGGG |
| | TTCACTCCGTTTGTCCTGTGCAGCTTCTGGCTTCGACCTCACTGGTTCTTACATGTGGGTGCGTCGTGCCCCGGGTAA |
| | GGGCGAGGAACTGGTTGCAGGTATTTCCGCTTCTGGAGGCGCTACTGCTTATGCCGATAGCGTCAAGGGCCGTTTCA |
| | CTATAAGCGCAGACACATCCAAAAACACAGCCTACCTACAAATGAACAGCTTAAGAGCTGAGGACACTGCCGTCTAT |
| | TATTGTGCTCGCTCTCGTTCTTCTTCTTACTCTTCTTCTGGTTGGCGTTACTACTCTGGTGCTTTTGACTACTGGGGTCA |
| | AGGAACCCTGGTCACCGTCTCCTCGGGTGGAGGTGGCTCCGGAGGAGGTGGCAGTGACAAAACTCACACATGCCC |
| | ACCGTGCCCAGCACCTGAACTCCTGGGGGGACCGTCAGTCTTCCTCTTCCCCCCAAAACCCAAGGACACCCTCATGAT |
| | CTCCCGGACCCCTGAGGTCACATGCGTGGTGGTGGACGTGAGCCACGAAGACCCTGAGGTCAAGTTCAACTGGTAC |
| | GTGGACGGCGTGGAGGTGCATAATGCCAAGACAAAGCCGCGGGAGGAGCAGTACAACAGCACGTACCGTGTGGTC |
| | AGCGTCCTCACCGTCCTGCACCAGGACTGGCTGAATGGCAAGGAGTACAAGTGCAAGGTCTCCAACAAAGCCCTCCC |
| | AGCCCCCATCGAGAAAACCATCTCCAAAGCCAAAGGGCAGCCCCGAGAACCACAGGTGTACACCCTGCCCCCATCCC |
| | GGGAGGAGATGACCAAGAACCAGGTCAGCCTGACCTGCCTGGTCAAAGGCTTCTATCCCAGCGACATCGCCGTGGA |
| | GTGGGAGAGCAATGGGCAGCCGGAGAACAACTACAAGACCACGCCTCCCGTGCTGGACTCCGACGGCTCCTTCTTC |
| | CTCTACAGCAAGCTCACCGTGGACAAGAGCAGGTGGCAGCAGGGGAACGTCTTCTCATGCTCCGTGATGCATGAGG |
| | CTCTGCACAACCACTACACGCAGAAGAGCCTCTCCCTGTCTCCGGGTAAA |
| VHH-VHH-CH2- | EVQLVESGGGLVQPGRSLRLSCAASGFDLTGSYMWVRRAPGKGEELVAGISASGGATAYADSVKGRFTISADTSKNTAYL |
| CH3 AA | QMNSLRAEDTAVYYCARSRSSSYSSSGWRYYSGAFDYWGQGTLVTVSSGGGGSGGGGSEVQLVESGGGLVQPGRSLRL |
| | SCAASGFDLTGSYMWVRRAPGKGEELVAGISASGGATAYADSVKGRFTISADTSKNTAYLQMNSLRAEDTAVYYCARSRS |
| | SSYSSSGWRYYSGAFDYWGQGTLVTVSSGGGGSGGGGSDKTHTCPPCPAPELLGGPSVFLFPPKPKOTLMISRTPEVTCV |
| | VVDVSIICDPCVKFNWYVDGVEVHNAKTKPREEQYNSTYRVVSVLTVLHQDWLNGKEYKCKVSNKALPAPIEKTISKAKG |
| | QPREPQVYTLPPSREEMTKNQVSLTCLVKGFYPSDIAVEWESNGQPENNYKTTPPVLDSDGSFFLYSKLTVDKSRWQQG |
| | NVFSCSVMHEALHNHYTQKSLSLSPGK |
| 16. LEAD VHH-Fc-VHH clone example | |
| 15031 VHH-Fc-VHH | |
| VHH-CH2-CH3- | GAGGTTCAGCTGGTGGAGTCTGGCGGTGGCCTGGTGCAGCCAGGGCGTTCACTCCGTTTGTCCTGTGCAGCTTCTG |
| VHH NT | GCTTCGACCTCACTGGTTCTTACATGTGGGTGCGTCGTGCCCCGGGTAAGGGCGAGGAACTGGTTGCAGGTATTTCC |
| | GCTTCTGGAGGCGCTACTGCTTATGCCGATAGCGTCAAGGGCCGTTTCACTATAAGCGCAGACACATCCAAAAACAC |
| | AGCCTACCTACAAATGAACAGCTTAAGAGCTGAGGACACTGCCGTCTATTATTGTGCTCGCTCTCGTTCTTCTTCTTAC |
| | TCTTCTTCTGGTTGGCGTTACTACTCTGGTGCTTTTGACTACTGGGGTCAAGGAACCCTGGTCACCGTCTCCTCGGGT |
| | GGAGGTGGCTCCGGAGGAGGTGGCAGTGACAAAACTCACACATGCCCACCGTGCCCAGCACCTGAACTCCTGGGG |
| | GGACCGTCAGTCTTCCTCTTCCCCCCAAAACCCAAGGACACCCTCATGATCTCCCGGACCCCTGAGGTCACATGCGTG |
| | GTGGTGGACGTGAGCCACGAAGACCCTGAGGTCAAGTTCAACTGGTACGTGGACGGCGTGGAGGTGCATAATGCC |
| | AAGACAAAGCCGCGGGAGGAGCAGTACAACAGCACGTACCGTGTGGTCAGCGTCCTCACCGTCCTGCACCAGGACT |
| | GGCTGAATGGCAAGGAGTACAAGTGCAAGGTCTCCAACAAAGCCCTCCCAGCCCCCATCGAGAAAACCATCTCCAA |
| | AGCCAAAGGGCAGCCCCGAGAACCACAGGTGTACACCCTGCCCCCATCCCGGGAGGAGATGACCAAGAACCAGGTC |
| | AGCCTGACCTGCCTGGTCAAAGGCTTCTATCCCAGCGACATCGCCGTGGAGTGGGAGAGCAATGGGCAGCCGGAGA |
| | ACAACTACAAGACCACGCCTCCCGTGCTGGACTCCGACGGCTCCTTCTTCCTCTACAGCAAGCTCACCGTGGACAAGA |
| | GCAGGTGGCAGCAGGGGAACGTCTTCTCATGCTCCGTGATGCATGAGGCTCTGCACAACCACTACACGCAGAAGAG |
| | CCTCTCCCTGTCTCCGGGTGGAGGAGGTGGGGGATCAGGTGGTGGGGGCTCTGGAGGCGGAACCGGTGAGGTTC |
| | AGCTGGTGGAGTCTGGCGGTGGCCTGGTGCAGCCAGGGCGTTCACTCCGTTTGTCCTGTGCAGCTTCTGGCTTCGAC |
| | CTCACTGGTTCTTACATGTGGGTGCGTCGTGCCCCGGGTAAGGGCGAGGAACTGGTTGCAGGTATTTCCGCTTCTGG |
| | AGGCGCTACTGCTTATGCCGATAGCGTCAAGGGCCGTTTCACTATAAGCGCAGACACATCCAAAAACACAGCCTACC |
| | TACAAATGAACAGCTTAAGAGCTGAGGACACTGCCGTCTATTATTGTGCTCGCTCTCGTTCTTCTTCTTACTCTTCTTCT |
| | GGTTGGCGTTACTACTCTGGTGCTTTTGACTACTGGGGTCAAGGAACCCTGGTCACCGTCTCCTCG |
| VHH-CH2-CH3- | EVQLVESGGGLVQPGRSLRLSCAASGFDLTGSYMWVRRAPGKGEELVAGISASGGATAYADSVKGRFTISADTSKNTAYL |
| VHH AA | QMNSLRAEDTAVYYCARSRSSSYSSSGWRYYSGAFDYWGQGTLVTVSSGGGGSGGGGSDKTHTCPPCPAPELLGGPSV |
| | FLFPPKPKDTLMISRTPEVTCVVVDVSHEDPEVKFNWYVDGVEVHNAKTKPREEQYNSTYRVVSVLTVLHQDWLNGKEY |
| | KCKVSNKALPAPIEKTISKAKGQPREPQVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGGGGGGGGGGGGGTGEVQLVESGGGLVQPG |
| | RSLRLSCAASGFDLTGSYMWVRRAPGKGEELVAGISASGGATAYADSVKGRFTISADTSKNTAYLQMNSLRAEDTAVYYC |
| | ARSRSSSYSSSGWRYYSGAFDYWGQGTLVTVSS |
| 17. LEAD VHH-Fc-scFv clone example | |
| 15031 VHH-Fc-scFv | |
| VHH-CH2-CH3- | GAGGTTCAGCTGGTGGAGTCTGGCGGTGGCCTGGTGCAGCCAGGGCGTTCACTCCGTTTGTCCTGTGCAGCTTCTG |
| VL-VH | GCTTCGACCTCACTGGTTCTTACATGTGGGTGCGTCGTGCCCCGGGTAAGGGCGAGGAACTGGTTGCAGGTATTTCC |
| NT | GCTTCTGGAGGCGCTACTGCTTATGCCGATAGCGTCAAGGGCCGTTTCACTATAAGCGCAGACACATCCAAAAACAC |
| | AGCCTACCTACAAATGAACAGCTTAAGAGCTGAGGACACTGCCGTCTATTATTGTGCTCGCTCTCGTTCTTCTTCTTAC |
| | TCTTCTTCTGGTTGGCGTTACTACTCTGGTGCTTTTGACTACTGGGGTCAAGGAACCCTGGTCACCGTCTCCTCGGGT |
| | GGAGGTGGCTCCGGAGGAGGTGGCAGTGACAAAACTCACACATGCCCACCGTGCCCAGCACCTGAACTCCTGGGG |
| | GGACCGTCAGTCTTCCTCTTCCCCCCAAAACCCAAGGACACCCTCATGATCTCCCGGACCCCTGAGGTCACATGCGTG |
| | GTGGTGGACGTGAGCCACGAAGACCCTGAGGTCAAGTTCAACTGGTACGTGGACGGCGTGGAGGTGCATAATGCC |
| | AAGACAAAGCCGCGGGAGGAGCAGTACAACAGCACGTACCGTGTGGTCAGCGTCCTCACCGTCCTGCACCAGGACT |
| | GGCTGAATGGCAAGGAGTACAAGTGCAAGGTCTCCAACAAAGCCCTCCCAGCCCCCATCGAGAAAACCATCTCCAA |
| | AGCCAAAGGGCAGCCCCGAGAACCACAGGTGTACACCCTGCCCCCATCCCGGGAGGAGATGACCAAGAACCAGGTC |
| | AGCCTGACCTGCCTGGTCAAAGGCTTCTATCCCAGCGACATCGCCGTGGAGTGGGAGAGCAATGGGCAGCCGGAGA |
| | ACAACTACAAGACCACGCCTCCCGTGCTGGACTCCGACGGCTCCTTCTTCCTCTACAGCAAGCTCACCGTGGACAAGA |
| | GCAGGTGGCAGCAGGGGAACGTCTTCTCATGCTCCGTGATGCATGAGGCTCTGCACAACCACTACACGCAGAAGAG |
| | CCTCTCCCTGTCTCCGGGTGGAGGAGGTGGGGGATCAGGTGGTGGGGGCTCTGGAGGCGGAACCGGTGATATCCA |
| | GATGACCCAGTCCCCGAGCTCCCTGTCCGCCTCTGTGGGCGATAGGGTCACCATCACCTGCCGTGCCAGTCAGTCCG |
| | TGTCCAGCGCTGTAGCCTGGTATCAACAGAAACCAGGAAAAGCTCCGAAGCTTCTGATTTACTCGGCATCCAGCCTC |
| | TACTCTGGAGTCCCTTCTCGCTTCTCTGGTAGCCGTTCCGGGACGGATTTCACTCTGACCATCAGCAGTCTGCAGCCG |
| | GAAGACTTCGCAACTTATTACTGTCAGCAAGGTTCTTACCTGTTCACGTTCGGACAGGGTACCAAGGTGGAGATCAA |
| | AGGTACTACTGCCGCTAGTGGTAGTAGTGGTGGCAGTAGCAGTGGTGCCGAGGTTCAGCTGGTGGAGTCTGGCGG |
| | TGGCCTGGTGCAGCCAGGGGGCTCACTCCGTTTGTCCTGTGCAGCTTCTGGCTTCGACCTCACTGGTTCTTACATGCA |
| | CTGGGTGCGTCAGGCCCCGGGTAAGGGCCTGGAATGGGTTGCAGGTATTTCCGCTTCTGGAGGCGCTACTGCTTAT |
| | GCCGATAGCGTCAAGGGCCGTTTCACTATAAGCGCAGACACATCCAAAAACACAGCCTACCTACAAATGAACAGCTT |
| | AAGAGCTGAGGACACTGCCGTCTATTATTGTGCTCGCTCTCGTTCTTCTTCTTACTCTTCTTCTGGTTGGCGTTACTACT |
| | CTGGTGCTTTTGACTACTGGGGTCAAGGAACCCTGGTCACCGTCTCCTCG |
| VHH-CH2-CH3- | EVQLVESGGGLVQPGRSLRLSCAASGFDLTGSYMWVRRAPGKGEELVAGISASGGATAYADSVKGRFTISADTSKNTAYL |
| VL-VH | QMNSLRAEDTAVYYCARSRSSSYSSSGWRYYSGAFDYWGQGTLVTVSSGGGGSGGGGSDKTHTCPPCPAPELLGGPSV |
| AA | FLFPPKPKDTLMISRTPEVTCVVVDVSHEDPEVKFNWYVDGVEVHNAKTKPREEQYNSTYRVVSVLTVLHQDWLNGKEY |
| | KCKVSNKALPAPIEKTISKAKGQPREPQVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGGGGGGSGGGGSGGGTGDIQMTQSPSSLSASV |
| | GDRVTITCRASQSVSSAVAWYQQKPGKAPKLLIYSASDLYSGVPSRFSGSRSGTDFTLTISSLQPEDFATYYCQQGSYLFTFG |
| | QGTKVEIKGTTAASGSSGGSSSGAEVQLVESGGGLVQPGGSLRLSCAASGFDLTGSYMHWVRQAPGKGLEWVAGISAS |
| | GGATAYADSVKGRFTISADTSKNTAYLQMNSLRAEDTAVYYCARSRSSSYSSSGWRYYSGAFDYWGQGTLVTVSS |
| 18. LEAD VHH-Fc clone example | |
| 15031 VHH-fc-Fab | |
| VL-CL NT | GATATCCAGATGACCCAGTCCCCGAGCTCCCTGTCCGCCTCTGTGGGCGATAGGGTCACCATCACCTGCCGTGCCAG |
| | TCAGTCCGTGTCCAGCGCTGTAGCCTGGTATCAACAGAAACCAGGAAAAGCTCCGAAGCTTCTGATTTACTCGGCAT |
| | CCGATCTCTACTCTGGAGTCCCTTCTCGCTTCTCTGGTAGCCGTTCCGGGACGGATTTCACTCTGACCATCAGCAGTCT |
| | GCAGCCGGAAGACTTCGCAACTTATTACTGTCAGCAAGGTTCTTACCTGTTCACGTTCGGACAGGGTACCAAGGTGG |
| | AGATCAAACGTACGGTGGCTGCACCATCTGTCTTCATCTTCCCGCCATCTGATGAGCAGTTGAAATCTGGAACTGCCT |
| | CTGTTGTGTGCCTGCTGAATAACTTCTATCCCAGAGAGGCCAAAGTACAGTGGAAGGTGGATAACGCCCTCCAATCG |
| | GGTAACTCCCAGGAGAGTGTCACAGAGCAGGACAGCAAGGACAGCACCTACAGCCTCAGCAGCACCCTGACGCTGA |
| | GCAAAGCAGACTACGAAAAACATAAAGTCTACGCCTGCGAAGTCACCCATCAGGGCCTGAGCTCGCCCGTCACAAA |
| | GAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTLTISSLQPEDF |
| | ATYYCQQGSYLFTFGQGTKVEIKRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| VHH-CH2-CH3- | GAGGTTCAGCTGGTGGAGTCTGGCGGTGGCCTGGTGCAGCCAGGGCGTTCACTCCGTTTGTCCTGTGCAGCTTCTG |
| VH-CH1 | GCTTCGACCTCACTGGTTCTTACATGTGGGTGCGTCGTGCCCCGGGTAAGGGCGAGGAACTGGTTGCAGGTATTTCC |
| NT | GCTTCTGGAGGCGCTACTGCTTATGCCGATAGCGTCAAGGGCCGTTTCACTATAAGCGCAGACACATCCAAAAACAC |
| | AGCCTACCTACAAATGAACAGCTTAAGAGCTGAGGACACTGCCGTCTATTATTGTGCTCGCTCTCGTTCTTCTTCTTAC |
| | TCTTCTTCTGGTTGGCGTTACTACTCTGGTGCTTTTGACTACTGGGGTCAAGGAACCCTGGTCACCGTCTCCTCGGGT |
| | GGAGGTGGCTCCGGAGGAGGTGGCAGTGACAAAACTCACACATGCCCACCGTGCCCAGCACCTGAACTCCTGGGG |
| | GGACCGTCAGTCTTCCTCTTCCCCCCAAAACCCAAGGACACCCTCATGATCTCCCGGACCCCTGAGGTCACATGCGTG |
| | GTGGTGGACGTGAGCCACGAAGACCCTGAGGTCAAGTTCAACTGGTACGTGGACGGCGTGGAGGTGCATAATGCC |
| | AAGACAAAGCCGCGGGAGGAGCAGTACAACAGCACGTACCGTGTGGTCAGCGTCCTCACCGTCCTGCACCAGGACT |
| | GGCTGAATGGCAAGGAGTACAAGTGCAAGGTCTCCAACAAAGCCCTCCCAGCCCCCATCGAGAAAACCATCTCCAA |
| | AGCCAAAGGGCAGCCCCGAGAACCACAGGTGTACACCCTGOCCCCATCCCGGGAGGAGATGACCAAGAACCAGGTC |
| | AGCCTGACCTGCCTGGTCAAAGGCTTCTATCCCAGCGACATCGCCGTGGAGTGGGAGAGCAATGGGCAGCCGGAGA |
| | ACAACTACAAGACCACGCCTCCCGTGCTGGACTCCGACGGCTCCTTCTTCCTCTACAGCAAGCTCACCGTGGACAAGA |
| | GCAGGTGGCAGCAGGGGAACGTCTTCTCATGCTCCGTGATGCATGAGGCTCTGCACAACCACTACACGCAGAAGAG |
| | CCTCTCCCTGTCTCCGGGTGGAGGAGGTGGGGGATCAGGTGGTGGGGGCTCTGGAGGCGGAACCGGTGAGGTTC |
| | AGCTGGTGGAGTCTGGCGGTGGCCTGGTGCAGCCAGGGGGCTCACTCCGTTTGTCCTGTGCAGCTTCTGGCTTCGAC |
| | CTCACTGGTTCTTACATGCACTGGGTGCGTCAGGCCCCGGGTAAGGGCCTGGAATGGGTTGCAGGTATTTCOGCTTC |
| | TGGAGGCGCTACTGCTTATGCCGATAGCGTCAAGGGCCGTTTCACTATAAGCGCAGACACATCCAAAAACACAGCCT |
| | ACCTACAAATGAACAGCTTAAGAGCTGAGGACACTGCCGTCTATTATTGTGCTCGCTCTCGTTCTTCTTCTTACTCTTC |
| | TTCTGGTTGGCGTTACTACTCTGGTGCTTTTGACTACTGGGGTCAAGGAACCCTGGTCACCGTCTCCTCGGCTAGCAC |
| | CAAGGGTCCATCGGTCTTCCCCCTGGCACCCTCCTCCAAGAGCACCTCTGGGGGCACAGCGGCCCTGGGCTGCCTGG |
| | TCAAGGACTACTTCCCCGAACCCGTGACGGTGTCGTGGAACTCAGGCGCCCTGACCAGCGGCGTGCACACCTTCCCG |
| | GCTGTCCTACAGTCCTCAGGACTCTACTCCCTCAGCAGCGTGGTGACCGTGCCCTCCAGCAGCTTGGGCACCCAGAC |
| | CTACATCTGCAACGTGAATCACAAGCCCAGCAACACCAAGGTCGACAAGAAAGTTGAGCCCAAATCTTGTGACAAAA |
| | CTCACAC |
| VHH-CH2-CH3 | EVQLVESGGGLVQPGRSLRLSCAASGFDLTGSYMWVRRAPGKGEELVAGISASGGATAYADSVKGRFTISADTSKNTAYL |
| VH-CH1 | QMNSLRAEDTAVYYCARSRSSSYSSSGWRYYSGAFDYWGQGTLVTVSSGGGGSGGGGSDKTHTCPPCPAPELLGGPSV |
| AA | FLFPPKPKDTLMISRTPEVTCVVVDVSHEDPEVKFNWYVDGVEVHNAKTKPREEQYNSTYRVVSVLTVLHQDWLNGKEY |
| | KCKVSNKALPAPIEKTISKAKGQPREPQVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGGGGGGSGGGGSGGGTGEVQLVESGGGLVQPG |
| | GSLRLSCAASGFDLTGSYMHWVRQAPGKGLEWVAGISASGGATAYADSVKGRFTISADTSKNTAYLQMNSLRAEDTAVY |
| | YCARSRSSSYSSSGWRYYSGAFDYWGQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKSCDKTHT |
| 19. LEAD scfv-fc clone example | |
| 15031 scFv-FC | |
| VL-VH-CH2-CH3 | GATATCCAGATGACCCAGTCCCCGAGCTCCCTGTCCGCCTCTGTGGGCGATAGGGTCACCATCACCTGCCGTGCCAG |
| NT | TCAGTCCGTGTCCAGCGCTGTAGCCTGGTATCAACAGAAACCAGGAAAAGCTCCGAAGCTTCTGATTTACTTCGGCAT |
| | CCGATCTCTACTCTGGAGTCCCTTCTCGCTTCTCTGGTAGCCGTTCCGGGACGGATTTCACTCTGACCATCAGCAGTCT |
| | GCAGCCGGAAGACTTCGCAACTTATTACTGTCAGCAAGGTTCTTACCTGTTCACGTTCGGACAGGGTACCAAGGTGG |
| | AGATCAAAGGTACTACTGCCGCTAGTGGTAGTAGTGGTGGCAGTAGCAGTGGTGCCGAGGTTCAGCTGGTGGAGT |
| | CTGGCGGTGGCCTGGTGCAGCCAGGGGGCTCACTCCGTTTGTCCTGTGCAGCTTCTGGCTTCGACCTCACTGGTTCTT |
| | ACATGCACTGGGTGCGTCAGGCCCCGGGTAAGGGCCTGGAATGGGTTGCAGGTATTTCCGCTTCTGGAGGCGCTAC |
| | TGCTTATGCCGATAGCGTCAAGGGCCGTTTCACTATAAGCGCAGACACATCCAAAAACACAGCCTACCTACAAATGA |
| | ACAGCTTAAGAGCTGAGGACACTGCCGTCTATTATTGTGCTCGCTCTCGTTCTTCTTCTTACTCTTCTTCTGGTTGGCG |
| | TTACTACTCTGGTGCTTTTGACTACTGGGGTCAAGGAACCCTGGTCACCGTCTCCTCGGGTGGAGGTGGCTCCGGAG |
| | GAGGTGGCAGTGACAAAACTCACACATGCCCACCGTGCCCAGCACCTGAACTCCTGGGGGGACCGTCAGTCTTCCTC |
| | TTCCCCCCAAAACCCAAGGACACCCTCATGATCTCCCGGACCCCTGAGGTCACATGCGTGGTGGTGGACGTGAGCCA |
| | CGAAGACCCTGAGGTCAAGTTCAACTGGTACGTGGACGGCGTGGAGGTGCATAATGCCAAGACAAAGCCGCGGGA |
| | GGAGCAGTACAACAGCACGTACCGTGTGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAACCATCTCCAAAGCCAAAGGGCAGCCCCG |
| | AGAACCACAGGTGTACACCCTGCCCCCATCCCGGGAGGAGATGACCAAGAACCAGGTCAGCCTGACCTGCCTGGTC |
| | AAAGGCTTCTATCCCAGCGACATCGCCGTGGAGTGGGAGAGCAATGGGCAGCCGGAGAACAACTACAAGACCACG |
| | CCTCCCGTGCTGGACTCCGACGGCTCCTTCTTCCTCTACAGCAAGCTCACCGTGGACAAGAGCAGGTGGCAGCAGGG |
| | GAACGTCTTCTCATGCTCCGTGATGCATGAGGCTCTGCACAACCACTACACGCAGAAGAGCCTCTCCCTGTCTCCGGG |
| | TAAA |
| VI-VH-CH2-CH | DIQMTQSPSSLSASVGDRVTITCRASQSVSSAVAWYQQKPGKAPKLLIYSASDLYSGVPSRFSGSRSGTDFTLTISSLQPEDF |
| AA | ATYYCQQGSYLFTFGQGTKVEIKGTTAASGSSGGSSSGAEVQLVESGGGLVQPGGSLRLSCAASGFDLTGSYMHWVRQA |
| | PGKGLEWVAGISASGGATAYADSVKGRFTISADTSKNTAYLQMNSLRAEDTAVYYCARSRSSSYSSSGWRYYSGAFDYW |
| | GQGTLVTVSSGGGGSGGGGSDKTHTCPPCPAPELLGGPSVELFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTISKAKGQPREPQVYTLPPSREEMT |
| | KNQVSLTCLVKGFYPSDIAVEWESNGQPENNYKTTPPVLDSDGSFFLYSKLTVDKSRWQQGNVFSCSVMHEALHNHYTQ |
| | KSLSLSPGK |
| 20. LEAD scFv-scFv-Fc clone example | |
| 15031 scFv-scFv-FC | |
| VL-VH-VL-VH- | GATATCCAGATGACCCAGTCCCCGAGCTCCCTGTCCGCCTCTGTGGGCGATAGGGTCACCATCACCTGCCGTGCCAG |
| CH2-CH3 | TCAGTCCGTGTCCAGCGCTGTAGCCTGGTATCAACAGAAACCAGGAAAAGCTCCGAAGCTTCTGATTTACTCGGCAT |
| NT | CCGATCTCTACTCTGGAGTCCCTTCTCGCTTCTCTGGTAGCCGTTCCGGGACGGATTTCACTCTGACCATCAGCAGTCT |
| | GCAGCCGGAAGACTTCGCAACTTATTACTGTCAGCAAGGTTCTTACCTGTTCACGTTCGGACAGGGTACCAAGGTGG |
| | AGATCAAAGGTACTACTGCCGCTAGTGGTAGTAGTGGTGGCAGTAGCAGTGGTGCCGAGGTTCAGCTGGTGGAGT |
| | CTGGCGGTGGCCTGGTGCAGCCAGGGGGCTCACTCCGTTTGTCCTGTGCAGCTTCTGGCTTCGACCTCACTGGTTCTT |
| | ACATGCACTGGGTGCGTCAGGCCCCGGGTAAGGGCCTGGAATGGGTTGCAGGTATTTCCGCTTCTGGAGGCGCTAC |
| | TGCTTATGCCGATAGCGTCAAGGGCCGTTTCACTATAAGCGCAGACACATCCAAAAACACAGCCTACCTACAAATGA |
| | ACAGCTTAAGAGCTGAGGACACTGCCGTCTATTATTGTGCTCGCTCTCGTTCTTCTTCTTACTCTTCTTCTGGTTGGCG |
| | TTACTACTCTGGTGCTTTTGACTACTGGGGTCAAGGAACCCTGGTCACCGTCTCCTCGGGTGGAGGTGGCTCCGGAG |
| | GAGGTGGCAGTGATATCCAGATGACCCAGTCCCCGAGCTCCCTGTCCGCCTCTGTGGGCGATAGGGTCACCATCACC |
| | TGCCGTGCCAGTCAGTCCGTGTCCAGCGCTGTAGCCTGGTATCAACAGAAACCAGGAAAAGCTCCGAAGCTTCTGAT |
| | TTACTCGGCATCCGATCTCTACTCTGGAGTCCCTTCTCGCTTCTCTGGTAGCCGTTCCGGGACGGATTTCACTCTGACC |
| | ATCAGCAGTCTGCAGCCGGAAGACTTCGCAACTTATTACTGTCAGCAAGGTTCTTACCTGTTCACGTTCGGACAGGGT |
| | ACCAAGGTGGAGATCAAAGGTACTACTGCCGCTAGTGGTAGTAGTGGTGGCAGTAGCAGTGGTGCCGAGGTTCAG |
| | CTGGTGGAGTCTGGCGGTGGCCTGGTGCAGCCAGGGGGCTCACTCCGTTTGTCCTGTGCAGCTTCTGGCTTCGACCT |
| | CACTGGTTCTTACATGCACTGGGTGCGTCAGGCCCCGGGTAAGGGCCTGGAATGGGTTGCAGGTATTTCCGCTTCTG |
| | GAGGCGCTACTGCTTATGCCGATAGCGTCAAGGGCCGTTTCACTATAAGCGCAGACACATCCAAAAACACAGCCTAC |
| | CTACAAATGAACAGCTTAAGAGCTGAGGACACTGCCGTCTATTATTGTGCTCGCTCTCGTTCTTCTTCTTACTCTTCTTC |
| | TGGTTGGCGTTACTACTCTGGTGCTTTTGACTACTGGGGTCAAGGAACCCTGGTCACCGTCTCCTCGGGTGGAGGTG |
| | GCTCCGGAGGAGGTGGCAGTGACAAAACTCACACATGCCCACCGTGCCCAGCACCTGAACTCCTGGGGGGACCGTC |
| | AGTCTTCCTCTTCCCCCCAAAACCCAAGGACACCCTCATGATCTCCCGGACCCCTGAGGTCACATGCGTGGTGGTGGA |
| | CGTGAGCCACGAAGACCCTGAGGTCAAGTTCAACTGGTACGTGGACGGCGTGGAGGTGCATAATGCCAAGACAAA |
| | GCCGCGGGAGGAGCAGTACAACAGCACGTACCGTGTGGTCAGCGTCCTCACCGTCCTGCACCAGGACTGGCTGAAT |
| | GGCAAGGAGTACAAGTGCAAGGTCTCCAACAAAGCCCTCCCAGCCCCCATCGAGAAAACCATCTCCAAAGCCAAAG |
| | GGCAGCCCCGAGAACCACAGGTGTACACCCTGCCCCCATCCCGGGAGGAGATGACCAAGAACCAGGTCAGCCTGAC |
| | CTGCCTGGTCAAAGGCTTCTATCCCAGCGACATCGCCGTGGAGTGGGAGAGCAATGGGCAGCCGGAGAACAACTAC |
| | AAGACCACGCCTCCCGTGCTGGACTCCGACGGCTCCTTCTTCCTCTACAGCAAGCTCACCGTGGACAAGAGCAGGTG |
| | GCAGCAGGGGAACGTCTTCTCATGCTCCGTGATGCATGAGGCTCTGCACAACCACTACACGCAGAAGAGCCTCTCCC |
| | TGTCTCCGGGTAAA |
| VL-VH-VL-VH- | DIQMTQSPSSLSASVGDRVTITCRASQSVSSAVAWYQQKPGKAPKLLIYSASDLYSGVPSRFSGSRSGTDFTITISSLQPEDF |
| CH2-CH3 | ATYYCQOGSYLFTFGQGTKVEIKGTTAASGSSGGSSSGAEVQLVESGGGLVQPGGSLRLSCAASGFDLTGSYMHWVRQA |
| AA | PGKGLEWVAGISASGGATAYADSVKGRFTISADTSKNTAYLQMNSLRAEDTAVYYCARSRSSSYSSSGWRYYSGAFDYW |
| | GQGTLVTVSSGGGGSGGGGSDIQMTQSPSSLSASVGDRVTITCRASQSVSSAVAWYQQKPGKAPKLLIYSASDLYSGVPS |
| | RFSGSRSGTDFTLTISSLQPEDFATYYCQQGSYLFTFGQGTKVEIKGTTAASGSSGGSSSGAEVQLVESGGGLVQPGGSLRL |
| | SCAASGFDLTGSYMHWVROAPGKGLEWVAGISASGGATAYADSVKGRFTISADTSKNTAYLQMNSLRAEDTAVYYCAR |
| | SRSSSYSSSGWRYYSGAFDYWGQGTLVTVSSGGGGGGGGSDKTHTCPPCPAPELLGGPSVFLFPPKPKDTLMISRTPEV |
| | TCVVVDVSHEDPEVKFNWYVDGVEVHNAKTKPREEQYNSTYRVVSVLTVLHQDWLNGKEYKCKVSNKALPAPIEKTISKA |
| | KGQPREPQVYTIPPSREEMTKNQVSLTCLVKGFYPSDIAVEWESNGQPENNYKTTPPVLDSDGSFFLYSKLTVDKSRWQQ |
| | GNVFSCSVMHEALHNHYTQKSLSLSPGK |
| 21. LEAD scFv-Fc-scFv clone example | |
| 15031 scFv-Fc-scFv | |
| VL-VH-CH2- | GATATCCAGATGACCCAGTCCCCGAGCTCCCTGTCCGCCTCTGTGGGCGATAGGGTCACCATCACCTGCCGTGCCAG |
| CH3-VL-VH | TCAGTCCGTGTCCAGCGCTGTAGCCTGGTATCAACAGAAACCAGGAAAAGCTCCGAAGCTTCTGATTTACTGGGCAT |
| NT | CCGATCTCTACTCTGGAGTCCCTTCTCGCTTCTCTGGTAGCCGTTCCGGGACGGATTTCACTCTGACCATCAGCAGTCT |
| | GCAGCCGGAAGACTTCGCAACTTATTACTGTCAGCAAGGTTCTTACCTGTTCACGTTCGGACAGGGTACCAAGGTGG |
| | AGATCAAAGGTACTACTGCCGCTAGTGGTAGTAGTGGTGGCAGTAGCAGTGGTGCCGAGGTTCAGCTGGTGGAGT |
| | CTGGCGGTGGCCTGGTGCAGCCAGGGGGCTCACTCCGTTTGTCCTGTGCAGCTTCTGGCTTCGACCTCACTGGTTCTT |
| | ACATGCACTGGGTGCGTCAGGCCCCGGGTAAGGGCCTGGAATGGGTTGCAGGTATTTCCGCTTCTGGAGGCGCTAC |
| | TGCTTATGCCGATAGCGTCAAGGGCCGTTTCACTATAAGCGCAGACACATCCAAAAACACAGCCTACCTACAAATGA |
| | ACAGCTTAAGAGCTGAGGACACTGCCGTCTATTATTGTGCTCGCTCTCGTTCTTCTTCTTACTCTTCTTCTGGTTGGCG |
| | TTACTACTCTGGTGCTTTTGACTACTGGGGTCAAGGAACCCTGGTCACCGTCTCCTCGGGTGGAGGTGGCTCCGGAG |
| | GAGGTGGCAGTGACAAAACTCACACATGCCCACCGTGCCCAGCACCTGAACTCCTGGGGGGACCGTCAGTCTTCCTC |
| | TTCCCCCCAAAACCCAAGGACACCCTCATGATCTCCCGGACCCCTGAGGTCACATGCGTGGTGGTGGACGTGAGCCA |
| | CGAAGACCCTGAGGTCAAGTTCAACTGGTACGTGGACGGCGTGGAGGTGCATAATGCCAAGACAAAGCCGCGGGA |
| | GGAGCAGTACAACAGCACGTACCGTGTGGTCAGCGTCCTCACCGTCCTGCACCAGGACTGGCTGAATGGCAAGGAG |
| | TACAAGTGCAAGGTCTCCAACAAAGCCCTCCCAGCCCCCATCGAGAAAACCATCTCCAAAGCCAAAGGGCAGCCCOG |
| | AGAACCACAGGTGTACACCCTGCCCCCATCCCGGGAGGAGATGACCAAGAACCAGGTCAGCCTGACCTGCCTGGTC |
| | AAAGGCTTCTATCCCAGCGACATCGCCGTGGAGTGGGAGAGCAATGGGCAGCCGGAGAACAACTACAAGACCACG |
| | CCTCCCGTGCTGGACTCCGACGGCTCCTTCTTCCTCTACAGCAAGCTCACCGTGGACAAGAGCAGGTGGCAGCAGGG |
| | GAACGTCTTCTCATGCTCCGTGATGCATGAGGCTCTGCACAACCACTACACGCAGAAGAGCCTCTCCCTGTCTCCGGG |
| | TGGAGGAGGTGGGGGATCAGGTGGTGGGGGCTCTGGAGGCGGAACCGGTGATATCCAGATGACCCAGTCCCCGA |
| | GCTCCCTGTCCGCCTCTGTGGGCGATAGGGTCACCATCACCTGCCGTGCCAGTCAGTCCGTGTCCAGCGCTGTAGCCT |
| | GGTATCAACAGAAACCAGGAAAAGCTCCGAAGCTTCTGATTTACTCGGCATCCGATCTCTACTCTGGAGTCCCTTCTC |
| | GCTTCTCTGGTAGCCGTTCCGGGACGGATTTCACTCTGACCATCAGCAGTCTGCAGCCGGAAGACTTCGCAACTTATT |
| | ACTGTCAGCAAGGTTCTTACCTGTTCACGTTCGGACAGGGTACCAAGGTGGAGATCAAAGGTACTACTGCCGCTAGT |
| | GGTAGTAGTGGTGGCAGTAGCAGTGGTGCCGAGGTTCAGCTGGTGGAGTCTGGCGGTGGCCTGGTGCAGCCAGG |
| | GGGCTCACTCCGTTTGTCCTGTGCAGCTTCTGGCTTCGACCTCACTGGTTCTTACATGCACTGGGTGCGTCAGGCCCC |
| | GGGTAAGGGCCTGGAATGGGTTGCAGGTATTTCCGCTTCTGGAGGCGCTACTGCTTATGCCGATAGCGTCAAGGGC |
| | CGTTTCACTATAAGCGCAGACACATCCAAAAACACAGCCTACCTACAAATGAACAGCTTAAGAGCTGAGGACACTGC |
| | CGTCTATTATTGTGCTCGCTCTCGTTCTTCTTCTTACTCTTCTTCTGGTTGGCGTTACTACTCTGGTGCTTTTGACTACTG |
| | GGGTCAAGGAACCCTGGTCACCGTCTCCTCG |
| VL-VH-CH2- | DIQMTQSPSSLSASVGDRVTITCRASQSVSSAVAWYQQKPGKAPKLLIYSASDLYSGVPSRFSGSRSGTDFTLTISSLQPEDF |
| CH3-VL-VH | ATYYCQOGSYLFTFGQGTKVEIKGTTAASGSSGGSSSGAEVQLVESGGGLVQPGGSLRLSCAASGFDLTGSYMHWVRQA |
| AA | PGKGLEWVAGISASGGATAYADSVKGRFTISADTSKNTAYLQMNSLRAEDTAVYYCARSRSSSYSSSGWRYYSGAFDYW |
| | GQGTLVTVSSGGGGSGGGGSDKTHTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTISKAKGQPREPQVYTLPPSREEMT |
| | KNQVSLTCLVKGFYPSDIAVEWESNGQPENNYKTTPPVLDSDGSFFLYSKLTVDKSRWQQGNVFSCSVMHEALHNHYTQ |
| | KSLSLSPGGGGGGSGGGGSGGGTGDIQMTQSPSSLSASVGDRVTITCRASQSVSSAVAWYQQKPGKAPKLLIYSASDLYS |
| | GVPSRFSGSRSGTDFTLTISSLQPEDFATYYCQQGSYLFTFGQGTKVEIKGTTAASGSSGGSSSGAEVQLVESGGGLVQPG |
| | GSLRLSCAASGFDLTGSYMHWVRQAPGKGLEWVAGISASGGATAYADSVKGRFTISADTSKNTAYLQMNSLRAEDTAVY |
| | YCARSRSSSYSSSGWRYYSGAFDYWGQGTLVTVSS |
| 22. LEAD scfv-Fc-VHH clone example | |
| 15031 scFv-Fc-VHH | |
| VL-VH-CH2- | GATATCCAGATGACCCAGTCCCCGAGCTCCCTGTCCGCCTCTGTGGGCGATAGGGTCACCATCACCTGCCGTGCC |
| CH3-VHH | AGTCAGTCCGTGTCCAGCGCTGTAGCCTGGTATCAACAGAAACCAGGAAAAGCTCCGAAGCTTCTGATTTACTCG |
| NT | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAAGGTACTACTGCCGCTAGTGGTAGTAGTGGTGGCAGTAGCAGTGGTGCCGAGGTTCA |
| | GCTGGTGGAGTCTGGCGGTGGCCTGGTGCAGCCAGGGGGCTCACTCCGTTTGTCCTGTGCAGCTTCTGGCTTCG |
| | ACCTCACTGGTTCTTACATGCACTGGGTGCGTCAGGCCCCGGGTAAGGGCCTGGAATGGGTTGCAGGTATTTCC |
| | GCTTCTGGAGGCGCTACTGCTTATGCCGATAGCGTCAAGGGCCGTTTCACTATAAGCGCAGACACATCCAAAAA |
| | CACAGCCTACCTACAAATGAACAGCTTAAGAGCTGAGGACACTGCCGTCTATTATTGTGCTCGCTCTCGTTCTTCT |
| | TCTTACTCTTCTTCTGGTTGGCGTTACTACTCTGGTGCTTTTGACTACTGGGGTCAAGGAACCCTGGTCACCGTCT |
| | CCTCGGGTGGAGGTGGCTCCGGAGGAGGTGGCAGTGACAAAACTCACACATGCCCACCGTGCCCAGCACCTGA |
| | ACTCCTGGGGGGACCGTCAGTCTTCCTCTTCCCCCCAAAACCCAAGGACACCCTCATGATCTCCCGGACCCCTGA |
| | GGTCACATGCGTGGTGGTGGACGTGAGCCACGAAGACCCTGAGGTCAAGTTCAACTGGTACGTGGACGGCGTG |
| | GAGGTGCATAATGCCAAGACAAAGCCGCGGGAGGAGCAGTACAACAGCACGTACCGTGTGGTCAGCGTCCTCA |
| | CCGTCCTGCACCAGGACTGGCTGAATGGCAAGGAGTACAAGTGCAAGGTCTCCAACAAAGCCCTCCCAGCCCCC |
| | ATCGAGAAAACCATCTCCAAAGCCAAAGGGCAGCCCCGAGAACCACAGGTGTACACCCTGCCCCCATCCCGGGA |
| | GGAGATGACCAAGAACCAGGTCAGCCTGACCTGCCTGGTCAAAGGCTTCTATCCCAGCGACATCGCCGTGGAGT |
| | GGGAGAGCAATGGGCAGCCGGAGAACAACTACAAGACCACGCCTCCCGTGCTGGACTCCGACGGCTCCTTCTTC |
| | CTCTACAGCAAGCTCACCGTGGACAAGAGCAGGTGGCAGCAGGGGAACGTCTTCTCATGCTCCGTGATGCATGA |
| | GGCTCTGCACAACCACTACACGCAGAAGAGCCTCTCCCTGTCTCCGGGTGGAGGAGGTGGGGGATCAGGTGGT |
| | GGGGGCTCTGGAGGCGGAACCGGTGAGGTTCAGCTGGTGGAGTCTGGCGGTGGCCTGGTGCAGCCAGGGCGT |
| | TCACTCCGTTTGTCCTGTGCAGCTTCTGGCTTCGACCTCACTGGTTCTTACATGCACTGGGTGCGTCGTGCCCCGG |
| | GTAAGGGCGAGGAACTGGTTGCAGGTATTTCCGCTTCTGGAGGCGCTACTGCTTATGCCGATAGCGTCAAGGGC |
| | CGTTTCACTATAAGCGCAGACACATCCAAAAACACAGCCTACCTACAAATGAACAGCTTAAGAGCTGAGGACACT |
| | GCCGTCTATTATTGTGCTCGCTCTCGTTCTTCTTCTTACTCTTCTTCTGGTTGGCGTTACTACTCTGGTGCTTTTGAC |
| | TACTGGGGTCAAGGAACCCTGGTCACCGTCTCCTCG |
| VL-VH-CH2- | DIQMTQSPSSLSASVGDRVTITCRASQSVSSAVAWYQQKPGKAPKLLIYSASDLYSGVPSRFSGSRSGTDFTLTISSLQP |
| CH3-VHH | EDFATYYCQQGSYLFTFGQGTKVEIKGTTAASGSSGGSSSGAEVQLVESGGGLVQPGGSLRLSCAASGFDLTGSYMH |
| AA | WVRQAPGKGLEWVAGISASGGATAYADSVKGRFTISADTSKNTAYLQMNSLRAEDTAVYYCARSRSSSYSSSGWRY |
| | YSGAFDYWGQGTLVTVSSGGGGSGGGGSDKTHTCPPCPAPELLGGPSVFLFPPKPKDTLMISRTPEVTCVVVDVSHE |
| | DPEVKFNWYVDGVEVHNAKTKPREEQYNSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPVLDSDGSFFLYSKLTVDKSRWQQGNVF |
| | SCSVMHEALHNHYTQKSLSLSPGGGGGGSGGGGSGGGTGEVQLVESGGGLVQPGRSLRLSCAASGFDLTGSYMH |
| | WVRRAPGKGEELVAGISASGGATAYADSVKGRFTISADTSKNTAYLQMNSLRAEDTAVYYCARSRSSSYSSSGWRYYS |
| | GAFDYWGQGTLVTVSS |
| 23. LEAD scFv-Fc-Fab clone example | |
| 15031 scFv-Fc-Fab | |
| VL-CL NT | GATATCCAGATGACCCAGTCCCCGAGCTCCCTGTCCGCCTCTGTGGGCGATAGGGTCACCATCACCTGCCGTGCC |
| | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTLTISSLQP |
| | EDFATYYCQQGSYLFTFGQGTKVEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGEC |
| VL-VH-CH2- | GATATCCAGATGACCCAGTCCCCGAGCTCCCTGTCCGCCTCTGTGGGCGATAGGGTCACCATCACCTGCCGTGCC |
| CH3-VH-CH1 | AGTCAGTCCGTGTCCAGCGCTGTAGCCTGGTATCAACAGAAACCAGGAAAAGCTCCGAAGCTTCTGATTTACTCG |
| NT | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAAGGTACTACTGCCGCTAGTGGTAGTAGTGGTGGCAGTAGCAGTGGTGCCGAGGTTCA |
| | GCTGGTGGAGTCTGGCGGTGGCCTGGTGCAGCCAGGGGGCTCACTCCGTTTGTCCTGTGCAGCTTCTGGCTTCG |
| | ACCTCACTGGTTCTTACATGCACTGGGTGCGTCAGGCCCCGGGTAAGGGCCTGGAATGGGTTGCAGGTATTTCC |
| | GCTTCTGGAGGCGCTACTGCTTATGCCGATAGCGTCAAGGGCCGTTTCACTATAAGCGCAGACACATCCAAAAA |
| | CACAGCCTACCTACAAATGAACAGCTTAAGAGCTGAGGACACTGCCGTCTATTATTGTGCTCGCTCTCGTTCTTCT |
| | TCTTACTCTTCTTCTGGTTGGCGTTACTACTCTGGTGCTTTTGACTACTGGGGTCAAGGAACCCTGGTCACCGTCT |
| | CCTCGGGTGGAGGTGGCTCCGGAGGAGGTGGCAGTGACAAAACTCACACATGCCCACCGTGCCCAGCACCTGA |
| | ACTCCTGGGGGGACCGTCAGTCTTCCTCTTCCCCCCAAAACCCAAGGACACCCTCATGATCTCCCGGACCCCTGA |
| | GGTCACATGCGTGGTGGTGGACGTGAGCCACGAAGACCCTGAGGTCAAGTTCAACTGGTACGTGGACGGCGTG |
| | GAGGTGCATAATGCCAAGACAAAGCCGCGGGAGGAGCAGTACAACAGCACGTACCGTGTGGTCAGCGTCCTCA |
| | CCGTCCTGCACCAGGACTGGCTGAATGGCAAGGAGTACAAGTGCAAGGTCTCCAACAAAGCCCTOCCAGCCCCC |
| | ATCGAGAAAACCATCTCCAAAGCCAAAGGGCAGCCCCGAGAACCACAGGTGTACACCCTGCCCCCATCCCGGGA |
| | GGAGATGACCAAGAACCAGGTCAGCCTGACCTGCCTGGTCAAAGGCTTCTATCCCAGCGACATCGCCGTGGAGT |
| | GGGAGAGCAATGGGCAGCCGGAGAACAACTACAAGACCACGCCTCCCGTGCTGGACTCCGACGGCTCCTTCTTC |
| | CTCTACAGCAAGCTCACCGTGGACAAGAGCAGGTGGCAGCAGGGGAACGTCTTCTCATGCTCCGTGATGCATGA |
| | GGCTCTGCACAACCACTACACGCAGAAGAGCCTCTCCCTGTCTCCGGGTGGAGGAGGTGGGGGATCAGGTGGT |
| | GGGGGCTCTGGAGGCGGAACCGGTGAGGTTCAGCTGGTGGAGTCTGGCGGTGGCCTGGTGCAGCCAGGGGG |
| | CTCACTCCGTTTGTCCTGTGCAGCTTCTGGCTTCGACCTCACTGGTTCTTACATGCACTGGGTGCGTCAGGCCCCG |
| | GGTAAGGGCCTGGAATGGGTTGCAGGTATTTCCGCTTCTGGAGGCGCTACTGCTTATGCCGATAGCGTCAAGGG |
| | CCGTTTCACTATAAGCGCAGACACATCCAAAAACACAGCCTACCTACAAATGAACAGCTTAAGAGCTGAGGACA |
| | CTGCCGTCTATTATTGTGCTCGCTCTCGTTCTTCTTCTTACTCTTCTTCTGGTTGGCGTTACTACTCTGGTGCTTTTG |
| | ACTACTGGGGTCAAGGAACCCTGGTCACCGTCTCCTCGGCTAGCACCAAGGGTCCATCGGTCTTCCCCCTGGCAC |
| | CCTCCTCCAAGAGCACCTCTGGGGGCACAGCGGCCCTGGGCTGCCTGGTCAAGGACTACTTCCCCGAACCCGTG |
| | ACGGTGTCGTGGAACTCAGGCGCCCTGACCAGCGGCGTGCACACCTTCCCGGCTGTCCTACAGTCCTCAGGACT |
| | CTACTCCCTCAGCAGCGTGGTGACCGTGCCCTCCAGCAGCTTGGGCACCCAGACCTACATCTGCAACGTGAATCA |
| | CAAGCCCAGCAACACCAAGGTCGACAAGAAAGTTGAGCCCAAATCTTGTGACAAAACTCACAC |
| VL-VH-CH2- | DIQMTQSPSSLSASVGDRVTITCRASQSVSSAVAWYQQKPGKAPKLLIYSASDLYSGVPSRFSGSRSGTDFTLTISSLQP |
| CH3-VH-CH1 | EDFATYYCQQGSYLFTFGQGTKVEIKGTTAASGSSGGSSSGAEVQLVESGGGLVQPGGSLRLSCAASGFDLTGSYMH |
| AA | WVRQAPGKGLEWVAGISASGGATAYADSVKGRFTISADTSKNTAYLQMNSLRAEDTAVYYCARSRSSSYSSSGWRY |
| | YSGAFDYWGQGTLVTVSSGGGGSGGGGSDKTHTCPPCPAPELLGGPSVFLFPPKPKDTLMISRTPEVTCVVVDVSHE |
| | DPEVKFNWYVDGVEVHNAKTKPREEQYNSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPVLDSDGSFFLYSKLTVDKSRWQQGNVF |
| | SCSVMHEALHNHYTQKSLSLSPGGGGGGSGGGGSGGGTGEVQLVESGGGLVQPGGSLRLSCAASGFDLTGSYMH |
| | WVRQAPGKGLEWVAGISASGGATAYADSVKGRFTISADTSKNTAYLQMNSLRAEDTAVYYCARSRSSSYSSSGWRY |
| | YSGAFDYWGQGTLVTVSSASTKGPSVFPLAPSSKSTSGGTAALGCLVKDYFPEPVTVSWNSGALTSGVHTFPAVLQSS |
| | GLYSLSSVVTVPSSSLGTQTYICNVNHKPSNTKVDKKVEPKSCDKTHT |
| 24. LEAD Diabody Fc clone example | |
| 15031 Diabody-Fc | |
| VH-VL- | GAGGTTCAGCTGGTGGAGTCTGGCGGTGGCCTGGTGCAGCCAGGGGGCTCACTCCGTTTGTCCTGTGCAGCTTC |
| CH2-CH3 | TGGCTTCGATCTCACTGGTTCTTACATGCACTGGGTGCGTCAGGCCCCGGGTAAGGGCCTGGAATGGGTTGCAG |
| NT | GTATTTCCGCTTCTGGAGGCGCTACTGCTTATGCCGATAGCGTCAAGGGCCGTTTCACTATAAGCGCAGACACAT |
| | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGGTTGGCGTTACTACTCTGGTGCTTTTGACTACTGGGGTCAAGGAACCCTGGTC |
| | ACCGTCTCCTCGGGTGGAGGTGGCAGTGATATCCAGATGACCCAGTCCCCGAGCTCCCTGTCCGCCTCTGTGGG |
| | CGATAGGGTCACCATCACCTGCCGTGCCAGTCAGTCCGTGTCCAGCGCTGTAGCCTGGTATCAACAGAAACCAG |
| | GAAAAGCTCCGAAGCTTCTGATTTACTCGGCATCCGATCTCTACTCTGGAGTCCCTTCTCGCTTCTCTGGTAGCCG |
| | TTCCGGGACGGATTTCACTCTGACCATCAGCAGTCTGCAGCCGGAAGACTTCGCAACTTATTACTGTCAGCAAGG |
| | TTCTTACCTGTTCACGTTCGGACAGGGTACCAAGGTGGAGATCAAAGGTGGAGGTGGCTCCGGAGGAGGTGGC |
| | AGTGACAAAACTCACACATGCCCACCGTGCCCAGCACCTGAACTCCTGGGGGGACCGTCAGTCTTCCTCTTCCCC |
| | CCAAAACCCAAGGACACCCTCATGATCTCCCGGACCCCTGAGGTCACATGCGTGGTGGTGGACGTGAGCCACGA |
| | AGACCCTGAGGTCAAGTTCAACTGGTACGTGGACGGCGTGGAGGTGCATAATGCCAAGACAAAGCCGCGGGAG |
| | GAGCAGTACAACAGCACGTACCGTGTGGTCAGCGTCCTCACCGTCCTGCACCAGGACTGGCTGAATGGCAAGGA |
| | GTACAAGTGCAAGGTCTCCAACAAAGCCCTCCCAGCCCCCATCGAGAAAACCATCTCCAAAGCCAAAGGGCAGC |
| | CCCGAGAACCACAGGTGTACACCCTGCCCCCATCCCGGGAGGAGATGACCAAGAACCAGGTCAGCCTGACCTGC |
| | CTGGTCAAAGGCTTCTATCCCAGCGACATCGCCGTGGAGTGGGAGAGCAATGGGCAGCCGGAGAACAACTACA |
| | AGACCACGCCTCCCGTGCTGGACTCCGACGGCTCCTTCTTCCTCTACAGCAAGCTCACCGTGGACAAGAGCAGGT |
| | GGCAGCAGGGGAACGTCTTCTCATGCTCCGTGATGCATGAGGCTCTGCACAACCACTACACGCAGAAGAGCCTC |
| | TCCCTGTCTCCGGGTAAA |
| VH-VL-CH2-CH3 | EVQLVESGGGLVQPGGSLRLSCAASGFDFSAASIHWVRQAPGKGLEWVAGISASGGATAYADSVKGRFTISADTSKN |
| AA | TAYLQMNSLRAEDTAVYYCARSRSSSYSSSGWRYYSGAFDYWGQGTLVTVSSGGGGSDIQMTQSPSSLSASVGDRV |
| | TITCRASQSVSSAVAWYQQKPGKAPKLLIYSASDLYSGVPSRFSGSRSGTDFTLTISSLQPEDFATYYCQQGSYLFTFGQ |
| | GTKVEIKGGGGSGGGGSDKTHTCPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFLYSKLTVDKSRWQQGNVFSCSVMHEALHN |
| | HYTQKSLSLSPGK |
| 25. LEAD Diabody-Fc-diabody clone example | |
| 15031 Diabody-Fc-diabody | |
| VH-VL-CH2- | GAGGTTCAGCTGGTGGAGTCTGGCGGTGGCCTGGTGCAGCCAGGGGGCTCACTCCGTTTGTCCTGTGCAGCTTC |
| CH3-VH-VLNT | TGGCTTCGACCTCACTGGTTCTTACATGCACTGGGTGCGTCAGGCCCCGGGTAAGGGCCTGGAATGGGTTGCAG |
| | GTATTTCCGCTTCTGGAGGCGCTACTGCTTATGCCGATAGCGTCAAGGGCCGTTTCACTATAAGCGCAGACACAT |
| | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGGTTGGCGTTACTACTCTGGTGCTTTTATGGACTACTGGGGTCAAGGAACCCTG |
| | GTCACCGTCTCCTCGGGTGGAGGTGGCAGTGATATCCAGATGACCCAGTCCCCGAGCTCCCTGTCCGCCTCTGT |
| | GGGCGATAGGGTCACCATCACCTGCCGTGCCAGTCAGTCCGTGTCCAGCGCTGTAGCCTGGTATCAACAGAAAC |
| | CAGGAAAAGCTCCGAAGCTTCTGATTTACTCGGCATCCGATCTCTACTCTGGAGTCCCTTCTCGCTTCTCTGGTAG |
| | CCGTTCCGGGACGGATTTCACTCTGACCATCAGCAGTCTGCAGCCGGAAGACTTCGCAACTTATTACTGTCAGCA |
| | AGGTTCTTACCTGTTCACGTTCGGACAGGGTACCAAGGTGGAGATCAAAGGTGGAGGTGGCTCCGGAGGAGGT |
| | GGCAGTGACAAGACACATACCTGTCCACCTTGTCCGGCACCTGAGCTGCTTGGAGGCCCTAGCGTTTTTCTCTTC |
| | CCGCCGAAGCCGAAGGATACGCTGATGATCAGTAGAACCCCTGAGGTTACCTGTGTCGTGGTTGATGTCTCACA |
| | CGAAGACCCCGAGGTAAAATTTAATTGGTATGTTGACGGGGTGGAAGTACATAATGCCAAGACCAAACCCCGCG |
| | AAGAACAATACAACAGTACATACAGGGTTGTCTCAGTTTTGACCGTTCTCCACCAAGATTGGCTCAATGGGAAAG |
| | AGTATAAATGCAAAGTGAGTAACAAAGCACTGCCGGCTCCAATAGAGAAGACGATCTCAAAAGCCAAGGGACA |
| | ACCACGAGAGCCCCAAGTCTACACCCTCCCGCCCTCCAGGGAAGAGATGACTAAGAACCAGGTTAGTTTGACGT |
| | GCTTGGTTAAGGGCTTTTACCCATCTGACATAGCCGTTGAATGGGAGTCAAATGGCCAACCAGAGAATAACTAT |
| | AAGACAACCCCCCCCGTACTTGACTCTGATGGCTCATTCTTCTTGTATTCCAAACTTACTGTCGATAAATCCCGCTG |
| | GCAACAAGGTAACGTCTTTTCTTGTAGTGTCATGCATGAGGCACTGCATAACCACTATACTCAAAAATCTCTGTCC |
| | CTCAGTCCAGGAGGAGGTGGGGGATCAGGTGGTGGGGGCTCTGGAGGCGGAACCGGTGAGGTTCAGCTGGTG |
| | GAGTCTGGCGGTGGCCTGGTGCAGCCAGGGGGCTCACTCCGTTTGTCCTGTGCAGCTTCTGGCTTCGACCTCACT |
| | GGTTCTTACATGCACTGGGTGCGTCAGGCCCCGGGTAAGGGCCTGGAATGGGTTGCAGGTATTTCCGCTTCTGG |
| | AGGCGCTACTGCTTATGCCGATAGCGTCAAGGGCCGTTTCACTATAAGCGCAGACACATCCAAAAACACAGCCT |
| | ACCTACAAATGAACAGCTTAAGAGCTGAGGACACTGCCGTCTATTATTGTGCTCGCTCTCGTTCTTCTTCTTACTCT |
| | TCTTCTGGTTGGCGTTACTACTCTGGTGCTTTTATGGACTACTGGGGTCAAGGAACCCTGGTCACCGTCTCCTCGG |
| | GTGGAGGTGGCAGTGATATCCAGATGACCCAGTCCCCGAGCTCCCTGTCCGCCTCTGTGGGCGATAGGGTCACC |
| | ATCACCTGCCGTGCCAGTCAGTCCGTGTCCAGCGCTGTAGCCTGGTATCAACAGAAACCAGGAAAAGCTCCGAA |
| | GCTTCTGATTTACTTCGGCATCCGATCTCTACTCTGGAGTCCCTTCTCGCTTCTCTGGTAGCCGTTCCGGGACGGAT |
| | TTCACTCTGACCATCAGCAGTCTGCAGCCGGAAGACTTCGCAACTTATTACTGTCAGCAAGGTTCTTACCTGTTCA |
| | CGTTCGGACAGGGTACCAAGGTGGAGATCAAA |
| VH-VL-CH2- | EVQLVESGGGLVQPGGSLRLSCAASGFDLTGSYMHWVRQAPGKGLEWVAGISASGGATAYADSVKGRFTISADTSK |
| CH3-VH-VLNT | NTAYLQMNSLRAEDTAVYYCARSRSSSYSSSGWRYYSGAFMDYWGQGTLVTVSSGGGGSDIQMTQSPSSLSASVG |
| AA | DRVTITCRASQSVSSAVAWYQQKPGKAPKLLIYSASDLYSGVPSRFSGSRSGTDFTLTISSLQPEDFATYYCQQGSYLFT |
| | FGQGTKVEIKGGGGSGGGGSDKTHTCPPCPAPELLGGPSVFLFPPKPKETLMISRTPEVTCVVVDVSHEDPEVKRNW |
| | YVDGVEVHNAKTKPREEQYNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKSRWQQGNVFSCSVMHEA |
| | LHNHYTQKSLSLSPGGGGGSGGGGSGGGTGEVQLVESGGGLVQPGGSLRLSCAASGFDLTGSYMHWVRQAPGKG |
| | LEWVAGISASGGATAYADSVKGRFTISADTSKNTAYLQMNSLRAEDTAVYYCARSRSSSYSSSGWRYYSGAFMDYW |
| | GQGTLVTVSSGGGGSDIQMTQSPSSLSASVGDRVTITCRASQSVSSAVAWYQQKPGKAPKLLIYSASDLYSGVPSRFS |
| | GSRSGTDFTLTISSLQPEDFATYYCQQGSYLFTFGQGTKVEIK |
| 26. LEAD Diabody-Fc-VHH clone example | |
| 15031 Diabody-Fc-VHH | |
| VH-VL-CH2- | GAGGTTCAGCTGGTGGAGTCTGGCGGTGGCCTGGTGCAGCCAGGGGGCTCACTCCGTTTGTCCTGTGCAGCT |
| CH3-VHH | TCTGGCTTCGACCTCACTGGTTCTTACATGCACTGGGTGCGTCAGGCCCCGGGTAAGGGCCTGGAATGGGTTG |
| | CAGGTATTTCCGCTTCTGGAGGCGCTACTGCTTATGCCGATAGCGTCAAGGGCCGTTTCACTATAAGCGCAGA |
| | CACATCCAAAAACACAGCCTACCTACAAATGAACAGCTTAAGAGCTGAGGACACTGCCGTCTATTATTGTGCT |
| | CGCTCTCGTTCTTCTTCTTACTCTTCTTCTGGTTGGCGTTACTACTCTGGTGCTTTTATGGACTACTGGGGTCAA |
| | GGAACCCTGGTCACCGTCTCCTCGGGTGGAGGTGGCAGTGATATCCAGATGACCCAGTCCCCGAGCTCCCTG |
| | TCCGCCTCTGTGGGCGATAGGGTCACCATCACCTGCCGTGCCAGTCAGTCCGTGTCCAGCGCTGTAGCCTGGT |
| | ATCAACAGAAACCAGGAAAAGCTCCGAAGCTTCTGATTTACTCGGCATCCGATCTCTACTCTGGAGTCCCTTCT |
| | CGCTTCTCTGGTAGCCGTTCCGGGACGGATTTCACTCTGACCATCAGCAGTCTGCAGCCGGAAGACTTCGCAA |
| | CTTATTACTGTCAGCAAGGTTCTTACCTGTTCACGTTCGGACAGGGTACCAAGGTGGAGATCAAAGGTGGAG |
| | GTGGCTCCGGAGGAGGTGGCAGTGACAAGACACATACCTGTCCACCTTGTCCGGCACCTGAGCTGCTTGGAG |
| | GCCCTAGCGTTTTTCTCTTCCCGCCGAAGCCGAAGGATACGCTGATGATCAGTAGAACCCCTGAGGTTACCTG |
| | TGTCGTGGTTGATGTCTCACACGAAGACCCCGAGGTAAAATTTAATTGGTATGTTGACGGGGTGGAAGTACA |
| | TAATGCCAAGACCAAACCCCGCGAAGAACAATACAACAGTACATACAGGGTTGTCTCAGTTTTGACCGTTCTC |
| | CACCAAGATTGGCTCAATGGGAAAGAGTATAAATGCAAAGTGAGTAACAAAGCACTGCCGGCTCCAATAGAG |
| | AAGACGATCTCAAAAGCCAAGGGACAACCACGAGAGCCCCAAGTCTACACCCTCCCGCCCTCCAGGGAAGAG |
| | ATGACTAAGAACCAGGTTAGTTTGACGTGCTTGGTTAAGGGCTTTTACCCATCTGACATAGCCGTTGAATGGG |
| | AGTCAAATGGCCAACCAGAGAATAACTATAAGACAACCCCCCCCGTACTTGACTCTGATGGCTCATTCTTCTTG |
| | TATTCCAAACTTACTGTCGATAAATCCCGCTGGCAACAAGGTAACGTCTTTTCTTGTAGTGTCATGCATGAGGC |
| | ACTGCATAACCACTATACTCAAAAATCTCTGTCCCTCAGTCCAGGAGGAGGTGGGGGATCAGGTGGTGGGG |
| | GCTCTGGAGGCGGAACCGGTGAGGTTCAGCTGGTGGAGTCTGGCGGTGGCCTGGTGCAGCCAGGGCGTTCA |
| | CTCCGTTTGTCCTGTGCAGCTTCTGGCTTCGACCTCACTGGTTCTTACATGCATTGGGTGCGTCGTGCCCCGGG |
| | TAAGGGCGAGGAACTGGTTGCAGGTATTTCCGCTTCTGGAGGCGCTACTGCTTATGCCGATAGCGTCAAGGG |
| | CCGTTTCACTATAAGCGCAGACACATCCAAAAACACAGCCTACCTACAAATGAACAGCTTAAGAGCTGAGGAC |
| | ACTGCCGTCTATTATTGTGCTCGCTCTCGTTCTTCTTCTTACTCTTCTTCTGGTTGGCGTTACTACTCTGGTGCTT |
| | TTGACTACTGGGGTCAAGGAACCCTGGTCACCGTCTCCTCG |
| VH-VL-CH2- | EVQLVESGGGLVQPGGSLRLSCAASGFDLTGSYMHWVRQAPGKGLEWVAGISASGGATAYADSVKGRFTISADT |
| CH3-VHH | SKNTAYLQMNSLRAEDTAVYYCARSRSSSYSSSGWRYYSGAFMDYWGQGTLVTVSSGGGGSDIQMTQSPSSLSA |
| AA | SVGDRVTITCRASQSVSSAVAWYQQKPGKAPKLLIYSASDLYSGVPSRFSGSRSGTDFTLTISSLQPEDFATYYCQQG |
| | SYLFTFGQGTKVEIKGGGGSGGGGSDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSNKALPAPIEKTISKAKGQPREPQ |
| | VYTLPPSREEMTKNQVSLTCLVKGFYPSDIAVEWESNGQPENNYKTTPPVLDSDGSFFLYSKLTVDKSRWQQGNV |
| | FSCSVMHEALHNHYTQKSLSLSPGGGGGSGGGGSGGGTGEVQLVESGGGLVQPGRSLRLSCAASGFDLTGSYM |
| | HWVRRAPGKGEELVAGISASGGATAYADSVKGRFTISADTSKNTAYLQMNSLRAEDTAVYYCARSRSSSYSSSGW |
| | RYYSGAFDYWGQGTLVTVSS |
| 27. LEAD Diabody-Fc-scFv clone example | |
| 15031 Diabody-Fc-scFv | |
| VH-VL-CH2- | GAGGTTCAGCTGGTGGAGTCTGGCGGTGGCCTGGTGCAGCCAGGGGGCTCACTCCGTTTGTCCTGTGCAGCTTC |
| CH3-VL-VH | TGGCTTCGATCTCACTGGTTCTTACATGCACTGGGTGCGTCAGGCCCCGGGTAAGGGCCTGGAATGGGTTGCAG |
| NT | GTATTTCCGCTTCTGGAGGCGCTACTGCTTATGCCGATAGCGTCAAGGGCCGTTTCACTATAAGCGCAGACACAT |
| | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGGTTGGCGTTACTACTCTGGTGCTTTTGACTACTGGGGTCAAGGAACCCTGGTC |
| | ACCGTCTCCTCGGGTGGAGGTGGCAGTGATATCCAGATGACCCAGTCCCCGAGCTCCCTGTCCGCCTCTGTGGG |
| | CGATAGGGTCACCATCACCTGCCGTGCCAGTCAGTCCGTGTCCAGCGCTGTAGCCTGGTATCAACAGAAACCAG |
| | GAAAAGCTCCGAAGCTTCTGATTTACTCGGCATCCGATCTCTACTCTGGAGTCCCTTCTCGCTTCTCTGGTAGCCG |
| | TTCCGGGACGGATTTCACTCTGACCATCAGCAGTCTGCAGCCGGAAGACTTCGCAACTTATTACTGTCAGCAAGG |
| | TTCTTACCTGTTCACGTTCGGACAGGGTACCAAGGTGGAGATCAAAGGTGGAGGTGGCTCCGGAGGAGGTGGC |
| | AGTGACAAAACTCACACATGCCCACCGTGCCCAGCACCTGAACTCCTGGGGGGACCGTCAGTCTTCCTCTTCCCC |
| | CCAAAACCCAAGGACACCCTCATGATCTCCCGGACCCCTGAGGTCACATGCGTGGTGGTGGACGTGAGCCACGA |
| | AGACCCTGAGGTCAAGTTCAACTGGTACGTGGACGGCGTGGAGGTGCATAATGCCAAGACAAAGCCGCGGGAG |
| | GAGCAGTACAACAGCACGTACCGTGTGGTCAGCGTCCTCACCGTCCTGCACCAGGACTGGCTGAATGGCAAGGA |
| | GTACAAGTGCAAGGTCTCCAACAAAGCCCTCCCAGCCCCCATCGAGAAAACCATCTCCAAAGCCAAAGGGCAGC |
| | CCCGAGAACCACAGGTGTACACCCTGCCCCCATCCCGGGAGGAGATGACCAAGAACCAGGTCAGCCTGACCTGC |
| | CTGGTCAAAGGCTTCTATCCCAGCGACATCGCCGTGGAGTGGGAGAGCAATGGGCAGCCGGAGAACAACTACA |
| | AGACCACGCCTCCCGTGCTGGACTCCGACGGCTCCTTCTTCCTCTACAGCAAGCTCACCGTGGACAAGAGCAGGT |
| | GGCAGCAGGGGAACGTCTTCTCATGCTCCGTGATGCATGAGGCTCTGCACAACCACTACACGCAGAAGAGCCTC |
| | TCCCTGTCTCCGGGTAAAGGAGGAGGTGGGGGATCAGGTGGTGGGGGCTCTGGAGGCGGAACCGGTGATATC |
| | CAGATGACCCAGTCCCCGAGCTCCCTGTCCGCCTCTGTGGGCGATAGGGTCACCATCACCTGCCGTGCCAGTCAG |
| | TCCGTGTCCAGCGCTGTAGCCTGGTATCAACAGAAACCAGGAAAAGCTCCGAAGCTTCTGATTTACTCGGCATCC |
| | GATCTCTACTCTGGAGTCCCTTCTCGCTTCTCTGGTAGCCGTTCCGGGACGGATTTCACTCTGACCATCAGCAGTC |
| | TGCAGCCGGAAGACTTCGCAACTTATTACTGTCAGCAAGGTTCTTACCTGTTCACGTTCGGACAGGGTACCAAGG |
| | TGGAGATCAAAGGTACTACTGCCGCTAGTGGTAGTAGTGGTGGCAGTAGCAGTGGTGCCGAGGTTCAGCTGGT |
| | GGAGTCTGGCGGTGGCCTGGTGCAGCCAGGGGGCTCACTCCGTTTGTCCTGTGCAGCTTCTGGCTTCGACCTCA |
| | CTGGTTCTTACATGCACTGGGTGCGTCAGGCCCCGGGTAAGGGCCTGGAATGGGTTGCAGGTATTTCCGCTTCT |
| | GGAGGCGCTACTGCTTATGCCGATAGCGTCAAGGGCCGTTTCACTATAAGCGCAGACACATCCAAAAACACAGC |
| | CTACCTACAAATGAACAGCTTAAGAGCTGAGGACACTGCCGTCTATTATTGTGCTCGCTCTCGTTCTTCTTCTTACT |
| | CTTCTTCTGGTTGGCGTTACTACTCTGGTGCTTTTGACTACTGGGGTCAAGGAACCCTGGTCACCGTCTCCTCG |
| VH-VL-CH2- | EVQLVESGGGLVQPGGSLRLSCAASGFDLTGSYMHWVRQAPGKGLEWVAGISASGGATAYADSVKGRFTISADTSK |
| CH3-VL-VH | NTAYLOMNSIRAEDTAVYYCARSRSSSYSSSGWRYYSGAFDYWGQGTLVTVSSGGGGSDIQMTQSPSSLSASVGDR |
| AA | VTITCRASQSVSSAVAWYQQKPGKAPKLLIYSASDLYSGVPSRFSGSRSGTDFTLTISSLOPEDFATYYCQQGSYLFTFG |
| | QGTKVEIKGGGGSGGGGSDKTHTCPPCPAPELLGGPSVFLFPPKPKDTLMISRTPEVTCVVVDVSHEDPEVKFNWYV |
| | DGVEVHNAKTKPREFQYNSTYRVVSVLTVLHQDWLNGKEYKCKVSNKALPAPIEKTISKAKGQPREPQVYTLPPSRFE |
| | MTKNQVSLTCLVKGFYPSDIAVEWESNGQPENNYKTTPPVLDSDGSFFLYSKLTVDKSRWQQGNVFSCSVMHEALH |
| | NHYTQKSLSLSPGKGGGGGSGGGGSGGGTGDIQMTQSPSSLSASVGDRVTITCRASQSVSSAVAWYQQKPGKAPK |
| | LLIYSASDLYSGVPSRFSGSRSGTDFTLTISSLQPEDFATYYCQQGSYLFTFGQGTKVEIKGTTAASGSSGGSSSGAEVQL |
| | VESGGGLVQPGGSLRISCAASGFDLTGSYMHWVRQAPGKGLEWVAGISASGGATAYADSVKGRFTISADTSKNTAY |
| | LQMNSLRAEDTAVYYCARSRSSSYSSSGWRYYSGAFDYWGQGTLVTVSS |
| 28. LEAD Diabody-Fc-Fav clone example | |
| 5031 Diabody-Fc-Fab | |
| VL-CL | GATATCCAGATGACCCAGTCCCCGAGCTCCCTGTCCGCCTCTGTGGGCGATAGGGTCACCATCACCTGCCGTGCC |
| NT | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL | DIQMTQSPSSLSASVGDRVTITCRASQSVSSAVAWYQQKPGKAPKLLIYSASDLYSGVPSRFSGSRSGTDFTLTISSLQP |
| AA | EDFATYYCQQGSYLFTFGQGTKVEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGEC |
| VH-VL-CH2- | GAGGTTCAGCTGGTGGAGTCTGGCGGTGGCCTGGTGCAGCCAGGGGGCTCACTCCGTTTGTCCTGTGCAGCTTC |
| CH3-VH-CH1 | TGGCTTCGATCTCACTGGTTCTTACATGCACTGGGTGCGTCAGGCCCCGGGTAAGGGCCTGGAATGGGTTGCAG |
| NT | GTATTTCCGCTTCTGGAGGCGCTACTGCTTATGCCGATAGCGTCAAGGGCCGTTTCACTATAAGCGCAGACACAT |
| | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGGTTGGCGTTACTACTCTGGTGCTTTTGACTACTGGGGTCAAGGAACCCTGGTC |
| | ACCGTCTCCTCGGGTGGAGGTGGCAGTGATATCCAGATGACCCAGTCCCCGAGCTCCCTGTCCGCCTCTGTGGG |
| | CGATAGGGTCACCATCACCTGCCGTGCCAGTCAGTCCGTGTCCAGCGCTGTAGCCTGGTATCAACAGAAACCAG |
| | GAAAAGCTCCGAAGCTTCTGATTTACTCGGCATCCGATCTCTACTCTGGAGTCCCTTCTCGCTTCTCTGGTAGCCG |
| | TTCCGGGACGGATTTCACTCTGACCATCAGCAGTCTGCAGCCGGAAGACTTCGCAACTTATTACTGTCAGCAAGG |
| | TTCTTACCTGTTCACGTTCGGACAGGGTACCAAGGTGGAGATCAAAGGTGGAGGTGGCTCCGGAGGAGGTGGC |
| | AGTGACAAGACACATACCTGTCCACCTTGTCCGGCACCTGAGCTGCTTGGAGGCCCTAGCGTTTTTCTCTTCCCGC |
| | CGAAGCCGAAGGATACGCTGATGATCAGTAGAACCCCTGAGGTTACCTGTGTCGTGGTTGATGTCTCACACGAA |
| | GACCCCGAGGTAAAATTTAATTGGTATGTTGACGGGGTGGAAGTACATAATGCCAAGACCAAACCCCGCGAAGA |
| | ACAATACAACAGTACATACAGGGTTGTCTCAGTTTTGACCGTTCTCCACCAAGATTGGCTCAATGGGAAAGAGTA |
| | TAAATGCAAAGTGAGTAACAAAGCACTGCCGGCTCCAATAGAGAAGACGATCTCAAAAGCCAAGGGACAACCA |
| | CGAGAGCCCCAAGTCTACACCCTCCCGCCCTCCAGGGAAGAGATGACTAAGAACCAGGTTAGTTTGACGTGCTT |
| | GGTTAAGGGCTTTTACCCATCTGACATAGCCGTTGAATGGGAGTCAAATGGCCAACCAGAGAATAACTATAAGA |
| | CAACCCCCCCCGTACTTGACTCTGATGGCTCATTCTTCTTGTATTCCAAACTTACTGTCGATAAATCCCGCTGGCAA |
| | CAAGGTAACGTCTTTTCTTGTAGTGTCATGCATGAGGCACTGCATAACCACTATACTCAAAAATCTCTGTCCCTCA |
| | GTCCAGGAGGAGGTGGGGGATCAGGTGGTGGGGGCTCTGGAGGCGGAACCGGTGAGGTTCAGCTGGTGGAG |
| | TCTGGCGGTGGCCTGGTGCAGCCAGGGGGCTCACTCCGTTTGTCCTGTGCAGCTTCTGGCTTCGATCTCACTGGT |
| | TCTTACATGCACTGGGTGCGTCAGGCCCCGGGTAAGGGCCTGGAATGGGTTGCAGGTATTTCCGCTTCTGGAGG |
| | CGCTACTGCTTATGCCGATAGCGTCAAGGGCCGTTTCACTATAAGCGCAGACACATCCAAAAACACAGCCTACCT |
| | ACAAATGAACAGCTTAAGAGCTGAGGACACTGCCGTCTATTATTGTGCTCGCTCTCGTTCTTCTTCTTACTCTTCTT |
| | CTGGTTGGCGTTACTACTCTGGTGCTTTTGACTACTGGGGTCAAGGAACCCTGGTCACCGTCTCCTCGGCTAGCA |
| | CCAAGGGTCCATCGGTCTTCCCCCTGGCACCCTCCTCCAAGAGCACCTCTGGGGGCACAGCGGCCCTGGGCTGC |
| | CTGGTCAAGGACTACTTCCCCGAACCCGTGACGGTGTCGTGGAACTCAGGCGCCCTGACCAGCGGCGTGCACAC |
| | CTTCCCGGCTGTCCTACAGTCCTCAGGACTCTACTCCCTCAGCAGCGTGGTGACCGTGCCCTCCAGCAGCTTGGG |
| | CACCCAGACCTACATCTGCAACGTGAATCACAAGCCCAGCAACACCAAGGTCGACAAGAAAGTTGAGCCCAAAT |
| | CTTGTGACAAAACTCACACA |
| VH-VL-CH2- | EVQLVESGGGLVQPGGSIRLSCAASGFDLTGSYMHWVRQAPGKGLEWVAGISASGGATAYADSVKGRFTISADTSK |
| CH3-VH-CH1 | NTAYLQMNSERAEDTAVYYCARSRSSSYSSSGWRYYSGAFDYWGQGTLVTVSSGGGGSDIQMTQSPSSLSASVGDR |
| AA | VTITCRASQSVSSAVAWYQQKPGKAPKLLIYSASDLYSGVPSRFSGSRSGTDFTLTISSLQPEDFATYYCQQGSYLFTFG |
| | QGTKVEIKGGGGSGGGGSDKTHTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSKLTVDKSRWQQGNVFSCSVMHEALH |
| | NHYTQKSLSLSPGGGGGSGGGGSGGGTGEVQLVESGGGLVQPGGSLRLSCAASGFDLTGSYMHWVRQAPGKGLE |
| | WVAGISASGGATAYADSVKGRFTISADISKNTAYLQMNSLRAEDTAVYYCARSRSSSYSSSGWRYYSGAFDYWGQG |
| | TLVTVSSASTKGPSVFPLAPSSKSTSGGTAALGCLVKDYFPEPVTVSWNSGALTSGVHTFPAVLQSSGLYSLSSVVTVPS |
| | SSLGTQTYICNVNHKPSNTKVDKKVEPKSCDKTHT |
| 29. LEAD Fab-diabody-Fc clone example | |
| 15031 Fab-Diabody Fc | |
| VL-CL NT | GATATCCAGATGACCCAGTCCCCGAGCTCCCTGTCCGCCTCTGTGGGCGATAGGGTCACCATCACCTGCCGTGCC |
| | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCASCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTLTISSLQP |
| | EDFATYYCQQGSYLFTFGQGTKVEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGEC |
| VH-CH1-VH- | GAAGTCCAACTCGTCGAATCCGGTGGTGGATTGGTGCAGCCAGGGGGCTCACTCCGTTTGTCCTGTGCAGCTTC |
| VL-CH2-CH3 | TGGCTTCGACCTCACTGGTTCTTACATGCACTGGGTGCGTCAGGCCCCGGGTAAGGGCCTGGAATGGGTTGCAG |
| NT | GTATTTCCGCTTCTGGAGGCGCTACTGCTTATGCCGATAGCGTCAAGGGCCGTTTCACTATAAGCGCAGACACAT |
| | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGGTTGGCGTTACTACTCTGGTGCTTTTGACTACTGGGGTCAAGGAACCCTGGTC |
| | ACCGTCTCCTCGGCTAGCACCAAGGGCCCATCGGTCTTCCCCCTGGCACCCTCCTCCAAGAGCACCTCTGGGGGC |
| | ACAGCGGCCCTGGGCTGCCTGGTCAAGGACTACTTCCCCGAACCGGTGACGGTGTCGTGGAACTCAGGCGCCCT |
| | GACCAGCGGCGTGCACACCTTCCCGGCTGTCCTACAGTCCTCAGGACTCTACTCCCTCAGCAGCGTGGTGACCGT |
| | GCCCTCCAGCAGCTTGGGCACCCAGACCTACATCTGCAACGTGAATCACAAGCCCAGCAACACCAAGGTGGACA |
| | AGAAAGTTGAGCCCAAATCTTGTGACAAAACTCACACAACCGGTGGAGGTGGCAGTGGCGGAGGTGGCAGCG |
| | GTGGAGGTGGAAGTGGAGGAGGTGGCAGTGGTGGAGGAGAGGTTCAGCTGGTGGAGTCTGGCGGTGGCCT |
| | GGTGCAGCCAGGGGGCTCACTCCGTTTGTCCTGTGCAGCTTCTGGCTTCGACCTCACTGGTTCTTACATGCACTG |
| | GGTGCGTCAGGCCCCGGGTAAGGGCCTGGAATGGGTTGCAGGTATTTCCGCTTCTGGAGGGGCTACTGCTTATG |
| | CCGATAGCGTCAAGGGCCGTTTCACTATAAGCGCAGACACATCCAAAAACACAGCCTACCTACAAATGAACAGC |
| | TTAAGAGCTGAGGACACTGCCGTCTATTATTGTGCTCGCTCTCGTTCTTCTTCTTACTCTTCTTCTGGTTGGCGTTA |
| | CTACTCTGGTGCTTTTGACTACTGGGGTCAAGGAACCCTGGTCACCGTCTCCTCGGGTGGAGGTGGCAGTGATA |
| | TCCAGATGACCCAGTCCCCGAGCTCCCTGTCCGCCTCTGTGGGCGATAGGGTCACCATCACCTGCCGTGCCAGTC |
| | AGTCCGTGTCCAGCGCTGTAGCCTGGTATCAACAGAAACCAGGAAAAGCTCCGAAGCTTCTGATTTACTCGGCAT |
| | CCGATCTCTACTCTGGAGTCCCTTCTCGCTTCTCTGGTAGCCGTTCCGGGACGGATTTCACTCTGACCATCAGCAG |
| | TCTGCAGCCGGAAGACTTCGCAACTTATTACTGTCAGCAAGGTTCTTACCTGTTCACGTTCGGACAGGGTACCAA |
| | GGTGGAGATCAAAGGTGGAGGTGGCTCCGGAGGAGGTGGCAGTGACAAAACTCACACATGCCCACCGTGCCC |
| | AGCACCTGAACTCCTGGGGGGACCGTCAGTCTTCCTCTTCCCCCCAAAACCCAAGGACACCCTCATGATCTCCCG |
| | GACCCCTGAGGTCACATGCGTGGTGGTGGACGTGAGCCACGAAGACCCTGAGGTCAAGTTCAACTGGTACGTG |
| | GACGGCGTGGAGGTGCATAATGCCAAGACAAAGCCGCGGGAGGAGCAGTACAACAGCACGTACCGTGTGGTC |
| | AGCGTCCTCACCGTCCTGCACCAGGACTGGCTGAATGGCAAGGAGTACAAGTGCAAGGTCTCCAACAAAGCCCT |
| | CCCAGCCCCCATCGAGAAAACCATCTCCAAAGCCAAAGGGCAGCCCCGAGAACCACAGGTGTACACCCTGCCCC |
| | CATCCCGGGAGGAGATGACCAAGAACCAGGTCAGCCTGACCTGCCTGGTCAAAGGCTTCTATCCCAGCGACATC |
| | GCCGTGGAGTGGGAGAGCAATGGGCAGCCGGAGAACAACTACAAGACCACGCCTCCCGTGCTGGACTCCGACG |
| | GCTCCTTCTTCCTCTACAGCAAGCTCACCGTGGACAAGAGCAGGTGGCAGCAGGGGAACGTCTTCTCATGCTCCG |
| | TGATGCATGAGGCTCTGCACAACCACTACACGCAGAAGAGCCTCTCCCTGTCTCCGGGTAAA |
| VH-CH1-VH- | EVQLVESGGGLVQPGGSLRLSCAASGFDLTGSYMHWVRQAPGKGLEWVAGISASGGATAYADSVKGRFTISADISK |
| VL-CH2-CH3 | NTAYLQMNSLRAEDTAVYYCARSRSSSYSSSGWRYYSGAFDYWGQGTLVTVSSASTKGPSVFPLAPSSKSTSGGTAAL |
| AA | GCLVKDYFPEPVTVSWNSGALTSGVHTFPAVLQSSGLYSLSSVVTVPSSSLGTQTYICNVNHKPSNTKVDKKVEPKSCD |
| | KTHTTGGGGSGGGGSGGGGSGGGGSGGGEVQLVESGGGLVQPGGSLRLSCAASGFDLTGSYMHWVRQAPGKGL |
| | EWVAGISASGGATAYADSVKGRFTISADTSKNTAYLQMNSLRAEDTAVYYCARSRSSSYSSSGWRYYSGAFDYWGQ |
| | GTLVTVSSGGGGSDIQMTQSPSSLSASVGDRVTITCRASQSVSSAVAWYQQKPGKAPKLLIYSASDLYSGVPSRFSGS |
| | RSGTDFTLTISSLQPEDFATYYCQQGSYLFTFGQGTKVEIKGGGGSGGGGSDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLHQDWLNGKEYKCKVS |
| | NKALPAPIEKTISKAKGQPREPQVYTIPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK |
| 30. LEAD VHH-hIgG1 clone example | |
| 15031 hIgG1 | |
| VL-CL NT | GATATCCAGATGACCCAGTCCCCGAGCTCCCTGTCCGCCTCTGTGGGCGATAGGGTCACCATCACCTGCCGTGCC |
| | AGTCAGTCCGTGTCCAGCGCTGTAGCCTGGTATCAACAGAAACCAGGAAAAGCTCCGAAGCTTCTGATTTACTTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTLTISSLQP |
| | EDFATYYCQQGSYLFTFGQGTKVEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGEC |
| VHH-hIgG1 | GAGGTTCAGCTGGTGGAGTCTGGCGGTGGCCTGGTGCAGCCAGGGCGTTCACTCCGTTTGTCCTGTGCAGCTTC |
| NT | TGGCTTCGACCTCACTGGTTCTTACATGCACTGGGTGCGTCGTGCCCCGGGTAAGGGCGAGGAACTGGTTGCAG |
| | GTATTTCCGCTTCTGGAGGCGCTACTGCTTATGCCGATAGCGTCAAGGGCCGTTTCACTATAAGCGCAGACACAT |
| | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGGTTGGCGTTACTACTCTGGTGCTTTTGACTACTGGGGTCAAGGAACCCTGGTC |
| | ACCGTCTCCTCGGGTGGAGGTGGCTCCGGAGGAGGTGGCAGTGAGGTTCAGCTGGTGGAGTCTGGCGGTGGC |
| | CTGGTGCAGCCAGGGGGCTCACTCCGTTTGTCCTGTGCAGCTTCTGGCTTCGACCTCACTGGTTCTTACATGCACT |
| | GGGTGCGTCAGGCCCCGGGTAAGGGCCTGGAATGGGTTGCAGGTATTTCCGCTTCTGGAGGCGCTACTGCTTAT |
| | GCCGATAGCGTCAAGGGCCGTTTCACTATAAGCGCAGACACATCCAAAAACACAGCCTACCTACAAATGAACAG |
| | CTTAAGAGCTGAGGACACTGCCGTCTATTATTGTGCTCGCTCTCGTTCTTCTTCTTACTCTTCTTCTGGTTGGCGTT |
| | ACTACTCTGGTGCTTTTGACTACTGGGGTCAAGGAACCCTGGTCACCGTCTCCTCGGCTAGCACCAAGGGCCCAT |
| | CGGTCTTCCCCCTGGCACCCTCCTCCAAGAGCACCTCTGGGGGCACAGCGGCCCTGGGCTGCCTGGTCAAGGAC |
| | TACTTCCCCGAACCGGTGACGGTGTCGTGGAACTCAGGCGCCCTGACCAGCGGCGTGCACACCTTCCCGGCTGT |
| | CCTACAGTCCTCAGGACTCTACTCCCTCAGCAGCGTGGTGACCGTGCCCTCCAGCAGCTTGGGCACCCAGACCTA |
| | CATCTGCAACGTGAATCACAAGCCCAGCAACACCAAGGTGGACAAGAAAGTTGAGCCCAAATCTTGTGACAAAA |
| | CTCACACATGCCCACCGTGCCCAGCACCTGAACTCCTGGGGGGACCGTCAGTCTTCCTCTTCCCCCCAAAACCCA |
| | AGGACACCCTCATGATCTCCCGGACCCCTGAGGTCACATGCGTGGTGGTGGACGTGAGCCACGAAGACCCTGAG |
| | GTCAAGTTCAACTGGTACGTGGACGGCGTGGAGGTGCATAATGCCAAGACAAAGCCGCGGGAGGAGCAGTACA |
| | ACAGCACGTACCGTGTGGTCAGCGTCCTCACCGTCCTGCACCAGGACTGGCTGAATGGCAAGGAGTACAAGTGC |
| | AAGGTCTCCAACAAAGCCCTOCCAGCCCCCATCGAGAAAACCATCTCCAAAGCCAAAGGGCAGCCCCGAGAACC |
| | ACAGGTGTACACCCTGCCCCCATCCCGGGAGGAGATGACCAAGAACCAGGTCAGCCTGACCTGCCTGGTCAAAG |
| | GCTTCTATCCCAGCGACATCGCCGTGGAGTGGGAGAGCAATGGGCAGCCGGAGAACAACTACAAGACCACGCC |
| | TCCCGTGCTGGACTCCGACGGCTCCTTCTTCCTCTACAGCAAGCTCACCGTGGACAAGAGCAGGTGGCAGCAGG |
| | GGAACGTCTTCTCATGCTCCGTGATGCATGAGGCTCTGCACAACCACTACACGCAGAAGAGCCTCTCCCTGTCTC |
| | CGGGTAAA |
| VHH-hIgG1 | EVQLVESGGGLVQPGRSLRLSCAASGFDLTGSYMHWVRRAPGKGEELVAGISASGGATAYADSVKGRFTISADTSKN |
| AA | TAYLQMNSLRAEDTAVYYCARSRSSSYSSSGWRYYSGAFDYWGQGTLVTVSSGGGGSGGGGSEVQLVESGGGLVQ |
| | PGGSLRLSCAASGFDLTGSYMHWVRQAPGKGLEWVAGISASGGATAYADSVKGRFTISADTSKNTAYLQMNSLRAE |
| | DTAVYYCARSRSSSYSSSGWRYYSGAFDYWGQGTLVTVSSASTKGPSVFPLAPSSKSTSGGTAALGCLVKDYFPEPVT |
| | VSWNSGALTSGVHTFPAVLQSSGLYSLSSVVTVPSSSLGTQTYICNVNHKPSNTKVDKKVEPKSCDKTHTCPPCPAPEL |
| | LGGPSVFLFPPKPKDTLMISRTPEVTCVVVDVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSREEMTKNQVSLTCLVKGFYPSDIAVEWESNGQPEN |
| | NYKTTPPVLDSDGSFFLYSKLTVDKSRWQQGNVFSCSVMHEALHNHYTQKSLSLSPGK |
| 31. LEAD VHH-VHH-hIgG1 clone example | |
| 15031 VHH-VHH-hIgG1 | |
| VL-CL NT | GATATCCAGATGACCCAGTCCCCGAGCTCCCTGTCCGCCTCTGTGGGCGATAGGGTCACCATCACCTGCCGTGCC |
| | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL AA | DIQMTQSPSSLSASVGDRVTITCRASQSVSSAVAWYQQKPGKAPKLLYSASDLYSGVPSRFSGSRSGTDFTLTISSLQP |
| | EDFATYYCQQGSYLFTFGQGTKVEIKRTVAAPSVFIFPPSDEQLKSGTASVVCLLNNFYPREAKVQWKVDNALQSGNS |
| | QESVTEQQSKDSTYSLSSTLTLSKADYEKHKVYACEVTHQGLSSPVTKSFNRGEC |
| VHH-VHH-hIgG1 | GAGGTTCAGCTGGTGGAGTCTGGCGGTGGCCTGGTGCAGCCAGGGCGTTCACTCCGTTTGTCCTGTGCAGCTTC |
| NT | TGGCTTCGACCTCACTGGTTCTTACATGCACTGGGTGCGTCGTGCCCCGGGTAAGGGCGAGGAACTGGTTGCAG |
| | GTATTTCCGCTTCTGGAGGCGCTACTGCTTATGCCGATAGCGTCAAGGGCCGTTTCACTATAAGCGCAGACACAT |
| | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGGTTGGCGTTACTACTCTGGTGCTTTTGACTACTGGGGTCAAGGAACCCTGGTC |
| | ACCGTCTCCTCGGGTGGAGGTGGCTCCGGAGGAGGTGGCAGTGAGGTTCAGCTGGTGGAGTCTGGCGGTGGC |
| | CTGGTGCAGCCAGGGCGTTCACTCCGTTTGTCCTGTGCAGCTTCTGGCTTCGACCTCACTGGTTCTTACATGCACT |
| | GGGTGCGTCGTGCCCCGGGTAAGGGCGAGGAACTGGTTGCAGGTATTTCCGCTTCTGGAGGCGCTACTGCTTAT |
| | GCCGATAGCGTCAAGGGCCGTTTCACTATAAGCGCAGACACATCCAAAAACACAGCCTACCTACAAATGAACAG |
| | CTTAAGAGCTGAGGACACTGCCGTCTATTATTGTGCTCGCTCTCGTTCTTCTTCTTACTCTTCTTCTGGTTGGCGTT |
| | ACTACTCTGGTGCTTTTGACTACTGGGGTCAAGGAACCCTGGTCACCGTCTCCTCGGGTGGAGGTGGCTCCGGA |
| | GGAGGTGGCAGTGAGGTTCAGCTGGTGGAGTCTGGCGGTGGCCTGGTGCAGCCAGGGGGCTCACTCCGTTTGT |
| | CCTGTGCAGCTTCTGGCTTCGACCTCACTGGTTCTTACATGCACTGGGTGCGTCAGGCCCCGGGTAAGGGCCTGG |
| | AATGGGTTGCAGGTATTTCCGCTTCTGGAGGCGCTACTGCTTATGCCGATAGCGTCAAGGGCCGTTTCACTATAA |
| | GCGCAGACACATCCAAAAACACAGCCTACCTACAAATGAACAGCTTAAGAGCTGAGGACACTGCCGTCTATTATT |
| | GTGCTCGCTCTCGTTCTTCTTCTTACTCTTCTTCTGGTTGGCGTTACTACTCTGGTGCTTTTGACTACTGGGGTCAA |
| | GGAACCCTGGTCACCGTCTCCTCGGCTAGCACCAAGGGCCCATCGGTCTTCCCCCTGGCACCCTCCTCCAAGAGC |
| | ACCTCTGGGGGCACAGCGGCCCTGGGCTGCCTGGTCAAGGACTACTTCCCCGAACCGGTGACGGTGTCGTGGA |
| | ACTCAGGCGCCCTGACCAGCGGCGTGCACACCTTCCCGGCTGTCCTACAGTCCTCAGGACTCTACTCCCTCAGCA |
| | GCGTGGTGACCGTGCCCTCCAGCAGCTTGGGCACCCAGACCTACATCTGCAACGTGAATCACAAGCCCAGCAAC |
| | ACCAAGGTGGACAAGAAAGTTGAGCCCAAATCTTGTGACAAAACTCACACATGCCCACCGTGCCCAGCACCTGA |
| | ACTCCTGGGGGGACCGTCAGTCTTCCTCTTCCCCCCAAAACCCAAGGACACCCTCATGATCTCCCGGACCCCTGA |
| | GGTCACATGCGTGGTGGTGGACGTGAGCCACGAAGACCCTGAGGTCAAGTTCAACTGGTACGTGGACGGCGTG |
| | GAGGTGCATAATGCCAAGACAAAGCCGCGGGAGGAGCAGTACAACAGCACGTACCGTGTGGTCAGCGTCCTCA |
| | CCGTCCTGCACCAGGACTGGCTGAATGGCAAGGAGTACAAGTGCAAGGTCTCCAACAAAGCCCTCCCAGCCCCC |
| | ATCGAGAAAACCATCTCCAAAGCCAAAGGGCAGCCCCGAGAACCACAGGTGTACACCCTGCCCCCATCCCGGGA |
| | GGAGATGACCAAGAACCAGGTCAGCCTGACCTGCCTGGTCAAAGGCTTCTATCCCAGCGACATCGCCGTGGAGT |
| | GGGAGAGCAATGGGCAGCCGGAGAACAACTACAAGACCACGCCTCCCGTGCTGGACTCCGACGGCTCCTTCTTC |
| | CTCTACAGCAAGCTCACCGTGGACAAGAGCAGGTGGCAGCAGGGGAACGTCTTCTCATGCTCCGTGATGCATGA |
| | GGCTCTGCACAACCACTACACGCAGAAGAGCCTCTCCCTGTCTCCGGGTAAA |
| VHH-VHH-hIgG1 | EVQLVESGGGLVQPGRSLRLSCAASGFDLTGSYMHWVRRAPGKGEELVAGISASGGATAYADSVKGRFTISADTSKN |
| AA | TAYLQMNSERAEDTAVYYCARSRSSSYSSSGWRYYSGAFDYWGQGTLVTVSSGGGGSGGGGSEVQLVESGGGLVQ |
| | PGRSLRLSCAASGFDLTGSYMHWVRRAPGKGEELVAGISASGGATAYADSVKGRFTISADTSKNTAYLQMNSLRAED |
| | TAVYYCARSRSSSYSSSGWRYYSGAFDYWGQGTLVTVSSGGGGSGGGGSEVQLVESGGGLVQPGGSLRLSCAASGF |
| | DLTGSYMHWVRQAPGKGLEWVAGISASGGATAYADSVKGRFTISADTSKNTAYLQMNSLRAEDTAVYYCARSRSSS |
| | YSSSGWRYYSGAFDYWGQGTLVTVSSASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPKSCDKTHTCPPCPAPELLGGPSVFLFPPKPK |
| | DTLMISRTPEVTCVVVDVSHEDPEVKFNWYVDGVEVHNAKTKPREEQYNSTYRVVSVLTVLHQDWLNGKEYKCKVS |
| | NKALPAPIEKTISKAKGQPREPQVYTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK |
| 32. LEAD scFv-hIgG1 clone example | |
| 15031 hIgG1 | |
| VL-CL NT | GATATCCAGATGACCCAGTCCCCGAGCTCCCTGTCCGCCTCTGTGGGCGATAGGGTCACCATCACCTGCCGTGCC |
| | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL AA | DIQMTQSPSSLSASVGDRVTITCRASQSVSSAVAWYQQKPGKAPKLLYSASDLYSGVPSRFSGSRSGTDFTLTISSLQP |
| | EDFATYYCQQGSYLFTFGQGTKVEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGEC |
| VL-VH-hIgG1 | GATATCCAGATGACCCAGTCCCCGAGCTCCCTGTCCGCCTCTGTGGGCGATAGGGTCACCATCACCTGCCGTGCC |
| NT | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAAGGTTACTACTGCCGCTAGTGGTAGTAGTGGTGGCAGTAGCAGTGGTGCCGAGGTTCA |
| | GCTGGTGGAGTCTGGCGGTGGCCTGGTGCAGCCAGGGGGCTCACTCCGTTTGTCCTGTGCAGCTTCTGGCTTCG |
| | ACCTCACTGGTTCTTACATGCACTGGGTGCGTCAGGCCCCGGGTAAGGGCCTGGAATGGGTTGCAGGTATTTCC |
| | GCTTCTGGAGGCGCTACTGCTTATGCCGATAGCGTCAAGGGCCGTTTCACTATAAGCGCAGACACATCCAAAAA |
| | CACAGCCTACCTACAAATGAACAGCTTAAGAGCTGAGGACACTGCCGTCTATTATTGTGCTCGCTCTCGTTCTTCT |
| | TCTTACTCTTCTTCTGGTTGGCGTTACTACTCTGGTGCTTTTGACTACTGGGGTCAAGGAACCCTGGTCACCGTCT |
| | CCTCGGGTGGAGGTGGCTCCGGAGGAGGTGGCAGTGAGGTTCAGCTGGTGGAGTCTGGCGGTGGCCTGGTGC |
| | AGCCAGGGGGCTCACTCCGTTTGTCCTGTGCAGCTTCTGGCTTCGACCTCACTGGTTCTTACATGCACTGGGTGC |
| | GTCAGGCCCCGGGTAAGGGCCTGGAATGGGTTGCAGGTATTTCCGCTTCTGGAGGCGCTACTGCTTATGCCGAT |
| | AGCGTCAAGGGCCGTTTCACTATAAGCGCAGACACATCCAAAAACACAGCCTACCTACAAATGAACAGCTTAAG |
| | AGCTGAGGACACTGCCGTCTATTATTGTGCTCGCTCTCGTTCTTCTTCTTACTCTTCTTCTGGTTGGCGTTACTACT |
| | CTGGTGCTTTTGACTACTGGGGTCAAGGAACCCTGGTCACCGTCTCCTCGGCTAGCACCAAGGGCCCATCGGTCT |
| | TCCCCCTGGCACCCTCCTCCAAGAGCACCTCTGGGGGCACAGCGGCCCTGGGCTGCCTGGTCAAGGACTACTTCC |
| | CCGAACCGGTGACGGTGTCGTGGAACTCAGGCGCCCTGACCAGCGGCGTGCACACCTTCCCGGCTGTCCTACAG |
| | TCCTCAGGACTCTACTCCCTCAGCAGCGTGGTGACCGTGCCCTCCAGCAGCTTGGGCACCCAGACCTACATCTGC |
| | AACGTGAATCACAAGCCCAGCAACACCAAGGTGGACAAGAAAGTTGAGCCCAAATCTTGTGACAAAACTCACAC |
| | ATGCCCACCGTGCCCAGCACCTGAACTCCTGGGGGGACCGTCAGTCTTCCTCTTCCCCCCAAAACCCAAGGACAC |
| | CCTCATGATCTCCCGGACCCCTGAGGTCACATGCGTGGTGGTGGACGTGAGCCACGAAGACCCTGAGGTCAAGT |
| | TCAACTGGTACGTGGACGGCGTGGAGGTGCATAATGCCAAGACAAAGCCGCGGGAGGAGCAGTACAACAGCA |
| | CGTACCGTGTGGTCAGCGTCCTCACCGTCCTGCACCAGGACTGGCTGAATGGCAAGGAGTACAAGTGCAAGGTC |
| | TCCAACAAAGCCCTCCCAGCCCCCATCGAGAAAACCATCTCCAAAGCCAAAGGGCAGCCCCGAGAACCACAGGT |
| | GTACACCCTGCCCCCATCCCGGGAGGAGATGACCAAGAACCAGGTCAGCCTGACCTGCCTGGTCAAAGGCTTCT |
| | ATCCCAGCGACATCGCCGTGGAGTGGGAGAGCAATGGGCAGCCGGAGAACAACTACAAGACCACGCCTCCCGT |
| | GCTGGACTCCGACGGCTCCTTCTTCCTCTACAGCAAGCTCACCGTGGACAAGAGCAGGTGGCAGCAGGGGAACG |
| | TCTTCTCATGCTCCGTGATGCATGAGGCTCTGCACAACCACTACACGCAGAAGAGCCTCTCCCTGTCTCCGGGTA |
| | AA |
| VL-VH-hIgG1 | DIQMTQSPSSLSASVGORVTITCRASQSVSSAVAWYQQKPGKAPKLLIYSASDLYSGVPSRFSGSRSGTDFTLTISSLQP |
| AA | EDFATYYCQQGSYLFTFGQGTKVEIKGTTAASGSSGGSSSGAEVQLVESGGGLVQPGGSLRLSCAASGFDLTGSYMH |
| | WVRQAPGKGLEWVAGISASGGATAYADSVKGRFTISADTSKNTAYLQMNSLRAEDTAVYYCARSRSSSYSSSGWRY |
| | YSGAFDYWGQGTLVTVSSGGGGSGGGGSEVQLVESGGGLVQPGGSLRLSCAASGFDLTGSYMHWVRQAPGKGLE |
| | WVAGISASGGATAYADSVKGRFTISADTSKNTAYLQMNSLRAEDTAVYYCARSRSSSYSSSGWRYYSGAFDYWGQG |
| | TLVTVSSASTKGPSVFPLAPSSKSTSGGTAALGCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPELLGGPSVFLFPPKPKDTLMISRTPEVTCVVVDVSHE |
| | DPEVKFNWYVDGVEVHNAKTKPREEQYNSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPVLDSDGSFFLYSKITVDKSRWQQGNVF |
| | SCSVMHEALHNHYTQKSLSLSPGK |
| 33. LEAD scFv-scFv-hIgG1 clone example | |
| 15031 scFv-scFv-hIgG1 | |
| VL-CL NT | GATATCCAGATGACCCAGTCCCCGAGCTCCCTGTCCGCCTCTGTGGGCGATAGGGTCACCATCACCTGCCGTGCC |
| | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTLTISSLQP |
| | EDFATYYCQQGSYLFTFGQGTKVEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGEC |
| VL-VH-VL-VH- | GATATCCAGATGACCCAGTCCCCGAGCTCCCTGTCCGCCTCTGTGGGCGATAGGGTCACCATCACCTGCCGTGCC |
| hIgG1 NT | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAAGGTACTACTGCCGCTAGTGGTAGTAGTGGTGGCAGTAGCAGTGGTGCCGAGGTTCA |
| | GCTGGTGGAGTCTGGCGGTGGCCTGGTGCAGCCAGGGGGCTCACTCCGTTTGTCCTGTGCAGCTTCTGGCTTCG |
| | ACCTCACTGGTTCTTACATGCACTGGGTGCGTCAGGCCCCGGGTAAGGGCCTGGAATGGGTTGCAGGTATTTCC |
| | GCTTCTGGAGGCGCTACTGCTTATGCCGATAGCGTCAAGGGCCGTTTCACTATAAGCGCAGACACATCCAAAAA |
| | CACAGCCTACCTACAAATGAACAGCTTAAGAGCTGAGGACACTGCCGTCTATTATTGTGCTCGCTCTCGTTCTTCT |
| | TCTTACTCTTCTTCTGGTTGGCGTTACTACTCTGGTGCTTTTGACTACTGGGGTCAAGGAACCCTGGTCACCGTCT |
| | CCTCGGGTGGAGGTGGCTCCGGAGGAGGTGGCAGTGATATCCAGATGACCCAGTCCCCGAGCTCCCTGTCCGC |
| | CTCTGTGGGCGATAGGGTCACCATCACCTGCCGTGCCAGTCAGTCCGTGTCCAGCGCTGTAGCCTGGTATCAACA |
| | GAAACCAGGAAAAGCTCCGAAGCTTCTGATTTACTCGGCATCCGATCTCTACTCTGGAGTCCCTTCTCGCTTCTCT |
| | GGTAGCCGTTCCGGGACGGATTTCACTCTGACCATCAGCAGTCTGCAGCCGGAAGACTTCGCAACTTATTACTGT |
| | CAGCAAGGTTCTTACCTGTTCACGTTCGGACAGGGTACCAAGGTGGAGATCAAAGGTACTACTGCCGCTAGTGG |
| | TAGTAGTGGTGGCAGTAGCAGTGGTGCCGAGGTTCAGCTGGTGGAGTCTGGCGGTGGCCTGGTGCAGCCAGG |
| | GGGCTCACTCCGTTTGTCCTGTGCAGCTTCTGGCTTCGACCTCACTGGTTCTTACATGCACTGGGTGCGTCAGGCC |
| | TTGACTACTGGGGTCAAGGAACCCTGGTCACCGTCTCCTCGGGTGGAGGTGGCTCCGGAGGAGGTGGCAGTGA |
| | CCGGGTAAGGGCCTGGAATGGGTTGCAGGTATTTCCGCTTCTGGAGGCGCTACTGCTTATGCCGATAGCGTCAA |
| | GGGCCGTTTCACTATAAGCGCAGACACATCCAAAAACACAGCCTACCTACAAATGAACAGCTTAAGAGCTGAGG |
| | ACACTGCCGTCTATTATTGTGCTCGCTCTCGTTCTTCTTCTTACTCTTCTTCTGGTTGGCGTTACTACTCTGGTGCTT |
| | TTGACTACTGGGGTCAAGGAACCCTGGTCACCGTCTCCTCGGGTGGAGGTGGCTCCGGAGGAGGTGGCAGTGA |
| | GGTTCAGCTGGTGGAGTCTGGCGGTGGCCTGGTGCAGCCAGGGGGCTCCTCCGTTTGTCCTGTCAGCTTCTG |
| | GCTTCGACCTCACTGGTTCTTACATGCACTGGGTGCGTCAGGCCCCGGGTAAGGGCCTGGAATGGGTTGCAGGT |
| | ATTTCCGCTTCTGGAGGCGCTACTGCTTATGCCGATAGCGTCAAGGGCCGTTTCACTATAAGCGCAGACACATCC |
| | AAAAACACAGCCTACCTACAAATGAACAGCTTAAGAGCTGAGGACACTGCCGTCTATTATTGTGCTCGCTCTCGT |
| | TCTTCTTCTTACTCTTCTTCTGGTTGGCGTTACTACTCTGGTGCTTTTGACTACTGGGGTCAAGGAACCCTGGTCAC |
| | CGTCTCCTCGGCTAGCACCAAGGGCCCATCGGTCTTCCCCCTGGCACCCTCCTCCAAGAGCACCTCTGGGGGCAC |
| | AGCGGCCCTGGGCTGCCTGGTCAAGGACTACTTCCCCGAACCGGTGACGGTGTCGTGGAACTCAGGCGCCCTGA |
| | CCAGCGGCGTGCACACCTTCCCGGCTGTCCTACAGTCCTCAGGACTCTACTCCCTCAGCAGCGTGGTGACCGTGC |
| | CCTCCAGCAGCTTGGGCACCCAGACCTACATCTGCAACGTGAATCACAAGCCCAGCAACACCAAGGTGGACAAG |
| | AAAGTTGAGCCCAAATCTTGTGACAAAACTCACACATGCCCACCGTGCCCAGCACCTGAACTCCTGGGGGGACC |
| | GTCAGTCTTCCTCTTCCCCCCAAAACCCAAGGACACCCTCATGATCTCCCGGACCCCTGAGGTCACATGCGTGGT |
| | GGTGGACGTGAGCCACGAAGACCCTGAGGTCAAGTTCAACTGGTACGTGGACGGCGTGGAGGTGCATAATGCC |
| | AAGACAAAGCCGCGGGAGGAGCAGTACAACAGCACGTACCGTGTGGTCAGCGTCCTCACCGTCCTGCACCAGG |
| | ACTGGCTGAATGGCAAGGAGTACAAGTGCAAGGTCTCCAACAAAGCCCTCCCAGCCCCCATCGAGAAAACCATC |
| | TCCAAAGCCAAAGGGCAGCCCCGAGAACCACAGGTGTACACCCTGCCCCCATCCCGGGAGGAGATGACCAAGA |
| | ACCAGGTCAGCCTGACCTGCCTGGTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACGGCTCCTTCTTCCTCTACAGCAAGCT |
| | CACCGTGGACAAGAGCAGGTGGCAGCAGGGGAACGTCTTCTCATGCTCCGTGATGCATGAGGCTCTGCACAACC |
| | ACTACACGCAGAAGAGCCTCTCCCTGTCTCCGGGTAAA |
| VL-VH-VL-VH- | DIQMTQSPSSLSASVGDRVTITCRASQSVSSAVAWYQQKPGKAPKLLIYSASDLYSGVPSRFSGSRSGTDFTLTISSLQP |
| hIgG1 AA | EDFATYYCQQGSYLFTFGQGTKVEIKGTTAASGSSGGSSSGAEVQLVESGGGLVQPGGSLRLSCAASGFDLTGSYMH |
| | WVRQAPGKGLEWVAGISASGGATAYADSVKGRFTISADTSKNTAYLQMNSLRAEDTAVYYCARSRSSSYSSSGWRY |
| | YSGAFDYWGQGTLVTVSSGGGGSGGGGSDIQMTQSPSSLSASVGDRVTITCRASQSVSSAVAWYQQKPGKAPKLLI |
| | YSASDLYSGVPSRPSGSRSGTDFTLTISSLQPEDFATYYCQQGSYLFTFGQGTKVEIKGTTAASGSSGGSSSGAEVQLVE |
| | SGGGLVQPGGSLRLSCAASGFDLTGSYMHWVRQAPGKGLEWVAGISASGGATAYADSVKGRFTISADTSKNTAYLQ |
| | MNSLRAEDTAVYYCARSRSSSYSSSGWRYYSGAFDYWGQGTLVTVSSGGGGSGGGGSEVOLVESGGGLVQPGGSL |
| | RLSCAASGFDLTGSYMHWVRQAPGKGLEWVAGISASGGATAYADSVKGRFTISADTSKNTAYLQMNSLRAEDTAVY |
| | YCARSRSSSYSSSGWRYYSGAFDYWGQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKVEPKSCDKTHTCPPCPAPELLGGPS |
| | VFLFPPKPKDTLMISRTPEVTCVVVDVSHEDPEVKFNWYVDGVEVHNAKTKPREEQYNSTYRVVSVLTVLHQDWLN |
| | GKEYKCKVSNKALPAPIEKTISKAKGQPREPQVYTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKSLSLSPGK |
| 34. LEAD Fab-hIgG1 clone example | |
| 15031 Fab-hIgG1 | |
| VL-CL NT | GATATCCAGATGACCCAGTCCCCGAGCTCCCTGTCCGCCTCTGTGGGCGATAGGGTCACCATCACCTGCCGTGCC |
| | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTLTISSLQP |
| | EDFATYYCQQGSYLFTFGQGTKVEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGEC |
| VH-CH1-hIg1 | GAGGTTCAGCTGGTGGAGTCTGGCGGTGGCCTGGTGCAGCCAGGGGGCTCACTCCGTTTGTCCTGTGCAGCTTC |
| NT | TGGCTTCGATCTCACTGGTTCTTACATGCACTGGGTGCGTCAGGCCCCGGGTAAGGGCCTGGAATGGGTTGCAG |
| | GTATTTCCGCTTCTGGAGGCGCTACTGCTTATGCCGATAGCGTCAAGGGCCGTTTCACTATAAGCGCAGACACAT |
| | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGSTTGGCGTTACTACTCTGGTGCTTTTGACTACTGGGGTCAAGGAACCCTGGTC |
| | ACCGTCTCCTCGGCTAGCACCAAGGGCCCATOGGTCTTCCCCCTGGCACCCTCCTCCAAGAGCACCTCTGGGGGC |
| | ACAGCGGCCCTGGGCTGCCTGGTCAAGGACTACTTCCCCGAACCGGTGACGGTGTCGTGGAACTCAGGCGCCCT |
| | GACCAGCGGCGTGCACACCTTCCCGGCTGTCCTACAGTCCTCAGGACTCTACTCCCTCAGCAGCGTGGTGACCGT |
| | GCCCTCCAGCAGCTTGGGCACCCAGACCTACATCTGCAACGTGAATCACAAGCCCAGCAACACCAAGGTGGACA |
| | AGAAAGTTGAGCCCAAATCTTGTGACAAAACTCACACATCCGGAGGAGGTGGCAGTGGCGGAGGTGGCAGCG |
| | GTGGAGGTGGCAGTGAGGTTCAGCTGGTGGAGTCTGGCGGTGGCCTGGTGCAGCCAGGGGGCTCACTCCGTTT |
| | GTCCTGTGCAGCTTCTGGCTTCGATCTCACTGGTTCTTACATGCACTGGGTGCGTCAGGCCCCGGGTAAGGGCCT |
| | GGAATGGGTTGCAGGTATTTCCGCTTCTGGAGGCGCTACTGCTTATGCCGATAGCGTCAAGGGCCGTTTCACTAT |
| | AAGCGCAGACACATCCAAAAACACAGCCTACCTACAAATGAACAGCTTAAGAGCTGAGGACACTGCCGTCTATT |
| | ATTGTGCTCGCTCTCGTTCTTCTTCTTACTCTTCTTCTGGTTGGCGTTACTACTCTGGTGCTTTTGACTACTGGGGT |
| | CAAGGAACCCTGGTCACCGTCTCCTCGGCTAGCACCAAGGGCCCATCGGTCTTCCCCCTGGCACCCTCCTCCAAG |
| | AGCACCTCTGGGGGCACAGCGGCCCTGGGCTGCCTGGTCAAGGACTACTTCCCCGAACCGGTGACGGTGTCGTG |
| | GAACTCAGGCGCCCTGACCAGCGGCGTGCACACCTTCCCGGCTGTCCTACAGTCCTCAGGACTCTACTCCCTCAG |
| | CAGCGTGGTGACCGTGCCCTCCAGCAGCTTGGGCACCCAGACCTACATCTGCAACGTGAATCACAAGCCCAGCA |
| | ACACCAAGGTGGACAAGAAAGTTGAGCCCAAATCTTGTGACAAAACTCACACATGCCCACCGTGCCCAGCACCT |
| | GAACTCCTGGGGGGACCGTCAGTCTTCCTCTTCCCCCCAAAACCCAAGGACACCCTCATGATCTCCCGGACCCCT |
| | GAGGTCACATGCGTGGTGGTGGACGTGAGCCACGAAGACCCTGAGGTCAAGTTCAACTGGTACGTGGACGGCG |
| | TGGAGGTGCATAATGCCAAGACAAAGCCGCGGGAGGAGCAGTACAACAGCACGTACCGTGTGGTCAGCGTCCT |
| | CACCGTCCTGCACCAGGACTGGCTGAATGGCAAGGAGTACAAGTGCAAGGTCTCCAACAAAGCCCTOCCAGCCC |
| | CCATCGAGAAAACCATCTCCAAAGCCAAAGGGCAGCCCCGAGAACCACAGGTGTACACCCTGCCCCCATCCCGG |
| | GAGGAGATGACCAAGAACCAGGTCAGCCTGACCTGCCTGGTCAAAGGCTTCTATCCCAGCGACATCGCCGTGGA |
| | GTGGGAGAGCAATGGGCAGCCGGAGAACAACTACAAGACCACGCCTCCCGTGCTGGACTCCGACGGCTCCTTCT |
| | TCCTCTACAGCAAGCTCACCGTGGACAAGAGCAGGTGGCAGCAGGGGAACGTCTTCTCATGCTCCGTGATGCAT |
| | GAGGCTCTGCACAACCACTACACGCAGAAGAGCCTCTCCCTGTCTCCGGGTAAA |
| VH-CHI-hIg1 | EVQLVESGGGLVQPGGSLRLSCAASGFDLTGSYMHWVRQAPGKGLEWVAGISASGGATAYADSVKGRFTISADTSK |
| AA | NTAYLQMNSLRAEDTAVYYCARSRSSSYSSSGWRYYSGAFDYWGQGTLVTVSSASTKGPSVFPLAPSSKSTSGGTAAL |
| | GCLVKDYFPEPVTVSWNSGALTSGVHTFPAVLQSSGLYSLSSVVTVPSSSIGTQTYICNVNHKPSNTKVDKKVEPKSCD |
| | KTHTSGGGGSGGGGSGGGGSEVOLVESGGGLVQPGGSLRLSCAASGFDLTGSYMHWVRQAPGKGLEWVAGISAS |
| | GGATAYADSVKGRFTISADTSKNTAYLQMNSLRAEDTAVYYCARSRSSSYSSSGWRYYSGAFDYWGQGTLVTVSSAS |
| | TKGPSVFPLAPSSKSTSGGTAALGCLVKDYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKSRWQQGNVFSCSVMHEA |
| | LHNHYTQKSLSLSPGK |
| 35. LEAD Fab-Fab-hIgG1 clone example | |
| 15031 Fab-Fab-hIgG1 | |
| VL-CL NT | GATATCCAGATGACCCAGTCCCCGAGCTCCCTGTCCGCCTCTGTGGGCGATAGGGTCACCATCACCTGCCGTGCC |
| | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTLTISSLQP |
| | EDFATYYCQQGSYLFTFGQGTKVEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGEC |
| VH-CH1-VH- | GAGGTTCAGCTGGTGGAGTCTGGCGGTGGCCTGGTGCAGCCAGGGGGCTCACTCCGTTTGTCCTGTGCAGCTTC |
| CH1-hIgG1 NT | TGGCTTCGACCTCACTGGTTCTTACATGCACTGGGTGCGTCAGGCCCCGGGTAAGGGCCTGGAATGGGTTGCAG |
| | GTATTTCCGCTTCTGGAGGCGCTACTGCTTATGCCGATAGCGTCAAGGGCCGTTTCACTATAAGCGCAGACACAT |
| | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGGTTGGCGTTACTACTCTGGTGCTTTTGACTACTGGGGTCAAGGAACCCTGGTC |
| | ACCGTCTCCTCGGCTAGCACCAAGGGCCCATCGGTCTTCCCCCTGGCACCCTCCTCCAAGAGCACCTCTGGGGGC |
| | ACAGCGGCCCTGGGCTGCCTGGTCAAGGACTACTTCCCCGAACCGGTGACGGTGTCGTGGAACTCAGGCGCCCT |
| | GACCAGCGGCGTGCACACCTTCCCGGCTGTCCTACAGTCCTCAGGACTCTACTCCCTCAGCAGCGTGGTGACCGT |
| | GCCCTCCAGCAGCTTGGGCACCCAGACCTACATCTGCAACGTGAATCACAAGCCCAGCAACACCAAGGTGGACA |
| | AGAAAGTTGAGCCCAAATCTTGTGACAAAACTCACACATCCGGAGGAGGTGGCAGTGGCGGAGGTGGCAGCG |
| | GTGGAGGTGGCAGTGAGGTTCAGCTGGTGGAGTCTGGCGGTGGCCTGGTGCAGCCAGGGGGCTCACTCCGTTT |
| | GTCCTGTGCAGCTTCTGGCTTCGACCTCACTGGTTCTTACATGCACTGGGTGCGTCAGGCCCCGGGTAAGGGCCT |
| | GGAATGGGTTGCAGGTATTTCCGCTTCTGGAGGCGCTACTGCTTATGCCGATAGCGTCAAGGGCCGTTTCACTAT |
| | AAGCGCAGACACATCCAAAAACACAGCCTACCTACAAATGAACAGCTTAAGAGCTGAGGACACTGCCGTCTATT |
| | ATTGTGCTCGCTCTCGTTCTTCTTCTTACTCTTCTTCTGGTTGGCGTTACTACTCTGGTGCTTTTGACTACTGGGGT |
| | CAAGGAACCCTGGTCACCGTCTCCTCGGCTAGCACCAAGGGCCCATCGGTCTTCCCCCTGGCACCCTCCTCCAAG |
| | AGCACCTCTGGGGGCACAGCGGCCCTGGGCTGCCTGGTCAAGGACTACTTCCCCGAACCGGTGACGGTGTCGTG |
| | GAACTCAGGCGCCCTGACCAGCGGCGTGCACACCTTCCCGGCTGTCCTACAGTCCTCAGGACTCTACTCCCTCAG |
| | CAGCGTGGTGACCGTGCCCTCCAGCAGCTTGGGCACCCAGACCTACATCTGCAACGTGAATCACAAGCCCAGCA |
| | ACACCAAGGTGGACAAGAAAGTTGAGCCCAAATCTTGTGACAAAACTCACACATCCGGAGGAGGTGGCAGTGG |
| | CGGAGGTGGCAGCGGTGGAGGTGGCAGTGAGGTTCAGCTGGTGGAGTCTGGCGGTGGCCTGGTGCAGCCAG |
| | GGGGCTCACTCCGTTTGTCCTGTGCAGCTTCTGGCTTCGACCTCACTGGTTCTTACATGCACTGGGTGCGTCAGG |
| | CCCCGGGTAAGGGCCTGGAATGGGTTGCAGGTATTTCCGCTTCTGGAGGCGCTACTGCTTATGCCGATAGCGTC |
| | AAGGGCCGTTTCACTATAAGCGCAGACACATCCAAAAACACAGCCTACCTACAAATGAACAGCTTAAGAGCTGA |
| | GGACACTGCCGTCTATTATTGTGCTCGCTCTCGTTCTTCTTCTTACTCTTCTTCTGGTTGGCGTTACTACTCTGGTG |
| | CTTTTGACTACTGGGGTCAAGGAACCCTGGTCACCGTCTCCTCGGCTAGCACCAAGGGCCCATCGGTCTTCCCCC |
| | TGGCACCCTCCTCCAAGAGCACCTCTGGGGGCACAGCGGCCCTGGGCTGCCTGGTCAAGGACTACTTCCCCGAA |
| | CCGGTGACGGTGTCGTGGAACTCAGGCGCCCTGACCAGCGGCGTGCACACCTTCCCGGCTGTCCTACAGTCCTC |
| | AGGACTCTACTCCCTCAGCAGCGTGGTGACCGTGCCCTCCAGCAGCTTGGGCACCCAGACCTACATCTGCAACGT |
| | GAATCACAAGCCCAGCAACACCAAGGTGGACAAGAAAGTTGAGCCCAAATCTTGTGACAAAACTCACACATGCC |
| | CACCGTGCCCAGCACCTGAACTCCTGGGGGGACCGTCAGTCTTCCTCTTCCCCCCAAAACCCAAGGACACCCTCA |
| | TGATCTCCCGGACCCCTGAGGTCACATGCGTGGTGGTGGACGTGAGCCACGAAGACCCTGAGGTCAAGTTCAAC |
| | TGGTACGTGGACGGCGTGGAGGTGCATAATGCCAAGACAAAGCCGCGGGAGGAGCAGTACAACAGCACGTAC |
| | CGTGTGGTCAGCGTCCTCACCGTCCTGCACCAGGACTGGCTGAATGGCAAGGAGTACAAGTGCAAGGTCTCCAA |
| | CAAAGCCCTCCCAGCCCCCATCGAGAAAACCATCTCCAAAGCCAAAGGGCAGCCCCGAGAACCACAGGTGTACA |
| | CCCTGCCCCCATCCCGGGAGGAGATGACCAAGAACCAGGTCAGCCTGACCTGCCTGGTCAAAGGCTTCTATCCC |
| | AGCGACATCGCCGTGGAGTGGGAGAGCAATGGGCAGCCGGAGAACAACTACAAGACCACGCCTCCCGTGCTGG |
| | ACTCCGACGGCTCCTTCTTCCTCTACAGCAAGCTCACCGTGGACAAGAGCAGGTGGCAGCAGGGGAACGTCTTC |
| | TCATGCTCCGTGATGCATGAGGCTCTGCACAACCACTACACGCAGAAGAGCCTCTCCCTGTCTCCGGGTAAA |
| VH-CH1-VH- | EVQLVESGGGLVQPGGSLRLSCAASGFDLTGSYMHWVRQAPGKGLEWVAGISASGGATAYADSVKGRFTISADTSK |
| CH1- | NTAYLQMNSLRAEDTAVYYCARSRSSSYSSSGWRYYSGAFDYWGQGTLVTVSSASTKGPSVFPLAPSSKSTSGGTAAL |
| hIgG1 AA | GCLVKDYFPEPVTVSWNSGALTSGVHTFPAVLQSSGLYSLSSVVTVPSSSLGTQTYICNVNHKPSNTKVDKKVEPKSCD |
| | KTHTSGGGGSGGGGSGGGGSEVQLVESGGGLVQPGGSLRLSCAASGFDTGSYMHWVRQAPGKGLEWVAGISAS |
| | GGATAYADSVKGRFTISADTSKNTAYLQMNSLRAEDTAVYYCARSRSSSYSSSGWRYYSGAFDYWGQGTLVTVSSAS |
| | TKGPSVFPLAPSSKSTSGGTAALGCLVKDYFPEPVTVSWNSGALTSGVHTFPAVLQSSGLYSLSSVVTVPSSSLGTQTYI |
| | CNVNHKPSNTKVDKKVEPKSCDKTHTSGGGGSGGGGSGGGGSEVQLVESGGGLVQPGGSLRLSCAASGFDLTGSY |
| | MHWVRQAPGKGLEWVAGISASGGATAYADSVKGRFTISADTSKNTAYLQMNSLRAEDTAVYYCARSRSSSYSSSGW |
| | RYYSGAFDYWGQGTLVTVSSASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLTCLVKGFYPSDIAVEWESNGQPENNYKTTPPVLDSDGSFFLYSKLT |
| | VDKSRWQQGNVFSCSVMHEALHNHYTQKSISLSPGK |
| 36. LEAD hIgG1-VHH clone example | |
| 15031 hIgG1-VHH | |
| VL-CL NT | GATATCCAGATGACCCAGTCCCCGAGCTCCCTGTCCGCCTCTGTGGGCGATAGGGTCACCATCACCTGCCGTGCC |
| | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL AA | DIQMTQSPSSLSASVGDRVTITCRASQSVSSAVAWYQQKPCKAPKLLIYSASDLYSGVPSRFSGSRSGTDFTLTISSLQP |
| | EDFATYYCQQGSYLFTFGQGTKVEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGEC |
| hIgG1-VHH | GAGGTTCAGCTGGTGGAGTCTGGCGGTGGCCTGGTGCAGCCAGGGGGCTCACTCCGTTTGTCCTGTGCAGCTTC |
| NT | TGGCTTCGACCTCACTGGTTCTTACATGCACTGGGTGCGTCAGGCCCCGGGTAAGGGCCTGGAATGGGTTGCAG |
| | GTATTTCCGCTTCTGGAGGCGCTACTGCTTATGCCGATAGCGTCAAGGGCCGTTTCACTATAAGCGCAGACACAT |
| | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGGTTGGCGTTACTACTCTGGTGCTTTTGACTACTGGGGTCAAGGAACCCTGGTC |
| | ACCGTCTCCTCGGCTAGCACCAAGGGCCCATCGGTCTTCCCCCTGGCACCCTCCTCCAAGAGCACCTCTGGGGGC |
| | ACAGCGGCCCTGGGCTGCCTGGTCAAGGACTACTTCCCCGAACCGGTGACGGTGTCGTGGAACTCAGGCGCCCT |
| | GACCAGCGGCGTGCACACCTTCCCGGCTGTCCTACAGTCCTCAGGACTCTACTCCCTCAGCAGCGTGGTGACCGT |
| | GCCCTCCAGCAGCTTGGGCACCCAGACCTACATCTGCAACGTGAATCACAAGCCCAGCAACACCAAGGTGGACA |
| | AGAAAGTTGAGCCCAAATCTTGTGACAAAACTCACACATGCCCACCGTGCCCAGCACCTGAACTCCTGGGGGGA |
| | CCGTCAGTCTTCCTCTTCCCCCCAAAACCCAAGGACACCCTCATGATCTCCCGGACCCCTGAGGTCACATGCGTGG |
| | TGGTGGACGTGAGCCACGAAGACCCTGAGGTCAAGTTCAACTGGTACGTGGACGGCGTGGAGGTGCATAATGC |
| | CAAGACAAAGCCGCGGGAGGAGCAGTACAACAGCACGTACCGTGTGGTCAGCGTCCTCACCGTCCTGCACCAG |
| | GACTGGCTGAATGGCAAGGAGTACAAGTGCAAGGTCTCCAACAAAGCCCTCCCAGCCCCCATCGAGAAAACCAT |
| | CTCCAAAGCCAAAGGGCAGCCCCGAGAACCACAGGTGTACACCCTGCCCCCATCCCGGGAGGAGATGACCAAG |
| | AACCAGGTCAGCCTGACCTGCCTGGTCAAAGGCTTCTATCCCAGCGACATCGCCGTGGAGTGGGAGAGCAATGG |
| | GCAGCCGGAGAACAACTACAAGACCACGCCTCCCGTGCTGGACTOCGACGGCTCCTTCTTCCTCTACAGCAAGCT |
| | CACCGTGGACAAGAGCAGGTGGCAGCAGGGGAACGTCTTCTCATGCTCCGTGATGCATGAGGCTCTGCACAACC |
| | ACTACACGCAGAAGAGCCTCTCCCTGTCTCCGGGTGGAGGAGGTGGGGGATCAGGTGGTGGGGGCTCTGGAG |
| | GCGGAACCGGTGAGGTTCAGCTGGTGGAGTCTGGCGGTGGCCTGGTGCAGCCAGGGCGTTCACTCCGTTTGTC |
| | CTGTGCAGCTTCTGGCTTCGACCTCACTGGTTCTTACATGCACTGGGTGCGTCGTGCCCCGGGTAAGGGCGAGG |
| | AACTGGTTGCAGGTATTTCCGCTTCTGGAGGCGCTACTGCTTATGCCGATAGCGTCAAGGGCCGTTTCACTATAA |
| | GCGCAGACACATCCAAAAACACAGCCTACCTACAAATGAACAGCTTAAGAGCTGAGGACACTGCCGTCTATTATT |
| | GTGCTCGCTCTCGTTCTTCTTCTTACTCTTCTTCTGGTTGGCGTTACTACTCTGGTGCTTTTGACTACTGGGGTCAA |
| | GGAACCCTGGTCACCGTCTCCTCG |
| hIgG1-VHH | EVQLVESGGGLVQPGGSLRLSCAASGFDLTGSYMHWVRQAPGKGLEWVAGISASGGATAYADSVKGRFTISADISK |
| AA | NTAYLQMNSLRAEDTAVYYCARSRSSSYSSSGWRYYSGAFDYWGQGTLVTVSSASTKGPSVFPLAPSSKSTSGGTAAL |
| | GCLVKDYFPEPVTVSWNSGALTSGVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKENWYVDGVEVHNAKTKPREEQYNST |
| | YRVVSVLTVLHQDWLNGKEYKCKVSNKALPAPIEKTISKAKGQPREPQVYTLPPSREEMTKNQVSLTCLVKGFYPSDIA |
| | VEWESNGQPENNYKTTPPVLDSDGSFFLYSKLTVDKSRWQQGNVFSCSVMHEALHNHYTQKSLSLSPGGGGGGSG |
| | GGGSGGGTGEVQLVESGGGLVQPGRSLRLSCAASGFDLTGSYMHWVRRAPGKGEELVAGISASGGATAYADSVKG |
| | RFTISADTSKNTAYLQMNSLRAEDTAVYYCARSRSSSYSSSGWRYYSGAFDYWGQGTLVTVSS |
| 37. LEAD hIgG1-VHH-VHH clone example | |
| 15031 hIgG1-VHH-VHH | |
| VL-CL NT | GATATCCAGATGACCCAGTCCCCGAGCTCCCTGTCCGCCTCTGTGGGCGATAGGGTCACCATCACCTGCCGTGCC |
| | AGTCAGTCCGTGTCCAGCGCTGTAGCCTGGTATCAACAGAAACCAGGAAAAGCTCCGAAGCTTCTGATTTACTTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTLTISSLQP |
| | EDFATYYCQQGSYLFTFGQGTKVEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGEC |
| hIgG1-VHH-VHH | GAGGTTCAGCTGGTGGAGTCTGGCGGTGGCCTGGTGCAGCCAGGGGGCTCACTCCGTTTGTCCTGTGCAGCTTC |
| NT | TGGCTTCGACCTCACTGGTTCTTACATGCACTGGGTGCGTCAGGCCCCGGGTAAGGGCCTGGAATGGGTTGCAG |
| | GTATTTCCGCTTCTGGAGGCGCTACTGCTTATGCCGATAGCGTCAAGGGCCGTTTCACTATAAGCGCAGACACAT |
| | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGGTTGGCGTTACTACTCTGGTGCTTTTGACTACTGGGGTCAAGGAACCCTGGTC |
| | ACCGTCTCCTCGGCTAGCACCAAGGGCCCATCGGTCTTCCCCCTGGCACCCTCCTCCAAGAGCACCTCTGGGGGC |
| | ACAGCGGCCCTGGGCTGCCTGGTCAAGGACTACTTCCCCGAACCGGTGACGGTGTCGTGGAACTCAGGCGCCCT |
| | GACCAGCGGCGTGCACACCTTCCCGGCTGTCCTACAGTCCTCAGGACTCTACTCCCTCAGCAGCGTGGTGACCGT |
| | GCCCTCCAGCAGCTTGGGCACCCAGACCTACATCTGCAACGTGAATCACAAGCCCAGCAACACCAAGGTGGACA |
| | AGAAAGTTGAGCCCAAATCTTGTGACAAAACTCACACATGCCCACCGTGCCCAGCACCTGAACTCCTGGGGGGA |
| | CCGTCAGTCTTCCTCTTCCCCCCAAAACCCAAGGACACCCTCATGATCTCCCGGACCCCTGAGGTCACATGCGTGG |
| | TGGTGGACGTGAGCCACGAAGACCCTGAGGTCAAGTTCAACTGGTACGTGGACGGCGTGGAGGTGCATAATGC |
| | CAAGACAAAGCCGCGGGAGGAGCAGTACAACAGCACGTACCGTGTGGTCAGCGTCCTCACCGTCCTGCACCAG |
| | GACTGGCTGAATGGCAAGGAGTACAAGTGCAAGGTCTCCAACAAAGCCCTCCCAGCCCCCATCGAGAAAACCAT |
| | CTCCAAAGCCAAAGGGCAGCCCCGAGAACCACAGGTGTACACCCTGCCCCCATCCCGGGAGGAGATGACCAAG |
| | AACCAGGTCAGCCTGACCTGCCTGGTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACGGCTCCTTCTTCCTCTACAGCAAGCT |
| | CACCGTGGACAAGAGCAGGTGGCAGCAGGGGAACGTCTTCTCATGCTCCGTGATGCATGAGGCTCTGCACAACC |
| | ACTACACGCAGAAGAGCCTCTCCCTGTCTCCGGGTGGAGGAGGTGGGGGATCAGGTGGTGGGGGCTCTGGAG |
| | GCGGAACCGGTGAGGTTCAGCTGGTGGAGTCTGGCGGTGGCCTGGTGCAGCCAGGGCGTTCACTCCGTTTGTC |
| | CTGTGCAGCTTCTGGCTTCGACCTCACTGGTTCTTACATGCACTGGGTGCGTCGTGCCCCGGGTAAGGGCGAGG |
| | AACTGGTTGCAGGTATTTCCGCTTCTGGAGGCGCTACTGCTTATGCCGATAGCGTCAAGGGCCGTTTCACTATAA |
| | GCGCAGACACATCCAAAAACACAGCCTACCTACAAATGAACAGCTTAAGAGCTGAGGACACTGCCGTCTATTATT |
| | GTGCTCGCTCTCGTTCTTCTTCTTACTCTTCTTCTGGTTGGCGTTACTACTCTGGTGCTTTTGACTACTGGGGTCAA |
| | GGAACCCTGGTCACCGTCTCCTCGGGTGGAGGTGGCTCCGGAGGAGGTGGCAGTGAGGTTCAGCTGGTGGAG |
| | TCTGGCGGTGGCCTGGTGCAGCCAGGGCGTTCACTCCGTTTGTCCTGTGCAGCTTCTGGCTTCGACCTCACTGGT |
| | TCTTACATGCACTGGGTGCGTCGTGCCCCGGGTAAGGGCGAGGAACTGGTTGCAGGTATTTCCGCTTCTGGAGG |
| | CGCTACTGCTTATGCCGATAGCGTCAAGGGCCGTTTCACTATAAGCGCAGACACATCCAAAAACACAGCCTACCT |
| | ACAAATGAACAGCTTAAGAGCTGAGGACACTGCCGTCTATTATTGTGCTCGCTCTCGTTCTTCTTCTTACTCTTCTT |
| | CTGGTTGGCGTTACTACTCTGGTGCTTTTGACTACTGGGGTCAAGGAACCCTGGTCACCGTCTCCTCG |
| hIgG1-VHH- | EVQLVESGGGLVQPGGSLRLSCAASGFDLTGSYMHWVRQAPGKGLEWVAGISASGGATAYADSVKGRFTISADTSK |
| VHH AA | NTAYLQMNSLRAEDTAVYYCARSRSSSYSSSGWRYYSGAFDYWGQGTLVTVSSASTKGPSVEPLAPSSKSTSGGTAAL |
| | GCLVKDYFPEPVTVSWNSGALTSGVHTFPAVLQSSGLYSLSSVVTVPSSSIGTQTYICNVNHKPSNTKVDKKVEPKSCD |
| | KTHTCPPCPAPELLGGPSVFLFPPKPKDTLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVYTLPPSREEMTKNQVSLTCLVKGFYPSDIA |
| | VEWESNGQPENNYKTTPPVLDSDGSFFLYSKLTVDKSRWQQGNVFSCSVMHEALHNHYTQKSLSLSPGGGGGGSG |
| | GGGSGGGTGEVQLVESGGGLVQPGRSLRLSCAASGFDLTGSYMHWVRRAPGKGEELVAGISASGGATAYADSVKG |
| | RFTISADTSKNTAYLQMNSLRAEDTAVYYCARSRSSSYSSSGWRYYSGAFDYWGQGTLVTVSSGGGGSGGGGSEVQ |
| | LVESGGGLVQPGRSLRLSCAASGFDLTGSYMHWVRRAPGKGEELVAGISASGGATAYADSVKGRFTISADTSKNTAYL |
| | QMNSLRAEDTAVYYCARSRSSSYSSSGWRYYSGAFDYWGQGTLVTVSS |
| 38. LEAD hIgG1-scFv clone example | |
| 15031 hIgG1-scFv | |
| VL-CL NT | GATATCCAGATGACCCAGTCCCCGAGCTCCCTGTCCGCCTCTGTGGGCGATAGGGTCACCATCACCTGCCGTGCC |
| | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTLTISSLQP |
| | EDFATYYCQQGSYLFTFGQGTKVEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGEC |
| hIgG1-VL-VH | GAGGTTCAGCTGGTGGAGTCTGGCGGTGGCCTGGTGCAGCCAGGGGGCTCACTCCGTTTGTCCTGTGCAGCTTC |
| NT | TGGCTTCGACCTCACTGGTTCTTACATGCACTGGGTGCGTCAGGCCCCGGGTAAGGGCCTGGAATGGGTTGCAG |
| | GTATTTCCGCTTCTGGAGGCGCTACTGCTTATGCCGATAGCGTCAAGGGCCGTTTCACTATAAGCGCAGACACAT |
| | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGGTTGGCGTTACTACTCTGGTGCTTTTGACTACTGGGGTCAAGGAACCCTGGTC |
| | ACCGTCTCCTCGGCTAGCACCAAGGGCCCATCGGTCTTCCCCCTGGCACCCTCCTCCAAGAGCACCTCTGGGGGC |
| | ACAGCGGCCCTGGGCTGCCTGGTCAAGGACTACTTCCCCGAACCGGTGACGGTGTCGTGGAACTCAGGCGCCCT |
| | GACCAGCGGCGTGCACACCTTCCCGGCTGTCCTACAGTCCTCAGGACTCTACTCCCTCAGCAGCGTGGTGACCGT |
| | GCCCTCCAGCAGCTTGGGCACCCAGACCTACATCTGCAACGTGAATCACAAGCCCAGCAACACCAAGGTGGACA |
| | AGAAAGTTGAGCCCAAATCTTGTGACAAAACTCACACATGCCCACCGTGCCCAGCACCTGAACTCCTGGGGGGA |
| | CCGTCAGTCTTCCTCTTCCCCCCAAAACCCAAGGACACCCTCATGATCTCCCGGACCCCTGAGGTCACATGCGTGG |
| | TGGTGGACGTGAGCCACGAAGACCCTGAGGTCAAGTTCAACTGGTACGTGGACGGCGTGGAGGTGCATAATGC |
| | CAAGACAAAGCCGCGGGAGGAGCAGTACAACAGCACGTACCGTGTGGTCAGCGTCCTCACCGTCCTGCACCAG |
| | GACTGGCTGAATGGCAAGGAGTACAAGTGCAAGGTCTCCAACAAAGCCCTCCCAGCCCCCATCGAGAAAACCAT |
| | CTCCAAAGCCAAAGGGCAGCCCCGAGAACCACAGGTGTACACCCTGCCCCCATCCCGGGAGGAGATGACCAAG |
| | AACCAGGTCAGCCTGACCTGCCTGGTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACGGCTCCTTCTTCCTCTACAGCAAGCT |
| | CACCGTGGACAAGAGCAGGTGGCAGCAGGGGAACGTCTTCTCATGCTCCGTGATGCATGAGGCTCTGCACAACC |
| | ACTACACGCAGAAGAGCCTCTCCCTGTCTCCGGGTGGAGGAGGTGGGGGATCAGGTGGTGGGGGCTCTGGAG |
| | GCGGAACCGGTGATATCCAGATGACCCAGTCCCCGAGCTCCCTGTCCGCCTCTGTGGGCGATAGGGTCACCATC |
| | ACCTGCCGTGCCAGTCAGTCCGTGTCCAGCGCTGTAGCCTGGTATCAACAGAAACCAGGAAAAGCTCCGAAGCT |
| | TCTGATTTACTCGGCATCCGATCTCTACTCTGGAGTCCCTTCTCGCTTCTCTGGTAGCCGTTCCGGGACGGATTTC |
| | ACTCTGACCATCAGCAGTCTGCAGCCGGAAGACTTCGCAACTTATTACTGTCAGCAAGGTTCTTACCTGTTCACGT |
| | TCGGACAGGGTACCAAGGTGGAGATCAAAGGTACTACTGCCGCTAGTGGTAGTAGTGGTGGCAGTAGCAGTG |
| | GTGCCGAGGTTCAGCTGGTGGAGTCTGGCGGTGGCCTGGTGCAGCCAGGGGGCTCACTCCGTTTGTCCTGTGCA |
| | GCTTCTGGCTTCGACCTCACTGGTTCTTACATGCACTGGGTGCGTCAGGCCCCGGGTAAGGGCCTGGAATGGGT |
| | TGCAGGTATTTCCGCTTCTGGAGGCGCTACTGCTTATGCCGATAGCGTCAAGGGCCGTTTCACTATAAGCGCAGA |
| | CACATCCAAAAACACAGCCTACCTACAAATGAACAGCTTAAGAGCTGAGGACACTGCCGTCTATTATTGTGCTCG |
| | CTCTCGTTCTTCTTCTTACTCTTCTTCTGGTTGGCGTTACTACTCTGGTGCTTTTGACTACTGGGGTCAAGGAACCC |
| | TGGTCACCGTCTCCTCG |
| hIgG1-VL-VH | EVQLVESGGGLVQPGGSLRLSCAASGFDLTGSYMHWVRQAPGKGLEWVAGISASGGATAYADSVKGRFTISADTSK |
| AA | NTAYLQMNSLRAEDTAVYYCARSRSSSYSSSGWRYYSGAFDYWGQGTLVTVSSASTKGPSVFPLAPSSKSTSGGTAAL |
| | GCLVKDYFPEPVTVSWNSGALTSGVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVYTLPPSREEMTKNQVSLTCLVKGFYPSDIA |
| | VEWESNGQPENNYKTTPPVLDSDGSFFLYSKLTVDKSRWQQGNVFSCSVMHEALHNHYTQKSLSLSPGGGGGGSG |
| | GGGSGGGTGDIQMTQSPSSLSASVGDRVTITCRASQSVSSAVAWYQQKPGKAPKLLYSASDLYSGVPSRFSGSRSGT |
| | DFTLTISSLQPEDFATYYCQQGSYLFTFGQGTKVEIKGTTAASGSSGGSSSGAEVQLVESGGGLVQPGGSLRLSCAASG |
| | FDLTGSYMHWVRQAPGKGLEWVAGISASGGATAYADSVKGRFTISADTSKNTAYLQMNSLRAEDTAVYYCARSRSS |
| | SYSSSGWRYYSGAFDYWGQGTLVTVSS |
| 39. LEAD hIgG1-scFv-scFv clone example | |
| 15031 hIgG1-scFv-scFv | |
| VL-CL NT | GATATCCAGATGACCCAGTCCCCGAGCTCCCTGTCCGCCTCTGTGGGCGATAGGGTCACCATCACCTGCCGTGCC |
| | AGTCAGTCCGIGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL AA | DIQMTQSPSSLSASVGDRVTITCRASQSVSSAVAWYQQKPGKAPKLLIYSASDLYSGVPSRESGSRSGTDFTLTISSLQP |
| | EDFATYYCQQGSYLFTFGQGTKVEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGEC |
| hIgG1- | GAGGTTCAGCTGGTGGAGTCTGGCGGTGGCCTGGTGCAGCCAGGGGGCTCACTCCGTTTGTCCTGTGCAGCTTC |
| VL-VH-VL-VH | TGGCTTCGACCTCACTGGTTCTTACATGCACTGGGTGCGTCAGGCCCCGGGTAAGGGCCTGGAATGGGTTGCAG |
| NT | GTATTTCCGCTTCTGGAGGCGCTACTGCTTATGCCGATAGCGTCAAGGGCCGTTTCACTATAAGCGCAGACACAT |
| | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGGTTGGCGTTACTACTCTGGTGCTTTTGACTACTGGGGTCAAGGAACCCTGGTC |
| | ACCGTCTCCTCGGCTAGCACCAAGGGCCCATCGGTCTTCCCCCTGGCACCCTCCTCCAAGAGCACCTCTGGGGGC |
| | ACAGCGGCCCTGGGCTGCCTGGTCAAGGACTACTTCCCCGAACCGGTGACGGTGTCGTGGAACTCAGGCGCCCT |
| | GACCAGCGGCGTGCACACCTTCCCGGCTGTCCTACAGTCCTCAGGACTCTACTCCCTCAGCAGCGTGGTGACCGT |
| | GCCCTCCAGCAGCTTGGGCACCCAGACCTACATCTGCAACGTGAATCACAAGCCCAGCAACACCAAGGTGGACA |
| | AGAAAGTTGAGCCCAAATCTTGTGACAAAACTCACACATGCCCACCGTGCCCAGCACCTGAACTCCTGGGGGGA |
| | CCGTCAGTCTTCCTCTTCCCCCCAAAACCCAAGGACACCCTCATGATCTCCCGGACCCCTGAGGTCACATGCGTGG |
| | TGGTGGACGTGAGCCACGAAGACCCTGAGGTCAAGTTCAACTGGTACGTGGACGGCGTGGAGGTGCATAATGC |
| | CAAGACAAAGCCGCGGGAGGAGCAGTACAACAGCACGTACCGTGTGGTCAGCGTCCTCACCGTCCTGCACCAG |
| | GACTGGCTGAATGGCAAGGAGTACAAGTGCAAGGTCTCCAACAAAGCCCTOCCAGCCCCCATCGAGAAAACCAT |
| | CTCCAAAGCCAAAGGGCAGCCCCGAGAACCACAGGTGTACACCCTGCCCCCATCCCGGGAGGAGATGACCAAG |
| | AACCAGGTCAGCCTGACCTGCCTGGTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACGGCTCCTTCTTCCTCTACAGCAAGCT |
| | CACCGTGGACAAGAGCAGGTGGCAGCAGGGGAACGTCTTCTCATGCTCCGTGATGCATGAGGCTCTGCACAACC |
| | ACTACACGCAGAAGAGCCTCTCCCTGTCTCCGGGTGGAGGAGGTGGGGGATCAGGTGGTGGGGGCTCTGGAG |
| | GCGGAACCGGTGATATCCAGATGACCCAGTCCCCGAGCTCCCTGTCCGCCTCTGTGGGCGATAGGGTCACCATC |
| | ACCTGCCGTGCCAGTCAGTCCGTGTCCAGCGCTGTAGCCTGGTATCAACAGAAACCAGGAAAAGCTCCGAAGCT |
| | TCTGATTTACTCGGCATCCAGCCTCTACTCTGGAGTCCCTTCTCGCTTCTCTGGTAGCCGTTCCGGGACGGATTTC |
| | ACTCTGACCATCAGCAGTCTGCAGCCGGAAGACTTCGCAACTTATTACTGTCAGCAAGGTTCTTACCTGTTCACGT |
| | TCGGACAGGGTACCAAGGTGGAGATCAAAGGTACTACTGCCGCTAGTGGTAGTAGTGGTGGCAGTAGCAGTG |
| | GTGCCGAGGTTCAGCTGGTGGAGTCTGGCGGTGGCCTGGTGCAGCCAGGGGGCTCACTCCGTTTGTCCTGTGCA |
| | GCTTCTGGCTTCGACCTCACTGGTTCTTACATGCACTGGGTGCGTCAGGCCCCGGGTAAGGGCCTGGAATGGGT |
| | TGCAGGTATTTCCGCTTCTGGAGGCGCTACTGCTTATGCCGATAGCGTCAAGGGCCGTTTCACTATAAGCGCAGA |
| | CACATCCAAAAACACAGCCTACCTACAAATGAACAGCTTAAGAGCTGAGGACACTGCCGTCTATTATTGTGCTCG |
| | CTCTCGTTCTTCTTCTTACTCTTCTTCTGGTTGGCGTTACTACTCTGGTGCTTTTGACTACTGGGGTCAAGGAACCC |
| | TGGTCACCGTCTCCTCGGGTGGAGGTGGCTCCGGAGGAGGTGGCAGTGATATCCAGATGACCCAGTCCCCGAG |
| | CTCCCTGTCCGCCTCTGTGGGCGATAGGGTCACCATCACCTGCCGTGCCAGTCAGTCCGTGTCCAGCGCTGTAGC |
| | CTGGTATCAACAGAAACCAGGAAAAGCTCCGAAGCTTCTGATTTACTCGGCATCCGATCTCTACTCTGGAGTCCC |
| | TTCTCGCTTCTCTGGTAGCCGTTCCGGGACGGATTTCACTCTGACCATCAGCAGTCTGCAGCCGGAAGACTTCGC |
| | AACTTATTACTGTCAGCAAGGTTCTTACCTGTTCACGTTCGGACAGGGTACCAAGGTGGAGATCAAAGGTACTAC |
| | TGCCGCTAGTGGTAGTAGTGGTGGCAGTAGCAGTGGTGCCGAGGTTCAGCTGGTGGAGTCTGGCGGTGGCCT |
| | GGTGCAGCCAGGGGGCTCACTCCGTTTGTCCTGTGCAGCTTCTGGCTTCGACCTCACTGGTTCTTACATGCACTG |
| | GGTGCGTCAGGCCCCGGGTAAGGGCCTGGAATGGGTTGCAGGTATTTCCGCTTCTGGAGGCGCTACTGCTTATG |
| | CCGATAGCGTCAAGGGCCGTTTCACTATAAGCGCAGACACATCCAAAAACACAGCCTACCTACAAATGAACAGC |
| | TTAAGAGCTGAGGACACTGCCGTCTATTATTGTGCTCGCTCTCGTTCTTCTTCTTACTCTTCTTCTGGTTGGCGTTA |
| | CTACTCTGGTGCTTTTGACTACTGGGGTCAAGGAACCCTGGTCACCGTCTCCTCG |
| hIgG1-VL-VH- | EVQLVESGGGLVQPGGSLRLSCAASGFDLTGSYMHWVRQAPGKGLEWVAGISASGGATAYADSVKGRFTISADTSK |
| VL-VH AA | NTAYLQMNSLRAEDTAVYYCARSRSSSYSSSGWRYYSGAFDYWGQGTLVTVSSASTKGPSVFPLAPSSKSTSGGTAAL |
| | GCLVKDYFPEPVTVSWNSGALTSGVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVYTLPPSREEMTKNQVSLTCLVKGFYPSDIA |
| | VEWESNGQPENNYKTTPPVLDSDGSFFLYSKLTVDKSRWQQGNVFSCSVMHEALHNHYTQKSLSLSPGGGGGGSG |
| | GGGSGGGTGDIQMTQSPSSLSASVGDRVTITCRASQSVSSAVAWYQQKPGKAPKLLIYSASDLYSGVPSRFSGSRSGT |
| | DFTLTISSLQPEDFATYYCQQGSYLFTFGQGTKVEIKGTTAASGSSGGSSSGAEVQLVESGGGLVQPGGSLRLSCAASG |
| | FDLTGSYMHWVRQAPGKGLEWVAGISASGGATAYADSVKGRFTISADTSKNTAYLQMNSLRAEDTAVYYCARSRSS |
| | SYSSSGWRYYSGAFDYWGQGTLVTVSSGGGGSGGGGSDIQMTQSPSSLSASVGDRVTITCRASQSVSSAVAWYQQ |
| | KPGKAPKLLIYSASDLYSGVPSRFSGSRSGTDFTLTISSLQPEDFATYYCQQGSYLFTFGQGTKVEIKGTTAASGSSGGSS |
| | SGAEVQLVESGGGLVQPGGSLRLSCAASGFDLTGSYMHWVRQAPGKGLEWVAGISASGGATAYADSVKGRFTISAD |
| | TSKNTAYLQMNSLRAEDTAVYYCARSRSSSYSSSGWRYYSGAFDYWGQGTLVTVSS |
| 40. LEAD hIgG1-diabody clone example | |
| 15031 hIgG1-diabody | |
| VL-CL NT | GATATCCAGATGACCCAGTCCCCGAGCTCCCTGTCCGCCTCTGTGGGCGATAGGGTCACCATCACCTGCCGTGCC |
| | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTLTISSLQP |
| | EDFATYYCQQGSYLFTFGQGTKVEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGEC |
| hIgG1-VH-VL | GAGGTTCAGCTGGTGGAGTCTGGCGGTGGCCTGGTGCAGCCAGGGGGCTCACTCCGTTTGTCCTGTGCAGCTTC |
| NT | TGGCTTCGACCTCACTGGTTCTTACATGCACTGGGTGCGTCAGGCCCCGGGTAAGGGCCTGGAATGGGTTGCAG |
| | GTATTTCCGCTTCTGGAGGCGCTACTGCTTATGCCGATAGCGTCAAGGGCCGTTTCACTATAAGCGCAGACACAT |
| | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGGTTGGCGTTACTACTCTGGTGCTTTTGACTACTGGGGTCAAGGAACCCTGGTC |
| | ACCGTCTCCTCGGCTAGCACCAAGGGCCCATCGGTCTTCCCCCTGGCACCCTCCTCCAAGAGCACCTCTGGGGGC |
| | ACAGCGGCCCTGGGCTGCCTGGTCAAGGACTACTTCCCCGAACCGGTGACGGTGTCGTGGAACTCAGGCGCCCT |
| | GACCAGCGGCGTGCACACCTTCCCGGCTGTCCTACAGTCCTCAGGACTCTACTCCCTCAGCAGCGTGGTGACCGT |
| | GCCCTCCAGCAGCTTGGGCACCCAGACCTACATCTGCAACGTGAATCACAAGCCCAGCAACACCAAGGTGGACA |
| | AGAAAGTTGAGCCCAAATCTTGTGACAAAACTCACACATGCCCACCGTGCCCAGCACCTGAACTCCTGGGGGGA |
| | CCGTCAGTCTTCCTCTTCCCCCCAAAACCCAAGGACACCCTCATGATCTCCCGGACCCCTGAGGTCACATGCGTGG |
| | TGGTGGACGTGAGCCACGAAGACCCTGAGGTCAAGTTCAACTGGTACGTGGACGGCGTGGAGGTGCATAATGC |
| | CAAGACAAAGCCGCGGGAGGAGCAGTACAACAGCACGTACCGTGTGGTCAGCGTCCTCACCGTCCTGCACCAG |
| | GACTGGCTGAATGGCAAGGAGTACAAGTGCAAGGTCTCCAACAAAGCCCTCCCAGCCCCCATCGAGAAAACCAT |
| | CTCCAAAGCCAAAGGGCAGCCCCGAGAACCACAGGTGTACACCCTGCCCCCATCCCGGGAGGAGATGACCAAG |
| | AACCAGGTCAGCCTGACCTGCCTGGTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACGGCTCCTTCTTCCTCTACAGCAAGCT |
| | CACCGTGGACAAGAGCAGGTGGCAGCAGGGGAACGTCTTCTCATGCTCCGTGATGCATGAGGCTCTGCACAACC |
| | ACTACACGCAGAAGAGCCTCTCCCTGTCTCCGGGTGGAGGTGGGGGATCAGGTGGTGGGGGCTCTGGAGGCG |
| | GAACCGGTGAGGTTCAGCTGGTGGAGTCTGGCGGTGGCCTGGTGCAGCCAGGGGGCTCACTCCGTTTGTCCTG |
| | TGCAGCTTCTGGCTTCGACCTCACTGGTTCTTACATGCACTGGGTGCGTCAGGCCCCGGGTAAGGGCCTGGAAT |
| | GGGTTGCAGGTATTTCCGCTTCTGGAGGCGCTACTGCTTATGCCGATAGCGTCAAGGGCCGTTTCACTATAAGCG |
| | CAGACACATCCAAAAACACAGCCTACCTACAAATGAACAGCTTAAGAGCTGAGGACACTGCCGTCTATTATTGTG |
| | CTCGCTCTCGTTCTTCTTCTTACTCTTCTTCTGGTTGGCGTTACTACTCTGGTGCTTTTATGGACTACTGGGGTCAA |
| | GGAACCCTGGTCACCGTCTCCTCGGGTGGAGGTGGCAGTGATATCCAGATGACCCAGTCCCCGAGCTCOCTGTC |
| | CGCCTCTGTGGGCGATAGGGTCACCATCACCTGCCGTGCCAGTCAGTCCGTGTCCAGCGCTGTAGCCTGGTATCA |
| | ACAGAAACCAGGAAAAGCTCCGAAGCTTCTGATTTACTTCGGCATCCGATCTCTACTCTGGAGTCCCTTCTCGCTTC |
| | TCTGGTAGCCGTTCCGGGACGGATTTCACTCTGACCATCAGCAGTCTGCAGCCGGAAGACTTCGCAACTTATTAC |
| | TGTCAGCAAGGTTCTTACCTGTTCACGTTCGGACAGGGTACCAAGGTGGAGATCAAA |
| hIgG1-VH-VL | EVQLVESGGGLVQPGGSLRLSCAASGFDLTGSYMHWVRQAPGKGLEWVAGISASGGATAYADSVKGRFTISADTSK |
| AA | NTAYLQMNSLRAEDTAVYYCARSRSSSYSSSGWRYYSGAFDYWGQGTLVTVSSASTKGPSVFPLAPSSKSTSGGTAAL |
| | GCLVKDYFPEPVTVSWNSGALTSGVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVYTLPPSREEMTKNQVSLTCLVKGFYPSDIA |
| | VEWESNGQPENNYKTTPPVLDSDGSFFLYSKLTVDKSRWQQGNVFSCSVMHEALHNHYTQKSLSLSPGGGGGSGG |
| | GGSGGGTGEVQLVESGGGLVQPGGSLRLSCAASGFDLTGSYMHWVRQAPGKGLEWVAGISASGGATAYADSVKG |
| | RFTISADTSKNTAYLQMNSLRAEDTAVYYCARSRSSSYSSSGWRYYSGAFMDYWGQGTLVTVSSGGGGSDIQMTQS |
| | PSSLSASVGDRVTITCRASQSVSSAVAWYQQKPGKAPKLLIYSASDLYSGVPSRFSGSRSGTDFTLTISSLQPEDFATYYC |
| | QQGSYLFTFGQGTKVEIK |
| 41. LEAD hIgG1-Fab clone example | |
| 15031 IgG-Fab | |
| VL-CL NT | GATATCCAGATGACCCAGTCCCCGAGCTCCCTGTCCGCCTCTGTGGGCGATAGGGTCACCATCACCTGCCGTGCC |
| | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTLTISSLQP |
| | EDFATYYCQQGSYLFTFGQGTKVEIKRTVAAPSVFIFPPSDEQLKSGTASVVCLNNFYPREAKVQWKVDNALQSGNS |
| | QESVTEQQSKDSTYSLSSTLTLSKADYEKHKVYACEVTHQGLSSPVTKSFNRGEC |
| hIgG1-VH-CH1 | GAGGTTCAGCTGGTGGAGTCTGGCGGTGGCCTGGTGCAGCCAGGGGGCTCACTCCGTTTGTCCTGTGCAGCTTC |
| NT | TGGCTTCGATCTCACTGGTTCTTACATGCACTGGGTGCGTCAGGCCCCGGGTAAGGGCCTGGAATGGGTTGCAG |
| | GTATTTCCGCTTCTGGAGGCGCTACTGCTTATGCCGATAGCGTCAAGGGCCGTTTCACTATAAGCGCAGACACAT |
| | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGGTTGGCGTTACTACTCTGGTGCTTTTGACTACTGGGGTCAAGGAACCCTGGTC |
| | ACCGTCTCCTCGGCTAGCACCAAGGGCCCATCGGTCTTCCCCCTGGCACCCTCCTCCAAGAGCACCTCTGGGGGC |
| | ACAGGGGCCCTGGGCTGCCTGGTCAAGGACTACTTCCCCGAACCGGTGACGGTGTCGTGGAACTCAGGCGCCCT |
| | GACCAGCGGCGTGCACACCTTCCCGGCTGTCCTACAGTCCTCAGGACTCTACTCCCTCAGCAGCGTGGTGACCGT |
| | GCCCTCCAGCAGCTTGGGCACCCAGACCTACATCTGCAACGTGAATCACAAGCCCAGCAACACCAAGGTGGACA |
| | AGAAAGTTGAGCCCAAATCTTGTGACAAAACTCACACATGCCCACCGTGCCCAGCACCTGAACTCCTGGGGGGA |
| | CCGTCAGTCTTCCTCTTCCCCCCAAAACCCAAGGACACCCTCATGATCTCCCGGACCCCTGAGGTCACATGCGTGG |
| | TGGTGGACGTGAGCCACGAAGACCCTGAGGTCAAGTTCAACTGGTACGTGGACGGCGTGGAGGTGCATAATGC |
| | CAAGACAAAGCCGCGGGAGGAGCAGTACAACAGCACGTACCGTGTGGTCAGCGTCCTCACCGTCCTGCACCAG |
| | GACTGGCTGAATGGCAAGGAGTACAAGTGCAAGGTCTCCAACAAAGCCCTOCCAGCCCCCATCGAGAAAACCAT |
| | CTCCAAAGCCAAAGGGCAGCCCCGAGAACCACAGGTGTACACCCTGCCCCCATCCCGGGAGGAGATGACCAAG |
| | AACCAGGTCAGCCTGACCTGCCTGGTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACGGCTCCTTCTTCCTCTACAGCAAGCT |
| | CACCGTGGACAAGAGCAGGTGGCAGCAGGGGAACGTCTTCTCATGCTCCGTGATGCATGAGGCTCTGCACAACC |
| | ACTACACGCAGAAGAGCCTCTCCCTGTCTCCGGGTGGAGGAGGTGGGGGATCAGGTGGTGGGGGCTCTGGAG |
| | GCGGAACCGGTGAGGTTCAGCTGGTGGAGTCTGGCGGTGGCCTGGTGCAGCCAGGGGGCTCACTCCGTTTGTC |
| | CTGTGCAGCTTCTGGCTTCGATCTCACTGGTTCTTACATGCACTGGGTGCGTCAGGCCCCGGGTAAGGGCCTGGA |
| | ATGGGTTGCAGGTATTTCCGCTTCTGGAGGCGCTACTGCTTATGCCGATAGCGTCAAGGGCCGTTTCACTATAAG |
| | CGCAGACACATCCAAAAACACAGCCTACCTACAAATGAACAGCTTAAGAGCTGAGGACACTGCCGTCTATTATTG |
| | TGCTCGCTCTCGTTCTTCTTCTTACTCTTCTTCTGGTTGGCGTTACTACTCTGGTGCTTTTGACTACTGGGGTCAAG |
| | GAACCCTGGTCACCGTCTCCTCGGCTAGCACCAAGGGTCCATCGGTCTTCCCCCTGGCACCCTCCTCCAAGAGCA |
| | CCTCTGGGGGCACAGCGGCCCTGGGCTGCCTGGTCAAGGACTACTTCCCCGAACCCGTGACGGTGTCGTGGAAC |
| | TCAGGCGCCCTGACCAGCGGCGTGCACACCTTCCCGGCTGTCCTACAGTCCTCAGGACTCTACTCCCTCAGCAGC |
| | GTGGTGACCGTGCCCTCCAGCAGCTTGGGCACCCAGACCTACATCTGCAACGTGAATCACAAGCCCAGCAACAC |
| | CAAGGTCGACAAGAAAGTTGAGCCCAAATCTTGTGACAAAACTCACACA |
| hIgG1-VH-CH1 | EVQLVESGGGLVQPGGSLRLSCAASGPDLTGSYMHWVRQAPGKGLEWVAGISASGGATAYADSVKGRFTISADTSK |
| AA | NTAYLQMNSLRAEDTAVYYCARSRSSSYSSSGWRYYSGAFDYWGQGTLVTVSSASTKGPSVFPLAPSSKSTSGGTAAL |
| | GCLVKDYFPEPVTVSWNSGALTSGVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVYTLPPSREEMTKNQVSLTCLVKGFYPSDIA |
| | VEWESNGQPENNYKTTPPVLDSDGSFFLYSKLTVDKSRWQQGNVFSCSVMHEALHNHYTQKSLSLSPGGGGGGSG |
| | GGGSGGGTGEVQLVESGGGLVQPGGSLRLSCAASGFDLTGSYMHWVRQAPGKGLEWVAGISASGGATAYADSVK |
| | GRFTISADTSKNTAYLQMNSLRAEDTAVYYCARSRSSSYSSSGWRYYSGAFDYWGQGTLVTVSSASTKGPSVFPLAPS |
| | SKSTSGGTAALGCLVKDYFPEPVTVSWNSGALTSGVHTFPAVLQSSGLYSLSSVVTVPSSSLGTQTYICNVNHKPSNTK |
| | VDKKVEPKSCDKTHT |
| 42. LEAD hIgG1-Fab-Fab clone example | |
| 15031 hIgG1-Fab-Fab | |
| VL-CL NT | GATATCCAGATGACCCAGTCCCCGAGCTCCCTGTCCGCCTCTGTGGGCGATAGGGTCACCATCACCTGCCGTGCC |
| | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTLTISSLQP |
| | EDFATYYCQQGSYLFTFGQGTKVEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGEC |
| hIgG1-VH- | GAGGTTCAGCTGGTGGAGTCTGGCGGTGGCCTGGTGCAGCCAGGGGGCTCACTCCGTTTGTCCTGTGCAGCTTC |
| CH1-VH-CH1 | TGGCTTCGACCTCACTGGTTCTTACATGCACTGGGTGCGTCAGGCCCCGGGTAAGGGCCTGGAATGGGTTGCAG |
| NT | GTATTTCCGCTTCTGGAGGCGCTACTGCTTATGCCGATAGCGTCAAGGGCCGTTTCACTATAAGCGCAGACACAT |
| | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGGTTGGCGTTACTACTCTGGTGCTTTTGACTACTGGGGTCAAGGAACCCTGGTC |
| | ACCGTCTCCTCGGCTAGCACCAAGGGCCCATCGGTCTTCCCCCTGGCACCCTCCTCCAAGAGCACCTCTGGGGGC |
| | ACAGCGGCCCTGGGCTGCCTGGTCAAGGACTACTTCCCCGAACCGGTGACGGTGTCGTGGAACTCAGGCGCCCT |
| | GACCAGCGGCGTGCACACCTTCCCGGCTGTCCTACAGTCCTCAGGACTCTACTCCCTCAGCAGCGTGGTGACCGT |
| | GCCCTCCAGCAGCTTGGGCACCCAGACCTACATCTGCAACGTGAATCACAAGCCCAGCAACACCAAGGTGGACA |
| | AGAAAGTTGAGCCCAAATCTTGTGACAAAACTCACACATGCCCACCGTGCCCAGCACCTGAACTCCTGGGGGGA |
| | CCGTCAGTCTTCCTCTTCCCCCCAAAACCCAAGGACACCCTCATGATCTCCCGGACCCCTGAGGTCACATGCGTGG |
| | TGGTGGACGTGAGCCACGAAGACCCTGAGGTCAAGTTCAACTGGTACGTGGACGGCGTGGAGGTGCATAATGC |
| | CAAGACAAAGCCGCGGGAGGAGCAGTACAACAGCACGTACCGTGTGGTCAGCGTCCTCACCGTCCTGCACCAG |
| | GACTGGCTGAATGGCAAGGAGTACAAGTGCAAGGTCTCCAACAAAGCCCTOCCAGCCCCCATCGAGAAAACCAT |
| | CTCCAAAGCCAAAGGGCAGCCCCGAGAACCACAGGTGTACACCCTGCCCCCATCCCGGGAGGAGATGACCAAG |
| | AACCAGGTCAGCCTGACCTGCCTGGTCAAAGGCTTCTATCCCAGCGACATCGCCGTGGAGTGGGAGAGCAATGG |
| | GCAGCCGGAGAACAACTACAAGACCACGCCTCCCGTGCTGGACTCCGACGGCTCCTTCTTCCTCTACAGCAAGCT |
| | CACCGTGGACAAGAGCAGGTGGCAGCAGGGGAACGTCTTCTCATGCTCCGTGATGCATGAGGCTCTGCACAACC |
| | ACTACACGCAGAAGAGCCTCTCCCTGTCTCCGGGTGGAGGAGGTGGGGGATCAGGTGGTGGGGGCTCTGGAG |
| | GCGGAACCGGTGAGGTTCAGCTGGTGGAGTCTGGCGGTGGCCTGGTGCAGCCAGGGGGCTCACTCCGTTTGTC |
| | CTGTGCAGCTTCTGGCTTCGACCTCACTGGTTCTTACATGCACTGGGTGCGTCAGGCCCCGGGTAAGGGCCTGG |
| | AATGGGTTGCAGGTATTTCCGCTTCTGGAGGCGCTACTGCTTATGCCGATAGCGTCAAGGGCCGTTTCACTATAA |
| | GCGCAGACACATCCAAAAACACAGCCTACCTACAAATGAACAGCTTAAGAGCTGAGGACACTGCCGTCTATTATT |
| | GTGCTCGCTCTCGTTCTTCTTCTTACTCTTCTTCTGGTTGGCGTTACTACTCTGGTGCTTTTGACTACTGGGGTCAA |
| | GGAACCCTGGTCACCGTCTCCTCGGCTAGCACCAAGGGTCCATCGGTCTTCCCCCTGGCACCCTCCTCCAAGAGC |
| | ACCTCTGGGGGCACAGCGGCCCTGGGCTGCCTGGTCAAGGACTACTTCCCCGAACCCGTGACGGTGTCGTGGAA |
| | CTCAGGCGCCCTGACCAGCGGCGTGCACACCTTCCCGGCTGTCCTACAGTCCTCAGGACTCTACTCCCTCAGCAG |
| | CGTGGTGACCGTGCCCTCCAGCAGCTTGGGCACCCAGACCTACATCTGCAACGTGAATCACAAGCCCAGCAACA |
| | CCAAGGTCGACAAGAAAGTTGAGCCCAAATCTTGTGACAAAACTCACACAGGTGGAGGTGGCTCCGGAGGAGG |
| | TGGCAGTGAGGTTCAGCTGGTGGAGTCTGGCGGTGGCCTGGTGCAGCCAGGGGGCTCACTCCGTTTGTCCTGT |
| | GCAGCTTCTGGCTTCGACCTCACTGGTTCTTACATGCACTGGGTGCGTCAGGCCCCGGGTAAGGGCCTGGAATG |
| | GGTTGCAGGTATTTCCGCTTCTGGAGGCGCTACTGCTTATGCCGATAGCGTCAAGGGCCGTTTCACTATAAGCGC |
| | AGACACATCCAAAAACACAGCCTACCTACAAATGAACAGCTTAAGAGCTGAGGACACTGCCGTCTATTATTGTGC |
| | TCGCTCTCGTTCTTCTTCTTACTCTTCTTCTGGTTGGCGTTACTACTCTGGTGCTTTTGACTACTGGGGTCAAGGAA |
| | CCCTGGTCACCGTCTCCTCGGCTAGCACCAAGGGTCCATCGGTCTTCCCCCTGGCACCCTCCTCCAAGAGCACCTC |
| | TGGGGGCACAGCGGCCCTGGGCTGCCTGGTCAAGGACTACTTCCCCGAACCCGTGACGGTGTCGTGGAACTCA |
| | GGCGCCCTGACCAGCGGCGTGCACACCTTCCCGGCTGTCCTACAGTCCTCAGGACTCTACTCCCTCAGCAGCGTG |
| | GTGACCGTGCCCTCCAGCAGCTTGGGCACCCAGACCTACATCTGCAACGTGAATCACAAGCCCAGCAACACCAA |
| | GGTCGACAAGAAAGTTGAGCCCAAATCTTGTGACAAAACTCACACA |
| hIgG1-VH- | EVQLVESGGGLVQPGGSLRLSCAASGFDLTGSYMHWVRQAPGKGLEWVAGISASGGATAYADSVKGRFTISADTSK |
| CH1-VH-CH1 | NTAYLQMNSLRAEDTAVYYCARSRSSSYSSSGWRYYSGAFDYWGQGTLVTVSSASTKGPSVFPLAPSSKSTSGGTAAL |
| AA | GCLVKDYFPEPVTVSWNSGALTSGVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVYTLPPSREEMTKNQVSLTCLVKGFYPSDIA |
| | VEWESNGQPENNYKTTPPVLDSDGSPFLYSKLTVDKSRWQQGNVFSCSVMHEALHNHYTQKSLSLSPGGGGGGSG |
| | GGGSGGGTGEVQLVESGGGLVQPGGSLRLSCAASGFDLTGSYMHWVRQAPGKGLEWVAGISASGGATAYADSVK |
| | GRFTISADTSKNTAYLQMNSLRAEDTAVYYCARSRSSSYSSSGWRYYSGAFDYWGQGTLVTVSSASTKGPSVFPLAPS |
| | SKSTSGGTAALGCLVKDYFPEPVTVSWNSGALTSGVHTFPAVLQSSGLYSLSSVVTVPSSSLGTQTYICNVNHKPSNTK |
| | VDKKVEPKSCDKTHTGGGGSGGGGSEVQLVESGGGLVQPGGSLRLSCAASGFDLTGSYMHWVRQAPGKGLEWVA |
| | GISASGGATAYADSVKGRFTISADTSKNTAYLQMNSLRAEDTAVYYCARSRSSSYSSSGWRYYSGAFDYWGQGTLVT |
| | VSSASTKGPSVFPLAPSSKSTSGGTAALGCLVKDYFPEPVTVSWNSGALTSGVHTFPAVLQSSGLYSLSSVVTVPSSSLG |
| | TQTYICNVNHKPSNTKVDKKVEPKSCDKTHT |
| 43. Fab-hIgG1 inner cross | |
| 15031 Fab-hIgG1 Inner cross | |
| VL-CL-VH-CH1 | GATATCCAGATGACCCAGTCCCCGAGCTCCCTGTCCGCCTCTGTGGGCGATAGGGTCACCATCACCTGCCGTGCC |
| NT | AGTCAGTCCGTGTCCAGCGCTGTAGCCTGGTATCAACAGAAACCAGGAAAAGCTCCGAAGCTTCTGATTTACTCG |
| (secondary) | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGTGGAGGAGGTGGCAGTGGCGGAGGTGGCAGC |
| | GGTGGAGGTGGCAGTGAGGTTCAGCTGGTGGAGTCTGGCGGTGGCCTGGTGCAGCCAGGGGGCTCACTCCGT |
| | TTGTCCTGTGCAGCTTCTGGCTTCGATCTCACTGGTTCTTACATGCACTGGGTGCGTCAGGCCCCGGGTAAGGGC |
| | CTGGAATGGGTTGCAGGTATTTCCGCTTCTGGAGGCGCTACTGCTTATGCCGATAGCGTCAAGGGCCGTTTCACT |
| | ATAAGCGCAGACACATCCAAAAACACAGCCTACCTACAAATGAACAGCTTAAGAGCTGAGGACACTGCCGTCTA |
| | TTATTGTGCTCGCTCTCGTTCTTCTTCTTACTCTTCTTCTGGTTGGCGTTACTACTCTGGTGCTTTTGACTACTGGGG |
| | TCAAGGAACCCTGGTCACCGTCTCCTCGGCTAGCACCAAGGGTCCATCGGTCTTCCCCCTGGCACCCTCCTCCAA |
| | GAGCACCTCTGGGGGCACAGCGGCCCTGGGCTGCCTGGTCAAGGACTACTTCCCCGAACCCGTGACGGTGTCGT |
| | GGAACTCAGGCGCCCTGACCAGCGGCGTGCACACCTTCCCGGCTGTCCTACAGTCCTCAGGACTCTACTCCCTCA |
| | GCAGCGTGGTGACCGTGCCCTCCAGCAGCTTGGGCACCCAGACCTACATCTGCAACGTGAATCACAAGCCCAGC |
| | AACACCAAGGTCGACAAGAAAGTTGAGCCCAAATCTTGTGACAAAACTCACACATGA |
| VL-CL-VH-CH1 | DIQMTQSPSSLSASVGDRVTITCRASQSVSSAVAWYQQKPGKAPKLLIYSASDLYSGVPSRFSGSRSGTDFTLTISSLQP |
| (2ndary) | EDFATYYCQQGSYLFTFGQGTKVEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGECGGGGSGGGGSGGGGSEVQLVES |
| | GGGLVQPGGSLRLSCAASGFDLTGSYMHWVRQAPGKGLEWVAGISASGGATAYADSVKGRFTISADTSKNTAYLQ |
| | MNSLRAEDTAVYYCARSRSSSYSSSGWRYYSGAFDYWGQGTLVTVSSASTKGPSVFPLAPSSKSTSGGTAALGCLVKD |
| | YFPEPVTVSWNSGALTSGVHTFPAVLQSSGLYSLSSVVTVPSSSLGTQTYICNVNHKPSNTKVDKKVEPKSCDKTHT |
| VH-CH1-VL- | GAGGTTCAGCTGGTGGAGTCTGGCGGTGGCCTGGTGCAGCCAGGGGGCTCACTCCGTTTGTCCTGTGCAGCTTC |
| CL-CH2-CH3 | TGGCTTCGATCTCACTGGTTCTTACATGCACTGGGTGCGTCAGGCCCCGGGTAAGGGCCTGGAATGGGTTGCAG |
| NT | GTATTTCCGCTTCTGGAGGCGCTACTGCTTATGCCGATAGCGTCAAGGGCCGTTTCACTATAAGCGCAGACACAT |
| (primary) | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGGTTGGGGTTACTACTCTGGTGCTTTTGACTACTGGGGTCAAGGAACCCTGGTC |
| | ACCGTCTCCTCGGCTAGCACCAAGGGCCCATCGGTCTTCCCCCTGGCACCCTCCTCCAAGAGCACCTCTGGGGGC |
| | ACAGCGGCCCTGGGCTGCCTGGTCAAGGACTACTTCCCCGAACCGGTGACGGTGTCGTGGAACTCAGGCGCCCT |
| | GACCAGCGGCGTGCACACCTTCCCGGCTGTCCTACAGTCCTCAGGACTCTACTCCCTCAGCAGCGTGGTGACCGT |
| | GCCCTCCAGCAGCTTGGGCACCCAGACCTACATCTGCAACGTGAATCACAAGCCCAGCAACACCAAGGTGGACA |
| | AGAAAGTTGAGCCCAAATCTTGTGACAAAACTCACACATCCGGAGGAGGTGGCAGTGGCGGAGGTGGCAGCG |
| | GTGGAGGTGGCAGTGATATCCAGATGACCCAGTCCCCGAGCTCCCTGTCCGCCTCTGTGGGCGATAGGGTCACC |
| | ATCACCTGCCGTGCCAGTCAGTCCGTGTCCAGCGCTGTAGCCTGGTATCAACAGAAACCAGGAAAAGCTCCGAA |
| | GCTTCTGATTTACTCGGCATCCGATCTCTACTCTGGAGTCCCTTCTCGCTTCTCTGGTAGCCGTTCCGGGACGGAT |
| | TTCACTCTGACCATCAGCAGTCTGCAGCCGGAAGACTTCGCAACTTATTACTGTCAGCAAGGTTCTTACCTGTTCA |
| | CGTTCGGACAGGGTACCAAGGTGGAGATCAAACGTACGGTGGCTGCACCATCTGTCTTCATCTTCCCGCCATCTG |
| | ATGAGCAGTTGAAATCTGGAACTGCCTCTGTTGTGTGCCTGCTGAATAACTTCTATCCCAGAGAGGCCAAAGTAC |
| | AGTGGAAGGTGGATAACGCCCTCCAATCGGGTAACTCCCAGGAGAGTGTCACAGAGCAGGACAGCAAGGACAG |
| | CACCTACAGCCTCAGCAGCACCCTGACGCTGAGCAAAGCAGACTACGAAAAACATAAAGTCTACGOCTGCGAAG |
| | TCACCCATCAGGGCCTGAGCTCGCCCGTCACAAAGAGCTTCAACAGGGGAGAGTGTGACAAAACTCACACATGC |
| | CCACCGTGCCCAGCACCTGAACTCCTGGGGGGACCGTCAGTCTTCCTCTTCCCCCCAAAACCCAAGGACACCCTC |
| | ATGATCTCCCGGACCCCTGAGGTCACATGCGTGGTGGTGGACGTGAGCCACGAAGACCCTGAGGTCAAGTTCAA |
| | CTGGTACGTGGACGGCGTGGAGGTGCATAATGCCAAGACAAAGCCGCGGGAGGAGCAGTACAACAGCACGTA |
| | CCGTGTGGTCAGCGTCCTCACCGTCCTGCACCAGGACTGGCTGAATGGCAAGGAGTACAAGTGCAAGGTCTCCA |
| | ACAAAGCCCTCCCAGCCCCCATCGAGAAAACCATCTCCAAAGCCAAAGGGCAGCCCCGAGAACCACAGGTGTAC |
| | ACCCTGCCCCCATCCCGGGAGGAGATGACCAAGAACCAGGTCAGCCTGACCTGCCTGGTCAAAGGCTTCTATCC |
| | CAGCGACATCGCCGTGGAGTGGGAGAGCAATGGGCAGCCGGAGAACAACTACAAGACCACGCCTCCCGTGCTG |
| | GACTCCGACGGCTCCTTCTTCCTCTACAGCAAGCTCACCGTGGACAAGAGCAGGTGGCAGCAGGGGAACGTCTT |
| | CTCATGCTCCGTGATGCATGAGGCTCTGCACAACCACTACACGCAGAAGAGCCTCTCCCTGTCTCCGGGTAAA |
| VH-CH1-VL- | EVQLVESGGGLVQPGGSLRLSCAASGFDLTGSYMHWVRQAPGKGLEWVAGISASGGATAYADSVKGRFTISADTSK |
| CL-CH2-CH3 | NTAYLQMNSLRAEDTAVYYCARSRSSSYSSSGWRYYSGAFDYWGQGTLVTVSSASTKGPSVFPLAPSSKSTSGGTAAL |
| AA | GCLVKDYPPEPVTVSWNSGALTSGVHTFPAVLQSSGLYSLSSVVTVPSSSLGTQTYICNVNHKPSNTKVDKKVEPKSCD |
| (primary) | KTHTSGGGGSGGGGSGGGGSDIQMTQSPSSLSASVGDRVTITCRASQSVSSAVAWYQQKPGKAPKLLIYSASDLYSG |
| | VPSRFSGSRSGTDFTLTISSLQPEDFATYYCQQGSYLFTFGQGTKVEIKRTVAAPSVFIFPPSDEQLKSGTASVVCLLNNF |
| | YPREAKVQWKVDNALQSGNSQESVTEQDSKOSTYSLSSTLTLSKADYEKHKVYACEVTHQGLSSPVTKSFNRGECDKT |
| | HTCPPCPAPELLGGPSVFLFPPKPKDTLMISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYTQKSLSLSPGK |
| 44. Fab-hIgG1 outer cross | |
| 15031 Fab-hIgG1 outer cross | |
| VH-CH1-VL-CL | GAGGTTCAGCTGGTGGAGTCTGGCGGTGGCCTGGTGCAGCCAGGGGGCTCACTCCGTTTGTCCTGTGCAGCTTC |
| NT | TGGCTTCGATCTCTCTTCTTATTCTATGCACTGGGTGCGTCAGGCCCCGGGTAAGGGCCTGGAATGGGTTGCAGG |
| (secondary) | TATTTCCGCTTCTGGAGGCGCTACTGCTTATGCCGATAGCGTCAAGGGCCGTTTCACTATAAGCGCAGACACATC |
| | CAAAAACACAGCCTACCTACAAATGAACAGCTTAAGAGCTGAGGACACTGCCGTCTATTATTGTGCTCGCTCTCG |
| | TTCTTCTTCTTACTCTTCTTCTGGTTGGCGTTACTACTCTGGTGCTTTTGACTACTGGGGTCAAGGAACCCTGGTCA |
| | CCGTCTCCTCGGCTAGCACCAAGGGTCCATCGGTCTTCCCCCTGGCACCCTCCTCCAAGAGCACCTCTGGGGGCA |
| | CAGCGGCCCTGGGCTGCCTGGTCAAGGACTACTTCCCCGAACCCGTGACGGTGTCGTGGAACTCAGGCGCCCTG |
| | ACCAGCGGCGTGCACACCTTCCCGGCTGTCCTACAGTCCTCAGGACTCTACTCCCTCAGCAGCGTGGTGACCGTG |
| | CCCTCCAGCAGCTTGGGCACCCAGACCTACATCTGCAACGTGAATCACAAGCCCAGCAACACCAAGGTCGACAA |
| | GAAAGTTGAGCCCAAATCTTGTGACAAAACTCACACATCCGGAGGAGGTGGCAGTGGCGGAGGTGGCAGCGG |
| | TGGAGGTGGCAGTGATATCCAGATGACCCAGTCCCCGAGCTCCCTGTCCGCCTCTGTGGGCGATAGGGTCACCA |
| | TCACCTGCCGTGCCAGTCAGTCCGTGTCCAGCGCTGTAGCCTGGTATCAACAGAAACCAGGAAAAGCTCCGAAG |
| | CTTCTGATTTACTCGGCATCCGATCTCTACTCTGGAGTCCCTTCTCGCTTCTCTGGTAGCCGTTCCGGGACGGATTT |
| | CACTCTGACCATCAGCAGTCTGCAGCCGGAAGACTTCGCAACTTATTACTGTCAGCAAGGTTCTTACCTGTTCACG |
| | TTCGGACAGGGTACCAAGGTGGAGATCAAACGTACGGTGGCTGCACCATCTGTCTTCATCTTCCCGCCATCTGAT |
| | GAGCAGTTGAAATCTGGAACTGCCTCTGTTGTGTGCCTGCTGAATAACTTCTATCCCAGAGAGGCCAAAGTACAG |
| | TGGAAGGTGGATAACGCCCTCCAATCGGGTAACTCCCAGGAGAGTGTCACAGAGCAGGACAGCAAGGACAGCA |
| | CCTACAGCCTCAGCAGCACCCTGACGCTGAGCAAAGCAGACTACGAAAAACATAAAGTCTACGCCTGCGAAGTC |
| | ACCCATCAGGGCCTGAGCTCGCCCGTCACAAAGAGCTTCAACAGGGGAGAGTGT |
| VH-CH1-VL-CL | EVQLVESGGGLVQPGGSLRLSCAASGFDLTGSYMHWVRQAPGKGLEWVAGISASGGATAYADSVKGRFTISADTSK |
| AA | NTAYLQMNSLRAEDTAVYYCARSRSSSYSSSGWRYYSGAFDYWGQGTLVTVSSASTKGPSVFPLAPSSKSTSGGTAAL |
| | GCLVKDYFPEPVTVSWNSGALTSGVHTFPAVLQSSGLYSLSSVVTVPSSSLGTQTYICNVNHKPSNTKVDKKVEPKSCD |
| | KTHTSGGGGSGGGGSGGGGSDIQMTQSPSSLSASVGDRVTITCRASQSVSSAVAWYQQKPGKAPKLLIYSASDLYSG |
| | VPSRFSGSRSGTDFTLTISSLQPEDFATYYCQQGSYLFTFGQGTKVEIKRTVAAPSVFIFPPSDEQLKSGTASVVCLLNNF |
| | YPREAKVQWKVDNALQSGNSQESVTEQDSKDSTYSLSSTLTLSKADYEKHKVYACEVTHQGLSSPVTKSFNRGEC |
| VL-CL- | GATATCCAGATGACCCAGTCCCCGAGCTCCCTGTCCGCCTCTGTGGGCGATAGGGTCACCATCACCTGCCGTGCC |
| hIgG1 Fc | AGTCAGTCCGTGTCCAGCGCTGTAGCCTGGTATCAACAGAAACCAGGAAAAGCTCCGAAGCTTCTGATTTACTCG |
| NT | SCATCCGATCTCTACTCTGGAGTCCCTTCTCGCTTCTCTGGTAGCCGTTCCGGGACGGATTTCACTCTGACCATCA |
| (primary) | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGTAGCGGAGGAGGTGGCAGTGGCGGAGGTGGC |
| | AGCGGTGGAGGTGGCAGTGAGGTTCAGCTGGTGGAGTCTGGCGGTGGCCTGGTGCAGCCAGGGGGCTCACTC |
| | CGTTTGTCCTGTGCAGCTTCTGGCTTCGATCTCACTGGTTCTTACATGCACTGGGTGCGTCAGGCCCCGGGTAAG |
| | GGCCTGGAATGGGTTGCAGGTATTTCCGCTTCTGGAGGCGCTACTGCTTATGCCGATAGCGTCAAGGGCCGTTT |
| | CACTATAAGCGCAGACACATCCAAAAACACAGCCTACCTACAAATGAACAGCTTAAGAGCTGAGGACACTGCCG |
| | TCTATTATTGTGCTCGCTCTCGTTCTTCTTCTTACTCTTCTTCTGGTTGGCGTTACTACTCTGGTGCTTTTGACTACT |
| | GGGGTCAAGGAACCCTGGTCACCGTCTCCTCGGCTAGCACCAAGGGTCCATCGGTCTTCCCCCTGGCACCCTCCT |
| | CCAAGAGCACCTCTGGGGGCACAGCGGCCCTGGGCTGCCTGGTCAAGGACTACTTCCCCGAACCCGTGACGGTG |
| | TCGTGGAACTCAGGCGCCCTGACCAGCGGCGTGCACACCTTCCCGGCTGTCCTACAGTCCTCAGGACTCTACTCC |
| | CTCAGCAGCGTGGTGACCGTGCCCTCCAGCAGCTTGGGCACCCAGACCTACATCTGCAACGTGAATCACAAGCC |
| | CAGCAACACCAAGGTCGACAAGAAAGTTGAGCCCAAATCTTGTGACAAAACTCACACATGCCCACCGTGCCCAG |
| | CACCTGAACTCCTGGGGGGACCGTCAGTCTTCCTCTTCCCCCCAAAACCCAAGGACACCCTCATGATCTCCCGGA |
| | CCCCTGAGGTCACATGCGTGGTGGTGGACGTGAGCCACGAAGACCCTGAGGTCAAGTTCAACTGGTACGTGGA |
| | CGGCGTGGAGGTGCATAATGCCAAGACAAAGCCGCGGGAGGAGCAGTACAACAGCACGTACCGTGTGGTCAG |
| | CGTCCTCACCGTCCTGCACCAGGACTGGCTGAATGGCAAGGAGTACAAGTGCAAGGTCTCCAACAAAGCCCTCC |
| | CAGCCCCCATCGAGAAAACCATCTCCAAAGCCAAAGGGCAGCCCCGAGAACCACAGGTGTACACCCTGCCCCCA |
| | TCCCGGGAGGAGATGACCAAGAACCAGGTCAGCCTGACCTGCCTGGTCAAAGGCTTCTATCCCAGCGACATOGC |
| | CGTGGAGTGGGAGAGCAATGGGCAGCCGGAGAACAACTACAAGACCACGCCTCCCGTGCTGGACTCCGACGGC |
| | TCCTTCTTCCTCTACAGCAAGCTCACCGTGGACAAGAGCAGGTGGCAGCAGGGGAACGTCTTCTCATGCTCCGTG |
| | ATGCATGAGGCTCTGCACAACCACTACACGCAGAAGAGCCTCTCCCTGTCTCCGGGTAAA |
| VL-CL- | DIQMTQSPSSLSASVGDRVTITCRASQSVSSAVAWYQQKPGKAPKLLIYSASDLYSGVPSRFSGSRSGTDFTLTISSLQP |
| hIgG1 | EDFATYYCQQGSYLFTFGQGTKVEIKRTVAAPSVFIFPPSDEQLKSGTASVVCLLNNFYPREAKVDWKVDNALQSGNS |
| AA | QESVTEQDSKDSTYSLSSTLTLSKADYEKHKVYACEVTHQGLSSPVTKSFNRGECSGGGGSGGGGSGGGGSEVQLVES |
| (primary) | GGGLVQPGGSLRLSCAASGFDLTGSYMHWVRQAPGKGLEWVAGISASGGATAYADSVKGRFTISADTSKNTAYLQ |
| | MNSLRAEDTAVYYCARSRSSSYSSSGWRYYSGAFDYWGQGTLVTVSSASTKGPSVFPLAPSSKSTSGGTAALGCLVKD |
| | YFPEPVTVSWNSGALTSGVHTFPAVLQSSGLYSLSSVVTVPSSSLGTQTYICNVNHKPSNTKVDKKVEPKSCDKTHTCP |
| | PCPAPELLGGPSVFLFPPKPKDTLMISRTPEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREEMTKNQVSLTCLVKGFYPSDIAVEWES |
| | NGQPENNYKTTPPVLDSDGSFFLYSKLTVDKSRWQQGNVFSCSVMHEALHNHYTQKSLSLSPGK |
| 45. LEAD VHH-fab-hIgG1 clone example | |
| 15031 VHH-Fab-hIgG1 | |
| VL-CL NT | GATATCCAGATGACCCAGTCCCCGAGCTCCCTGTCCGCCTCTGTGGGCGATAGGGTCACCATCACCTGCCGTGCC |
| | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTLTISSLQP |
| | EDFATYYCQQGSYLFTFGQGTKVEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGEC |
| VHH-VH-CH1- | GAGGTTCAGCTGGTGGAGTCTGGCGGTGGCCTGGTGCAGCCAGGGCGTTCACTCCGTTTGTCCTGTGCAGCTTC |
| hIgG1 | TGGCTTCGACCTCACTGGTTCTTACATGCACTGGGTGCGTCGTGCCCCGGGTAAGGGCGAGGAACTGGTTGCAG |
| NT | GTATTTCCGCTTCTGGAGGCGCTACTGCTTATGCCGATAGCGTCAAGGGCCGTTTCACTATAAGCGCAGACACAT |
| | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGGTTGGCGTTACTACTCTGGTGCTTTTGACTACTGGGGTCAAGGAACCCTGGTC |
| | ACCGTCTCCTCGGGTGGAGGTGGCTCCGGAGGAGGTGGCAGTGAGGTTCAGCTGGTGGAGTCTGGCGGTGGC |
| | CTGGTGCAGCCAGGGGGCTCACTCCGTTTGTCCTGTGCAGCTTCTGGCTTCGACCTCACTGGTTCTTACATGCACT |
| | GGGTGCGTCAGGCCCCGGGTAAGGGCCTGGAATGGGTTGCAGGTATTTCCGCTTCTGGAGGCGCTACTGCTTAT |
| | GCCGATAGCGTCAAGGGCCGTTTCACTATAAGCGCAGACACATCCAAAAACACAGCCTACCTACAAATGAACAG |
| | CTTAAGAGCTGAGGACACTGCCGTCTATTATTGTGCTCGCTCTCGTTCTTCTTCTTACTCTTCTTCTGGTTGGCGTT |
| | ACTACTCTGGTGCTTTTGACTACTGGGGTCAAGGAACCCTGGTCACCGTCTCCTCGGCTAGCACCAAGGGCCCAT |
| | CGGTCTTCCCCCTGGCACCCTCCTCCAAGAGCACCTCTGGGGGCACAGCGGCCCTGGGCTGCCTGGTCAAGGAC |
| | TACTTCCCCGAACCGGTGACGGTGTCGTGGAACTCAGGCGCCCTGACCAGCGGCGTGCACACCTTCCCGGCTGT |
| | CCTACAGTCCTCAGGACTCTACTCCCTCAGCAGCGTGGTGACCGTGCCCTCCAGCAGCTTGGGCACCCAGACCTA |
| | CATCTGCAACGTGAATCACAAGCCCAGCAACACCAAGGTGGACAAGAAAGTTGAGCCCAAATCTTGTGACAAAA |
| | CTCACACATCCGGAGGAGGTGGCAGTGGCGGAGGTGGCAGCGGTGGAGGTGGCAGTGAGGTTCAGCTGGTG |
| | GAGTCTGGCGGTGGCCTGGTGCAGCCAGGGGGCTCACTCCGTTTGTCCTGTGCAGCTTCTGGCTTCGACCTCACT |
| | GGTTCTTACATGCACTGGGTGCGTCAGGCCCCGGGTAAGGGCCTGGAATGGGTTGCAGGTATTTCCGCTTCTGG |
| | AGGCGCTACTGCTTATGCCGATAGCGTCAAGGGCCGTTTCACTATAAGCGCAGACACATCCAAAAACACAGCCT |
| | ACCTACAAATGAACAGCTTAAGAGCTGAGGACACTGCCGTCTATTATTGTGCTCGCTCTCGTTCTTCTTCTTACTCT |
| | TCTTCTGGTTGGCGTTACTACTCTGGTGCTTTTGACTACTGGGGTCAAGGAACCCTGGTCACCGTCTCCTCGGCTA |
| | GCACCAAGGGCCCATCGGTCTTCCCCCTGGCACCCTCCTCCAAGAGCACCTCTGGGGGCACAGCGGCCCTGGGC |
| | TGCCTGGTCAAGGACTACTTCCCCGAACCGGTGACGGTGTCGTGGAACTCAGGCGCCCTGACCAGCGGCGTGCA |
| | CACCTTCCCGGCTGTCCTACAGTCCTCAGGACTCTACTCCCTCAGCAGCGTGGTGACCGTGCCCTCCAGCAGCTTG |
| | GGCACCCAGACCTACATCTGCAACGTGAATCACAAGCCCAGCAACACCAAGGTGGACAAGAAAGTTGAGCCCAA |
| | ATCTTGTGACAAAACTCACACATGCCCACCGTGCCCAGCACCTGAACTCCTGGGGGGACCGTCAGTCTTCCTCTT |
| | CCCCCCAAAACCCAAGGACACCCTCATGATCTCCCGGACCCCTGAGGTCACATGCGTGGTGGTGGACGTGAGCC |
| | ACGAAGACCCTGAGGTCAAGTTCAACTGGTACGTGGACGGCGTGGAGGTGCATAATGCCAAGACAAAGCCGCG |
| | GGAGGAGCAGTACAACAGCACGTACCGTGTGGTCAGCGTCCTCACCGTCCTGCACCAGGACTGGCTGAATGGC |
| | AAGGAGTACAAGTGCAAGGTCTCCAACAAAGCCCTCCCAGCCCCCATCGAGAAAACCATCTCCAAAGCCAAAGG |
| | GCAGCCCCGAGAACCACAGGTGTACACCCTGCCCCCATCCCGGGAGGAGATGACCAAGAACCAGGTCAGCCTG |
| | ACCTGCCTGGTCAAAGGCTTCTATCCCAGCGACATCGCCGTGGAGTGGGAGAGCAATGGGCAGCCGGAGAACA |
| | ACTACAAGACCACGCCTCCCGTGCTGGACTCCGACGGCTCCTTCTTCCTCTACAGCAAGCTCACCGTGGACAAGA |
| | GCAGGTGGCAGCAGGGGAACGTCTTCTCATGCTCCGTGATGCATGAGGCTCTGCACAACCACTACACGCAGAAG |
| | AGCCTCTCCCTGTCTCCGGGTAAA |
| VHH-VH-CH1- | EVQLVESGGGLVQPGRSLRLSCAASGFDLTGSYMHWVRRAPGKGEELVAGISASGGATAYADSVKGRFTISADTSKN |
| hIgG1 | TAYLQMNSLRAEDTAVYYCARSRSSSYSSSGWRYYSGAFDYWGQGTLVTVSSGGGGSGGGGSEVQLVESGGGLVQ |
| AA | PGGSLRLSCAASGFDLTGSYMHWVRQAPGKGLEWVAGISASGGATAYADSVKGRFTISADTSKNTAYLQMNSLRAE |
| | DTAVYYCARSRSSSYSSSGWRYYSGAFDYWGQGTLVTVSSASTKGPSVFPLAPSSKSTSGGTAALGCLVKDYFPEPVT |
| | VSWNSGALTSGVHTFPAVLQSSGLYSLSSVVTVPSSSLGTQTYICNVNHKPSNTKVDKKVEPKSCDKTHTSGGGGSGG |
| | GGSGGGGSEVQLVESGGGLVQPGGSLRLSCAASGFDLTGSYMHWVRQAPGKGLEWVAGISASGGATAYADSVKG |
| | RFTISADTSKNTAYLQMNSLRAEDTAVYYCARSRSSSYSSSGWRYYSGAFDYWGQGTLVTVSSASTKGPSVFPLAPSS |
| | KSTSGGTAALGCLVKDYFPEPVTVSWNSGALTSGVHTPPAVLQSSGLYSLSSVVTVPSSSIGTQTYICNVNHKPSNTKV |
| | DKKVEPKSCDKTHTCPPCPAPELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQVYTLPPSREEMTKNQVSLTC |
| | LVKGFVPSDIAVEWESNGQPENNYKTTPPVLDSDGSFFLYSKLTVDKSRWQQGNVFSCSVMHEALHNHYTQKSLSLS |
| | PGK |
| 46. LEAD scFv-Fab-hIgG1 clone example | |
| 15031 scFv-Fab-hIgG1 | |
| VL-CL NT | GATATCCAGATGACCCAGTCCCCGAGCTCCCTGTCCGCCTCTGTGGGCGATAGGGTCACCATCACCTGCCGTGCC |
| | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTITISSLQP |
| | EDFATYYCQQGSYLFTFGQGTKVEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGEC |
| VL-VH-VH- | GATATCCAGATGACCCAGTCCCCGAGCTCCCTGTCCGCCTCTGTGGGCGATAGGGTCACCATCACCTGCCGTGCC |
| CH1-hIgG1 NT | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAAGGTACTACTGCCGCTAGTGGTAGTAGTGGTGGCAGTAGCAGTGGTGCCGAGGTTCA |
| | GCTGGTGGAGTCTGGCGGTGGCCTGGTGCAGCCAGGGGGCTCACTCCGTTTGTCCTGTGCAGCTTCTGGCTTCG |
| | ACCTCACTGGTTCTTACATGCACTGGGTGCGTCAGGCCCCGGGTAAGGGCCTGGAATGGGTTGCAGGTATTTCC |
| | GCTTCTGGAGGCGCTACTGCTTATGCCGATAGCGTCAAGGGCCGTTTCACTATAAGCGCAGACACATCCAAAAA |
| | CACAGCCTACCTACAAATGAACAGCTTAAGAGCTGAGGACACTGCCGTCTATTATTGTGCTCGCTCTCGTTCTTCT |
| | TCTTACTCTTCTTCTGGTTGGCGTTACTACTCTGGTGCTTTTGACTACTGGGGTCAAGGAACCCTGGTCACCGTCT |
| | CCTCGGGTGGAGGTGGCTCCGGAGGAGGTGGCAGTGAGGTTCAGCTGGTGGAGTCTGGCGGTGGCCTGGTGC |
| | AGCCAGGGGGCTCACTCCGTTTGTCCTGTGCAGCTTCTGGCTTCGACCTCACTGGTTCTTACATGCACTGGGTGC |
| | GTCAGGCCCCGGGTAAGGGCCTGGAATGGGTTGCAGGTATTTCCGCTTCTGGAGGCGCTACTGCTTATGCCGAT |
| | AGCGTCAAGGGCCGTTTCACTATAAGCGCAGACACATCCAAAAACACAGCCTACCTACAAATGAACAGCTTAAG |
| | AGCTGAGGACACTGCCGTCTATTATTGTGCTCGCTCTCGTTCTTCTTCTTACTCTTCTTCTGGTTGGCGTTACTACT |
| | CTGGTGCTTTTGACTACTGGGGTCAAGGAACCCTGGTCACCGTCTCCTCGGCTAGCACCAAGGGCCCATCGGTCT |
| | TCCCCCTGGCACCCTCCTCCAAGAGCACCTCTGGGGGCACAGCGGCCCTGGGCTGCCTGGTCAAGGACTACTTCC |
| | CCGAACCGGTGACGGTGTCGTGGAACTCAGGCGCCCTGACCAGCGGCGTGCACACCTTCCCGGCTGTCCTACAG |
| | TCCTCAGGACTCTACTCCCTCAGCAGCGTGGTGACCGTGCCCTCCAGCAGCTTGGGCACCCAGACCTACATCTGC |
| | AACGTGAATCACAAGCCCAGCAACACCAAGGTGGACAAGAAAGTTGAGCCCAAATCTTGTGACAAAACTCACAC |
| | ATCCGGAGGAGGTGGCAGTGGCGGAGGTGGCAGCGGTGGAGGTGGCAGTGAGGTTCAGCTGGTGGAGTCTG |
| | GCGGTGGCCTGGTGCAGCCAGGGGGCTCACTCCGTTTGTCCTGTGCAGCTTCTGGCTTCGACCTCACTGGTTCTT |
| | ACATGCACTGGGTGCGTCAGGCCCCGGGTAAGGGCCTGGAATGGGTTGCAGGTATTTCCGCTTCTGGAGGCGCT |
| | ACTGCTTATGCCGATAGCGTCAAGGGCCGTTTCACTATAAGCGCAGACACATCCAAAAACACAGCCTACCTACAA |
| | ATGAACAGCTTAAGAGCTGAGGACACTGCCGTCTATTATTGTGCTCGCTCTCGTTCTTCTTCTTACTCTTCTTCTGG |
| | TTGGCGTTACTACTCTGGTGCTTTTGACTACTGGGGTCAAGGAACCCTGGTCACCGTCTCCTCGGCTAGCACCAA |
| | GGGCCCATCGGTCTTCCCCCTGGCACCCTCCTCCAAGAGCACCTCTGGGGGCACAGCGGCCCTGGGCTGCCTGG |
| | TCAAGGACTACTTCCCCGAACCGGTGACGGTGTCGTGGAACTCAGGCGCCCTGACCAGCGGCGTGCACACCTTC |
| | CCGGCTGTCCTACAGTCCTCAGGACTCTACTCCCTCAGCAGCGTGGTGACCGTGCCCTCCAGCAGCTTGGGCACC |
| | CAGACCTACATCTGCAACGTGAATCACAAGCCCAGCAACACCAAGGTGGACAAGAAAGTTGAGCCCAAATCTTG |
| | TGACAAAACTCACACATGCCCACCGTGCCCAGCACCTGAACTCCTGGGGGGACCGTCAGTCTTCCTCTTCCCCCC |
| | AAAACCCAAGGACACCCTCATGATCTCCCGGACCCCTGAGGTCACATGCGTGGTGGTGGACGTGAGCCACGAAG |
| | ACCCTGAGGTCAAGTTCAACTGGTACGTGGACGGCGTGGAGGTGCATAATGCCAAGACAAAGCCGCGGGAGGA |
| | GCAGTACAACAGCACGTACCGTGTGGTCAGCGTCCTCACCGTCCTGCACCAGGACTGGCTGAATGGCAAGGAGT |
| | ACAAGTGCAAGGTCTCCAACAAAGCCCTCCCAGCCCCCATCGAGAAAACCATCTCCAAAGCCAAAGGGCAGCCC |
| | CGAGAACCACAGGTGTACACCCTGCCCCCATCCCGGGAGGAGATGACCAAGAACCAGGTCAGCCTGACCTGCCT |
| | GGTCAAAGGCTTCTATCCCAGCGACATCGCCGTGGAGTGGGAGAGCAATGGGCAGCCGGAGAACAACTACAAG |
| | ACCACGCCTCCCGTGCTGGACTCCGACGGCTCCTTCTTCCTCTACAGCAAGCTCACCGTGGACAAGAGCAGGTGG |
| | CAGCAGGGGAACGTCTTCTCATGCTCCGTGATGCATGAGGCTCTGCACAACCACTACACGCAGAAGAGCCTCTC |
| | CCTGTCTCCGGGTAAA |
| VL-VH-VH-CH1- | DIQMTQSPSSLSASVGDRVTITCRASQSVSSAVAWYQQKPGKAPKLLIYSASDLYSGVPSRFSGSRSGTDFTLTISSLQP |
| hIgG1 AA | EDFATYYCQQGSYLFTFGQGTKVEIKGTTAASGSSGGSSSGAEVQLVESGGGLVQPGGSLRLSCAASGFDLTGSYMH |
| | WVRQAPGKGLEWVAGISASGGATAYADSVKGRFTISADTSKNTAYLQMNSLRAEDTAVYYCARSRSSSYSSSGWRY |
| | YSGAFDYWGQGTLVTVSSGGGGSGGGGSEVQLVESGGGLVQPGGSLRLSCAASGFDLTGSYMHWVRQAPGKGLE |
| | WVAGISASGGATAYADSVKGRFTISADTSKNTAYLQMNSLRAEDTAVYYCARSRSSSYSSSGWRYYSGAFDYWGQG |
| | TLVTVSSASTKGPSVFPLAPSSKSTSGGTAALGCLVKDYFPEPVTVSWNSGALTSGVHTFPAVLQSSGLYSLSSVVTVPS |
| | SSLGTQTYICNVNHKPSNTKVDKKVEPKSCDKTHTSGGGGSGGGGSGGGGSEVQLVESGGGLVQPGGSLRLSCAAS |
| | GFDLTGSYMHWVRAAPGKGLEWVAGISASGGATAYADSVKGRFTISADTSKNTAYLQMNSLRAEDTAVYYCARSRS |
| | SSYSSSGWRYYSGAFDYWGQGTLVTVSSASTKGPSVFPLAPSSKSTSGGTAALGCLVKDYFPEPVTVSWNSGALTSGV |
| | HTFPAVLQSSGLYSLSSVVTVPSSSLGTQTYICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 47. LEAD Fab-VHH-hIgG1 clone example | |
| 15031 Fab-VHH-hIgG1 | |
| VL-CL NT | GATATCCAGATGACCCAGTCCCCGAGCTCCCTGTCCGCCTCTGTGGGCGATAGGGTCACCATCACCTGCCGTGCC |
| | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTLTISSLQP |
| | EDFATYYCQQGSYLFTFGQGTKVEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGEC |
| VH-CH1-VHH- | GAGGTTCAGCTGGTGGAGTCTGGCGGTGGCCTGGTGCAGCCAGGGGGCTCACTCCGTTTGTCCTGTGCAGCTTC |
| hIgG1 | TGGCTTCGACCTCACTGGTTCTTACATGCACTGGGTGCGTCAGGCCCCGGGTAAGGGCCTGGAATGGGTTGCAG |
| NT | GTATTTCCGCTTCTGGAGGCGCTACTGCTTATGCCGATAGCGTCAAGGGCCGTTTCACTATAAGCGCAGACACAT |
| | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGGTTGGCGTTACTACTCTGGTGCTTTTGACTACTGGGGTCAAGGAACCCTGGTC |
| | ACCGTCTCCTCGGCTAGCACCAAGGGCCCATCGGTCTTCCCCCTGGCACCCTCCTCCAAGAGCACCTCTGGGGGC |
| | ACAGCGGCCCTGGGCTGCCTGGTCAAGGACTACTTCCCCGAACCGGTGACGGTGTCGTGGAACTCAGGCGCCCT |
| | GACCAGCGGCGTGCACACCTTCCCGGCTGTCCTACAGTCCTCAGGACTCTACTCCCTCAGCAGCGTGGTGACCGT |
| | GCCCTCCAGCAGCTTGGGCACCCAGACCTACATCTGCAACGTGAATCACAAGCCCAGCAACACCAAGGTGGACA |
| | AGAAAGTTGAGCCCAAATCTTGTGACAAAACTCACACAGGTGGAGGTGGCTCCGGAGGAGGTGGCAGTGAGG |
| | TTCAGCTGGTGGAGTCTGGCGGTGGCCTGGTGCAGCCAGGGCGTTCACTCCGTTTGTCCTGTGCAGCTTCTGGCT |
| | TCGACCTCACTGGTTCTTACATGCACTGGGTGCGTCGTGCCCCGGGTAAGGGCGAGGAACTGGTTGCAGGTATT |
| | TCCGCTTCTGGAGGCGCTACTGCTTATGCCGATAGCGTCAAGGGCCGTTTCACTATAAGCGCAGACACATCCAAA |
| | AACACAGCCTACCTACAAATGAACAGCTTAAGAGCTGAGGACACTGCCGTCTATTATTGTGCTCGCTCTCGTTCTT |
| | CTTCTTACTCTTCTTCTGGTTGGCGTTACTACTCTGGTGCTTTTGACTACTGGGGTCAAGGAACCCTGGTCACCGT |
| | CTCCTCGGGTGGAGGTGGCTCCGGAGGAGGTGGCAGTGAGGTTCAGCTGGTGGAGTCTGGCGGTGGCCTGGT |
| | GCAGCCAGGGGGCTCACTCCGTTTGTCCTGTGCAGCTTCTGGCTTCGACCTCACTGGTTCTTACATGCACTGGGT |
| | GCGTCAGGCCCCGGGTAAGGGCCTGGAATGGGTTGCAGGTATTTCCGCTTCTGGAGGCGCTACTGCTTATGCCG |
| | ATAGCGTCAAGGGCCGTTTCACTATAAGCGCAGACACATCCAAAAACACAGCCTACCTACAAATGAACAGCTTAA |
| | GAGCTGAGGACACTGCCGTCTATTATTGTGCTCGCTCTGGTTCTTCTTCTTACTCTTCTTCTGGTTGGCGTTACTAC |
| | TCTGGTGCTTTTGACTACTGGGGTCAAGGAACCCTGGTCACCGTCTCCTCGGCTAGCACCAAGGGCCCATCGGTC |
| | TTCCCCCTGGCACCCTCCTCCAAGAGCACCTCTGGGGGCACAGCGGCCCTGGGCTGCCTGGTCAAGGACTACTTC |
| | CCCGAACCGGTGACGGTGTCGTGGAACTCAGGCGCCCTGACCAGCGGCGTGCACACCTTCCCGGCTGTCCTACA |
| | GTCCTCAGGACTCTACTCCCTCAGCAGCGTGGTGACCGTGCCCTCCAGCAGCTTGGGCACCCAGACCTACATCTG |
| | CAACGTGAATCACAAGCCCAGCAACACCAAGGTGGACAAGAAAGTTGAGCCCAAATCTTGTGACAAAACTCACA |
| | CATGCCCACCGTGCCCAGCACCTGAACTCCTGGGGGGACCGTCAGTCTTCCTCTTCCCCCCAAAACCCAAGGACA |
| | CCCTCATGATCTCCCGGACCCCTGAGGTCACATGCGTGGTGGTGGACGTGAGCCACGAAGACCCTGAGGTCAAG |
| | TTCAACTGGTACGTGGACGGCGTGGAGGTGCATAATGCCAAGACAAAGCCGCGGGAGGAGCAGTACAACAGCA |
| | CGTACCGTGTGGTCAGCGTCCTCACCGTCCTGCACCAGGACTGGCTGAATGGCAAGGAGTACAAGTGCAAGGTC |
| | TCCAACAAAGCCCTOCCAGCCCCCATCGAGAAAACCATCTCCAAAGCCAAAGGGCAGCCCCGAGAACCACAGGT |
| | GTACACCCTGCCCCCATCCCGGGAGGAGATGACCAAGAACCAGGTCAGCCTGACCTGCCTGGTCAAAGGCTTCT |
| | ATCCCAGCGACATCGCCGTGGAGTGGGAGAGCAATGGGCAGCCGGAGAACAACTACAAGACCACGCCTCCCGT |
| | GCTGGACTCCGACGGCTCCTTCTTCCTCTACAGCAAGCTCACCGTGGACAAGAGCAGGTGGCAGCAGGGGAACG |
| | TCTTCTCATGCTCCGTGATGCATGAGGCTCTGCACAACCACTACACGCAGAAGAGCCTCTCCCTGTCTCCGGGTA |
| | AA |
| VH-CH1-VHH- | EVQLVESGGGLVQPGGSLRLSCAASGFDLTGSYMHWVRQAPGKGLEWVAGISASGGATAYADSVKGRFTISADTSK |
| hIgG1 | NTAYLQMNSLRAEDTAVYYCARSRSSSYSSSGWRYYSGAFDYWGQGTLVTVSSASTKGPSVPPLAPSSKSTSGGTAAL |
| AA | GCLVKDYFPEPVTVSWNSGALTSGVHTFPAVLQSSGLYSLSSVVTVPSSSLGTQTYICNVNHKPSNTKVDKKVEPKSCD |
| | KTHTGGGGSGGGGSEVQLVESGGGLVQPGRSLRLSCAASGFDLTGSYMHWVRRAPGKGEELVAGISASGGATAYA |
| | DSVKGAFTISADTSKNTAYLQMNSLRAEDTAVYYCARSRSSSYSSSGWRYYSGAFDYWGQGTLVTVSSGGGGSGGG |
| | GSEVQLVESGGGLVQPGGSLRLSCAASGFDLTGSYMHWVRQAPGKGLEWVAGISASGGATAYADSVKGRFTISADT |
| | SKNTAYLQMNSLRAEDTAVYYCARSRSSSYSSSGWRYYSGAFDYWGQGTLVTVSSASTKGPSVFPLAPSSKSTSGGTA |
| | ALGCLVKDYFPEPVTVSWNSGALTSGVHTFPAVLQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCSVMHEALHNHYTQKSLSLSPGK |
| 48. LEAD Fab-scFv-hIgG1 clone example | |
| 15031 Fab-scFv-hIgG1 | |
| VL-CL NT | GATATCCAGATGACCCAGTCCCCGAGCTCCCTGTCCGCCTCTGTGGGCGATAGGGTCACCATCACCTGCCGTGCC |
| | AGTCAGTCCGTGTCCAGCGCTGTAGCCTGGTATCAACAGAAACCAGGAAAAGCTCCGAAGCTTCTGATTTACTCG |
| | GCATCCGATCTCTACTCTGGAGTCCCTTCTCGCTTCTCTGGTAGCCGTTCCGGGACGGATTTCACTCTGACCATCA |
| | GCAGTCTGCAGCCGGAAGACTTCGCAACTTATTACTGTCAGCAAGGTTCTTACCTGTTCACGTTCGGACAGGGTA |
| | CCAAGGTGGAGATCAAACGTACGGTGGCTGCACCATCTGTCTTCATCTTCCCGCCATCTGATGAGCAGTTGAAAT |
| | CTGGAACTGCCTCTGTTGTGTGCCTGCTGAATAACTTCTATCCCAGAGAGGCCAAAGTACAGTGGAAGGTGGAT |
| | AACGCCCTCCAATCGGGTAACTCCCAGGAGAGTGTCACAGAGCAGGACAGCAAGGACAGCACCTACAGCCTCA |
| | GCAGCACCCTGACGCTGAGCAAAGCAGACTACGAAAAACATAAAGTCTACGCCTGCGAAGTCACCCATCAGGGC |
| | CTGAGCTCGCCCGTCACAAAGAGCTTCAACAGGGGAGAGTGT |
| VL-CL AA | DIQMTQSPSSLSASVGDRVTITCRASQSVSSAVAWYQQKPGKAPKLLIYSASDLYSGVPSRFSGSRSGTDFTLTISSLQP |
| | EDFATYYCQQGSYLFTFGQGTKVEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGEC |
| VH-CH1-VL-VH- | GAGGTTCAGCTGGTGGAGTCTGGCGGTGGCCTGGTGCAGCCAGGGGGCTCACTCCGTTTGTCCTGTGCAGCTTC |
| hIgG1 | TGGCTTCGACCTCACTGGTTCTTACATGCACTGGGTGCGTCAGGCCCCGGGTAAGGGCCTGGAATGGGTTGCAG |
| NT | GTATTTCCGCTTCTGGAGGCGCTACTGCTTATGCCGATAGCGTCAAGGGCCGTTTCACTATAAGCGCAGACACAT |
| | CCAAAAACACAGCCTACCTACAAATGAACAGCTTAAGAGCTGAGGACACTGCCGTCTATTATTGTGCTCGCTCTC |
| | GTTCTTCTTCTTACTCTTCTTCTGGTTGGCGTTACTACTCTGGTGCTTTTGACTACTGGGGTCAAGGAACCCTGGTC |
| | ACCGTCTCCTCGGCTAGCACCAAGGGCCCATCGGTCTTCCCCCTGGCACCCTCCTCCAAGAGCACCTCTGGGGGC |
| | ACAGCGGCCCTGGGCTGCCTGGTCAAGGACTACTTCCCCGAACCGGTGACGGTGTCGTGGAACTCAGGCGCCCT |
| | GACCAGCGGCGTGCACACCTTCCCGGCTGTCCTACAGTCCTCAGGACTCTACTCCCTCAGCAGCGTGGTGACCGT |
| | GCCCTCCAGCAGCTTGGGCACCCAGACCTACATCTGCAACGTGAATCACAAGCCCAGCAACACCAAGGTGGACA |
| | AGAAAGTTGAGCCCAAATCTTGTGACAAAACTCACACAGGTGGAGGTGGCTCCGGAGGAGGTGGCAGTGATAT |
| | CCAGATGACCCAGTCCCCGAGCTCOCTGTCCGCCTCTGTGGGCGATAGGGTCACCATCACCTGCCGTGCCAGTCA |
| | GTCCGTGTCCAGCGCTGTAGCCTGGTATCAACAGAAACCAGGAAAAGCTCCGAAGCTTCTGATTTACTCGGCATC |
| | CGATCTCTACTCTGGAGTCCCTTCTCGCTTCTCTGGTAGCCGTTCCGGGACGGATTTCACTCTGACCATCAGCAGT |
| | CTGCAGCCGGAAGACTTCGCAACTTATTACTGTCAGCAAGGTTCTTACCTGTTCACGTTCGGACAGGGTACCAAG |
| | GTGGAGATCAAAGGTACTACTGCCGCTAGTGGTAGTAGTGGTGGCAGTAGCAGTGGTGCCGAGGTTCAGCTG |
| | GTGGAGTCTGGCGGTGGCCTGGTGCAGCCAGGGGGCTCACTCCGTTTGTCCTGTGCAGCTTCTGGCTTCGACCT |
| | CACTGGTTCTTACATGCACTGGGTGCGTCAGGCCCCGGGTAAGGGCCTGGAATGGGTTGCAGGTATTTCCGCTT |
| | CTGGAGGCGCTACTGCTTATGCCGATAGCGTCAAGGGCCGTTTCACTATAAGCGCAGACACATCCAAAAACACA |
| | GCCTACCTACAAATGAACAGCTTAAGAGCTGAGGACACTGCCGTCTATTATTGTGCTCGCTCTCGTTCTTCTTCTT |
| | ACTCTTCTTCTGGTTGGCGTTACTACTCTGGTGCTTTTGACTACTGGGGTCAAGGAACCCTGGTCACCGTCTCCTC |
| | GGGTGGAGGTGGCTCCGGAGGAGGTGGCAGTGAGGTTCAGCTGGTGGAGTCTGGCGGTGGCCTGGTGCAGC |
| | CAGGGGGCTCACTCCGTTTGTCCTGTGCAGCTTCTGGCTTCGACCTCACTGGTTCTTACATGCACTGGGTGCGTC |
| | AGGCCCCGGGTAAGGGCCTGGAATGGGTTGCAGGTATTTCCGCTTCTGGAGGCGCTACTGCTTATGCCGATAGC |
| | GTCAAGGGCCGTTTCACTATAAGCGCAGACACATCCAAAAACACAGCCTACCTACAAATGAACAGCTTAAGAGC |
| | TGAGGACACTGCCGTCTATTATTGTGCTCGCTCTCGTTCTTCTTCTTACTCTTCTTCTGGTTGGCGTTACTACTCTG |
| | GTGCTTTTGACTACTGGGGTCAAGGAACCCTGGTCACCGTCTCCTCGGCTAGCACCAAGGGCCCATCGGTCTTCC |
| | CCCTGGCACCCTCCTCCAAGAGCACCTCTGGGGGCACAGCGGCCCTGGGCTGCCTGGTCAAGGACTACTTCCCC |
| | GAACCGGTGACGGTGTCGTGGAACTCAGGCGCCCTGACCAGCGGCGTGCACACCTTCCCGGCTGTCCTACAGTC |
| | CTCAGGACTCTACTCCCTCAGCAGCGTGGTGACCGTGCCCTCCAGCAGCTTGGGCACCCAGACCTACATCTGCAA |
| | CGTGAATCACAAGCCCAGCAACACCAAGGTGGACAAGAAAGTTGAGCCCAAATCTTGTGACAAAACTCACACAT |
| | GCCCACCGTGCCCAGCACCTGAACTCCTGGGGGGACCGTCAGTCTTCCTCTTCCCCCCAAAACCCAAGGACACCC |
| | TCATGATCTCCCGGACCCCTGAGGTCACATGCGTGGTGGTGGACGTGAGCCACGAAGACCCTGAGGTCAAGTTC |
| | AACTGGTACGTGGACGGCGTGGAGGTGCATAATGCCAAGACAAAGCCGCGGGAGGAGCAGTACAACAGCACG |
| | TACCGTGTGGTCAGCGTCCTCACCGTCCTGCACCAGGACTGGCTGAATGGCAAGGAGTACAAGTGCAAGGTCTC |
| | CAACAAAGCCCTCCCAGCCCCCATCGAGAAAACCATCTCCAAAGCCAAAGGGCAGCCCCGAGAACCACAGGTGT |
| | ACACCCTGCCCCCATCCCGGGAGGAGATGACCAAGAACCAGGTCAGCCTGACCTGCCTGGTCAAAGGCTTCTAT |
| | CCCAGCGACATCGCCGTGGAGTGGGAGAGCAATGGGCAGCCGGAGAACAACTACAAGACCACGCCTCCCGTGC |
| | TGGACTCCGACGGCTCCTTCTTCCTCTACAGCAAGCTCACCGTGGACAAGAGCAGGTGGCAGCAGGGGAACGTC |
| | TTCTCATGCTCCGTGATGCATGAGGCTCTGCACAACCACTACACGCAGAAGAGCCTCTCCCTGTCTCCGGGTAAA |
| VH-CH1-VL- | EVQLVESGGGLVQPGGSLRLSCAASGFDLTGSYMHWVRQAPGKGLEWVAGISASGGATAYADSVKGRFTISADTSK |
| VH-hIgG1 AA | NTAYLQMNSLRAEDTAVYYCARSRSSSYSSSGWRYYSGAFDYWGQGTLVTVSSASTKGPSVFPLAPSSKSTSGGTAAL |
| | GCLVKDYFPEPVTVSWNSGALTSGVHTFPAVLQSSGLYSLSSVVTVPSSSLGTQTYICNVNHKPSNTKVDKKVEPKSCD |
| | KTHTGGGGSGGGGSDIQMTQSPSSLSASVGDRVTITCRASQSVSSAVAWYQQKPGKAPKILIYSASDLYSGVPSRFS |
| | GSRSGTDFTLTISSLQPEDFATYYCQQGSYLFTFGQGTKVEIKGTTAASGSSGGSSSGAEVQLVESGGGLVQPGGSLAL |
| | SCAASGFDLTGSYMHWVRQAPGKGLEWVAGISASGGATAYADSVKGRFTISADTSKNTAYLQMNSLRAEDTAVYYC |
| | ARSRSSSYSSSGWRYYSGAFDYWGQGTLVTVSSGGGGSGGGGSEVQLVESGGGLVQPGGSLRLSCAASGFDLTGSY |
| | MHWVRQAPGKGLEWVAGISASGGATAYADSVKGRFTISADTSKNTAYLQMNSLRAEDTAVYYCARSRSSSYSSSGW |
| | RYYSGAFDYWGQGTLVTVSSASTKGPSVFPLAPSSKSTSGGTAALGCLVKDYFPEPVTVSWNSGALTSGVHTFPAVLQ |
| | SSGLYSLSSVVTVPSSSLGTQTYICNVNMKPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLTCLVKGFYPSDIAVEWESNGQPENNYKTTPPVLDSDGSFFLYSKLT |
| | VDKSRWQQGNVFSCSVMHEALHNHYTQKSLSLSPGK |

TABLE 8
The peptide sequences identified in FIG. 2 of
this application are shown below in table form.

| Peptide | | | | | | | | | | | | | | | | | | | | | |
|---|---|---|---|---|---|---|---|---|---|---|---|---|---|---|---|---|---|---|---|---|---|
| number | Peptide family | | | | | | | | | | | | | | | | | | | | |
| | Unconstrained (WE) | | | | | | | | | | | | | | | | | | | | |
| Consensus | X | H | H | X | M | I | X | W | E | X | Q | L | V | Q | M | L | X | X | H | L | I |
| 1 | N | H | L | A | D | P | S | W | E | W | M | L | V | Q | M | L | — | — | — | — | — |
| 2 | P | E | A | S | V | I | A | W | E | W | F | L | F | P | E | W | — | — | — | — | — |
| 3 | Q | P | M | Y | T | H | E | W | E | W | V | L | Q | H | I | M | — | — | — | — | — |
| 4 | D | W | H | M | G | Y | V | W | E | A | E | L | I | S | I | M | — | — | — | — | — |
| 5 | T | H | H | E | D | M | Y | W | D | H | Q | L | V | F | I | M | — | — | — | — | — |
| 6 | P | F | V | N | N | D | N | W | E | S | Q | L | V | D | M | M | — | — | — | — | — |
| 7 | E | M | D | R | H | W | N | W | E | Y | W | I | V | Q | M | M | — | — | — | — | — |
| 8 | N | Q | E | K | Y | I | Q | W | E | Y | R | L | V | Q | M | M | — | — | — | — | — |
| 9 | W | H | W | T | M | E | H | W | E | S | S | L | I | E | M | L | — | — | — | — | — |
| 10 | S | K | S | D | M | H | P | W | E | R | Q | L | I | W | M | L | — | — | — | — | — |
| 11 | Q | H | H | D | M | Q | W | W | E | V | Q | I | V | N | M | L | — | — | — | — | — |
| 12 | — | P | F | F | T | A | M | W | E | E | W | L | V | E | V | L | G | — | — | — | — |
| 13 | V | R | S | P | P | P | H | W | E | E | F | L | V | D | M | L | — | — | — | — | — |
| 14 | — | — | — | — | — | I | P | W | E | I | D | L | E | Q | W | I | Q | M | H | L | I |
| 15 | — | — | M | S | W | V | A | W | E | E | M | L | E | S | W | L | W | T | — | — | — |
| | Unconstrained (Wφ) | | | | | | | | | | | | | | | | | | | | |
| Consensus | D | W | X | E | X | E | E | W | M | X | Q | L | V | Y | M | L | X | P | X | | |
| 16 | D | F | D | F | A | E | N | W | M | E | Q | L | K | V | M | L | — | — | — | | |
| 17 | — | — | E | D | D | M | L | W | T | A | I | F | G | Q | M | W | E | I | — | | |
| 18 | — | — | — | H | N | L | M | W | Y | S | N | F | M | H | M | W | I | P | I | | |
| 19 | Q | W | E | L | F | E | Q | W | V | M | N | N | Y | N | T | V | — | — | — | | |
| 20 | E | I | D | F | F | Y | Q | W | L | D | Q | M | Q | L | N | N | — | — | — | | |
| 21 | — | — | W | E | P | E | P | W | M | L | Q | A | F | W | E | W | Q | L | — | | |
| 22 | D | D | W | E | F | L | F | W | Q | V | M | F | P | E | W | Y | — | — | — | | |
| 23 | D | D | T | W | V | M | E | W | I | Q | S | M | G | W | S | Q | — | — | — | | |
| 24 | D | I | W | W | H | P | S | W | L | E | S | F | T | H | D | Y | — | — | — | | |
| 25 | S | Y | V | H | D | P | S | W | M | M | E | M | M | H | I | L | — | — | — | | |
| 26 | — | — | — | E | Y | H | A | W | M | E | M | L | A | S | W | A | A | P | H | | |
| 27 | — | W | I | E | Q | F | A | I | L | M | G | A | E | M | F | L | P | — | — | | |
| 28 | — | P | V | E | Q | W | N | W | M | D | V | F | I | T | Q | L | G | — | — | | |
| 29 | G | L | E | L | S | D | G | W | M | Q | Q | L | S | V | M | M | — | — | — | | |
| 30 | W | Q | F | S | E | Q | P | W | M | Q | H | L | I | M | V | M | — | — | — | | |
| 31 | Q | Q | I | Q | D | L | E | W | M | H | T | L | M | Q | M | M | — | — | — | | |
| 32 | N | S | E | V | H | M | E | W | M | A | H | L | M | F | M | M | — | — | — | | |
| 33 | P | R | Q | G | N | D | Y | W | I | D | H | L | V | E | M | L | — | — | — | | |
| 34 | Q | M | D | Q | N | V | H | W | M | Q | M | F | V | E | L | L | — | — | — | | |
| 35 | Q | W | A | D | P | Q | E | W | M | D | Q | M | V | A | M | L | — | — | — | | |
| 36 | N | M | A | P | S | E | D | W | M | M | T | L | V | Y | A | L | — | — | — | | |
| 37 | E | N | W | P | S | D | D | W | M | W | Q | V | V | A | A | L | — | — | — | | |
| 38 | E | W | I | V | I | D | Q | W | I | P | Q | L | V | Y | M | M | — | — | — | | |
| 39 | D | K | Q | F | W | V | S | S | Y | H | Q | L | V | Y | M | M | — | — | — | | |
| 40 | S | M | Q | E | T | E | V | W | Q | W | Q | L | V | Y | M | M | — | — | — | | |
| 41 | D | W | X | E | X | E | E | W | M | X | Q | L | V | Y | M | L | X | P | X | | |
| | Unconstrained (W) | | | | | | | | | | | | | | | | | | | | |
| Consensus | M | E | P | X | W | W | Q | D | E | E | E | L | V | Q | M | L | M | M | M | | |
| 42 | S | W | L | P | V | F | F | D | E | G | G | V | M | R | M | L | — | — | — | | |
| 43 | — | H | A | D | W | M | V | M | E | E | S | Y | F | I | Q | A | L | — | — | | |
| 44 | — | T | H | W | W | M | A | D | P | Q | E | L | I | A | I | M | G | — | — | | |
| 45 | M | K | P | K | F | F | Q | D | E | F | A | L | W | M | M | M | — | — | — | | |
| 46 | — | — | — | K | W | G | P | S | P | H | E | M | A | Q | F | V | W | M | M | | |
| 47 | N | D | P | V | W | Y | M | D | A | W | Y | A | M | Q | M | F | — | — | — | | |
| 48 | M | S | D | M | Y | M | F | D | E | W | K | M | V | Y | A | L | — | — | — | | |
| 49 | E | Y | L | S | F | W | Q | S | E | W | A | F | V | E | A | L | — | — | — | | |
| 50 | P | H | I | Y | W | H | R | P | E | E | Y | L | M | Q | M | L | — | — | — | | |
| 51 | D | F | P | T | W | I | I | H | E | Q | Q | L | M | F | M | L | — | — | — | | |
| 52 | — | — | — | W | W | N | M | D | P | L | Q | E | K | F | L | V | M | M | T | | |
| 53 | — | — | — | W | W | Y | Q | D | N | Y | D | I | S | Q | L | I | E | M | M | | |
| 54 | I | E | W | E | W | W | L | E | N | E | F | M | H | I | D | P | — | — | — | | |
| 55 | T | Q | R | E | W | P | A | E | W | E | F | L | S | F | M | L | — | — | — | | |
| 56 | M | E | M | L | W | W | Q | E | E | A | M | M | Q | L | V | M | — | — | — | | |
| 57 | — | — | L | L | W | H | S | E | D | A | E | M | L | L | H | V | M | M | — | | |
| 58 | Q | E | V | I | W | W | M | E | Q | Q | D | L | V | H | V | M | — | — | — | | |
| 59 | Q | E | P | M | W | W | I | N | E | E | E | F | V | R | L | L | — | — | — | | |
| 60 | L | D | V | M | W | H | I | N | E | L | D | F | V | N | Y | F | — | — | — | | |
| | C4xC | | | | | | | | | | | | | | | | | | | | |
| Consensus | X | E | I | D | W | C | Q | I | W | Q | C | X | E | X | E | L | D | | | | |
| 61 | I | M | T | D | W | C | K | I | W | E | C | H | E | E | Q | V | — | | | | |
| 62 | D | Q | V | D | W | C | L | I | W | Q | C | V | P | Q | D | D | — | | | | |
| 63 | Q | E | A | M | W | C | E | I | W | Q | C | V | T | E | M | L | — | | | | |
| 64 | Q | G | I | D | W | C | V | L | W | N | C | L | F | D | H | E | — | | | | |
| 65 | L | E | Q | I | V | C | M | W | M | V | C | E | E | D | M | H | — | | | | |
| 66 | P | A | M | D | W | C | Q | I | F | Q | C | T | I | Q | V | L | D | | | | |
| 67 | M | W | P | E | W | C | Q | V | W | I | C | A | L | P | E | I | — | | | | |
| 68 | L | E | I | D | W | C | Q | I | W | D | C | F | P | T | H | K | — | | | | |
| 69 | E | G | E | P | W | C | N | I | F | Q | C | T | Y | T | A | M | — | | | | |
| 70 | A | L | I | D | W | C | L | I | W | T | C | V | L | D | P | S | — | | | | |
| 71 | V | M | V | D | W | C | Q | I | F | G | C | K | W | V | P | I | — | | | | |
| 72 | E | D | W | D | W | C | A | I | W | N | C | L | E | H | H | L | — | | | | |
| 73 | I | W | P | D | W | C | W | I | W | E | C | V | S | R | Q | I | — | | | | |
| 74 | T | L | I | D | W | C | N | L | W | S | C | V | D | S | F | P | — | | | | |
| 75 | I | Q | V | D | W | C | Q | W | F | I | C | T | Q | E | N | L | — | | | | |
| 76 | Y | H | V | T | W | C | L | I | W | E | C | L | T | A | V | P | — | | | | |
| 77 | D | I | W | H | W | C | F | F | H | Q | C | E | V | Q | I | E | — | | | | |
| 78 | Q | W | P | D | W | C | S | I | W | S | C | L | P | T | E | A | — | | | | |
| 79 | S | E | I | D | W | C | S | W | W | H | C | I | E | L | A | D | — | | | | |
| 80 | N | W | V | D | W | C | Q | Y | W | V | C | V | Q | D | V | D | — | | | | |
| 81 | L | D | M | D | W | C | Q | I | W | D | C | I | Q | V | Y | N | — | | | | |
| 82 | W | N | S | D | P | C | D | F | W | Q | C | I | Q | P | T | A | — | | | | |
| 83 | M | H | I | E | W | C | E | L | F | Q | C | W | H | V | D | Q | — | | | | |
| 84 | I | M | W | D | P | C | M | V | W | E | C | I | D | A | T | Y | — | | | | |
| 85 | H | E | L | W | F | C | Q | L | W | T | C | I | D | I | P | G | — | | | | |
| 86 | H | A | L | D | W | C | Q | I | W | T | C | M | T | P | Q | N | — | | | | |
| 87 | M | Y | D | E | W | C | M | I | W | E | C | L | P | E | S | P | — | | | | |
| 88 | P | D | F | N | W | C | H | W | W | E | C | M | E | V | E | L | — | | | | |
| 89 | D | T | L | Q | W | C | E | W | W | T | C | I | W | D | H | H | — | | | | |
| 90 | M | W | I | E | W | C | D | I | W | T | C | I | E | Y | E | P | — | | | | |
| 91 | L | D | Q | A | W | C | G | I | W | N | C | I | H | V | T | P | — | | | | |
| 92 | Y | H | I | S | W | C | D | I | W | D | C | V | V | S | E | Q | — | | | | |
| 93 | Y | E | V | N | W | C | S | F | W | T | C | V | Q | E | V | A | — | | | | |
| 94 | Q | L | V | D | W | C | N | I | W | Q | C | Y | P | S | D | L | — | | | | |
| 95 | W | S | W | D | P | C | E | M | F | S | C | M | L | Q | Y | H | — | | | | |
| | C7xC | | | | | | | | | | | | | | | | | | | | |
| Consensus | E | W | Y | I | C | X | Q | F | P | E | W | X | C | X | X | X | | | | | |
| 96 | T | A | Y | E | C | F | Q | F | P | E | W | S | C | E | S | E | | | | | |
| 97 | S | L | N | I | C | E | Q | F | P | E | F | Q | C | I | Y | K | | | | | |
| 98 | L | W | D | I | C | G | Q | F | P | E | W | M | C | I | D | N | | | | | |
| 99 | E | W | E | S | C | Y | D | S | D | W | H | I | C | Q | L | Y | | | | | |
| 100 | Y | P | I | W | C | Q | W | F | P | E | W | E | C | D | D | M | | | | | |
| 101 | E | G | Y | T | C | R | P | E | D | W | H | A | C | Q | L | A | | | | | |
| | C12xC | | | | | | | | | | | | | | | | | | | | |
| Consensus | E | C | W | E | W | D | X | E | M | Q | Z | W | V | P | C | D | | | | | |
| 102 | E | C | W | E | W | S | Y | Q | L | Q | E | W | S | P | C | P | | | | | |
| 103 | D | C | W | E | Y | D | Y | K | Q | M | S | W | E | R | C | D | | | | | |
| 104 | E | C | W | E | W | E | F | Q | Y | Q | L | G | W | K | C | I | | | | | |
| 105 | F | C | W | Q | Q | K | N | D | E | Y | I | R | I | E | C | A | | | | | |
| 106 | F | C | M | E | W | I | P | A | T | M | E | W | V | P | C | D | | | | | |
| 107 | S | C | M | E | F | M | W | D | T | Q | T | W | E | L | C | A | | | | | |
| 108 | M | C | W | E | F | M | W | E | F | E | S | W | Q | P | C | S | | | | | |
| 109 | E | C | W | E | W | I | H | N | M | Q | E | W | K | S | C | V | | | | | |
| 110 | S | C | E | W | W | G | P | E | S | R | Q | I | Y | M | C | E | | | | | |
| 111 | E | C | W | E | W | S | H | I | W | G | E | F | L | E | C | T | | | | | |
| 112 | E | C | W | E | Y | L | P | H | W | G | I | W | Q | D | C | G | | | | | |
| 113 | Q | C | W | E | Y | Q | W | T | M | Q | M | W | V | E | C | E | | | | | |
| 114 | S | C | W | E | Y | M | F | E | A | Q | Q | W | V | A | C | M | | | | | |
| 115 | Q | C | W | Q | Y | V | F | D | M | A | M | W | T | T | C | G | | | | | |
| 116 | M | C | W | E | W | F | Q | E | T | R | T | W | V | Y | C | D | | | | | |
| 117 | M | C | Y | E | W | E | W | M | S | H | T | W | V | D | C | Q | | | | | |
| 118 | Q | C | W | E | W | M | S | V | V | N | G | W | E | P | C | G | | | | | |
| 119 | M | C | W | E | W | S | S | L | Q | D | N | W | M | P | C | D | | | | | |
| 120 | Q | C | W | E | W | D | K | A | M | S | Q | W | V | Y | C | A | | | | | |
| 121 | Q | C | W | Q | F | N | F | S | L | L | Q | W | E | V | C | G | | | | | |
| 122 | I | C | E | I | V | V | Q | T | E | E | H | V | W | E | C | M | | | | | |
| 123 | Q | C | W | M | L | E | Q | T | S | L | T | W | V | P | C | P | | | | | |
| 124 | Q | C | W | E | M | N | W | T | V | N | M | W | E | P | C | A | | | | | |
| 125 | Q | C | W | E | W | M | Y | Q | W | S | E | W | R | Q | C | M | | | | | |
| 126 | M | C | W | E | W | E | L | T | I | M | D | W | I | S | C | D | | | | | |
| 127 | E | C | W | E | F | Y | Q | E | H | W | S | M | F | D | C | E | | | | | |
| 128 | N | C | W | S | W | Q | Y | L | L | G | Q | W | V | E | C | D | | | | | |
| 129 | Q | C | W | V | W | D | F | E | M | W | E | W | Y | Q | C | S | | | | | |
| 130 | D | C | W | Q | W | L | Y | V | E | K | M | W | I | P | C | Y | | | | | |
| 131 | E | C | W | Q | W | D | F | E | Q | Q | S | W | I | P | C | R | | | | | |
| 132 | E | C | W | E | W | I | S | Q | M | Q | V | W | V | Q | C | P | | | | | |
| 133 | E | C | W | I | Y | D | W | M | N | Q | T | W | Q | Y | C | N | | | | | |
| 134 | N | C | W | E | W | V | W | H | A | Q | Q | W | V | P | C | E | | | | | |
| 135 | E | C | W | Q | Y | D | F | Y | H | Q | M | W | S | A | C | I | | | | | |
| | C10xC | | | | | | | | | | | | | | | | | | | | |
| Consensus | Q | E | C | W | E | W | V | D | G | Q | W | Q | E | C | H | E | | | | | |
| 136 | Y | E | C | W | E | W | M | E | G | E | W | I | S | C | G | G | | | | | |
| 137 | V | E | C | W | E | L | F | T | H | G | W | Q | P | C | Q | H | | | | | |
| 138 | F | M | C | Q | E | V | E | P | G | Y | I | W | E | C | S | M | | | | | |
| 139 | T | N | C | W | E | W | I | Y | Q | M | W | Q | S | C | Q | P | | | | | |
| 140 | Q | A | C | D | E | G | P | Y | I | W | D | F | M | C | M | I | | | | | |
| 141 | V | I | C | W | E | L | I | D | N | Q | W | M | Q | C | H | M | | | | | |
| 142 | P | Q | C | W | E | Y | I | F | G | I | W | Q | P | C | M | D | | | | | |
| 143 | V | I | C | W | E | L | I | E | A | Q | W | T | H | C | A | P | | | | | |
| 144 | Q | A | C | W | F | F | Q | D | G | N | W | Y | D | C | Y | E | | | | | |
| 145 | G | G | C | W | Q | W | V | D | W | D | W | I | Q | C | H | E | | | | | |
| 146 | W | W | C | G | E | M | H | G | Q | T | L | M | E | C | F | Y | | | | | |
| 147 | W | E | C | W | E | F | L | M | D | V | W | V | P | C | V | S | | | | | |
| 148 | T | E | C | W | E | L | I | N | F | T | W | Q | Q | C | L | A | | | | | |
| 149 | I | I | C | D | V | E | W | R | V | T | I | P | E | C | W | V | | | | | |
| 150 | P | M | C | W | E | M | F | E | W | G | W | S | Q | C | F | T | | | | | |
| 151 | E | I | C | Y | P | T | A | W | P | G | V | M | Q | C | L | L | | | | | |
| 152 | Y | E | C | W | E | Y | Y | V | G | I | W | V | H | C | N | V | | | | | |
| 153 | W | A | C | W | A | W | N | G | M | N | W | M | E | C | V | D | | | | | |
| 154 | Q | Q | C | W | E | W | T | D | F | G | W | S | E | C | H | D | | | | | |
| 155 | Q | E | C | W | S | L | V | D | W | Q | W | Q | R | C | D | D | | | | | |
| 156 | W | P | C | W | E | W | M | Q | G | Q | W | W | D | C | V | E | | | | | |
| 157 | Q | I | C | W | E | K | V | Q | D | L | W | V | M | C | T | D | | | | | |
| 158 | I | D | C | W | E | W | V | M | T | E | W | Y | P | C | I | Y | | | | | |
| 159 | Q | S | C | P | Y | K | E | D | G | V | W | Y | D | C | L | I | | | | | |
| 160 | P | E | C | W | Q | W | A | S | P | E | W | V | E | C | T | D | | | | | |
| 161 | V | D | C | W | E | M | M | D | Q | Q | W | L | P | C | R | D | | | | | |
| 162 | P | T | C | W | D | L | V | D | M | N | W | I | P | C | A | V | | | | | |
| 163 | M | E | C | W | Q | W | V | E | F | Q | W | M | A | C | S | E | | | | | |
| 164 | Q | S | C | W | E | W | V | M | G | H | W | R | V | C | Q | E | | | | | |
| 165 | V | E | C | W | M | Y | F | Q | E | Y | W | I | P | C | P | Q | | | | | |
| 166 | T | V | C | W | E | W | V | D | H | V | W | A | E | C | P | T | | | | | |
| 167 | M | E | C | W | E | M | I | Q | G | Q | W | A | M | C | M | N | | | | | |
| 168 | W | E | C | F | Q | L | P | D | S | I | M | Y | E | C | Y | M | | | | | |
| 169 | N | N | C | W | Q | Y | V | S | D | S | W | M | P | C | T | A | | | | | |
| 170 | A | S | C | W | E | M | I | E | T | V | W | V | Q | C | N | M | | | | | |
| 171 | M | E | C | W | E | W | A | Q | G | E | W | N | Y | C | M | P | | | | | |
| 172 | E | Y | C | W | E | L | L | Q | S | V | W | T | V | C | T | E | | | | | |
| 173 | V | Q | C | W | Q | W | Q | D | H | Q | W | I | E | C | I | T | | | | | |
| 174 | M | T | C | W | M | W | V | Q | E | S | W | Q | E | C | M | D | | | | | |
| 175 | Q | F | C | W | E | L | V | D | G | W | W | N | M | C | D | R | | | | | |
| 176 | D | Q | C | W | M | W | Y | S | G | A | W | Q | P | C | A | T | | | | | |
| 177 | F | E | C | W | E | Y | F | G | E | S | W | Q | A | C | H | N | | | | | |
| 178 | E | E | C | W | V | F | I | E | T | W | W | Q | P | C | H | L | | | | | |
| 179 | Q | M | C | W | E | W | M | Y | T | E | W | V | E | C | E | T | | | | | |
| 180 | P | P | C | W | E | L | I | V | D | H | W | I | I | C | E | E | | | | | |
| 181 | P | E | C | W | E | W | F | P | W | G | W | D | Q | C | H | M | | | | | |
| 182 | A | Q | C | W | E | Y | V | I | D | H | W | V | M | C | T | E | | | | | |
| 183 | L | E | C | W | E | W | A | H | I | E | W | M | P | C | I | E | | | | | |
| 184 | P | N | C | W | E | W | V | Q | K | E | W | I | E | C | A | I | | | | | |
| 185 | I | E | C | W | E | W | Q | K | D | K | W | V | E | C | S | S | | | | | |
| 186 | Y | E | C | W | E | M | V | N | N | F | W | Q | T | C | D | H | | | | | |
| 187 | P | E | C | W | E | Y | I | H | N | G | W | M | Q | C | D | G | | | | | |
| 188 | R | E | C | W | E | Y | V | H | Q | M | W | Q | T | C | Q | E | | | | | |
| 189 | F | E | C | W | E | W | N | G | M | D | W | G | A | C | N | S | | | | | |

TABLE 9
Additional exemplary peptide variants, including
those identified in FIG. 34 of this
application, are shown below in table form.

| | * | * | | | * | * | * | | | * | | | * | * | | |
|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 |
| | D | F | D | F | A | E | N | W | M | E | Q | L | K | V | M | L |
| p16a | Q | Y | D | H | H | D | N | W | M | E | Q | L | V | L | M | L |
| p16b | D | Y | D | F | H | D | K | W | M | E | Q | L | I | L | M | L |
| p16c | D | Y | E | F | H | E | H | W | M | D | Q | L | I | L | M | M |
| p16d | E | Y | D | L | H | D | Q | W | M | E | Q | L | V | L | M | L |
| | Q | L | D | F | T | D | D | W | M | E | Q | L | V | I | M | L |
| | D | V | D | L | H | D | K | W | M | D | D | L | I | H | M | L |
| | H | N | D | Y | S | E | D | W | M | D | Q | L | V | V | M | L |
| | H | F | E | V | Y | D | N | W | M | D | Q | L | V | Q | M | L |
| | H | Y | D | F | P | E | H | W | M | D | Q | L | I | V | M | M |
| | D | Y | E | Y | N | E | D | W | M | E | Q | L | I | L | M | L |
| | Q | H | D | V | S | D | D | W | L | D | Q | L | V | L | M | L |
| | D | F | D | F | N | D | K | W | I | E | E | L | I | L | M | L |
| | Q | I | D | F | S | D | N | W | M | D | Q | L | V | L | M | M |
| | Q | Y | D | F | N | E | D | W | M | E | Q | L | I | L | M | M |
| | E | L | D | V | N | D | K | W | M | D | Q | L | I | L | M | M |
| | H | F | E | F | D | D | H | W | M | D | Q | L | V | L | M | M |
| | E | F | E | F | N | E | N | W | M | E | Q | L | V | L | M | M |
| | Q | D | D | V | Y | D | D | W | M | D | Q | L | I | L | M | L |
| | E | I | D | Y | H | D | N | W | L | D | Q | L | V | V | M | M |
| | E | F | E | F | Y | E | D | W | M | D | Q | L | I | L | M | L |
| | Q | Y | D | V | N | E | Q | W | M | D | Q | L | I | Q | M | L |
| | D | Y | D | V | N | E | Q | W | M | D | Q | L | I | L | M | L |
| | H | V | E | Y | D | E | N | W | M | E | Q | L | I | L | M | L |
| | E | I | E | Y | Y | E | H | W | M | E | Q | L | V | H | M | L |
| | E | D | D | F | N | E | H | W | M | E | Q | L | I | L | L | L |
| | H | F | D | V | N | D | Q | W | M | D | Q | L | V | L | M | L |
| | D | F | D | Y | H | E | H | W | M | E | D | L | I | L | M | L |
| | Q | Y | D | Y | S | D | N | W | M | E | Q | F | V | L | M | L |
| | Q | Y | E | V | T | D | N | W | M | D | Q | L | V | L | M | L |
| | E | Y | E | D | A | D | N | W | M | E | Q | L | V | V | M | M |
| | E | Y | D | V | Y | D | H | W | M | D | Q | L | I | V | M | M |
| | Q | Y | D | V | H | D | Q | W | M | E | Q | L | I | L | M | L |
| | E | H | D | Y | Y | D | N | W | M | D | Q | L | V | V | M | L |
| | H | L | D | F | H | E | D | W | M | E | Q | L | V | L | M | M |
| | H | N | D | F | N | E | K | W | M | E | Q | L | V | L | M | M |
| | H | Y | D | H | Y | E | H | W | M | D | Q | L | I | V | M | M |
| | D | F | E | V | Y | D | N | W | M | E | Q | L | I | V | M | L |
| | E | Y | E | D | H | D | H | W | L | D | Q | L | V | L | M | M |
| | H | Y | D | F | D | E | Q | W | M | D | H | L | I | L | M | L |
| | H | V | D | F | H | D | D | W | M | D | Q | L | V | L | M | M |
| | Q | V | E | Y | Y | D | N | W | M | D | H | L | I | V | M | L |
| | Q | H | D | H | H | D | D | W | M | D | Q | L | I | V | M | L |
| | E | Y | D | H | H | D | H | W | M | D | E | L | V | L | M | M |
| | D | F | E | F | S | D | H | W | I | D | Q | L | V | L | M | L |
| | Q | F | D | H | A | D | H | W | M | E | H | L | V | L | M | M |
| | D | V | D | F | A | D | K | W | M | E | H | L | I | V | M | L |
| | H | Y | D | H | D | E | H | W | M | E | Q | L | I | H | M | L |
| | E | F | D | H | Y | D | N | W | M | D | Q | L | I | L | M | L |
| | D | H | D | H | Y | D | D | W | M | D | Q | L | I | V | M | L |
| | Q | Y | E | V | Y | E | D | W | M | E | Q | L | V | L | M | L |
| | E | I | D | H | D | D | K | W | M | E | Q | L | I | L | M | L |
| | E | Y | E | F | N | E | H | W | M | D | H | L | V | L | M | L |
| | E | V | D | F | Y | D | D | W | M | D | Q | L | V | L | M | M |
| | D | N | D | F | D | E | K | W | M | E | Q | L | I | L | L | L |
| | Q | V | D | Y | D | E | H | W | M | E | Q | L | V | L | M | L |
| | H | D | D | D | Y | D | D | W | L | D | Q | L | V | V | M | M |
| | D | Y | D | F | S | E | E | W | M | D | Q | L | V | L | M | L |
| | H | Y | E | V | Y | D | N | W | M | D | Q | L | V | Q | M | M |
| | E | F | D | V | H | D | Q | W | M | E | Q | L | V | L | M | M |
| | E | F | E | H | N | E | Q | W | M | D | Q | L | I | L | M | L |
| | Q | Y | D | Y | D | D | H | W | M | E | Q | L | I | V | M | L |
| | H | N | D | F | N | E | N | W | M | D | Q | L | V | V | M | M |
| | E | H | E | V | H | E | Q | W | M | D | Q | L | I | H | M | L |
| | Q | Y | E | Y | P | E | Q | W | M | D | Q | L | V | L | M | L |
| | E | Y | E | F | H | E | K | W | M | E | E | L | I | H | M | L |
| | D | F | D | Y | N | E | E | W | M | D | Q | L | V | V | M | L |
| | D | Y | D | V | H | E | Q | W | M | D | H | L | I | H | M | L |
| | E | Y | D | V | A | D | K | W | M | D | Q | L | I | L | M | M |
| | H | F | D | Y | S | D | N | W | M | D | H | L | V | L | M | M |
| | E | L | D | V | H | D | D | W | L | D | Q | L | I | L | M | M |
| | D | Y | E | V | Y | D | D | W | L | E | Q | L | V | V | M | L |
| | E | H | E | F | N | D | D | W | M | D | Q | L | V | L | M | L |
| | D | Y | D | L | Y | D | N | W | M | E | Q | L | I | V | M | M |
| | H | V | D | F | H | E | D | W | M | D | Q | L | V | V | M | L |
| | E | F | D | F | S | D | D | W | M | D | Q | L | V | L | M | M |
| | E | Y | E | F | N | E | H | W | M | E | Q | L | I | V | M | M |
| | E | N | E | Y | T | E | H | W | M | E | Q | L | I | L | M | L |
| | D | F | D | Y | A | D | Q | W | M | D | Q | L | V | L | M | L |
| | E | F | D | F | P | E | H | W | M | D | Q | L | V | L | L | L |
| | E | F | E | F | Y | D | N | W | L | D | Q | L | V | V | M | M |
| | Q | F | E | V | H | E | E | W | M | E | Q | L | I | L | M | L |
| | Q | F | E | F | H | D | H | W | M | D | Q | L | I | H | M | L |
| | H | Y | D | F | D | E | K | W | M | E | D | L | I | L | M | L |
| | H | N | D | Y | H | D | D | W | M | D | Q | L | I | L | M | L |
| | Q | Y | D | Y | S | D | N | W | M | E | Q | L | V | L | M | M |
| | | | | | | | * | * | * | | | | * | | | * |
| | 1 | 2 | 3 | 4 | 5 | 6 | 7 | 8 | 9 | 10 | 11 | 12 | 13 | 14 | 15 | 16 |
| | E | C | W | E | W | S | Y | Q | L | Q | E | W | S | P | C | P |
| p102a | E | C | W | E | W | S | F | H | F | Q | E | W | G | P | C | D |
| p102b | E | C | W | E | W | N | F | H | F | Q | E | W | G | P | C | D |
| | E | C | W | E | W | K | F | H | L | Q | E | W | L | A | C | D |
| | E | C | W | E | W | R | F | H | I | Q | E | W | M | P | C | E |
| | E | C | W | Q | W | N | Y | H | L | Q | E | W | G | Q | C | D |
| | E | C | W | E | W | N | F | Q | L | H | E | W | G | P | C | E |
| | E | C | W | E | W | S | Y | Q | F | Q | E | W | G | P | C | E |
| | E | C | W | E | W | S | F | Q | L | Q | E | W | G | A | C | A |
| | E | C | W | E | W | S | F | Q | F | Q | E | W | G | P | C | D |
| | D | C | W | E | W | K | Y | E | I | Q | E | W | V | A | C | E |
| | E | C | W | E | W | K | Y | D | L | Q | E | W | V | A | C | Q |
| | E | C | W | E | W | N | Y | D | F | Q | E | W | G | P | C | Q |
| | E | C | W | Q | W | S | Y | E | F | Q | E | W | G | Q | C | A |
| | E | C | W | E | W | K | F | H | M | Q | E | W | V | H | C | D |
| | E | C | W | E | W | N | F | D | F | Q | E | W | G | H | C | Q |
| | E | C | W | E | W | S | Y | H | M | Q | E | W | G | P | C | P |
| | E | C | W | E | W | S | F | H | F | Q | E | W | G | Q | C | Q |
| | E | C | W | E | W | K | F | E | I | Q | E | W | L | P | C | Q |
| | E | C | W | E | W | N | Y | H | L | Q | E | W | G | A | C | Q |
| | E | C | W | E | W | S | F | Q | F | H | E | W | G | P | C | A |
| | E | C | W | E | W | S | Y | Q | F | H | E | W | G | A | C | Q |
| | E | C | W | E | W | N | F | H | F | Q | E | W | G | P | C | A |
| | E | C | W | E | W | S | F | Q | F | Q | E | W | G | A | C | Q |
| | E | C | W | E | W | N | F | Q | M | H | E | W | G | P | C | A |

We claim:

1. A fusion molecule comprising one or more peptides that binds the SARS-CoV-2-S protein fused to an anti-SARS-CoV-2 antibody or an anti-SARS-CoV-2 synthetic molecule.

2. The fusion molecule of claim 1, wherein the one or more peptides are fused to the N-terminus of either the light chain or the heavy chain of the anti-SARS-CoV-2 antibody.

3. The fusion molecule of claim 1, where the peptide binds either the RBD or the non-RBD regions of the S protein.

4. The fusion molecule of claim 1, where the peptide is selected from the peptides identified in FIG. 2, Table 8, and Table 9.

5. The fusion molecule of claim 1, where the anti-SARS-CoV-2 antibody binds the RBD.

6. The fusion molecule of claim 1, where the fusion comprises at least one peptide fused to a multivalent binding molecule comprising two or more antibody binding fragments that bind a coronavirus, wherein the binding fragments of the multivalent binding molecule are joined by a peptide linker.

7. The fusion molecule of claim 1, wherein the peptide is at least one selected from the peptides described in FIG. 2, Table 8, and Table 9, and the antibody is Antibody 15033-7.

8. The fusion molecule of claim 1, where the peptide binds either the RBD or the NTD regions of the S protein.

9. The fusion molecule of claim 1, where the peptide binds three neutralizing epitopes on a single S protein trimer.

10. The fusion molecule of claim 1, where the peptide is p102 and/or p16.

11. The fusion molecule of claim 1, wherein the peptide is at least one selected from the peptides described in FIG. 2, Table 8, and Table 9, and the antibody is Antibody 15033-7.

12. The fusion molecule of claim 1, wherein the peptide is p102 and/or p16, and the antibody is Antibody 15033-7.

13. The fusion molecule of claim 12, wherein the peptides are fused to the N-terminus of either the light chain or the heavy chain of the anti-SARS-CoV-2 antibody.

14. The fusion molecule of claim 12, wherein the peptides are fused to the N-terminus of either the light chain or the heavy chain of the anti-SARS-CoV-2 Antibody 15033-7.


SEQUENCE LISTING (519 sequences)

SEQ ID NO: 1 - protein, 20 aa, Artificial Sequence
  Description of Artificial Sequence Synthetic peptide
GGGGSGGGGSGGGGSGGGGS

SEQ ID NO: 2 - protein, 4 aa, Artificial Sequence
  Description of Artificial Sequence Synthetic peptide
GKGK

SEQ ID NO: 3 - protein, 9 aa, Artificial Sequence
  Description of Artificial Sequence Synthetic peptide
  MOD_RES (2)..(2): Any hydrophobic amino acid
  MOD_RES (3)..(3): Any amino acid
  MOD_RES (4)..(4): Any amino acid
  MOD_RES (6)..(6): Any amino acid
  MOD_RES (7)..(7): Any amino acid
WXXXLXXMM

SEQ ID NO: 4 - protein, 9 aa, Artificial Sequence
  Description of Artificial Sequence Synthetic peptide
  MOD_RES (3)..(3): Any amino acid
  MOD_RES (4)..(4): Any amino acid
  MOD_RES (7)..(7): Any amino acid
WEXXLVXML

SEQ ID NO: 5 - protein, 12 aa, Artificial Sequence
  Description of Artificial Sequence Synthetic peptide
  MOD_RES (2)..(2): Any amino acid
  MOD_RES (3)..(3): Any amino acid
  MOD_RES (5)..(5): Any amino acid
  MOD_RES (6)..(6): Any amino acid
  MOD_RES (7)..(7): Any amino acid
  MOD_RES (9)..(9): Any amino acid
  MOD_RES (10)..(10): Any amino acid
WXXDXXXLXXML

SEQ ID NO: 6 - protein, 8 aa, Artificial Sequence
  Description of Artificial Sequence Synthetic peptide
  MOD_RES (4)..(4): Any amino acid
  MOD_RES (5)..(5): Any hydrophobic amino acid
  MOD_RES (7)..(7): Any amino acid
DWCXXWXC

SEQ ID NO: 7 - protein, 4 aa, Artificial Sequence
  Description of Artificial Sequence Synthetic peptide
FPEW

SEQ ID NO: 8 - protein, 11 aa, Artificial Sequence
  Description of Artificial Sequence Synthetic peptide
  MOD_RES (4)..(4): Any amino acid
  MOD_RES (5)..(5): Any amino acid
  MOD_RES (6)..(6): Any amino acid
  MOD_RES (7)..(7): Any amino acid
  MOD_RES (8)..(8): Any amino acid
  MOD_RES (10)..(10): Any amino acid
CWEXXXXXWXC

SEQ ID NO: 9 - protein, 14 aa, Artificial Sequence
  Description of Artificial Sequence Synthetic peptide
  MOD_RES (5)..(5): Any amino acid
  MOD_RES (6)..(6): Any amino acid
  MOD_RES (7)..(7): Any amino acid
  MOD_RES (8)..(8): Any amino acid
  MOD_RES (9)..(9): Any amino acid
  MOD_RES (10)..(10): Any amino acid
  MOD_RES (12)..(12): Any amino acid
  MOD_RES (13)..(13): Any amino acid
CWEWXXXXXXWXXC

SEQ ID NO: 10 - protein, 6 aa, Artificial Sequence
  Description of Artificial Sequence Synthetic 6xHis tag
HHHHHH

SEQ ID NO: 11 - protein, 4 aa, Artificial Sequence
  Description of Artificial Sequence Synthetic peptide
RRAR

SEQ ID NO: 12 - protein, 4 aa, Artificial Sequence
  Description of Artificial Sequence Synthetic peptide
SSAS

SEQ ID NO: 13 - protein, 16 aa, Artificial Sequence
  Description of Artificial Sequence Synthetic peptide
SGGGGSGGGGSGGGGS

SEQ ID NO: 14 - protein, 16 aa, Artificial Sequence
  Description of Artificial Sequence Synthetic peptide
GGGGGSGGGGSGGGTG

SEQ ID NO: 15 - protein, 32 aa, Artificial Sequence
  Description of Artificial Sequence Synthetic polypeptide
GGGGSSSSGGGGSSSSGGGGSSSSGGGGSSSS

SEQ ID NO: 16 - protein, 22 aa, Artificial Sequence
  Description of Artificial Sequence Synthetic peptide
GDFDFAENWMEQLKVMLGGKGK

SEQ ID NO: 17 - protein, 22 aa, Artificial Sequence
  Description of Artificial Sequence Synthetic peptide
GQYDHHDNWMEQLVLMLGGKGK

SEQ ID NO: 18 - protein, 22 aa, Artificial Sequence
  Description of Artificial Sequence Synthetic peptide
GDYDFHDKWMEQLILMLGGKGK

SEQ ID NO: 19 - protein, 22 aa, Artificial Sequence
  Description of Artificial Sequence Synthetic peptide
GDYEFHEHWMDQLILMMGGKGK

SEQ ID NO: 20 - protein, 22 aa, Artificial Sequence
  Description of Artificial Sequence Synthetic peptide
GEYDLHDQWMEQLVLMLGGKGK

SEQ ID NO: 21 - protein, 22 aa, Artificial Sequence
  Description of Artificial Sequence Synthetic peptide
GECWEWSYQLQEWSPCPGGKGK

SEQ ID NO: 22 - protein, 22 aa, Artificial Sequence
  Description of Artificial Sequence Synthetic peptide
GECWEWSFHFQEWGPCDGGKGK

SEQ ID NO: 23 - protein, 22 aa, Artificial Sequence
  Description of Artificial Sequence Synthetic peptide
GECWEWNFHFQEWGPCDGGKGK

SEQ ID NO: 24 - protein, 5 aa, Artificial Sequence
  Description of Artificial Sequence Synthetic peptide
GGKGK

SEQ ID NO: 25 - protein, 6 aa, Artificial Sequence
  Description of Artificial Sequence Synthetic peptide
LTGSYM

SEQ ID NO: 26 - protein, 5 aa, Artificial Sequence
  Description of Artificial Sequence Synthetic peptide
SVSSA

SEQ ID NO: 27 - protein, 6 aa, Artificial Sequence
  Description of Artificial Sequence Synthetic peptide
FSAASI

SEQ ID NO: 28 - protein, 6 aa, Artificial Sequence
  Description of Artificial Sequence Synthetic peptide
LGGYSM

SEQ ID NO: 29 - protein, 5 aa, Artificial Sequence
  Description of Artificial Sequence Synthetic peptide
DVAYA

SEQ ID NO: 30 - protein, 6 aa, Artificial Sequence
  Description of Artificial Sequence Synthetic peptide
IGGDSI

SEQ ID NO: 31 - protein, 6 aa, Artificial Sequence
  Description of Artificial Sequence Synthetic peptide
FTAYDI

SEQ ID NO: 32 - protein, 6 aa, Artificial Sequence
  Description of Artificial Sequence Synthetic peptide
FGSYAM

SEQ ID NO: 33 - protein, 6 aa, Artificial Sequence
  Description of Artificial Sequence Synthetic peptide
FSSSDI

SEQ ID NO: 34 - protein, 10 aa, Artificial Sequence
  Description of Artificial Sequence Synthetic peptide
GISASGGATA

SEQ ID NO: 35 - protein, 10 aa, Artificial Sequence
  Description of Artificial Sequence Synthetic peptide
GIYASGGATD

SEQ ID NO: 36 - protein, 10 aa, Artificial Sequence
  Description of Artificial Sequence Synthetic peptide
GIYASGGATA

SEQ ID NO: 37 - protein, 10 aa, Artificial Sequence
  Description of Artificial Sequence Synthetic peptide
GIYASGGSTA

SEQ ID NO: 38 - protein, 10 aa, Artificial Sequence
  Description of Artificial Sequence Synthetic peptide
GISASGGYTA

SEQ ID NO: 39 - protein, 10 aa, Artificial Sequence
  Description of Artificial Sequence Synthetic peptide
GIYASGGYTD

SEQ ID NO: 40 - protein, 10 aa, Artificial Sequence
  Description of Artificial Sequence Synthetic peptide
RIYASGGYTD

SEQ ID NO: 41 - protein, 18 aa, Artificial Sequence
  Description of Artificial Sequence Synthetic peptide
SRSSSYSSSGWRYYSGAF

SEQ ID NO: 42 - protein, 5 aa, Artificial Sequence
  Description of Artificial Sequence Synthetic peptide
GSYLF

SEQ ID NO: 43 - protein, 17 aa, Artificial Sequence
  Description of Artificial Sequence Synthetic peptide
SSYRYSSGASFHYYYGF

SEQ ID NO: 44 - protein, 6 aa, Artificial Sequence
  Description of Artificial Sequence Synthetic peptide
YYHSPF

SEQ ID NO: 45 - protein, 9 aa, Artificial Sequence
  Description of Artificial Sequence Synthetic peptide
SYYYGGFGM

SEQ ID NO: 46 - protein, 6 aa, Artificial Sequence
  Description of Artificial Sequence Synthetic peptide
SYRYPI

SEQ ID NO: 47 - protein, 6 aa, Artificial Sequence
  Description of Artificial Sequence Synthetic peptide
SHTYPI

SEQ ID NO: 48 - protein, 14 aa, Artificial Sequence
  Description of Artificial Sequence Synthetic peptide
GGWGPYSYYYHYGL

SEQ ID NO: 49 - protein, 8 aa, Artificial Sequence
  Description of Artificial Sequence Synthetic peptide
YSYYYDLI

SEQ ID NO: 50 - protein, 8 aa, Artificial Sequence
  Description of Artificial Sequence Synthetic peptide
DGFHSYAL

SEQ ID NO: 51 - protein, 7 aa, Artificial Sequence
  Description of Artificial Sequence Synthetic peptide
YSYDSLI

SEQ ID NO: 52 - protein, 8 aa, Artificial Sequence
  Description of Artificial Sequence Synthetic peptide
GWSYYWGF

SEQ ID NO: 53 - protein, 6 aa, Artificial Sequence
  Description of Artificial Sequence Synthetic peptide
YDYGLF

SEQ ID NO: 54 - protein, 7 aa, Artificial Sequence
  Description of Artificial Sequence Synthetic peptide
SSPSDLF

SEQ ID NO: 55 - protein, 12 aa, Artificial Sequence
  Description of Artificial Sequence Synthetic peptide
WGHGYGYFYYGF

SEQ ID NO: 56 - protein, 6 aa, Artificial Sequence
  Description of Artificial Sequence Synthetic peptide
SSGDLI

SEQ ID NO: 57 - DNA, 18 bp, Artificial Sequence
  Description of Artificial Sequence Synthetic oligonucleotide
ctcactggttcttacatg

SEQ ID NO: 58 - DNA, 15 bp, Artificial Sequence
  Description of Artificial Sequence Synthetic oligonucleotide
tccgtgtccagcgct

SEQ ID NO: 59 - DNA, 18 bp, Artificial Sequence
  Description of Artificial Sequence Synthetic oligonucleotide
ttcagtgctgcttcgatc

SEQ ID NO: 60 - DNA, 18 bp, Artificial Sequence
  Description of Artificial Sequence Synthetic oligonucleotide
ctcggtggttattcgatg

SEQ ID NO: 61 - DNA, 15 bp, Artificial Sequence
  Description of Artificial Sequence Synthetic oligonucleotide
gatgtggcttatgct

SEQ ID NO: 62 - DNA, 18 bp, Artificial Sequence
  Description of Artificial Sequence Synthetic oligonucleotide
atcggtggtgattcgatc

SEQ ID NO: 63 - DNA, 18 bp, Artificial Sequence
  Description of Artificial Sequence Synthetic oligonucleotide
ttcactgcttatgacatc

SEQ ID NO: 64 - DNA, 18 bp, Artificial Sequence
  Description of Artificial Sequence Synthetic oligonucleotide
ttcggtagttatgccatg

SEQ ID NO: 65 - DNA, 18 bp, Artificial Sequence
  Description of Artificial Sequence Synthetic oligonucleotide
ttcagtagttctgacatc

SEQ ID NO: 66 - DNA, 30 bp, Artificial Sequence
  Description of Artificial Sequence Synthetic oligonucleotide
ggtatttccgcttctggaggcgctactgct

SEQ ID NO: 67 - DNA, 30 bp, Artificial Sequence
  Description of Artificial Sequence Synthetic oligonucleotide
ggtatttacgcttctggaggcgctactgat

SEQ ID NO: 68 - DNA, 30 bp, Artificial Sequence
  Description of Artificial Sequence Synthetic oligonucleotide
ggtatttacgcttctggaggcgctactgct

SEQ ID NO: 69 - DNA, 30 bp, Artificial Sequence
  Description of Artificial Sequence Synthetic oligonucleotide
ggtatttacgcttctggaggctctactgct

SEQ ID NO: 70 - DNA, 30 bp, Artificial Sequence
  Description of Artificial Sequence Synthetic oligonucleotide
ggtatttccgcttctggaggctatactgct

SEQ ID NO: 71 - DNA, 30 bp, Artificial Sequence
  Description of Artificial Sequence Synthetic oligonucleotide
ggtatttacgcttctggaggctatactgat

SEQ ID NO: 72 - DNA, 30 bp, Artificial Sequence
  Description of Artificial Sequence Synthetic oligonucleotide
cgtatttacgcttctggaggctatactgat

SEQ ID NO: 73 - DNA, 54 bp, Artificial Sequence
  Description of Artificial Sequence Synthetic oligonucleotide
tctcgttcttcttcttactcttcttctggttggcgttactactctggtgctttt

SEQ ID NO: 74 - DNA, 15 bp, Artificial Sequence
  Description of Artificial Sequence Synthetic oligonucleotide
ggttcttacctgttc

SEQ ID NO: 75 - DNA, 48 bp, Artificial Sequence
  Description of Artificial Sequence Synthetic oligonucleotide
tcttcttaccgttactcttctggtgcttctttccattactactacggt

SEQ ID NO: 76 - DNA, 18 bp, Artificial Sequence
  Description of Artificial Sequence Synthetic oligonucleotide
tactaccattctccgttc

SEQ ID NO: 77 - DNA, 27 bp, Artificial Sequence
  Description of Artificial Sequence Synthetic oligonucleotide
tcttactactacggtggtttcggtatg

SEQ ID NO: 78 - DNA, 18 bp, Artificial Sequence
  Description of Artificial Sequence Synthetic oligonucleotide
tcttaccgttacccgatc

SEQ ID NO: 79 - DNA, 18 bp, Artificial Sequence
  Description of Artificial Sequence Synthetic oligonucleotide
tctcatacttacccgatc

SEQ ID NO: 80 - DNA, 42 bp, Artificial Sequence
  Description of Artificial Sequence Synthetic oligonucleotide
ggtggttggggtccgtactcttactactaccattacggtttg

SEQ ID NO: 81 - DNA, 24 bp, Artificial Sequence
  Description of Artificial Sequence Synthetic oligonucleotide
tactcttactactacgacctgatc

SEQ ID NO: 82 - DNA, 24 bp, Artificial Sequence
  Description of Artificial Sequence Synthetic oligonucleotide
gacggtttccattcttacgctttg

SEQ ID NO: 83 - DNA, 21 bp, Artificial Sequence
  Description of Artificial Sequence Synthetic oligonucleotide
tactcttacgactctctgatc

SEQ ID NO: 84 - DNA, 24 bp, Artificial Sequence
  Description of Artificial Sequence Synthetic oligonucleotide
ggttggtcttactactggggtttt

SEQ ID NO: 85 - DNA, 18 bp, Artificial Sequence
  Description of Artificial Sequence Synthetic oligonucleotide
tacgactacggtctgttc

SEQ ID NO: 86 - DNA, 21 bp, Artificial Sequence
  Description of Artificial Sequence Synthetic oligonucleotide
tcttctccatctgatctgttc

SEQ ID NO: 87 - DNA, 36 bp, Artificial Sequence
  Description of Artificial Sequence Synthetic oligonucleotide
tggggtcatggttacggttacttctactacggtttt

SEQ ID NO: 88 - DNA, 18 bp, Artificial Sequence
  Description of Artificial Sequence Synthetic oligonucleotide
tcttctggtgacctgatc

SEQ ID NO: 89 - DNA, 387 bp, Artificial Sequence
  Description of Artificial Sequence Synthetic polynucleotide
gaggttcagctggtggagtctggcggtggcctggtgcagccagggcgttcactccgtttgtcctgtgcagcttctggcttcgacctcactggttcttacatgcactgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcg

SEQ ID NO: 90 - protein, 129 aa, Artificial Sequence
  Description of Artificial Sequence Synthetic polypeptide
EVQLVESGGGLVQPGRSLRLSCAASGFDLTGSYMHWVRRAPGKGEELVAGISASGGATAYADSVKGRFTISADTSKNTAYLQMNSLRAEDTAVYYCARSRSSSYSSSGWRYYSGAFDYWGQGTLVTVSS

SEQ ID NO: 91 - DNA, 753 bp, Artificial Sequence
  Description of Artificial Sequence Synthetic polynucleotide
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcg

SEQ ID NO: 92 - protein, 251 aa, Artificial Sequence
  Description of Artificial Sequence Synthetic polypeptide
DIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSS

SEQ ID NO: 93 - DNA, 639 bp, Artificial Sequence
  Description of Artificial Sequence Synthetic polynucleotide
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgaaaaacataaagtctacgcctgcgaagtcacccatcagggcctgagctcgcccgtcacaaagagcttcaacaggggagagtgt

SEQ ID NO: 94 - protein, 213 aa, Artificial Sequence
  Description of Artificial Sequence Synthetic polypeptide
DIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - DNA, 711 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgatctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacaca

SEQ ID NO: 96 - protein, 237 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 97 - DNA, 720 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaa

SEQ ID NO: 98 - protein, 240 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIK

SEQ ID NO: 99 - DNA, 1380 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgatctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 100 - protein, 459 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - DNA, 1128 bp, Artificial Sequence
  Description of Artificial Sequence Synthetic polynucleotide
gaggttcagctggtggagtctggcggtggcctggtgcagccagggcgttcactccgtttgtcctgtgcagcttctggcttcgacctcactggttcttacatgcactgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgaggttcagctggtggagtctggcggtggcctggtgcagccagggggctcactccgtttgtcctgtgcagcttctggcttcgatctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacaca

SEQ ID NO: 102 - protein, 376 aa, Artificial Sequence
  Description of Artificial Sequence Synthetic polypeptide
EVQLVESGGGLVQPGRSLRLSCAASGFDLTGSYMHWVRRAPGKGEELVAGISASGGATAYADSVKGRFTISADTSKNTAYLQMNSLRAEDTAVYYCARSRSSSYSSSGWRYYSGAFDYWGQGTLVTVSSGGGGSGGGGSEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 103 - DNA, 1128 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgatctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacaggtggaggtggctccggaggaggtggcagtgaggttcagctggtggagtctggcggtggcctggtgcagccagggcgttcactccgtttgtcctgtgcagcttctggcttcgacctcactggttcttacatgcactgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcg

SEQ ID NO: 104 - protein, 376 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSEVQLVESGGGLVQPGRSLRLSCAASGFDLTGSYMHWVRRAPGKGEELVAGISASGGATAYADSVKGRFTISADTSKNTAYLQMNSLRAEDTAVYYCARSRSSSYSSSGWRYYSGAFDYWGQGTLVTVSS

SEQ ID NO: 105 - DNA, 1422 bp, Artificial Sequence
  Description of Artificial Sequence Synthetic polynucleotide
gatatccagatgacccagtccccgagctccctgtccgcctctgtgggcgatagggtcaccatcacctgccgtgccagccagagcgtgtccagcgccgtagcctggtatcaacagaaaccaggaaaagctccgaagcttctgatttacagcgccagcgacctgtacagcggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgattactggggtcaaggaaccctggtcaccgtctcctcgggcggcggaggctccggcggcggaggatccgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgacctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaacgtacggtggctgcacccagcgtgttcatcttcccacccagcgacgagcagctgaagtccggcacagccagcgtggtctgcctgctgaacaacttctacccccgcgaggccaaggtgcagtggaaggtggacaacgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgt

SEQ ID NO: 106 - protein, 474 aa, Artificial Sequence
  Description of Artificial Sequence Synthetic polypeptide
DIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSGGGGSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - DNA, 1407 bp, Artificial Sequence
  Description of Artificial Sequence Synthetic polynucleotide
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgaaaaacataaagtctacgcctgcgaagtcacccatcagggcctgagctcgcccgtcacaaagagcttcaacaggggagagtgtggcggcggaggatccgatatccagatgacccagtccccgagctccctgtccgcctctgtgggcgatagggtcaccatcacctgccgtgccagccagagcgtgtccagcgccgtagcctggtatcaacagaaaccaggaaaagctccgaagcttctgatttacagcgccagcgacctgtacagcggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgattactggggtcaaggaaccctggtcaccgtctcctcg

SEQ ID NO: 108 - protein, 469 aa, Artificial Sequence
  Description of Artificial Sequence Synthetic polypeptide
DIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSS

SEQ ID NO: 109 - DNA, 1374 bp, Artificial Sequence
  Description of Artificial Sequence Synthetic polynucleotide
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgacctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaacgtacggtggctgcacccagcgtgttcatcttcccacccagcgacgagcagctgaagtccggcacagccagcgtggtctgcctgctgaacaacttctacccccgcgaggccaaggtgcagtggaaggtggacaacgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgtggcggcggaggatccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgattactggggtcaaggaaccctggtcaccgtctcctcgggcggcggaggatccgatatccagatgacccagtccccgagctccctgtccgcctctgtgggcgatagggtcaccatcacctgccgtgccagccagagcgtgtccagcgccgtagcctggtatcaacagaaaccaggaaaagctccgaagcttctgatttacagcgccagcgacctgtacagcggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaa

SEQ ID NO: 110 - protein, 458 aa, Artificial Sequence
  Description of Artificial Sequence Synthetic polypeptide
DIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIK

SEQ ID NO: 111 - DNA, 1449 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacaggtggatccggcggcgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgacctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaa

SEQ ID NO: 112 - protein, 483 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSGGEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFMDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIK

SEQ ID NO: 113 - DNA, 1146 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacaggaggaggtgggggatcaggtggtgggggctctggaggcggaaccggtgaggttcagctggtggagtctggcggtggcctggtgcagccagggcgttcactccgtttgtcctgtgcagcttctggcttcgacctcactggttcttacatgcactgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcg

SEQ ID NO: 114 - protein, 382 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGGSGGGGSGGGTGEVQLVESGGGLVQPGRSLRLSCAASGFDLTGSYMHWVRRAPGKGEELVAGISASGGATAYADSVKGRFTISADTSKNTAYLQMNSLRAEDTAVYYCARSRSSSYSSSGWRYYSGAFDYWGQGTLVTVSS

SEQ ID NO: 115 - DNA, 1128 bp, Artificial Sequence
  Description of Artificial Sequence Synthetic polynucleotide
gaggttcagctggtggagtctggcggtggcctggtgcagccagggcgttcactccgtttgtcctgtgcagcttctggcttcgacctcactggttcttacatgcactgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacaca

SEQ ID NO: 116 - DNA, 1824 bp, Artificial Sequence
  Description of Artificial Sequence Synthetic polynucleotide
gaggttcagctggtggagtctggcggtggcctggtgcagccagggcgttcactccgtttgtcctgtgcagcttctggcttcgacctcactggttcttacatgcactgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgaaaaacataaagtctacgcctgcgaagtcacccatcagggcctgagctcgcccgtcacaaagagcttcaacaggggagagtgtggcggcggaggatccgatatccagatgacccagtccccgagctccctgtccgcctctgtgggcgatagggtcaccatcacctgccgtgccagccagagcgtgtccagcgccgtagcctggtatcaacagaaaccaggaaaagctccgaagcttctgatttacagcgccagcgacctgtacagcggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgattactggggtcaaggaaccctggtcaccgtctcctcg

SEQ ID NO: 117 - protein, 608 aa, Artificial Sequence
  Description of Artificial Sequence Synthetic polypeptide
EVQLVESGGGLVQPGRSLRLSCAASGFDLTGSYMHWVRRAPGKGEELVAGISASGGATAYADSVKGRFTISADTSKNTAYLQMNSLRAEDTAVYYCARSRSSSYSSSGWRYYSGAFDYWGQGTLVTVSSGGGGSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSS

SEQ ID NO: 118 - DNA, 711 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacaca

SEQ ID NO: 119 - DNA, 1095 bp, Artificial Sequence
  Description of Artificial Sequence Synthetic polynucleotide
gaggttcagctggtggagtctggcggtggcctggtgcagccagggcgttcactccgtttgtcctgtgcagcttctggcttcgacctcactggttcttacatgtgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 120 - protein, 365 aa, Artificial Sequence
  Description of Artificial Sequence Synthetic polypeptide
EVQLVESGGGLVQPGRSLRLSCAASGFDLTGSYMWVRRAPGKGEELVAGISASGGATAYADSVKGRFTISADTSKNTAYLQMNSLRAEDTAVYYCARSRSSSYSSSGWRYYSGAFDYWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - DNA, 1509 bp, Artificial Sequence
  Description of Artificial Sequence Synthetic polynucleotide
gaggttcagctggtggagtctggcggtggcctggtgcagccagggcgttcactccgtttgtcctgtgcagcttctggcttcgacctcactggttcttacatgtgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgaggttcagctggtggagtctggcggtggcctggtgcagccagggcgttcactccgtttgtcctgtgcagcttctggcttcgacctcactggttcttacatgtgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 122 - protein, 503 aa, Artificial Sequence
  Description of Artificial Sequence Synthetic polypeptide
EVQLVESGGGLVQPGRSLRLSCAASGFDLTGSYMWVRRAPGKGEELVAGISASGGATAYADSVKGRFTISADTSKNTAYLQMNSLRAEDTAVYYCARSRSSSYSSSGWRYYSGAFDYWGQGTLVTVSSGGGGSGGGGSEVQLVESGGGLVQPGRSLRLSCAASGFDLTGSYMWVRRAPGKGEELVAGISASGGATAYADSVKGRFTISADTSKNTAYLQMNSLRAEDTAVYYCARSRSSSYSSSGWRYYSGAFDYWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - DNA, 1524 bp, Artificial Sequence
  Description of Artificial Sequence Synthetic polynucleotide
gaggttcagctggtggagtctggcggtggcctggtgcagccagggcgttcactccgtttgtcctgtgcagcttctggcttcgacctcactggttcttacatgtgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggaggaggtgggggatcaggtggtgggggctctggaggcggaaccggtgaggttcagctggtggagtctggcggtggcctggtgcagccagggcgttcactccgtttgtcctgtgcagcttctggcttcgacctcactggttcttacatgtgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcg

SEQ ID NO: 124 - protein, 508 aa, Artificial Sequence
  Description of Artificial Sequence Synthetic polypeptide
EVQLVESGGGLVQPGRSLRLSCAASGFDLTGSYMWVRRAPGKGEELVAGISASGGATAYADSVKGRFTISADTSKNTAYLQMNSLRAEDTAVYYCARSRSSSYSSSGWRYYSGAFDYWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSGGGTGEVQLVESGGGLVQPGRSLRLSCAASGFDLTGSYMWVRRAPGKGEELVAGISASGGATAYADSVKGRFTISADTSKNTAYLQMNSLRAEDTAVYYCARSRSSSYSSSGWRYYSGAFDYWGQGTLVTVSS

SEQ ID NO: 125 - DNA, 1893 bp, Artificial Sequence
  Description of Artificial Sequence Synthetic polynucleotide
gaggttcagctggtggagtctggcggtggcctggtgcagccagggcgttcactccgtttgtcctgtgcagcttctggcttcgacctcactggttcttacatgtgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggaggaggtgggggatcaggtggtgggggctctggaggcggaaccggtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcg

SEQ ID NO: 126 - protein, 631 aa, Artificial Sequence
  Description of Artificial Sequence Synthetic polypeptide
EVQLVESGGGLVQPGRSLRLSCAASGFDLTGSYMWVRRAPGKGEELVAGISASGGATAYADSVKGRFTISADTSKNTAYLQMNSLRAEDTAVYYCARSRSSSYSSSGWRYYSGAFDYWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSGGGTGDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSS

SEQ ID NO: 127 - DNA, 1850 bp, Artificial Sequence
  Description of Artificial Sequence Synthetic polynucleotide
gaggttcagctggtggagtctggcggtggcctggtgcagccagggcgttcactccgtttgtcctgtgcagcttctggcttcgacctcactggttcttacatgtgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggaggaggtgggggatcaggtggtgggggctctggaggcggaaccggtgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggtccatcggtcttccccctggcaccctcctccaagagcacctctgggggcacagcggccctgggctgcctggtcaaggactacttccccgaacccgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacac

SEQ ID NO: 128 - protein, 617 aa, Artificial Sequence
  Description of Artificial Sequence Synthetic polypeptide
EVQLVESGGGLVQPGRSLRLSCAASGFDLTGSYMWVRRAPGKGEELVAGISASGGATAYADSVKGRFTISADTSKNTAYLQMNSLRAEDTAVYYCARSRSSSYSSSGWRYYSGAFDYWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSGGGTGEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 129 - DNA, 1464 bp, Artificial Sequence
  Description of Artificial Sequence Synthetic polynucleotide
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 130 - protein, 488 aa, Artificial Sequence
  Description of Artificial Sequence Synthetic polypeptide
DIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - DNA, 2247 bp, Artificial Sequence
  Description of Artificial Sequence Synthetic polynucleotide
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 132 - protein, 749 aa, Artificial Sequence
  Description of Artificial Sequence Synthetic polypeptide
DIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSGGGGSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - DNA, 2262 bp, Artificial Sequence
  Description of Artificial Sequence Synthetic polynucleotide
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggaggaggtgggggatcaggtggtgggggctctggaggcggaaccggtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcg

SEQ ID NO: 134 - protein, 754 aa, Artificial Sequence
  Description of Artificial Sequence Synthetic polypeptide
DIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSGGGTGDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSS

SEQ ID NO: 135 - DNA, 1896 bp, Artificial Sequence
  Description of Artificial Sequence Synthetic polynucleotide
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggaggaggtgggggatcaggtggtgggggctctggaggcggaaccggtgaggttcagctggtggagtctggcggtggcctggtgcagccagggcgttcactccgtttgtcctgtgcagcttctggcttcgacctcactggttcttacatgcactgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcg

SEQ ID NO: 136 - protein, 632 aa, Artificial Sequence
  Description of Artificial Sequence Synthetic polypeptide
DIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSGGGTGEVQLVESGGGLVQPGRSLRLSCAASGFDLTGSYMHWVRRAPGKGEELVAGISASGGATAYADSVKGRFTISADTSKNTAYLQMNSLRAEDTAVYYCARSRSSSYSSSGWRYYSGAFDYWGQGTLVTVSS

SEQ ID NO: 137 - DNA, 2219 bp, Artificial Sequence
  Description of Artificial Sequence Synthetic polynucleotide
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggaggaggtgggggatcaggtggtgggggctctggaggcggaaccggtgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggtccatcggtcttccccctggcaccctcctccaagagcacctctgggggcacagcggccctgggctgcctggtcaaggactacttccccgaacccgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacac

SEQ ID NO: 138 - protein, 740 aa, Artificial Sequence
  Description of Artificial Sequence Synthetic polypeptide
DIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSGGGTGEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 139 - DNA, 1431 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgatctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtggaggtggctccggaggaggtggcagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 140 - protein, 477 aa, Artificial Sequence
  Description of Artificial Sequence Synthetic polypeptide
EVQLVESGGGLVQPGGSLRLSCAASGFDFSAASIHWVRQAPGKGLEWVAGISASGGATAYADSVKGRFTISADTSKNTAYLQMNSLRAEDTAVYYCARSRSSSYSSSGWRYYSGAFDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - DNA, 2199 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtggaggtggctccggaggaggtggcagtgacaagacacatacctgtccaccttgtccggcacctgagctgcttggaggccctagcgtttttctcttcccgccgaagccgaaggatacgctgatgatcagtagaacccctgaggttacctgtgtcgtggttgatgtctcacacgaagaccccgaggtaaaatttaattggtatgttgacggggtggaagtacataatgccaagaccaaaccccgcgaagaacaatacaacagtacatacagggttgtctcagttttgaccgttctccaccaagattggctcaatgggaaagagtataaatgcaaagtgagtaacaaagcactgccggctccaatagagaagacgatctcaaaagccaagggacaaccacgagagccccaagtctacaccctcccgccctccagggaagagatgactaagaaccaggttagtttgacgtgcttggttaagggcttttacccatctgacatagccgttgaatgggagtcaaatggccaaccagagaataactataagacaaccccccccgtacttgactctgatggctcattcttcttgtattccaaacttactgtcgataaatcccgctggcaacaaggtaacgtcttttcttgtagtgtcatgcatgaggcactgcataaccactatactcaaaaatctctgtccctcagtccaggaggaggtgggggatcaggtggtgggggctctggaggcggaaccggtgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaa

SEQ ID NO: 142 - protein, 733 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFMDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGTGEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFMDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIK

SEQ ID NO: 143 - DNA, 1863 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtggaggtggctccggaggaggtggcagtgacaagacacatacctgtccaccttgtccggcacctgagctgcttggaggccctagcgtttttctcttcccgccgaagccgaaggatacgctgatgatcagtagaacccctgaggttacctgtgtcgtggttgatgtctcacacgaagaccccgaggtaaaatttaattggtatgttgacggggtggaagtacataatgccaagaccaaaccccgcgaagaacaatacaacagtacatacagggttgtctcagttttgaccgttctccaccaagattggctcaatgggaaagagtataaatgcaaagtgagtaacaaagcactgccggctccaatagagaagacgatctcaaaagccaagggacaaccacgagagccccaagtctacaccctcccgccctccagggaagagatgactaagaaccaggttagtttgacgtgcttggttaagggcttttacccatctgacatagccgttgaatgggagtcaaatggccaaccagagaataactataagacaaccccccccgtacttgactctgatggctcattcttcttgtattccaaacttactgtcgataaatcccgctggcaacaaggtaacgtcttttcttgtagtgtcatgcatgaggcactgcataaccactatactcaaaaatctctgtccctcagtccaggaggaggtgggggatcaggtggtgggggctctggaggcggaaccggtgaggttcagctggtggagtctggcggtggcctggtgcagccagggcgttcactccgtttgtcctgtgcagcttctggcttcgacctcactggttcttacatgcattgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcg

SEQ ID NO: 144 - protein, 621 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFMDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGTGEVQLVESGGGLVQPGRSLRLSCAASGFDLTGSYMHWVRRAPGKGEELVAGISASGGATAYADSVKGRFTISADTSKNTAYLQMNSLRAEDTAVYYCARSRSSSYSSSGWRYYSGAFDYWGQGTLVTVSS

SEQ ID NO: 145 - DNA, 2232 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgatctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtggaggtggctccggaggaggtggcagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggaggtgggggatcaggtggtgggggctctggaggcggaaccggtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcg

SEQ ID NO: 146 - protein, 744 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGSGGGGSGGGTGDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSS

SEQ ID NO: 147 - DNA, 2184 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgatctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtggaggtggctccggaggaggtggcagtgacaagacacatacctgtccaccttgtccggcacctgagctgcttggaggccctagcgtttttctcttcccgccgaagccgaaggatacgctgatgatcagtagaacccctgaggttacctgtgtcgtggttgatgtctcacacgaagaccccgaggtaaaatttaattggtatgttgacggggtggaagtacataatgccaagaccaaaccccgcgaagaacaatacaacagtacatacagggttgtctcagttttgaccgttctccaccaagattggctcaatgggaaagagtataaatgcaaagtgagtaacaaagcactgccggctccaatagagaagacgatctcaaaagccaagggacaaccacgagagccccaagtctacaccctcccgccctccagggaagagatgactaagaaccaggttagtttgacgtgcttggttaagggcttttacccatctgacatagccgttgaatgggagtcaaatggccaaccagagaataactataagacaaccccccccgtacttgactctgatggctcattcttcttgtattccaaacttactgtcgataaatcccgctggcaacaaggtaacgtcttttcttgtagtgtcatgcatgaggcactgcataaccactatactcaaaaatctctgtccctcagtccaggaggaggtgggggatcaggtggtgggggctctggaggcggaaccggtgaggttcagctggtggagtctggcggtggcctggtgcagccagggggctcactccgtttgtcctgtgcagcttctggcttcgatctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggtccatcggtcttccccctggcaccctcctccaagagcacctctgggggcacagcggccctgggctgcctggtcaaggactacttccccgaacccgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacaca

SEQ ID NO: 148 - protein, 728 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGTGEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 149 - DNA, 2214 bp, Artificial Sequence
  Description of Artificial Sequence Synthetic polynucleotide
gaagtccaactcgtcgaatccggtggtggattggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacaaccggtggaggtggcagtggcggaggtggcagcggtggaggtggaagtggaggaggtggcagtggtggaggagaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtggaggtggctccggaggaggtggcagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 150 - protein, 738 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTTGGGGSGGGGSGGGGSGGGGSGGGEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - DNA, 1794 bp, Artificial Sequence
  Description of Artificial Sequence Synthetic polynucleotide
gaggttcagctggtggagtctggcggtggcctggtgcagccagggcgttcactccgtttgtcctgtgcagcttctggcttcgacctcactggttcttacatgcactgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 152 - protein, 598 aa, Artificial Sequence
  Description of Artificial Sequence Synthetic polypeptide
EVQLVESGGGLVQPGRSLRLSCAASGFDLTGSYMHWVRRAPGKGEELVAGISASGGATAYADSVKGRFTISADTSKNTAYLQMNSLRAEDTAVYYCARSRSSSYSSSGWRYYSGAFDYWGQGTLVTVSSGGGGSGGGGSEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - DNA, 2211 bp, Artificial Sequence
  Description of Artificial Sequence Synthetic polynucleotide
gaggttcagctggtggagtctggcggtggcctggtgcagccagggcgttcactccgtttgtcctgtgcagcttctggcttcgacctcactggttcttacatgcactgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgaggttcagctggtggagtctggcggtggcctggtgcagccagggcgttcactccgtttgtcctgtgcagcttctggcttcgacctcactggttcttacatgcactgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 154 - protein, 737 aa, Artificial Sequence
  Description of Artificial Sequence Synthetic polypeptide
EVQLVESGGGLVQPGRSLRLSCAASGFDLTGSYMHWVRRAPGKGEELVAGISASGGATAYADSVKGRFTISADTSKNTAYLQMNSLRAEDTAVYYCARSRSSSYSSSGWRYYSGAFDYWGQGTLVTVSSGGGGSGGGGSEVQLVESGGGLVQPGRSLRLSCAASGFDLTGSYMHWVRRAPGKGEELVAGISASGGATAYADSVKGRFTISADTSKNTAYLQMNSLRAEDTAVYYCARSRSSSYSSSGWRYYSGAFDYWGQGTLVTVSSGGGGSGGGGSEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - DNA, 2160 bp, Artificial Sequence
  Description of Artificial Sequence Synthetic polynucleotide
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 156 - protein, 720 aa, Artificial Sequence
  Description of Artificial Sequence Synthetic polypeptide
DIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSGGGGSGGGGSEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - DNA, 2943 bp, Artificial Sequence
  Description of Artificial Sequence Synthetic polynucleotide
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 158 - protein, 981 aa, Artificial Sequence
  Description of Artificial Sequence Synthetic polypeptide
DIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSGGGGSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSGGGGSGGGGSEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - DNA, 2136 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgatctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatccggaggaggtggcagtggcggaggtggcagcggtggaggtggcagtgaggttcagctggtggagtctggcggtggcctggtgcagccagggggctcactccgtttgtcctgtgcagcttctggcttcgatctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 160 - protein, 712 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSGGGGSGGGGSGGGGSEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - DNA, 2895 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatccggaggaggtggcagtggcggaggtggcagcggtggaggtggcagtgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatccggaggaggtggcagtggcggaggtggcagcggtggaggtggcagtgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 162 - protein, 965 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSGGGGSGGGGSGGGGSEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSGGGGSGGGGSGGGGSEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - DNA, 1809 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggaggaggtgggggatcaggtggtgggggctctggaggcggaaccggtgaggttcagctggtggagtctggcggtggcctggtgcagccagggcgttcactccgtttgtcctgtgcagcttctggcttcgacctcactggttcttacatgcactgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcg

SEQ ID NO: 164 - protein, 603 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSGGGTGEVQLVESGGGLVQPGRSLRLSCAASGFDLTGSYMHWVRRAPGKGEELVAGISASGGATAYADSVKGRFTISADTSKNTAYLQMNSLRAEDTAVYYCARSRSSSYSSSGWRYYSGAFDYWGQGTLVTVSS

SEQ ID NO: 165 - DNA, 2226 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggaggaggtgggggatcaggtggtgggggctctggaggcggaaccggtgaggttcagctggtggagtctggcggtggcctggtgcagccagggcgttcactccgtttgtcctgtgcagcttctggcttcgacctcactggttcttacatgcactgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgaggttcagctggtggagtctggcggtggcctggtgcagccagggcgttcactccgtttgtcctgtgcagcttctggcttcgacctcactggttcttacatgcactgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcg

SEQ ID NO: 166 - protein, 742 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSGGGTGEVQLVESGGGLVQPGRSLRLSCAASGFDLTGSYMHWVRRAPGKGEELVAGISASGGATAYADSVKGRFTISADTSKNTAYLQMNSLRAEDTAVYYCARSRSSSYSSSGWRYYSGAFDYWGQGTLVTVSSGGGGSGGGGSEVQLVESGGGLVQPGRSLRLSCAASGFDLTGSYMHWVRRAPGKGEELVAGISASGGATAYADSVKGRFTISADTSKNTAYLQMNSLRAEDTAVYYCARSRSSSYSSSGWRYYSGAFDYWGQGTLVTVSS

SEQ ID NO: 167 - DNA, 2175 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggaggaggtgggggatcaggtggtgggggctctggaggcggaaccggtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcg

SEQ ID NO: 168 - protein, 725 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSGGGTGDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSS

SEQ ID NO: 169 - DNA, 2958 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggaggaggtgggggatcaggtggtgggggctctggaggcggaaccggtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcg

SEQ ID NO: 170 - protein, 986 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSGGGTGDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSGGGGSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSS

SEQ ID NO: 171 - DNA, 2142 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggaggtgggggatcaggtggtgggggctctggaggcggaaccggtgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttatggactactggggtcaaggaaccctggtcaccgtctcctcgggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaa

SEQ ID NO: 172 - protein, 714 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGTGEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFMDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIK

SEQ ID NO: 173 - DNA, 2133 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgatctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggaggaggtgggggatcaggtggtgggggctctggaggcggaaccggtgaggttcagctggtggagtctggcggtggcctggtgcagccagggggctcactccgtttgtcctgtgcagcttctggcttcgatctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggtccatcggtcttccccctggcaccctcctccaagagcacctctgggggcacagcggccctgggctgcctggtcaaggactacttccccgaacccgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacaca

SEQ ID NO: 174 - protein, 711 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSGGGTGEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 175 - DNA, 2874 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggaggaggtgggggatcaggtggtgggggctctggaggcggaaccggtgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggtccatcggtcttccccctggcaccctcctccaagagcacctctgggggcacagcggccctgggctgcctggtcaaggactacttccccgaacccgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacacaggtggaggtggctccggaggaggtggcagtgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggtccatcggtcttccccctggcaccctcctccaagagcacctctgggggcacagcggccctgggctgcctggtcaaggactacttccccgaacccgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacaca

SEQ ID NO: 176 - protein, 958 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGGSGGGGSGGGTGEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 177 - DNA, 1398 bp, Artificial Sequence
  Description of Artificial Sequence Synthetic polynucleotide
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgaaaaacataaagtctacgcctgcgaagtcacccatcagggcctgagctcgcccgtcacaaagagcttcaacaggggagagtgtggaggaggtggcagtggcggaggtggcagcggtggaggtggcagtgaggttcagctggtggagtctggcggtggcctggtgcagccagggggctcactccgtttgtcctgtgcagcttctggcttcgatctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggtccatcggtcttccccctggcaccctcctccaagagcacctctgggggcacagcggccctgggctgcctggtcaaggactacttccccgaacccgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacacatga

SEQ ID NO: 178 - protein, 465 aa, Artificial Sequence
  Description of Artificial Sequence Synthetic polypeptide
DIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 179 - DNA, 2079 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgatctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatccggaggaggtggcagtggcggaggtggcagcggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgaaaaacataaagtctacgcctgcgaagtcacccatcagggcctgagctcgcccgtcacaaagagcttcaacaggggagagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 180 - protein, 693 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSGGGGSGGGGSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - DNA, 1398 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgatctctcttcttattctatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggtccatcggtcttccccctggcaccctcctccaagagcacctctgggggcacagcggccctgggctgcctggtcaaggactacttccccgaacccgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacacatccggaggaggtggcagtggcggaggtggcagcggtggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgaaaaacataaagtctacgcctgcgaagtcacccatcagggcctgagctcgcccgtcacaaagagcttcaacaggggagagtgt

SEQ ID NO: 182 - protein, 466 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSGGGGSGGGGSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - DNA, 2064 bp, Artificial Sequence
  Description of Artificial Sequence Synthetic polynucleotide
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgaaaaacataaagtctacgcctgcgaagtcacccatcagggcctgagctcgcccgtcacaaagagcttcaacaggggagagtgtagcggaggaggtggcagtggcggaggtggcagcggtggaggtggcagtgaggttcagctggtggagtctggcggtggcctggtgcagccagggggctcactccgtttgtcctgtgcagcttctggcttcgatctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggtccatcggtcttccccctggcaccctcctccaagagcacctctgggggcacagcggccctgggctgcctggtcaaggactacttccccgaacccgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 184 - protein, 688 aa, Artificial Sequence
  Description of Artificial Sequence Synthetic polypeptide
DIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSGGGGSEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - DNA, 2553 bp, Artificial Sequence
  Description of Artificial Sequence Synthetic polynucleotide
gaggttcagctggtggagtctggcggtggcctggtgcagccagggcgttcactccgtttgtcctgtgcagcttctggcttcgacctcactggttcttacatgcactgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatccggaggaggtggcagtggcggaggtggcagcggtggaggtggcagtgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 186 - protein, 851 aa, Artificial Sequence
  Description of Artificial Sequence Synthetic polypeptide
EVQLVESGGGLVQPGRSLRLSCAASGFDLTGSYMHWVRRAPGKGEELVAGISASGGATAYADSVKGRFTISADTSKNTAYLQMNSLRAEDTAVYYCARSRSSSYSSSGWRYYSGAFDYWGQGTLVTVSSGGGGSGGGGSEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSGGGGSGGGGSGGGGSEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - DNA, 2919 bp, Artificial Sequence
  Description of Artificial Sequence Synthetic polynucleotide
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatccggaggaggtggcagtggcggaggtggcagcggtggaggtggcagtgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 188 - protein, 973 aa, Artificial Sequence
  Description of Artificial Sequence Synthetic polypeptide
DIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSGGGGSGGGGSEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSGGGGSGGGGSGGGGSEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - DNA, 2535 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacaggtggaggtggctccggaggaggtggcagtgaggttcagctggtggagtctggcggtggcctggtgcagccagggcgttcactccgtttgtcctgtgcagcttctggcttcgacctcactggttcttacatgcactgggtgcgtcgtgccccgggtaagggcgaggaactggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 190 - protein, 845 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSEVQLVESGGGLVQPGRSLRLSCAASGFDLTGSYMHWVRRAPGKGEELVAGISASGGATAYADSVKGRFTISADTSKNTAYLQMNSLRAEDTAVYYCARSRSSSYSSSGWRYYSGAFDYWGQGTLVTVSSGGGGSGGGGSEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - DNA, 2901 bp, Artificial Sequence
  Description of Artificial Sequence Synthetic polynucleotide
gaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacaggtggaggtggctccggaggaggtggcagtgatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccgatctctactctggagtcccttctcgcttctctggtagccgttccgggacggatttcactctgaccatcagcagtctgcagccggaagacttcgcaacttattactgtcagcaaggttcttacctgttcacgttcggacagggtaccaaggtggagatcaaaggtactactgccgctagtggtagtagtggtggcagtagcagtggtgccgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcgggtggaggtggctccggaggaggtggcagtgaggttcagctggtggagtctggcggtggcctggtgcagccagggggctcactccgtttgtcctgtgcagcttctggcttcgacctcactggttcttacatgcactgggtgcgtcaggccccgggtaagggcctggaatgggttgcaggtatttccgcttctggaggcgctactgcttatgccgatagcgtcaagggccgtttcactataagcgcagacacatccaaaaacacagcctacctacaaatgaacagcttaagagctgaggacactgccgtctattattgtgctcgctctcgttcttcttcttactcttcttctggttggcgttactactctggtgcttttgactactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 192 - protein, 967 aa, Artificial Sequence
  Description of Artificial Sequence Synthetic polypeptide
EVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQGSYLFTFGQGTKVEIKGTTAASGSSGGSSSGAEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSGGGGSGGGGSEVQLVESGGGLVQPGGSLRLSCAASGFDLTGSYMHWVRQAPGKGLEWVAGISASGGATAYADSVKGRFTISADTSKNTAYLQMNSLRAEDTAVYYCARSRSSSYSSSGWRYYSGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 21 aa, Artificial Sequence
  Description of Artificial Sequence Synthetic peptide
  MOD_RES (1)..(1): N, P, Q, D, T, E, W, S, V or absent
  MOD_RES (4)..(4): A, S, Y, M, E, N, R, K, T, D, F, P or absent
  MOD_RES (7)..(7): S, A, E, V, Y, N, Q, H, P, W or M
  MOD_RES (10)..(10): W, A, H, S, Y, R, V, E or I
  MOD_RES (17)..(17): G, Q, W or absent
  MOD_RES (18)..(18): M, T or absent
XHHXMIXWEXQLVQMLXXHLI

SEQ ID NO: 194 - protein, 16 aa, Artificial Sequence
  Description of Artificial Sequence Synthetic peptide
NHLADPSWEWMLVQML

SEQ ID NO: 195 - protein, 16 aa, Artificial Sequence
  Description of Artificial Sequence Synthetic peptide
PEASVIAWEWFLFPEW

SEQ ID NO: 196 - protein, 16 aa, Artificial Sequence
  Description of Artificial Sequence Synthetic peptide
QPMYTHEWEWVLQHIM

SEQ ID NO: 197 - protein, 16 aa, Artificial Sequence
  Description of Artificial Sequence Synthetic peptide
DWHMGYVWEAELISIM

SEQ ID NO: 198 - protein, 16 aa, Artificial Sequence
  Description of Artificial Sequence Synthetic peptide
THHEDMYWDHQLVFIM

SEQ ID NO: 199 - protein, 16 aa, Artificial Sequence
  Description of Artificial Sequence Synthetic peptide
PFVNNDNWESQLVDMM

SEQ ID NO: 200 - protein, 16 aa, Artificial Sequence
  Description of Artificial Sequence Synthetic peptide
EMDRHWNWEYWIVQMM

SEQ ID NO: 201 - protein, 16 aa, Artificial Sequence
  Description of Artificial Sequence Synthetic peptide
NQEKYIQWEYRLVQMM

SEQ ID NO: 202 - protein, 16 aa, Artificial Sequence
  Description of Artificial Sequence Synthetic peptide
WHWTMEHWESSLIEML

SEQ ID NO: 203 - protein, 16 aa, Artificial Sequence
  Description of Artificial Sequence Synthetic peptide
SKSDMHPWERQLIWML

SEQ ID NO: 204 - protein, 16 aa, Artificial Sequence
  Description of Artificial Sequence Synthetic peptide
QHHDMQWWEVQIVNML

SEQ ID NO: 205 - protein, 16 aa, Artificial Sequence
  Description of Artificial Sequence Synthetic peptide
PFFTAMWEEWLVEVLG

SEQ ID NO: 206 - protein, 16 aa, Artificial Sequence
  Description of Artificial Sequence Synthetic peptide
VRSPPPHWEEFLVDML

SEQ ID NO: 207 - protein, 16 aa, Artificial Sequence
  Description of Artificial Sequence Synthetic peptide
IPWEIDLEQWIQMHLI

SEQ ID NO: 208 - protein, 16 aa, Artificial Sequence
  Description of Artificial Sequence Synthetic peptide
MSWVAWEEMLESWLWT

SEQ ID NO: 209 - protein, 19 aa, Artificial Sequence
  Description of Artificial Sequence Synthetic peptide
  MOD_RES (3)..(3): D, E, W, T, V, I, F, Q, A or absent
  MOD_RES (5)..(5): A, D, N, F, P, V, H, Y, Q, S, E, I, W or T
  MOD_RES (10)..(10): E, A, S, M, D, L, V, Q, H, W or P
  MOD_RES (17)..(17): E, I, Q, A, P, G or absent
  MOD_RES (19)..(19): I, H or absent
DWXEXEEWMXQLVYMLXPX

SEQ ID NO: 210 - protein, 16 aa, Artificial Sequence
  Description of Artificial Sequence Synthetic peptide
DFDFAENWMEQLKVML

SEQ ID NO: 211 - protein, 16 aa, Artificial Sequence
  Description of Artificial Sequence Synthetic peptide
EDDMLWTAIFGQMWEI

SEQ ID NO: 212 - protein, 16 aa, Artificial Sequence
  Description of Artificial Sequence Synthetic peptide
HNLMWYSNFMHMWIPI

SEQ ID NO: 213 - protein, 16 aa, Artificial Sequence
  Description of Artificial Sequence Synthetic peptide
QWELFEQWVMNNYNTV

SEQ ID NO: 214 - protein, 16 aa, Artificial Sequence
  Description of Artificial Sequence Synthetic peptide
EIDFFYQWLDQMQLNN

SEQ ID NO: 215 - protein, 16 aa, Artificial Sequence
  Description of Artificial Sequence Synthetic peptide
WEPEPWMLQAFWEWQL

SEQ ID NO: 216 - protein, 16 aa, Artificial Sequence
  Description of Artificial Sequence Synthetic peptide
DDWEFLFWQVMFPEWY

SEQ ID NO: 217 - protein, 16 aa, Artificial Sequence
  Description of Artificial Sequence Synthetic peptide
DDTWVMEWIQSMGWSQ

SEQ ID NO: 218 - protein, 16 aa, Artificial Sequence
  Description of Artificial Sequence Synthetic peptide
DIWWHPSWLESFTHDY

SEQ ID NO: 219 - protein, 16 aa, Artificial Sequence
  Description of Artificial Sequence Synthetic peptide
SYVHDPSWMMEMMHIL

SEQ ID NO: 220 - protein, 16 aa, Artificial Sequence
  Description of Artificial Sequence Synthetic peptide
EYHAWMEMLASWAAPH

SEQ ID NO: 221 - protein, 16 aa, Artificial Sequence
  Description of Artificial Sequence Synthetic peptide
WIEQFAILMGAEMFLP

SEQ ID NO: 222 - protein, 16 aa, Artificial Sequence
  Description of Artificial Sequence Synthetic peptide
PVEQWNWMDVFITQLG

SEQ ID NO: 223 - protein, 16 aa, Artificial Sequence
  Description of Artificial Sequence Synthetic peptide
GLELSDGWMQQLSVMM

SEQ ID NO: 224 - protein, 16 aa, Artificial Sequence
  Description of Artificial Sequence Synthetic peptide
WQFSEQPWMQHLIMVM

SEQ ID NO: 225 - protein, 16 aa, Artificial Sequence
  Description of Artificial Sequence Synthetic peptide
QQIQDLEWMHTLMQMM

SEQ ID NO: 226 - protein, 16 aa, Artificial Sequence
  Description of Artificial Sequence Synthetic peptide
NSEVHMEWMAHLMFMM

SEQ ID NO: 227 - protein, 16 aa, Artificial Sequence
  Description of Artificial Sequence Synthetic peptide
PRQGNDYWIDHLVEML

SEQ ID NO: 228 - protein, 16 aa, Artificial Sequence
  Description of Artificial Sequence Synthetic peptide
QMDQNVHWMQMFVELL

SEQ ID NO: 229 - protein, 16 aa, Artificial Sequence
  Description of Artificial Sequence Synthetic peptide
QWADPQEWMDQMVAML

SEQ ID NO: 230 - protein, 16 aa, Artificial Sequence
  Description of Artificial Sequence Synthetic peptide
NMAPSEDWMMTLVYAL

SEQ ID NO: 231 - protein, 16 aa, Artificial Sequence
  Description of Artificial Sequence Synthetic peptide
ENWPSDDWMWQVVAAL

SEQ ID NO: 232 - protein, 16 aa, Artificial Sequence
  Description of Artificial Sequence Synthetic peptide
EWIVIDQWIPQLVYMM

SEQ ID NO: 233 - protein, 16 aa, Artificial Sequence
  Description of Artificial Sequence Synthetic peptide
DKQFWVSSYHQLVYMM

SEQ ID NO: 234 - protein, 16 aa, Artificial Sequence
  Description of Artificial Sequence Synthetic peptide
SMQETEVWQWQLVYMM

SEQ ID NO: 235 - protein, 19 aa, Artificial Sequence
  Description of Artificial Sequence Synthetic peptide
  MOD_RES (4)..(4): P, D, W, K, V, M, S, Y, T, E, L or I
MEPXWWQDEEELVQMLMMM

SEQ ID NO: 236 - protein, 16 aa, Artificial Sequence
  Description of Artificial Sequence Synthetic peptide
SWLPVFFDEGGVMRML

SEQ ID NO: 237 - protein, 16 aa, Artificial Sequence
  Description of Artificial Sequence Synthetic peptide
HADWMVMEESYFIQAL

SEQ ID NO: 238 - protein, 16 aa, Artificial Sequence
  Description of Artificial Sequence Synthetic peptide
THWWMADPQELIAIMG

SEQ ID NO: 239 - protein, 16 aa, Artificial Sequence
  Description of Artificial Sequence Synthetic peptide
MKPKFFQDEFALWMMM

SEQ ID NO: 240 - protein, 16 aa, Artificial Sequence
  Description of Artificial Sequence Synthetic peptide
KWGPSPHEMAQFVWMM

SEQ ID NO: 241 - protein, 16 aa, Artificial Sequence
  Description of Artificial Sequence Synthetic peptide
NDPVWYMDAWYAMQMF

SEQ ID NO: 242 - protein, 16 aa, Artificial Sequence
  Description of Artificial Sequence Synthetic peptide
MSDMYMFDEWKMVYAL

SEQ ID NO: 243 - protein, 16 aa, Artificial Sequence
  Description of Artificial Sequence Synthetic peptide
EYLSFWQSEWAFVEAL

SEQ ID NO: 244 - protein, 16 aa, Artificial Sequence
  Description of Artificial Sequence Synthetic peptide
PHIYWHRPEEYLMQML

SEQ ID NO: 245 - protein, 16 aa, Artificial Sequence
  Description of Artificial Sequence Synthetic peptide
DFPTWIIHEQQLMFML

SEQ ID NO: 246 - protein, 16 aa, Artificial Sequence
  Description of Artificial Sequence Synthetic peptide
WWNMDPLQEKFLVMMT

SEQ ID NO: 247 - protein, 16 aa, Artificial Sequence
  Description of Artificial Sequence Synthetic peptide
WWYQDNYDISQLIEMM

SEQ ID NO: 248 - protein, 16 aa, Artificial Sequence
  Description of Artificial Sequence Synthetic peptide
IEWEWWLENEFMHIDP

SEQ ID NO: 249 - protein, 16 aa, Artificial Sequence
  Description of Artificial Sequence Synthetic peptide
TQREWPAEWEFLSFML

SEQ ID NO: 250 - protein, 16 aa, Artificial Sequence
  Description of Artificial Sequence Synthetic peptide
MEMLWWQEEAMMQLVM

SEQ ID NO: 251 - protein, 16 aa, Artificial Sequence
  Description of Artificial Sequence Synthetic peptide
LLWHSEDAEMLLHVMM

SEQ ID NO: 252 - protein, 16 aa, Artificial Sequence
  Description of Artificial Sequence Synthetic peptide
QEVIWWMEQQDLVHVM

SEQ ID NO: 253 - protein, 16 aa, Artificial Sequence
  Description of Artificial Sequence Synthetic peptide
QEPMWWINEEEFVRLL

SEQ ID NO: 254 - protein, 16 aa, Artificial Sequence
  Description of Artificial Sequence Synthetic peptide
LDVMWHINELDFVNYF

SEQ ID NO: 255 - protein, 17 aa, Artificial Sequence
  Description of Artificial Sequence Synthetic peptide
  MOD_RES (1)..(1): I, D, Q, L, P, M, E, A, V, T, Y, S, N, W or H
  MOD_RES (12)..(12): H, V, L, E, T, A, F, K, I, W, M or Y
  MOD_RES (14)..(14): E, Q, D, P, T, V, H, R, S, A, L, I or Y
XEIDWCQIWQCXEXELD

SEQ ID NO: 256 - protein, 16 aa, Artificial Sequence
  Description of Artificial Sequence Synthetic peptide
IMTDWCKIWECHEEQV

SEQ ID NO: 257 - protein, 16 aa, Artificial Sequence
  Description of Artificial Sequence Synthetic peptide
DQVDWCLIWQCVPQDD

SEQ ID NO: 258 - protein, 16 aa, Artificial Sequence
  Description of Artificial Sequence Synthetic peptide
QEAMWCEIWQCVTEML

SEQ ID NO: 259 - protein, 16 aa, Artificial Sequence
  Description of Artificial Sequence Synthetic peptide
QGIDWCVLWNCLFDHE

SEQ ID NO: 260 - protein, 16 aa, Artificial Sequence
  Description of Artificial Sequence Synthetic peptide
LEQIVCMWMVCEEDMH

SEQ ID NO: 261 - protein, 17 aa, Artificial Sequence
  Description of Artificial Sequence Synthetic peptide
PAMDWCQIFQCTIQVLD

SEQ ID NO: 262 - protein, 16 aa, Artificial Sequence
  Description of Artificial Sequence Synthetic peptide
MWPEWCQVWICALPEI

SEQ ID NO: 263 - protein, 16 aa, Artificial Sequence
  Description of Artificial Sequence Synthetic peptide
LEIDWCQIWDCFPTHK

SEQ ID NO: 264 - protein, 16 aa, Artificial Sequence
  Description of Artificial Sequence Synthetic peptide
EGEPWCNIFQCTYTAM

SEQ ID NO: 265 - protein, 16 aa, Artificial Sequence
  Description of Artificial Sequence Synthetic peptide
ALIDWCLIWTCVLDPS

SEQ ID NO: 266 - protein, 16 aa, Artificial Sequence
  Description of Artificial Sequence Synthetic peptide
VMVDWCQIFGCKWVPI

SEQ ID NO: 267 - protein, 16 aa, Artificial Sequence
  Description of Artificial Sequence Synthetic peptide
EDWDWCAIWNCLEHHL

SEQ ID NO: 268 - protein, 16 aa, Artificial Sequence
  Description of Artificial Sequence Synthetic peptide
IWPDWCWIWECVSRQI

SEQ ID NO: 269 - protein, 16 aa, Artificial Sequence
  Description of Artificial Sequence Synthetic peptide
TLIDWCNLWSCVDSFP

SEQ ID NO: 270 - protein, 16 aa, Artificial Sequence
  Description of Artificial Sequence Synthetic peptide
IQVDWCQWFICTQENL

SEQ ID NO: 271 - protein, 16 aa, Artificial Sequence
  Description of Artificial Sequence Synthetic peptide
YHVTWCLIWECLTAVP

SEQ ID NO: 272 - protein, 16 aa, Artificial Sequence
  Description of Artificial Sequence Synthetic peptide
DIWHWCFFHQCEVQIE

SEQ ID NO: 273 - protein, 16 aa, Artificial Sequence
  Description of Artificial Sequence Synthetic peptide
QWPDWCSIWSCLPTEA

SEQ ID NO: 274 - protein, 16 aa, Artificial Sequence
  Description of Artificial Sequence Synthetic peptide
SEIDWCSWWHCIELAD

SEQ ID NO: 275 - protein, 16 aa, Artificial Sequence
  Description of Artificial Sequence Synthetic peptide
NWVDWCQYWVCVQDVD

SEQ ID NO: 276 - protein, 16 aa, Artificial Sequence
  Description of Artificial Sequence Synthetic peptide
LDMDWCQIWDCIQVYN

SEQ ID NO: 277 - protein, 16 aa, Artificial Sequence
  Description of Artificial Sequence Synthetic peptide
WNSDPCDFWQCIQPTA

SEQ ID NO: 278 - protein, 16 aa, Artificial Sequence
  Description of Artificial Sequence Synthetic peptide
MHIEWCELFQCWHVDQ

SEQ ID NO: 279 - protein, 16 aa, Artificial Sequence
  Description of Artificial Sequence Synthetic peptide
IMWDPCMVWECIDATY

SEQ ID NO: 280 - protein, 16 aa, Artificial Sequence
  Description of Artificial Sequence Synthetic peptide
HELWFCQLWTCIDIPG

SEQ ID NO: 281 - protein, 16 aa, Artificial Sequence
  Description of Artificial Sequence Synthetic peptide
HALDWCQIWTCMTPQN

SEQ ID NO: 282 - protein, 16 aa, Artificial Sequence
  Description of Artificial Sequence Synthetic peptide
MYDEWCMIWECLPESP

SEQ ID NO: 283 - protein, 16 aa, Artificial Sequence
  Description of Artificial Sequence Synthetic peptide
PDFNWCHWWECMEVEL

SEQ ID NO: 284 - protein, 16 aa, Artificial Sequence
  Description of Artificial Sequence Synthetic peptide
DTLQWCEWWTCIWDHH

SEQ ID NO: 285 - protein, 16 aa, Artificial Sequence
  Description of Artificial Sequence Synthetic peptide
MWIEWCDIWTCIEYEP

SEQ ID NO: 286 - protein, 16 aa, Artificial Sequence
  Description of Artificial Sequence Synthetic peptide
LDQAWCGIWNCIHVTP

SEQ ID NO: 287 - protein, 16 aa, Artificial Sequence
  Description of Artificial Sequence Synthetic peptide
YHISWCDIWDCVVSEQ

SEQ ID NO: 288 - protein, 16 aa, Artificial Sequence
  Description of Artificial Sequence Synthetic peptide
YEVNWCSFWTCVQEVA

SEQ ID NO: 289 - protein, 16 aa, Artificial Sequence
  Description of Artificial Sequence Synthetic peptide
QLVDWCNIWQCYPSDL

SEQ ID NO: 290 - protein, 16 aa, Artificial Sequence
  Description of Artificial Sequence Synthetic peptide
WSWDPCEMFSCMLQYH

SEQ ID NO: 291 - protein, 16 aa, Artificial Sequence
  Description of Artificial Sequence Synthetic peptide
  MOD_RES (6)..(6): F, E, G, Y, Q or R
  MOD_RES (12)..(12): S, Q, M, I, E or A
  MOD_RES (14)..(14): E, I, Q or D
  MOD_RES (15)..(15): S, Y, D or L
  MOD_RES (16)..(16): E, K, N, Y, M or A
EWYICXQFPEWXCXXX

SEQ ID NO: 292 - protein, 16 aa, Artificial Sequence
  Description of Artificial Sequence Synthetic peptide
TAYECFQFPEWSCESE

SEQ ID NO: 293 - protein, 16 aa, Artificial Sequence
  Description of Artificial Sequence Synthetic peptide
SLNICEQFPEFQCIYK

SEQ ID NO: 294 - protein, 16 aa, Artificial Sequence
  Description of Artificial Sequence Synthetic peptide
LWDICGQFPEWMCIDN

SEQ ID NO: 295 - protein, 16 aa, Artificial Sequence
  Description of Artificial Sequence Synthetic peptide
EWESCYDSDWHICQLY

SEQ ID NO: 296 - protein, 16 aa, Artificial Sequence
  Description of Artificial Sequence Synthetic peptide
YPIWCQWFPEWECDDM

SEQ ID NO: 297 - protein, 16 aa, Artificial Sequence
  Description of Artificial Sequence Synthetic peptide
EGYTCRPEDWHACQLA

SEQ ID NO: 298 - protein, 16 aa, Artificial Sequence
  Description of Artificial Sequence Synthetic peptide
  MOD_RES (7)..(7): Y, F, N, P, W, H, Q, S, K or L
ECWEWDXEMQZWVPCD

SEQ ID NO: 299 - protein, 16 aa, Artificial Sequence
  Description of Artificial Sequence Synthetic peptide
ECWEWSYQLQEWSPCP

SEQ ID NO: 300 - protein, 16 aa, Artificial Sequence
  Description of Artificial Sequence Synthetic peptide
DCWEYDYKQMSWERCD

SEQ ID NO: 301 - protein, 16 aa, Artificial Sequence
  Description of Artificial Sequence Synthetic peptide
ECWEWEFQYQLGWKCI

SEQ ID NO: 302 - protein, 16 aa, Artificial Sequence
  Description of Artificial Sequence Synthetic peptide
FCWQQKNDEYIRIECA

SEQ ID NO: 303 - protein, 16 aa, Artificial Sequence
  Description of Artificial Sequence Synthetic peptide
FCMEWIPATMEWVPCD

SEQ ID NO: 304 - protein, 16 aa, Artificial Sequence
  Description of Artificial Sequence Synthetic peptide
SCMEFMWDTQTWELCA

SEQ ID NO: 305 - protein, 16 aa, Artificial Sequence
  Description of Artificial Sequence Synthetic peptide
MCWEFMWEFESWQPCS

SEQ ID NO: 306 - protein, 16 aa, Artificial Sequence
  Description of Artificial Sequence Synthetic peptide
ECWEWIHNMQEWKSCV

SEQ ID NO: 307 - protein, 16 aa, Artificial Sequence
  Description of Artificial Sequence Synthetic peptide
SCEWWGPESRQIYMCE

SEQ ID NO: 308 - protein, 16 aa, Artificial Sequence
  Description of Artificial Sequence Synthetic peptide
ECWEWSHIWGEFLECT

SEQ ID NO: 309 - protein, 16 aa, Artificial Sequence
  Description of Artificial Sequence Synthetic peptide
ECWEYLPHWGIWQDCG

SEQ ID NO: 310 - protein, 16 aa, Artificial Sequence
  Description of Artificial Sequence Synthetic peptide
QCWEYQWTMQMWVECE

SEQ ID NO: 311 - protein, 16 aa, Artificial Sequence
  Description of Artificial Sequence Synthetic peptide
SCWEYMFEAQQWVACM

SEQ ID NO: 312 - protein, 16 aa, Artificial Sequence
  Description of Artificial Sequence Synthetic peptide
QCWQYVFDMAMWTTCG

SEQ ID NO: 313 - protein, 16 aa, Artificial Sequence
  Description of Artificial Sequence Synthetic peptide
MCWEWFQETRTWVYCD

SEQ ID NO: 314 - protein, 16 aa, Artificial Sequence
  Description of Artificial Sequence Synthetic peptide
MCYEWEWMSHTWVDCQ

SEQ ID NO: 315 - protein, 16 aa, Artificial Sequence
  Description of Artificial Sequence Synthetic peptide
QCWEWMSVVNGWEPCG

SEQ ID NO: 316 - protein, 16 aa, Artificial Sequence
  Description of Artificial Sequence Synthetic peptide
MCWEWSSLQDNWMPCD

SEQ ID NO: 317 - protein, 16 aa, Artificial Sequence
  Description of Artificial Sequence Synthetic peptide
QCWEWDKAMSQWVYCA

SEQ ID NO: 318 - protein, 16 aa, Artificial Sequence
  Description of Artificial Sequence Synthetic peptide
QCWQFNFSLLQWEVCG

SEQ ID NO: 319 - protein, 16 aa, Artificial Sequence
  Description of Artificial Sequence Synthetic peptide
ICEIVVQTEEHVWECM

SEQ ID NO: 320 - protein, 16 aa, Artificial Sequence
  Description of Artificial Sequence Synthetic peptide
QCWMLEQTSLTWVPCP

SEQ ID NO: 321 - protein, 16 aa, Artificial Sequence
  Description of Artificial Sequence Synthetic peptide
QCWEMNWTVNMWEPCA

SEQ ID NO: 322 - protein, 16 aa, Artificial Sequence
  Description of Artificial Sequence Synthetic peptide
QCWEWMYQWSEWRQCM

SEQ ID NO: 323 - protein, 16 aa, Artificial Sequence
  Description of Artificial Sequence Synthetic peptide
MCWEWELTIMDWISCD

SEQ ID NO: 324 - protein, 16 aa, Artificial Sequence
  Description of Artificial Sequence Synthetic peptide
ECWEFYQEHWSMFDCE

SEQ ID NO: 325 - protein, 16 aa, Artificial Sequence
  Description of Artificial Sequence Synthetic peptide
NCWSWQYLLGQWVECD

SEQ ID NO: 326 - protein, 16 aa, Artificial Sequence
  Description of Artificial Sequence Synthetic peptide
QCWVWDFEMWEWYQCS

SEQ ID NO: 327 - protein, 16 aa, Artificial Sequence
  Description of Artificial Sequence Synthetic peptide
DCWQWLYVEKMWIPCY

SEQ ID NO: 328 - protein, 16 aa, Artificial Sequence
  Description of Artificial Sequence Synthetic peptide
ECWQWDFEQQSWIPCR

SEQ ID NO: 329 - protein, 16 aa, Artificial Sequence
  Description of Artificial Sequence Synthetic peptide
ECWEWISQMQVWVQCP

SEQ ID NO: 330 - protein, 16 aa, Artificial Sequence
  Description of Artificial Sequence Synthetic peptide
ECWIYDWMNQTWQYCN

SEQ ID NO: 331 - protein, 16 aa, Artificial Sequence
  Description of Artificial Sequence Synthetic peptide
NCWEWVWHAQQWVPCE

SEQ ID NO: 332 - protein, 16 aa, Artificial Sequence
  Description of Artificial Sequence Synthetic peptide
ECWQYDFYHQMWSACI

SEQ ID NO: 333 - protein, 16 aa, Artificial Sequence
  Description of Artificial Sequence Synthetic peptide
QECWEWVDGQWQECHE

SEQ ID NO: 334 - protein, 16 aa, Artificial Sequence
  Description of Artificial Sequence Synthetic peptide
YECWEWMEGEWISCGG

SEQ ID NO: 335 - protein, 16 aa, Artificial Sequence
  Description of Artificial Sequence Synthetic peptide
VECWELFTHGWQPCQH

SEQ ID NO: 336 - protein, 16 aa, Artificial Sequence
  Description of Artificial Sequence Synthetic peptide
FMCQEVEPGYIWECSM

SEQ ID NO: 337 - protein, 16 aa, Artificial Sequence
  Description of Artificial Sequence Synthetic peptide
TNCWEWIYQMWQSCQP

SEQ ID NO: 338 - protein, 16 aa, Artificial Sequence
  Description of Artificial Sequence Synthetic peptide
QACDEGPYIWDFMCMI

SEQ ID NO: 339 - protein, 16 aa, Artificial Sequence
  Description of Artificial Sequence Synthetic peptide
VICWELIDNQWMQCHM

SEQ ID NO: 340 - protein, 16 aa, Artificial Sequence
  Description of Artificial Sequence Synthetic peptide
PQCWEYIFGIWQPCMD

SEQ ID NO: 341 - protein, 16 aa, Artificial Sequence
  Description of Artificial Sequence Synthetic peptide
VICWELIEAQWTHCAP

SEQ ID NO: 342 - protein, 16 aa, Artificial Sequence
  Description of Artificial Sequence Synthetic peptide
QACWFFQDGNWYDCYE

SEQ ID NO: 343 - protein, 16 aa, Artificial Sequence
  Description of Artificial Sequence Synthetic peptide
GGCWQWVDWDWIQCHE

SEQ ID NO: 344 - protein, 16 aa, Artificial Sequence
  Description of Artificial Sequence Synthetic peptide
WWCGEMHGQTLMECFY

SEQ ID NO: 345 - protein, 16 aa, Artificial Sequence
  Description of Artificial Sequence Synthetic peptide
WECWEFLMDVWVPCVS

SEQ ID NO: 346 - protein, 16 aa, Artificial Sequence
  Description of Artificial Sequence Synthetic peptide
TECWELINFTWQQCLA

SEQ ID NO: 347 - protein, 16 aa, Artificial Sequence
  Description of Artificial Sequence Synthetic peptide
IICDVEWRVTIPECWV

SEQ ID NO: 348 - protein, 16 aa, Artificial Sequence
  Description of Artificial Sequence Synthetic peptide
PMCWEMFEWGWSQCFT

SEQ ID NO: 349 - protein, 16 aa, Artificial Sequence
  Description of Artificial Sequence Synthetic peptide
EICYPTAWPGVMQCLL

SEQ ID NO: 350 - protein, 16 aa, Artificial Sequence
  Description of Artificial Sequence Synthetic peptide
YECWEYYVGIWVHCNV

SEQ ID NO: 351 - protein, 16 aa, Artificial Sequence
  Description of Artificial Sequence Synthetic peptide
WACWAWNGMNWMECVD

SEQ ID NO: 352 - protein, 16 aa, Artificial Sequence
  Description of Artificial Sequence Synthetic peptide
QQCWEWTDFGWSECHD

SEQ ID NO: 353 - protein, 16 aa, Artificial Sequence
  Description of Artificial Sequence Synthetic peptide
QECWSLVDWQWQRCDD

SEQ ID NO: 354 - protein, 16 aa, Artificial Sequence
  Description of Artificial Sequence Synthetic peptide
WPCWEWMQGQWWDCVE

SEQ ID NO: 355 - protein, 16 aa, Artificial Sequence
  Description of Artificial Sequence Synthetic peptide
QICWEKVQDLWVMCTD

SEQ ID NO: 356 - protein, 16 aa, Artificial Sequence
  Description of Artificial Sequence Synthetic peptide
IDCWEWVMTEWYPCIY

SEQ ID NO: 357 - protein, 16 aa, Artificial Sequence
  Description of Artificial Sequence Synthetic peptide
QSCPYKEDGVWYDCLI

SEQ ID NO: 358 - protein, 16 aa, Artificial Sequence
  Description of Artificial Sequence Synthetic peptide
PECWQWASPEWVECTD

SEQ ID NO: 359 - protein, 16 aa, Artificial Sequence
  Description of Artificial Sequence Synthetic peptide
VDCWEMMDQQWLPCRD

SEQ ID NO: 360 - protein, 16 aa, Artificial Sequence
  Description of Artificial Sequence Synthetic peptide
PTCWDLVDMNWIPCAV

SEQ ID NO: 361 - protein, 16 aa, Artificial Sequence
  Description of Artificial Sequence Synthetic peptide
MECWQWVEFQWMACSE

SEQ ID NO: 362 - protein, 16 aa, Artificial Sequence
  Description of Artificial Sequence Synthetic peptide
QSCWEWVMGHWRVCQE

SEQ ID NO: 363 - protein, 16 aa, Artificial Sequence
  Description of Artificial Sequence Synthetic peptide
VECWMYFQEYWIPCPQ

SEQ ID NO: 364 - protein, 16 aa, Artificial Sequence
  Description of Artificial Sequence Synthetic peptide
TVCWEWVDHVWAECPT

SEQ ID NO: 365 - protein, 16 aa, Artificial Sequence
  Description of Artificial Sequence Synthetic peptide
MECWEMIQGQWAMCMN

SEQ ID NO: 366 - protein, 16 aa, Artificial Sequence
  Description of Artificial Sequence Synthetic peptide
WECFQLPDSIMYECYM

SEQ ID NO: 367 - protein, 16 aa, Artificial Sequence
  Description of Artificial Sequence Synthetic peptide
NNCWQYVSDSWMPCTA

SEQ ID NO: 368 - protein, 16 aa, Artificial Sequence
  Description of Artificial Sequence Synthetic peptide
ASCWEMIETVWVQCNM

SEQ ID NO: 369 - protein, 16 aa, Artificial Sequence
  Description of Artificial Sequence Synthetic peptide
MECWEWAQGEWNYCMP

SEQ ID NO: 370 - protein, 16 aa, Artificial Sequence
  Description of Artificial Sequence Synthetic peptide
EYCWELLQSVWTVCTE

SEQ ID NO: 371 - protein, 16 aa, Artificial Sequence
  Description of Artificial Sequence Synthetic peptide
VQCWQWQDHQWIECIT

SEQ ID NO: 372 - protein, 16 aa, Artificial Sequence
  Description of Artificial Sequence Synthetic peptide
MTCWMWVQESWQECMD

SEQ ID NO: 373 - protein, 16 aa, Artificial Sequence
  Description of Artificial Sequence Synthetic peptide
QFCWELVDGWWNMCDR

SEQ ID NO: 374 - protein, 16 aa, Artificial Sequence
  Description of Artificial Sequence Synthetic peptide
DQCWMWYSGAWQPCAT

SEQ ID NO: 375 - protein, 16 aa, Artificial Sequence
  Description of Artificial Sequence Synthetic peptide
FECWEYFGESWQACHN

SEQ ID NO: 376 - protein, 16 aa, Artificial Sequence
  Description of Artificial Sequence Synthetic peptide
EECWVFIETWWQPCHL

SEQ ID NO: 377 - protein, 16 aa, Artificial Sequence
  Description of Artificial Sequence Synthetic peptide
QMCWEWMYTEWVECET

SEQ ID NO: 378 - protein, 16 aa, Artificial Sequence
  Description of Artificial Sequence Synthetic peptide
PPCWELIVDHWIICEE

SEQ ID NO: 379 - protein, 16 aa, Artificial Sequence
  Description of Artificial Sequence Synthetic peptide
PECWEWFPWGWDQCHM

SEQ ID NO: 380 - protein, 16 aa, Artificial Sequence
  Description of Artificial Sequence Synthetic peptide
AQCWEYVIDHWVMCTE

SEQ ID NO: 381 - protein, 16 aa, Artificial Sequence
  Description of Artificial Sequence Synthetic peptide
LECWEWAHIEWMPCIE

SEQ ID NO: 382 - protein, 16 aa, Artificial Sequence
  Description of Artificial Sequence Synthetic peptide
PNCWEWVQKEWIECAI

SEQ ID NO: 383 - protein, 16 aa, Artificial Sequence
  Description of Artificial Sequence Synthetic peptide
IECWEWQKDKWVECSS

SEQ ID NO: 384 - protein, 16 aa, Artificial Sequence
  Description of Artificial Sequence Synthetic peptide
YECWEMVNNFWQTCDH

SEQ ID NO: 385 - protein, 16 aa, Artificial Sequence
  Description of Artificial Sequence Synthetic peptide
PECWEYIHNGWMQCDG

SEQ ID NO: 386 - protein, 16 aa, Artificial Sequence
  Description of Artificial Sequence Synthetic peptide
RECWEYVHQMWQTCQE

SEQ ID NO: 387 - protein, 16 aa, Artificial Sequence
  Description of Artificial Sequence Synthetic peptide
FECWEWNGMDWGACNS

SEQ ID NO: 388 - protein, 16 aa, Artificial Sequence
  Description of Artificial Sequence Synthetic peptide
QYDHHDNWMEQLVLML

SEQ ID NO: 389 - protein, 16 aa, Artificial Sequence
  Description of Artificial Sequence Synthetic peptide
DYDFHDKWMEQLILML

SEQ ID NO: 390 - protein, 16 aa, Artificial Sequence
  Description of Artificial Sequence Synthetic peptide
DYEFHEHWMDQLILMM

SEQ ID NO: 391 - protein, 16 aa, Artificial Sequence
  Description of Artificial Sequence Synthetic peptide
EYDLHDQWMEQLVLML

SEQ ID NO: 392 - protein, 16 aa, Artificial Sequence
  Description of Artificial Sequence Synthetic peptide
QLDFTDDWMEQLVIML

SEQ ID NO: 393 - protein, 16 aa, Artificial Sequence
  Description of Artificial Sequence Synthetic peptide
DVDLHDKWMDDLIHML

SEQ ID NO: 394 - protein, 16 aa, Artificial Sequence
  Description of Artificial Sequence Synthetic peptide
HNDYSEDWMDQLVVML

SEQ ID NO: 395 - protein, 16 aa, Artificial Sequence
  Description of Artificial Sequence Synthetic peptide
HFEVYDNWMDQLVQML

SEQ ID NO: 396 - protein, 16 aa, Artificial Sequence
  Description of Artificial Sequence Synthetic peptide
HYDFPEHWMDQLIVMM

SEQ ID NO: 397 - protein, 16 aa, Artificial Sequence
  Description of Artificial Sequence Synthetic peptide
DYEYNEDWMEQLILML

SEQ ID NO: 398 - protein, 16 aa, Artificial Sequence
  Description of Artificial Sequence Synthetic peptide
QHDVSDDWLDQLVLML

SEQ ID NO: 399 - protein, 16 aa, Artificial Sequence
  Description of Artificial Sequence Synthetic peptide
DFDFNDKWIEELILML

SEQ ID NO: 400 - protein, 16 aa, Artificial Sequence
  Description of Artificial Sequence Synthetic peptide
QIDFSDNWMDQLVLMM

SEQ ID NO: 401 - protein, 16 aa, Artificial Sequence
  Description of Artificial Sequence Synthetic peptide
QYDFNEDWMEQLILMM

SEQ ID NO: 402 - protein, 16 aa, Artificial Sequence
  Description of Artificial Sequence Synthetic peptide
ELDVNDKWMDQLILMM

SEQ ID NO: 403 - protein, 16 aa, Artificial Sequence
  Description of Artificial Sequence Synthetic peptide
HFEFDDHWMDQLVLMM

SEQ ID NO: 404 - protein, 16 aa, Artificial Sequence
  Description of Artificial Sequence Synthetic peptide
EFEFNENWMEQLVLMM

SEQ ID NO: 405 - protein, 16 aa, Artificial Sequence
  Description of Artificial Sequence Synthetic peptide
QDDVYDDWMDQLILML

SEQ ID NO: 406 - protein, 16 aa, Artificial Sequence
  Description of Artificial Sequence Synthetic peptide
EIDYHDNWLDQLVVMM

SEQ ID NO: 407 - protein, 16 aa, Artificial Sequence
  Description of Artificial Sequence Synthetic peptide
EFEFYEDWMDQLILML

SEQ ID NO: 408 - protein, 16 aa, Artificial Sequence
  Description of Artificial Sequence Synthetic peptide
QYDVNEQWMDQLIQML

SEQ ID NO: 409 - protein, 16 aa, Artificial Sequence
  Description of Artificial Sequence Synthetic peptide
DYDVNEQWMDQLILML

SEQ ID NO: 410 - protein, 16 aa, Artificial Sequence
  Description of Artificial Sequence Synthetic peptide
HVEYDENWMEQLILML

SEQ ID NO: 411 - protein, 16 aa, Artificial Sequence
  Description of Artificial Sequence Synthetic peptide
EIEYYEHWMEQLVHML

SEQ ID NO: 412 - protein, 16 aa, Artificial Sequence
  Description of Artificial Sequence Synthetic peptide
EDDFNEHWMEQLILLL

SEQ ID NO: 413 - protein, 16 aa, Artificial Sequence
  Description of Artificial Sequence Synthetic peptide
HFDVNDQWMDQLVLML

SEQ ID NO: 414 - protein, 16 aa, Artificial Sequence
  Description of Artificial Sequence Synthetic peptide
DFDYHEHWMEDLILML

SEQ ID NO: 415 - protein, 16 aa, Artificial Sequence
  Description of Artificial Sequence Synthetic peptide
QYDYSDNWMEQFVLML

SEQ ID NO: 416 - protein, 16 aa, Artificial Sequence
  Description of Artificial Sequence Synthetic peptide
QYEVTDNWMDQLVLML

SEQ ID NO: 417 - protein, 16 aa, Artificial Sequence
  Description of Artificial Sequence Synthetic peptide
EYEDADNWMEQLVVMM

SEQ ID NO: 418 - protein, 16 aa, Artificial Sequence
  Description of Artificial Sequence Synthetic peptide
EYDVYDHWMDQLIVMM

SEQ ID NO: 419 - protein, 16 aa, Artificial Sequence
  Description of Artificial Sequence Synthetic peptide
QYDVHDQWMEQLILML

SEQ ID NO: 420 - protein, 16 aa, Artificial Sequence
  Description of Artificial Sequence Synthetic peptide
EHDYYDNWMDQLVVML

SEQ ID NO: 421 - protein, 16 aa, Artificial Sequence
  Description of Artificial Sequence Synthetic peptide
HLDFHEDWMEQLVLMM

SEQ ID NO: 422 - protein, 16 aa, Artificial Sequence
  Description of Artificial Sequence Synthetic peptide
HNDFNEKWMEQLVLMM

SEQ ID NO: 423 - protein, 16 aa, Artificial Sequence
  Description of Artificial Sequence Synthetic peptide
HYDHYEHWMDQLIVMM

SEQ ID NO: 424 - protein, 16 aa, Artificial Sequence
  Description of Artificial Sequence Synthetic peptide
DFEVYDNWMEQLIVML

SEQ ID NO: 425 - protein, 16 aa, Artificial Sequence
  Description of Artificial Sequence Synthetic peptide
EYEDHDHWLDQLVLMM

SEQ ID NO: 426 - protein, 16 aa, Artificial Sequence
  Description of Artificial Sequence Synthetic peptide
HYDFDEQWMDHLILML

SEQ ID NO: 427 - protein, 16 aa, Artificial Sequence
  Description of Artificial Sequence Synthetic peptide
HVDFHDDWMDQLVLMM

SEQ ID NO: 428 - protein, 16 aa, Artificial Sequence
  Description of Artificial Sequence Synthetic peptide
QVEYYDNWMDHLIVML

SEQ ID NO: 429 - protein, 16 aa, Artificial Sequence
  Description of Artificial Sequence Synthetic peptide
QHDHHDDWMDQLIVML

SEQ ID NO: 430 - protein, 16 aa, Artificial Sequence
  Description of Artificial Sequence Synthetic peptide
EYDHHDHWMDELVLMM

SEQ ID NO: 431 - protein, 16 aa, Artificial Sequence
  Description of Artificial Sequence Synthetic peptide
DFEFSDHWIDQLVLML

SEQ ID NO: 432 - protein, 16 aa, Artificial Sequence
  Description of Artificial Sequence Synthetic peptide
QFDHADHWMEHLVLMM

SEQ ID NO: 433 - protein, 16 aa, Artificial Sequence
  Description of Artificial Sequence Synthetic peptide
DVDFADKWMEHLIVML

SEQ ID NO: 434 - protein, 16 aa, Artificial Sequence
  Description of Artificial Sequence Synthetic peptide
HYDHDEHWMEQLIHML

SEQ ID NO: 435 - protein, 16 aa, Artificial Sequence
  Description of Artificial Sequence Synthetic peptide
EFDHYDNWMDQLILML

SEQ ID NO: 436 - protein, 16 aa, Artificial Sequence
  Description of Artificial Sequence Synthetic peptide
DHDHYDDWMDQLIVML

SEQ ID NO: 437 - protein, 16 aa, Artificial Sequence
  Description of Artificial Sequence Synthetic peptide
QYEVYEDWMEQLVLML

SEQ ID NO: 438 - protein, 16 aa, Artificial Sequence
  Description of Artificial Sequence Synthetic peptide
EIDHDDKWMEQLILML

SEQ ID NO: 439 - protein, 16 aa, Artificial Sequence
  Description of Artificial Sequence Synthetic peptide
EYEFNEHWMDHLVLML

SEQ ID NO: 440 - protein, 16 aa, Artificial Sequence
  Description of Artificial Sequence Synthetic peptide
EVDFYDDWMDQLVLMM

SEQ ID NO: 441 - protein, 16 aa, Artificial Sequence
  Description of Artificial Sequence Synthetic peptide
DNDFDEKWMEQLILLL

SEQ ID NO: 442 - protein, 16 aa, Artificial Sequence
  Description of Artificial Sequence Synthetic peptide
QVDYDEHWMEQLVLML

SEQ ID NO: 443 - protein, 16 aa, Artificial Sequence
  Description of Artificial Sequence Synthetic peptide
HDDDYDDWLDQLVVMM

SEQ ID NO: 444 - protein, 16 aa, Artificial Sequence
  Description of Artificial Sequence Synthetic peptide
DYDFSEEWMDQLVLML

SEQ ID NO: 445 - protein, 16 aa, Artificial Sequence
  Description of Artificial Sequence Synthetic peptide
HYEVYDNWMDQLVQMM

SEQ ID NO: 446 - protein, 16 aa, Artificial Sequence
  Description of Artificial Sequence Synthetic peptide
EFDVHDQWMEQLVLMM

SEQ ID NO: 447 - protein, 16 aa, Artificial Sequence
  Description of Artificial Sequence Synthetic peptide
EFEHNEQWMDQLILML

SEQ ID NO: 448 - protein, 16 aa, Artificial Sequence
  Description of Artificial Sequence Synthetic peptide
QYDYDDHWMEQLIVML

SEQ ID NO: 449 - protein, 16 aa, Artificial Sequence
  Description of Artificial Sequence Synthetic peptide
HNDFNENWMDQLVVMM

SEQ ID NO: 450 - protein, 16 aa, Artificial Sequence
  Description of Artificial Sequence Synthetic peptide
EHEVHEQWMDQLIHML

SEQ ID NO: 451 - protein, 16 aa, Artificial Sequence
  Description of Artificial Sequence Synthetic peptide
QYEYPEQWMDQLVLML

SEQ ID NO: 452 - protein, 16 aa, Artificial Sequence
  Description of Artificial Sequence Synthetic peptide
EYEFHEKWMEELIHML

SEQ ID NO: 453 - protein, 16 aa, Artificial Sequence
  Description of Artificial Sequence Synthetic peptide
DFDYNEEWMDQLVVML

SEQ ID NO: 454 - protein, 16 aa, Artificial Sequence
  Description of Artificial Sequence Synthetic peptide
DYDVHEQWMDHLIHML

SEQ ID NO: 455 - protein, 16 aa, Artificial Sequence
  Description of Artificial Sequence Synthetic peptide
EYDVADKWMDQLILMM

SEQ ID NO: 456 - protein, 16 aa, Artificial Sequence
  Description of Artificial Sequence Synthetic peptide
HFDYSDNWMDHLVLMM

SEQ ID NO: 457 - protein, 16 aa, Artificial Sequence
  Description of Artificial Sequence Synthetic peptide
ELDVHDDWLDQLILMM

SEQ ID NO: 458 - protein, 16 aa, Artificial Sequence
  Description of Artificial Sequence Synthetic peptide
DYEVYDDWLEQLVVML

SEQ ID NO: 459 - protein, 16 aa, Artificial Sequence
  Description of Artificial Sequence Synthetic peptide
EHEFNDDWMDQLVLML

SEQ ID NO: 460 - protein, 16 aa, Artificial Sequence
  Description of Artificial Sequence Synthetic peptide
DYDLYDNWMEQLIVMM

SEQ ID NO: 461 - protein, 16 aa, Artificial Sequence
  Description of Artificial Sequence Synthetic peptide
HVDFHEDWMDQLVVML

SEQ ID NO: 462 - protein, 16 aa, Artificial Sequence
  Description of Artificial Sequence Synthetic peptide
EFDFSDDWMDQLVLMM

SEQ ID NO: 463 - protein, 16 aa, Artificial Sequence
  Description of Artificial Sequence Synthetic peptide
EYEFNEHWMEQLIVMM

SEQ ID NO: 464 - protein, 16 aa, Artificial Sequence
  Description of Artificial Sequence Synthetic peptide
ENEYTEHWMEQLILML

SEQ ID NO: 465 - protein, 16 aa, Artificial Sequence
  Description of Artificial Sequence Synthetic peptide
DFDYADQWMDQLVLML

SEQ ID NO: 466 - protein, 16 aa, Artificial Sequence
  Description of Artificial Sequence Synthetic peptide
EFDFPEHWMDQLVLLL

SEQ ID NO: 467 - protein, 16 aa, Artificial Sequence
  Description of Artificial Sequence Synthetic peptide
EFEFYDNWLDQLVVMM

SEQ ID NO: 468 - protein, 16 aa, Artificial Sequence
  Description of Artificial Sequence Synthetic peptide
QFEVHEEWMEQLILML

SEQ ID NO: 469 - protein, 16 aa, Artificial Sequence
  Description of Artificial Sequence Synthetic peptide
QFEFHDHWMDQLIHML

SEQ ID NO: 470 - protein, 16 aa, Artificial Sequence
  Description of Artificial Sequence Synthetic peptide
HYDFDEKWMEDLILML

SEQ ID NO: 471 - protein, 16 aa, Artificial Sequence
  Description of Artificial Sequence Synthetic peptide
HNDYHDDWMDQLILML

SEQ ID NO: 472 - protein, 16 aa, Artificial Sequence
  Description of Artificial Sequence Synthetic peptide
QYDYSDNWMEQLVLMM

SEQ ID NO: 473 - protein, 16 aa, Artificial Sequence
  Description of Artificial Sequence Synthetic peptide
ECWEWSFHFQEWGPCD

SEQ ID NO: 474 - protein, 16 aa, Artificial Sequence
  Description of Artificial Sequence Synthetic peptide
ECWEWNFHFQEWGPCD

SEQ ID NO: 475 - protein, 16 aa, Artificial Sequence
  Description of Artificial Sequence Synthetic peptide
ECWEWKFHLQEWLACD

SEQ ID NO: 476 - protein, 16 aa, Artificial Sequence
  Description of Artificial Sequence Synthetic peptide
ECWEWRFHIQEWMPCE

SEQ ID NO: 477 - protein, 16 aa, Artificial Sequence
  Description of Artificial Sequence Synthetic peptide
ECWQWNYHLQEWGQCD

SEQ ID NO: 478 - protein, 16 aa, Artificial Sequence
  Description of Artificial Sequence Synthetic peptide
ECWEWNFQLHEWGPCE

SEQ ID NO: 479 - protein, 16 aa, Artificial Sequence
  Description of Artificial Sequence Synthetic peptide
ECWEWSYQFQEWGPCE

SEQ ID NO: 480 - protein, 16 aa, Artificial Sequence
  Description of Artificial Sequence Synthetic peptide
ECWEWSFQLQEWGACA

SEQ ID NO: 481 - protein, 16 aa, Artificial Sequence
  Description of Artificial Sequence Synthetic peptide
ECWEWSFQFQEWGPCD

SEQ ID NO: 482 - protein, 16 aa, Artificial Sequence
  Description of Artificial Sequence Synthetic peptide
DCWEWKYEIQEWVACE

SEQ ID NO: 483 - protein, 16 aa, Artificial Sequence
  Description of Artificial Sequence Synthetic peptide
ECWEWKYDLQEWVACQ

SEQ ID NO: 484 - protein, 16 aa, Artificial Sequence
  Description of Artificial Sequence Synthetic peptide
ECWEWNYDFQEWGPCQ

SEQ ID NO: 485 - protein, 16 aa, Artificial Sequence
  Description of Artificial Sequence Synthetic peptide
ECWQWSYEFQEWGQCA

SEQ ID NO: 486 - protein, 16 aa, Artificial Sequence
  Description of Artificial Sequence Synthetic peptide
ECWEWKFHMQEWVHCD

SEQ ID NO: 487 - protein, 16 aa, Artificial Sequence
  Description of Artificial Sequence Synthetic peptide
ECWEWNFDFQEWGHCQ

SEQ ID NO: 488 - protein, 16 aa, Artificial Sequence
  Description of Artificial Sequence Synthetic peptide
ECWEWSYHMQEWGPCP

SEQ ID NO: 489 - protein, 16 aa, Artificial Sequence
  Description of Artificial Sequence Synthetic peptide
ECWEWSFHFQEWGQCQ

SEQ ID NO: 490 - protein, 16 aa, Artificial Sequence
  Description of Artificial Sequence Synthetic peptide
ECWEWKFEIQEWLPCQ

SEQ ID NO: 491 - protein, 16 aa, Artificial Sequence
  Description of Artificial Sequence Synthetic peptide
ECWEWNYHLQEWGACQ

SEQ ID NO: 492 - protein, 16 aa, Artificial Sequence
  Description of Artificial Sequence Synthetic peptide
ECWEWSFQFHEWGPCA

SEQ ID NO: 493 - protein, 16 aa, Artificial Sequence
  Description of Artificial Sequence Synthetic peptide
ECWEWSYQFHEWGACQ

SEQ ID NO: 494 - protein, 16 aa, Artificial Sequence
  Description of Artificial Sequence Synthetic peptide
ECWEWNFHFQEWGPCA

SEQ ID NO: 495 - protein, 16 aa, Artificial Sequence
  Description of Artificial Sequence Synthetic peptide
ECWEWSFQFQEWGACQ

SEQ ID NO: 496 - protein, 16 aa, Artificial Sequence
  Description of Artificial Sequence Synthetic peptide
ECWEWNFQMHEWGPCA

SEQ ID NO: 497 - protein, 6 aa, Artificial Sequence
  Description of Artificial Sequence Synthetic peptide
AHSYPI

SEQ ID NO: 498 - protein, 6 aa, Artificial Sequence
  Description of Artificial Sequence Synthetic peptide
GSYYPI

SEQ ID NO: 499 - protein, 6 aa, Artificial Sequence
  Description of Artificial Sequence Synthetic peptide
SAGYPI

SEQ ID NO: 500 - protein, 6 aa, Artificial Sequence
  Description of Artificial Sequence Synthetic peptide
SAYYPI

SEQ ID NO: 501 - protein, 6 aa, Artificial Sequence
  Description of Artificial Sequence Synthetic peptide
SDFYPI

SEQ ID NO: 502 - protein, 6 aa, Artificial Sequence
  Description of Artificial Sequence Synthetic peptide
SHSYPI

SEQ ID NO: 503 - protein, 6 aa, Artificial Sequence
  Description of Artificial Sequence Synthetic peptide
SSHYPI

SEQ ID NO: 504 - protein, 6 aa, Artificial Sequence
  Description of Artificial Sequence Synthetic peptide
SSSYPI

SEQ ID NO: 505 - protein, 6 aa, Artificial Sequence
  Description of Artificial Sequence Synthetic peptide
SSWYPI

SEQ ID NO: 506 - protein, 6 aa, Artificial Sequence
  Description of Artificial Sequence Synthetic peptide
SSYYPI

SEQ ID NO: 507 - protein, 6 aa, Artificial Sequence
  Description of Artificial Sequence Synthetic peptide
SSYYPM

SEQ ID NO: 508 - protein, 6 aa, Artificial Sequence
  Description of Artificial Sequence Synthetic peptide
SYFYPI

SEQ ID NO: 509 - protein, 6 aa, Artificial Sequence
  Description of Artificial Sequence Synthetic peptide
SYHYPI

SEQ ID NO: 510 - protein, 6 aa, Artificial Sequence
  Description of Artificial Sequence Synthetic peptide
SYSYPI

SEQ ID NO: 511 - protein, 6 aa, Artificial Sequence
  Description of Artificial Sequence Synthetic peptide
SYWYPI

SEQ ID NO: 512 - protein, 6 aa, Artificial Sequence
  Description of Artificial Sequence Synthetic peptide
SYYYPI

SEQ ID NO: 513 - protein, 16 aa, Artificial Sequence
  Description of Artificial Sequence Synthetic peptide
VMDPVYWFAYGWEEML

SEQ ID NO: 514 - protein, 16 aa, Artificial Sequence
  Description of Artificial Sequence Synthetic peptide
LHPPDFWMAFAHVDFY

SEQ ID NO: 515 - protein, 16 aa, Artificial Sequence
  Description of Artificial Sequence Synthetic peptide
YLDDAWHAWIQREAAF

SEQ ID NO: 516 - protein, 16 aa, Artificial Sequence
  Description of Artificial Sequence Synthetic peptide
LEFWHGPSWLEDMGFI

SEQ ID NO: 517 - protein, 16 aa, Artificial Sequence
  Description of Artificial Sequence Synthetic peptide
PAMDWCQIFQCTIQVL

SEQ ID NO: 518 - protein, 16 aa, Artificial Sequence
  Description of Artificial Sequence Synthetic peptide
  MOD_RES (1)..(1): D, E, H or Q
  MOD_RES (2)..(2): F, D, H, I, L, N, V or Y
  MOD_RES (3)..(3): D or E
  MOD_RES (4)..(4): F, D, H, L, V or Y
  MOD_RES (5)..(5): A, D, H, N, P, S, T or Y
  MOD_RES (6)..(6): E or D
  MOD_RES (7)..(7): N, D, E, H, K or Q
  MOD_RES (9)..(9): M, I, L or V
  MOD_RES (10)..(10): E or D
  MOD_RES (11)..(11): Q, D, E or H
  MOD_RES (12)..(12): L or F
  MOD_RES (13)..(13): K, E, I, L, Q or V
  MOD_RES (14)..(14): V, D, E, H, L or Q
  MOD_RES (15)..(15): M, F, I or L
  MOD_RES (16)..(16): L, F, I or M
XXXXXXXWXXXXXXXX

SEQ ID NO: 519 - protein, 16 aa, Artificial Sequence
  Description of Artificial Sequence Synthetic peptide
  MOD_RES (1)..(1): E, D, H or Q
  MOD_RES (4)..(4): E or Q
  MOD_RES (6)..(6): S, D, E, G, K, N or R
  MOD_RES (7)..(7): Y, F or S
  MOD_RES (8)..(8): Q, D, E or H
  MOD_RES (9)..(9): L, F, I or M
  MOD_RES (10)..(10): Q, D, E or H
  MOD_RES (11)..(11): E or Q
  MOD_RES (13)..(13): S, D, E, G, H, L, Q, R or V
  MOD_RES (14)..(14): P, A, D, E, H or Q
  MOD_RES (16)..(16): P, A, D, E, H or Q
XCWXWXXXXXXWXXCX